(19) United States
(12) Patent Application Publication
(10) Pub. No.: US 2023/0340142 A1
(43) Pub. Date: Oct. 26, 2023

(54) PD-L1 ANTIBODIES, FUSION PROTEINS, AND USES THEREOF

(71) Applicant: Suzhou Neologics Bioscience Co., Ltd., Suzhou City (CN)

(72) Inventors: Binbin Wang, SuZhou (CN); Dong Wang, SuZhou (CN); Jinyu Dong, SuZhou (CN); Yu Zhang, Hefei (CN); Zhou Zhou, SuZhou (CN); Liegang Shao, SuZhou (CN); Lianqi Zhao, SuZhou (CN); Xin Dong, SuZhou (CN); Baiyang Wang, Princeton, NJ (US)

(21) Appl. No.: 18/159,688

(22) Filed: Jan. 26, 2023

Related U.S. Application Data
(63) Continuation of application No. PCT/CN2021/118481, filed on Sep. 15, 2021.

(30) Foreign Application Priority Data
Sep. 16, 2020 (WO) PCT/CN2020/115572
Nov. 10, 2020 (WO) PCT/CN2020/127811
Nov. 30, 2020 (WO) PCT/CN2020/132740

(51) Int. Cl. C12N 15/86; C07K 16/28; C07K 14/71; A61P 35/00

(52) U.S. Cl. CPC C07K 16/2896; A61P 35/00; C07K 14/71; C12N 15/86; C07K 2317/565; C07K 2319/30

(57) ABSTRACT

Disclosed herein are anti-PD-L1 antibodies and antigen-binding fragments thereof, fusion proteins comprising a first domain comprising an anti-PD-L1 antibodies or an antigen-binding fragment thereof and a second domain comprising a fragment of TGFβRII that binds TGFβ or a variant thereof, as well as polynucleotides that encode such antibodies or fusion proteins. Disclosed herein are also their uses in treatment of diseases such as cancers.

### CROSS-REFERENCE TO RELATED APPLICATIONS

This application is a continuation of International Patent Application No. PCT/CN2021/118481, filed on Sep. 15, 2021, which claims benefit of priority of International Patent Application No. PCT/CN2020/115572 filed on Sep. 16, 2020, of International Patent Application No. PCT/CN2020/127811 filed on Nov. 10, 2020, and of International Patent Application No. PCT/CN2020/132740 filed on Nov. 30, 2020. The entire contents of these preceding applications are hereby incorporated by reference.

### REFERENCE TO SEQUENCE LISTING

This application incorporates by reference a Sequence Listing with this application entitled “135A001US04_SL.XML. created on Jan. 26, 2023, and having a size of 385,302 bytes.

### FIELD

The present disclosure relates to the fields of molecular biology, cell biology, and cancer biology.

### BACKGROUND

The immunosuppressive Programmed Death-Programmed Death Ligand 1 (PD-1/ PD-L1) signal pathway and the Transforming growth factor-β (TGFβ) signaling pathway are known targets in cancer treatment. Current therapies targeting either or both pathways, however, have only had limited success. Thus, additional PD-1/ PD-L1 and/or TGFβ targeting therapeutic options represent unmet needs. The compositions and methods provided herein meet these needs and provide relative advantages.

### BRIEF SUMMARY OF THE INVENTION

Provided herein are fusion proteins comprising (1) a first domain comprising an antibody that binds Programmed Death Ligand 1 (PD-L1), or an antigen-binding fragment thereof, (2) a transferrin linker, and (3) a second domain comprising a fragment of transforming growth factor β receptor type 2 (TGFβRII) that binds transforming growth factor β (TGFβ), or a variant thereof.

In some embodiments of the fusion proteins provided herein, the transferrin linker links the C-terminus of the first domain to the N-terminus of the second domain. In some embodiments, the transferrin linker is (PEAPTD)m, m=1, 2, 3, 4, or 5 (SEQ ID NO:18) or (PEAPTDE)n, n=1, 2, 3, 4, or 5 (SEQ ID NO: 19). In some embodiments, the transferrin linker has an amino acid sequence selected from the group consisting of SEQ ID NOs:220-230. In some embodiments, the transferrin linker is (PEAPTD)₃(SEQ ID NO: 147).

In some embodiments of the fusion proteins provided herein, the second domain comprises the ECD of TGFβRII isoform 1 (SEQ ID NO:8) or the ECD of TGFβRII isoform 2 (SEQ ID NO: 14), or a variant thereof that has at least 85%, at least 90%, at least 95%, or at least 98% sequence identity to SEQ ID NO:8 or 14. In some embodiments, the second domain has the amino acid sequence of SEQ ID NO:8. In some embodiments, the second domain has the amino acid sequence of SEQ ID NO:9. In some embodiments, the second domain comprises a variant of the ECD of TGFβRII isoform 1 (SEQ ID NO:8), wherein the variant comprises an amino acid mutation at Q6, K7, N19 or G20 of SEQ ID NO:8. In some embodiments, the second domain has an amino acid sequence selected from the group consisting of SEQ ID NOs:201-203.

In some embodiments, second domain comprises a truncated form of the ECD of TGFβRII isoform 1 (SEQ ID NO:8), which lacks amino acid residues 1 to n of SEQ ID NO: 8, wherein n ranges from 2 to 30, or a variant thereof. In some embodiments, n is 19. In some embodiments, the second domain comprises a truncated form of the ECD of TGFβRII isoform 1 (SEQ ID NO:8), which lacks amino acid residues m to 136 of SEQ ID NO:8, wherein m ranges from 80 to 135, or a variant thereof. In some embodiments, m is 131. In some embodiments, m is 128. In some embodiments, the second domain has an amino acid sequence selected from the group consisting of SEQ ID NOs:204, 205 and 232.

In some embodiments of the fusion proteins provided herein, the first domain is an PD-L1 antibody selected from the group consisting of durvalumab, avelumab, atezolizumab, envafolimab, BMS-936559, CK-301, CS-1001, SHR-1316, and BGB-A333.

Provided herein are also an antibodies or antigen-binding fragments thereof that bind PD-L1, comprising: (a) a heavy chain variable region (VH) comprising (1) a heavy chain CDR1 (VH CDR1) having an amino acid sequence selected from the group consisting of SEQ ID NOs:20, 26, 32, 38, 44, 50, 56, 62, 68, 74, 80, 86, and 92; (2) a heavy chain CDR2 (VH CDR2) having an amino acid sequence selected from the group consisting of SEQ ID NOs:21, 27, 33, 39, 45, 51, 57, 63, 69, 75, 81, 87 and 93; and (3) a heavy chain CDR3 (VH CDR3) having an amino acid sequence selected from the group consisting of SEQ ID NOs:22, 28, 34, 40, 46, 52, 58, 64, 70, 76, 82, 89 and 94; or a variant thereof having up to about 5 amino acid substitutions, additions, and/or deletions in the VH CDRs; and/or (b) a light chain variable region (VL) comprising (1) a light chain CDR1 (VL CDR1) having an amino acid sequence selected from the group consisting of SEQ ID NOs:23, 29, 35, 41, 47, 53, 59, 65, 71, 77, 83, 89, and 95; (2) a light chain CDR2 (VL CDR2) having an amino acid sequence selected from the group consisting of SEQ ID NOs:24, 30, 36, 42, 48, 54, 60, 66, 72, 78, 84, 90, and 96; and (3) a light chain CDR3 (VL CDR3) having an amino acid sequence selected from the group consisting of SEQ ID NOs:25, 31, 37, 43, 49, 55, 61, 67, 63, 79, 85, 91, and 97; or a variant thereof having up to about 5 amino acid substitutions, additions, and/or deletions in the VL CDRs.

In some embodiments, the anti-PD-L1 antibodies or antigen-binding fragments provided herein comprise (a) the VH CDR1, CDR2 and CDR3 have (1) the amino acid sequences of SEQ ID NOs:20, 21, and 22, respectively; (2) the amino acid sequences of SEQ ID NOs:26, 27, and 28, respectively; (3) the amino acid sequences of SEQ ID NOs:32, 33, and 34, respectively; (4) the amino acid sequences of SEQ ID NOs:38, 39, and 40, respectively; (5) the amino acid sequences of SEQ ID NOs:44, 45 and 46, respectively; (6) the amino acid sequences of SEQ ID NOs:50, 51, and 52, respectively; (7) the amino acid sequences of SEQ ID NOs:56, 57, and 58, respectively; (8) the amino acid sequences of SEQ ID NOs:62, 63, and 64, respectively; (9) the amino acid sequences of SEQ ID NOs:68, 69, and 70, respectively; (10) the amino acid sequences of SEQ ID NOs:74, 75, and 76, respectively; (11) the amino acid sequences of SEQ ID NOs:80, 81, and 82, respectively; (12) the amino acid sequences of SEQ ID NOs:86, 87, and 88, respectively; or (13) the amino acid sequences of SEQ ID NOs:92, 93 and 94, respectively; or a variant thereof having up to about 5 amino acid substitutions, additions, and/or deletions in the VH CDRs; and/or (b) the VL CDR1, CDR2 and CDR3 have (1) the amino acid sequences of SEQ ID NOs:23, 24 and 25, respectively; (2) the amino acid sequences of SEQ ID NOs:29, 30, and 31, respectively; (3) the amino acid sequences of SEQ ID NOs:35, 36 and 37, respectively; (4) the amino acid sequences of SEQ ID NOs:41, 42 and 43, respectively; (5) the amino acid sequences of SEQ ID NOs:47, 48 and 49, respectively; (6) the amino acid sequences of SEQ ID NOs:53, 54, and 55, respectively; (7) the amino acid sequences of SEQ ID NOs:59, 60, and 61, respectively; (8) the amino acid sequences of SEQ ID NOs:65, 66, and 67, respectively; (9) the amino acid sequences of SEQ ID NOs:71, 72 and 73, respectively; (10) the amino acid sequences of SEQ ID NOs:77, 78 and 79, respectively; (11) the amino acid sequences of SEQ ID NOs:83, 84, and 85, respectively; (12) the amino acid sequences of SEQ ID NOs:89, 90 and 91, respectively; or (13) the amino acid sequences of SEQ ID NOs:95, 96 and 97, respectively; or a variant thereof having up to about 5 amino acid substitutions, additions, and/or deletions in the VL CDRs.

In some embodiments, the anti-PD-L1 antibodies or antigen-binding fragments provided herein comprise (1) the VH CDR1, CDR2, and CDR3 have the amino acid sequences of SEQ ID NOs:20, 21, and 22, respectively; and/or the VL CDR1, CDR2, and CDR3 have the amino acid sequences of SEQ ID NOs:23, 24 and 25, respectively; (2) the VH CDR1, CDR2, and CDR3 have the amino acid sequences of SEQ ID NOs:26, 27, and 28, respectively; and/or the VL CDR1, CDR2, and CDR3 have the amino acid sequences of SEQ ID NOs:29, 30, and 31, respectively; (3) the VH CDR1, CDR2, and CDR3 have the amino acid sequences of SEQ ID NOs:32, 33, and 34, respectively; and/or the VL CDR1, CDR2, and CDR3 have the amino acid sequences of SEQ ID NOs:35, 36 and 37, respectively; (4) the VH CDR1, CDR2, and CDR3 have the amino acid sequences of SEQ ID NOs:38, 39, and 40, respectively; and/or the VL CDR1, CDR2, and CDR3 have the amino acid sequences of SEQ ID NOs:41, 42 and 43, respectively; (5) the VH CDR1, CDR2, and CDR3 have the amino acid sequences of SEQ ID NOs:44, 45 and 46, respectively; and/or the VL CDR1, CDR2, and CDR3 have the amino acid sequences of SEQ ID NOs:47, 48 and 49, respectively; (6) the VH CDR1, CDR2, and CDR3 have the amino acid sequences of SEQ ID NOs:50, 51, and 52, respectively; and/or the VL CDR1, CDR2, and CDR3 have the amino acid sequences of SEQ ID NOs:53, 54, and 55, respectively; (7) the VH CDR1, CDR2, and CDR3 have the amino acid sequences of SEQ ID NOs:56, 57, and 58, respectively; and/or the VL CDR1, CDR2, and CDR3 have the amino acid sequences of SEQ ID NOs:59, 60, and 61, respectively; (8) the VH CDR1, CDR2, and CDR3 have the amino acid sequences of SEQ ID NOs:62, 63, and 64, respectively; and/or the VL CDR1, CDR2, and CDR3 have the amino acid sequences of SEQ ID NOs:65, 66, and 67, respectively; (9) the VH CDR1, CDR2, and CDR3 have the amino acid sequences of SEQ ID NOs:68, 69, and 70, respectively; and/or the VL CDR1, CDR2, and CDR3 have the amino acid sequences of SEQ ID NOs:71, 72 and 73, respectively; (10) the VH CDR1, CDR2, and CDR3 have the amino acid sequences of SEQ ID NOs:74, 75, and 76, respectively; and/or the VL CDR1, CDR2, and CDR3 have the amino acid sequences of SEQ ID NOs:77, 78 and 79, respectively; (11) the VH CDR1, CDR2, and CDR3 have the amino acid sequences of SEQ ID NOs:80, 81, and 82, respectively; and/or the VL CDR1, CDR2, and CDR3 have the amino acid sequences of SEQ ID NOs:83, 84, and 85, respectively; (12) the VH CDR1, CDR2, and CDR3 have the amino acid sequences of SEQ ID NOs:86, 87, and 88, respectively; and/or the VL CDR1, CDR2, and CDR3 have the amino acid sequences of SEQ ID NOs:89, 90 and 91, respectively; or (13) the VH CDR1, CDR2, and CDR3 have the amino acid sequences of SEQ ID NOs:92, 93 and 94, respectively; and/or the VL CDR1, CDR2, and CDR3 have the amino acid sequences of SEQ ID NOs:95, 96 and 97, respectively.

In some embodiments, the anti-PD-L1 antibodies or antigen-binding fragments provided herein comprise (a) a VH CDR1 having the amino acid sequence of SEQ ID NO:26; a VH CDR2 having the amino acid sequence of SEQ ID NOs:27; and a VH CDR3 having the amino acid sequence selected of SEQ ID NO:28; and (b) a VL CDR1 having the amino acid sequence of SEQ ID NO:29; a VL CDR2 having the amino acid sequence of SEQ ID NOs:30; and a VL CDR3 having the amino acid sequence selected of SEQ ID NO:31.

In some embodiments, the anti-PD-L1 antibodies or antigen-binding fragments provided herein comprise (a) a VH CDR1 having the amino acid sequence of SEQ ID NO:38; a VH CDR2 having the amino acid sequence of SEQ ID NOs:39; and a VH CDR3 having the amino acid sequence selected of SEQ ID NO:40; and (b) a VL CDR1 having the amino acid sequence of SEQ ID NO:41; a VL CDR2 having the amino acid sequence of SEQ ID NOs:42; and a VL CDR3 having the amino acid sequence selected of SEQ ID NO:43.

In some embodiments, the anti-PD-L1 antibodies or antigen-binding fragments provided herein comprise (a) a VH CDR1 having the amino acid sequence of SEQ ID NO:32; a VH CDR2 having the amino acid sequence of SEQ ID NOs:33; and a VH CDR3 having the amino acid sequence selected of SEQ ID NO:34; and (b) a VL CDR1 having the amino acid sequence of SEQ ID NO:35; a VL CDR2 having the amino acid sequence of SEQ ID NOs:36; and a VL CDR3 having the amino acid sequence selected of SEQ ID NO:37.

In some embodiments, provided herein are antibodies or antigen-binding fragments thereof that binds PD-L1, comprising: (a) a VH having at least 85%, at least 90%, at least 95%, at least 98%, or 100% sequence identity to an amino acid sequence selected from the group consisting of SEQ ID NOs:98-110, 124, 126-128, 131-136, and 174-178; and/or (b) a VL having at least 85%, at least 90%, at least 95%, at least 98%, or 100% sequence identity to an amino acid sequence selected from the group consisting of SEQ ID NOs: 111-123, 125, 129-130, 137-144, and 179-181.

In some embodiments, the anti-PD-L1 antibodies or antigen-binding fragments provided herein comprise a VH and a VL, wherein the VH and VL have (1) the amino acid sequences of SEQ ID NOs:98 and 111, respectively; (2) the amino acid sequences of SEQ ID NOs:99 and 112, respectively; (3) the amino acid sequences of SEQ ID NOs:100 and 113, respectively; (4) the amino acid sequences of SEQ ID NOs: 101 and 114, respectively; (5) the amino acid sequences of SEQ ID NOs:102 and 115:, respectively; (6) the amino acid sequences of SEQ ID NOs: 103 and 116, respectively; (7) the amino acid sequences of SEQ ID NOs:104 and 117, respectively; (8) the amino acid sequences of SEQ ID NOs: 105 and 118, respectively; (9) the amino acid sequences of SEQ ID NOs: 106 and 119, respectively; (10) the amino acid sequences of SEQ ID NOs: 107 and 120, respectively; (11) the amino acid sequences of SEQ ID NOs:108 and 121, respectively; (12) the amino acid sequences of SEQ ID NOs: 109 and 122, respectively; (13) the amino acid sequences of SEQ ID NOs: 110 and 123, respectively; or (14) the amino acid sequences of SEQ ID NOs: 124 and 125, respectively.

In some embodiments of the anti-PD-L1 antibodies or antigen-binding fragments provided herein, the VH has an amino acid sequence selected from the group consisting of SEQ ID NOs:126-128, and/or the VL has the amino acid sequence of SEQ ID NO:129 or 130. In some embodiments of the anti-PD-L1 antibodies or antigen-binding fragments provided herein, the VH has an amino acid sequence selected from the group consisting of SEQ ID NOs: 131-136, and/or the VL has an amino acid sequence selected from the group consisting of SEQ ID NOs: 137-144. In some embodiments of the anti-PD-L1 antibodies or antigen-binding fragments provided herein, the VH has an amino acid sequence selected from the group consisting of SEQ ID NOs: 174-178, and/or the VL has an amino acid sequence selected from the group consisting of SEQ ID NOs: 179-181.

In some embodiments of the anti-PD-L1 antibodies or antigen-binding fragments provided herein comprise (a) a VH comprising VH CDRs 1, 2, and 3 from a VH having an amino acid sequence selected from the group consisting of SEQ ID NOs:98-110, 124, 126-128, 131-136, and 174-178; and/or (b) a VL comprising VL CDRs 1, 2, and 3 from a VL having an amino acid sequence selected from group consisting of SEQ ID NOs: 111-123, 125, 129-130, 137-144, and 179-181.

In some embodiments, the anti-PD-L1 antibodies or antigen-binding fragments provided herein comprise (1) a VH comprising VH CDRs 1, 2, and 3 from a VH having the amino acid sequence of SEQ ID NO: 98, and/or a VL comprising VL CDRs 1, 2, and 3 from a VL having the amino acid sequence of SEQ ID NO: 111; (2) a VH comprising VH CDRs 1, 2, and 3 from a VH having the amino acid sequence of SEQ ID NO:99, and/or a VL comprising VL CDRs 1, 2, and 3 from a VL having the amino acid sequence of SEQ ID NO: 112; (3) a VH comprising VH CDRs 1, 2, and 3 from a VH having the amino acid sequence of SEQ ID NO: 100, and/or a VL comprising VL CDRs 1, 2, and 3 from a VL having the amino acid sequence of SEQ ID NO: 113; (4) a VH comprising VH CDRs 1, 2, and 3 from a VH having the amino acid sequence of SEQ ID NO: 101, and/or a VL comprising VL CDRs 1, 2, and 3 from a VL having the amino acid sequence of SEQ ID NO: 114; (5) a VH comprising VH CDRs 1, 2, and 3 from a VH having the amino acid sequence of SEQ ID NO: 102, and/or a VL comprising VL CDRs 1, 2, and 3 from a VL having the amino acid sequence of SEQ ID NO: 115; (6) a VH comprising VH CDRs 1, 2, and 3 from a VH having the amino acid sequence of SEQ ID NO: 103, and/or a VL comprising VL CDRs 1, 2, and 3 from a VL having the amino acid sequence of SEQ ID NO: 116; (7) a VH comprising VH CDRs 1, 2, and 3 from a VH having the amino acid sequence of SEQ ID NO: 104, and/or a VL comprising VL CDRs 1, 2, and 3 from a VL having the amino acid sequence of SEQ ID NO: 117; (8) a VH comprising VH CDRs 1, 2, and 3 from a VH having the amino acid sequence of SEQ ID NO: 105, and/or a VL comprising VL CDRs 1, 2, and 3 from a VL having the amino acid sequence of SEQ ID NO: 118; (9) a VH comprising VH CDRs 1, 2, and 3 from a VH having the amino acid sequence of SEQ ID NO: 106, and/or a VL comprising VL CDRs 1, 2, and 3 from a VL having the amino acid sequence of SEQ ID NO: 119; (10) a VH comprising VH CDRs 1, 2, and 3 from a VH having the amino acid sequence of SEQ ID NO: 107, and/or a VL comprising VL CDRs 1, 2, and 3 from a VL having the amino acid sequence of SEQ ID NO: 120; (11) a VH comprising VH CDRs 1, 2, and 3 from a VH having the amino acid sequence of SEQ ID NO: 108, and/or a VL comprising VL CDRs 1, 2, and 3 from a VL having the amino acid sequence of SEQ ID NO: 121; (12) a VH comprising VH CDRs 1, 2, and 3 from a VH having the amino acid sequence of SEQ ID NO: 109, and/or a VL comprising VL CDRs 1, 2, and 3 from a VL having the amino acid sequence of SEQ ID NO: 122; or (13) a VH comprising VH CDRs 1, 2, and 3 from a VH having the amino acid sequence of SEQ ID NO:110, and/or a VL comprising VL CDRs 1, 2, and 3 from a VL having the amino acid sequence of SEQ ID NO: 123.

In some embodiments, provided herein are antibodies or antigen-binding fragments thereof that bind PD-L1 comprising a VH and a VL, wherein the VH comprises VH CDRs 1, 2, and 3 from a VH having the amino acid sequence of SEQ ID NO:99, and the VL comprises VL CDRs 1, 2, and 3 from a VL having the amino acid sequence of SEQ ID NO: 112.

In some embodiments, provided herein are antibodies or antigen-binding fragments thereof that bind PD-L1 comprising a VH and a VL, wherein the VH comprises VH CDRs 1, 2, and 3 from a VH having the amino acid sequence of SEQ ID NO: 101, and the VL comprises VL CDRs 1, 2, and 3 from a VL having the amino acid sequence of SEQ ID NO: 114.

In some embodiments, provided herein are antibodies or antigen-binding fragments thereof that bind PD-L1 comprising a VH and a VL, wherein the VH comprises VH CDRs 1, 2, and 3 from a VH having the amino acid sequence of SEQ ID NO: 100, and the VL comprises VL CDRs 1, 2, and 3 from a VL having the amino acid sequence of SEQ ID NO: 113.

In some embodiments, antibodies or antigen-binding fragments provided herein compete with an anti-PD-L1 antibody or antigen-binding fragment disclosed herein for binding to human PD-L1. In some embodiments, antibodies or antigen-binding fragments provided herein bind glycosylated PD-L1.

In some embodiments, anti-PD-L1 antibodies or antigen-binding fragments provided herein are monoclonal antibodies or antigen-binding fragments.

In some embodiments, anti-PD-L1 antibodies or antigen-binding fragments provided herein are bispecific or multispecific antibodies.

In some embodiments, anti-PD-L1 antibodies or antigen-binding fragments provided herein are selected from the group consisting of a Fab, a Fab′, a F(ab′)₂, a Fv, a scFv, a (scFv)₂, a single domain antibody (sdAb), and a heavy chain antibody (HCAb).

In some embodiments, anti-PD-L1 antibodies or antigen-binding fragments provided herein are selected from the group consisting of IgG1 antibodies, IgG2 antibodies, IgG3 antibodies, or IgG4 antibodies.

In some embodiments, anti-PD-L1 antibodies or antigen-binding fragments provided herein are chimeric antibodies or antigen-binding fragments, humanized antibodies or antigen-binding fragments, or human antibodies or antigen-binding fragments. In some embodiments, anti-PD-L1 antibodies or antigen-binding fragments provided herein humanized antibodies or antigen-binding fragments.

In some embodiments, provided herein are fusion proteins comprising a first domain comprising an anti-PD-L1 antibody or antigen-binding fragment disclosed herein and a second domain comprising a fragment of TGFβRII that binds TGFβ, or a variant thereof. In some embodiments, the first and second domains are linked via a transferrin linker. In some embodiments, the transferrin link links the C-terminus of the first domain to the N-terminus of the second domain. In some embodiments, the transferrin linker is (PEAPTD)m, m=1, 2, 3, 4, or 5 (SEQ ID NO:18) or (PEAPTDE)n, n=1, 2, 3, 4, or 5 (SEQ ID NO:19). In some embodiments, the transferrin linker has an amino acid sequence selected from the group consisting of SEQ ID NOs:220-230. In some embodiments, the transferrin linker is (PEAPTD)₃(SEQ ID NO: 147).

In some embodiments of the fusion proteins provided herein, the second domain comprises the ECD of TGFβRII isoform 1 (SEQ ID NO: 8) or the ECD of TGF β RII isoform 2 (SEQ ID NO: 14), or a variant thereof that has at least 85%, at least 90%, at least 95%, or at least 98% sequence identity to SEQ ID NO:8 or 14. In some embodiments, the second domain has the amino acid sequence of SEQ ID NO:8. In some embodiments, the second domain has the amino acid sequence of SEQ ID NO:9. In some embodiments, the second domain comprises a variant of the ECD of TGFβRII isoform 1 (SEQ ID NO:8), wherein the variant comprises an amino acid mutation at Q6, K7, N19 or G20 of SEQ ID NO:8. In some embodiments, the second domain has an amino acid sequence selected from the group consisting of SEQ ID NOs:201-203.

In some embodiments of the fusion proteins provided herein, the second domain comprises a truncated form of the ECD of TGFβRII isoform 1 (SEQ ID NO:8), which lacks amino acid residues 1 to n of SEQ ID NO:8, wherein n ranges from 2 to 30, or a variant thereof. In some embodiments, n is 19. In some embodiments of the fusion proteins provided herein, the second domain comprises a truncated form of the ECD of TGFβRII isoform 1 (SEQ ID NO:8), which lacks amino acid residues m to 136 of SEQ ID NO:8, wherein m ranges from 80 to 135, or a variant thereof. In some embodiments, m is 131. In some embodiments, m is 128. In some embodiments, the second domain has an amino acid sequence selected from the group consisting of SEQ ID NOs:204, 205 and 232.

In some embodiments, provided herein are fusion proteins comprising a heavy chain that has at least 85%, at least 90%, at least 95%, at least 98% or 100% sequence identity to an amino acid sequence selected from the group consisting of SEQ ID NOs: 155-161, 166-172, 206-212, and 233-240.

In some embodiments, fusion proteins provided herein further comprise a light chain that has at least 85%, at least 90%, at least 95%, at least 98%, or 100% sequence identity to an amino acid sequence selected from the group consisting of SEQ ID NOs: 162-165.

In some embodiments, provided herein are fusion proteins comprising a heavy chain and a light chain, wherein the heavy chain and light chain have (1) the amino acid sequences of SEQ ID NOs: 155 and 162, respectively; (2) the amino acid sequences of SEQ ID NOs: 156 and 162, respectively; (3) the amino acid sequences of SEQ ID NOs: 157 and 162, respectively; (4) the amino acid sequences of SEQ ID NOs: 158 and 162, respectively; (5) the amino acid sequences of SEQ ID NOs: 166 and 162, respectively; (6) the amino acid sequences of SEQ ID NOs: 167 and 162, respectively; (7) the amino acid sequences of SEQ ID NOs: 168 and 162, respectively; (8) the amino acid sequences of SEQ ID NOs: 169 and 162, respectively; (9) the amino acid sequences of SEQ ID NOs:206 and 162, respectively; (10) the amino acid sequences of SEQ ID NOs:207 and 162, respectively; (11) the amino acid sequences of SEQ ID NOs:208 and 162, respectively; (12) the amino acid sequences of SEQ ID NOs:209 and 162, respectively; (13) the amino acid sequences of SEQ ID NOs:210 and 162, respectively; (14) the amino acid sequences of SEQ ID NOs:211 and 162, respectively; (15) the amino acid sequences of SEQ ID NOs:212 and 162, respectively; (16) the amino acid sequences of SEQ ID NOs: 159 and 163, respectively; (17) the amino acid sequences of SEQ ID NOs: 170 and 163, respectively; (18) the amino acid sequences of SEQ ID NOs: 160 and 164, respectively; (19) the amino acid sequences of SEQ ID NOs: 171 and 164, respectively; (20) the amino acid sequences of SEQ ID NOs: 161 and 165, respectively; (21) the amino acid sequences of SEQ ID NOs: 172 and 165, respectively; (22) the amino acid sequences of SEQ ID NOs:233 and 162, respectively; (23) the amino acid sequences of SEQ ID NOs:234 and 162, respectively; (24) the amino acid sequences of SEQ ID NOs:235 and 162, respectively; (25) the amino acid sequences of SEQ ID NOs:236 and 162, respectively; (26) the amino acid sequences of SEQ ID NOs:237 and 162, respectively; (27) the amino acid sequences of SEQ ID NOs:238 and 162, respectively; (28) the amino acid sequences of SEQ ID NOs:239 and 162, respectively; or (29) the amino acid sequences of SEQ ID NOs:240 and 162, respectively.

In some embodiments, provided herein are polynucleotides that encode an anti-PD-L1 antibody or antigen-binding fragment disclosed herein. In some embodiments, provided herein are polynucleotides that encode a fusion protein disclosed herein.

In some embodiments, provided herein are vectors comprising a polynucleotide disclosed herein. In some embodiments, the vector is a viral vector.

In some embodiments, provided herein are isolated cells comprising a polynucleotide disclosed herein. In some embodiments, provided herein are isolated cells comprising a vector disclosed herein.

In some embodiments, provided herein are pharmaceutical compositions comprising a therapeutically effective amount of an anti-PD-L1 antibody or antigen-binding fragment disclosed herein and a pharmaceutically acceptable carrier. In some embodiments, provided herein are pharmaceutical compositions comprising a therapeutically effective amount of a fusion protein of disclosed herein and a pharmaceutically acceptable carrier.

In some embodiments, provided herein are methods of treating tumor or cancer in a subject in need thereof, comprising administering to the subject a therapeutically effective amount of the pharmaceutical composition disclosed herein. In some embodiments, provided herein are uses of a pharmaceutical composition disclosed herein in treatment of tumor or cancer. In some embodiments, provided herein are uses of a pharmaceutical composition disclosed herein for the preparation of a medicament for the treatment of tumor or cancer. In some embodiments, the tumor or cancer is a PD-L1 expressing tumor or cancer. In some embodiments, the pharmaceutical composition is administered in combination with a second therapy. In some embodiments, the second therapy is a chemotherapy, a radiation therapy, an immune therapy, or a cell therapy. In some embodiments, the second therapy is administered before, concurrently with, or after the administration of the pharmaceutical composition.

### BRIEF DESCRIPTON OF THE DRAWINGS

FIG. 1 shows the binding affinities of various anti-PD-L1 antibodies to tumor cell lines of different tissue origin and glycosylation status.

FIG. 2 shows that various anti-PD-L1 antibodies blocked PD-1 or CD80 binding to PD-L1.

FIG. 3 shows various anti-PD-L1 antibodies enhanced Jurkat report cell activation.

FIG. 4 shows various anti-PD-L1 antibodies enhanced IFN-γ secretion in MLR assay.

FIG. 5 shows that the fusion proteins could simultaneously bind both PD-L1 and TGFβ.

FIG. 6 shows the structure of an exemplary fusion protein.

FIG. 7 shows that fusion proteins inhibited SMAD signaling pathway.

FIG. 8 shows that fusion protein B significantly inhibited tumor growth.

FIGS. 9A-9C show capillary electrophoresis results demonstrating the stress stability of fusion proteins. FIG. 9A: fusion protein-1; FIG. 9B: fusion protein-24; and FIG. 9C: fusion protein-25.

FIGS. 10A-10B show capillary electrophoresis results demonstrating the superior stability of fusion protein with a rigid linker. FIG. 10A: M7824; FIG. 10B: M7824 with a rigid linker.

### DETAILED DESCRIPTION

Provided herein are novel anti-PD-L1 antibodies and antigen-binding fragments thereof, and fusion proteins targeting both PD-L1 and TGFβ, as well as their uses in the treatment of tumor or cancer. Both PD-L1 and TGFβ can be targeted in cancer treatment. The expression of PD-L1 by tumor cells can inhibit T cell activities and induce T cell apoptosis, which is beneficial to tumor growth. PD-1/PD-L1 checkpoint inhibitors can block the PD-1/PD-L1 signal and suppress tumor growth by restoring T cell activity. TGFβ has a variety of biological functions, including regulating cell growth, proliferation, differentiation, apoptosis, migration, and immunity. The tumor microenvironment can have a variety of cells that secrete TGF-β, which blocks T cell differentiation into Th1 and promotes their transformation into immunoinhibitory regulatory T cells (Tregs). TGFβ also promotes epithelial cell-to-mesenchymal transition (Epithelial-Mesenchymal Transition, EMT), and thereby promote tumor invasion and migration. TGFβ further enhances cell fibrosis and reduces immune cell tumor infiltration. Therefore, reducing TGFβ levels in the tumor microenvironment can suppress tumor growth.

Fusion proteins provided herein target both PD-L1 and TGFβ to inhibit tumor growth. A transferrin linker is used to connect the PD-L1-targetting domain and the TGFβ targeting domain. The transferrin linkers disclosed herein are rigid linkers that were surprisingly found to both improve the stability of the fusion protein and reduce its immunogenicity. Additionally, the novel anti-PD-L1 antibodies and antigen-binding fragments thereof provided herein can bind glycosylated PD-L1 and block PD-1/PD-L1 interaction and promote the internalization and degradation of PD-L1. In some embodiments, the anti-PD-L1 antibodies and antigen-binding fragments provided herein can serve as the PD-L1 targeting domain in the fusion proteins provided herein, and fusion proteins provided herein have superior stability and activity as compared to other PD-L1 and TGFβ-targeting therapeutic agents known in the art

### 1. Definitions

Unless otherwise defined herein, technical and scientific terms used in the present description have the meanings that are commonly understood by those of ordinary skill in the art.

The articles “a” and “an” as used herein refer to one or to more than one (i.e., to at least one) of the grammatical object of the article. By way of example, “an antibody” means one antibody or more than one antibody.

The term “fusion protein” and its grammatical equivalents as used herein refer to a protein, peptide or polypeptide that has an amino acid sequence derived from two or more separate proteins, peptides or polypeptides. In some embodiments, a fusion protein also includes linking regions of amino acids between amino acid portions derived from separate proteins, peptides or polypeptides. Such linking region of amino acids is referred herein as a “linker.” For example, a transferrin linker refers to a linker derived from transferrin protein (SEQ ID NO: 17), such as (PEAPTD)₃ (SEQ ID NO:147).

The term “binds,” as used herein, means that a polypeptide or molecule interacts more frequently, more rapidly, with greater duration, with greater affinity, or with some combination of the above to the epitope, protein, or target molecule than with alternative substances, including related and unrelated proteins. A binding moiety (e.g., antibody) that binds a target molecule (e.g., antigen) can be identified, for example, by immunoassays, ELISAs, SPR (e.g., Biacore), or other techniques known to those of skill in the art. Typically, a specific reaction will be at least twice background signal or noise and can be more than 10 times background. See, e.g., Paul, ed., 1989, Fundamental Immunology Second Edition, Raven Press, New York at pages 332-336 for a discussion regarding antibody specificity. In some embodiments, an antibody that binds a target molecule can bind the target molecule with an affinity that is at least 20 times greater, at least 30 times greater, at least 40 times greater, at least 50 times greater, at least 60 times greater, at least 70 times greater, at least 80 times greater, at least 90 times greater, or at least 100 times greater, than its affinity for a different molecule. The “binding affinity” generally refers to the strength of the sum total of noncovalent interactions between a binding moiety and a target molecule. The binding of a binding moiety and a target molecule is a reversible process, and the affinity of the binding is typically reported as an equilibrium dissociation constant (K_{D}). A variety of methods of measuring binding affinity are known in the art, any of which can be used for purposes of the present disclosure. In some embodiments, K_{D} can be measured by using surface plasmon resonance assays by Biacore, using, for example, a BIAcoreTM-2000 or a BIAcoreTM-3000 BIAcore, Inc., Piscataway, NJ), or by biolayer interferometry using, for example, the OctetQK384 system (ForteBio, Menlo Park, CA). In some embodiments, a binding moiety binds a molecule target with a K_{D} of about 0.1 mM or less, about 10 µM or less, about 1 µM or less, about 0.1 µM or less, about 0.01 µM or less, or about 1 nM or less. It is understood that, in some embodiments, a binding moiety that binds a first target may or may not bind a second target. Thus, a binding moiety can, in some embodiments, bind more than one target. For example, an antibody can, in certain instances, comprise two identical antigen-binding sites, each of which binds the same epitope on two or more proteins. In certain alternative embodiments, an antibody can be bispecific and comprise at least two antigen-binding sites with differing specificities.

The term “antibody” as used herein refers to an immunoglobulin molecule that recognizes and specifically binds a target, such as a protein, polypeptide, peptide, carbohydrate, polynucleotide, lipid, or a combination of any of the foregoing, through at least one antigen-binding site wherein the antigen-binding site is usually within the variable region of the immunoglobulin molecule. As used herein, the term encompasses intact polyclonal antibodies, intact monoclonal antibodies, single-domain antibodies (sdAbs; e.g., camelid antibodies, alpaca antibodies), single-chain Fv (scFv) antibodies, heavy chain antibodies (HCAbs), light chain antibodies (LCAbs), diabodies, tribodies, tetrabodies, multispecific antibodies, bispecific antibodies, monospecific antibodies, monovalent antibodies, fusion proteins comprising an antigen-binding site of an antibody, and any other modified immunoglobulin molecule comprising an antigen-binding site (e.g., dual variable domain immunoglobulin molecules) as long as the antibodies exhibit the desired biological activity. Antibodies also include, but are not limited to, mouse antibodies, camel antibodies, chimeric antibodies, humanized antibodies, and human antibodies. An antibody can be any of the five major classes of immunoglobulins: IgA, IgD, IgE, IgG, and IgM, or subclasses (isotypes) thereof (e.g., IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well-known subunit structures and three-dimensional configurations. Antibodies can be naked or conjugated to other molecules, including but not limited to, toxins and radioisotopes. Unless expressly indicated otherwise, the term “antibody” as used herein include “antigen-binding fragments” of intact antibodies.

The term “antigen-binding fragment” as used in connection with an antibody refers to a portion of an intact antibody and refers to the antigenic determining variable regions of an intact antibody. Examples of antibody fragments include, but are not limited to, Fab, Fab′, F(ab′)₂, Fv, linear antibodies, single chain antibody molecules (e.g., scFv), heavy chain antibodies (HCAbs), nanobodies, light chain antibodies (LCAbs), disulfide-linked scFv (dsscFv), diabodies, tribodies, tetrabodies, minibodies, dual variable domain antibodies (DVD), single variable domain antibodies (sdAbs; e.g., camelid antibodies, alpaca antibodies), single variable domain of heavy chain antibodies (VHH), and multispecific antibodies formed from antibody fragments.

The term “variable region” of an antibody as used herein refers to the variable region of an antibody light chain (“VL”), or the variable region of an antibody heavy chain (“VH”), either alone or in combination. Generally, the variable region of heavy and light chains each consist of four framework regions (FRs) and three complementarity determining regions (CDRs), also known as “hypervariable regions.” The CDRs of the light and heavy chains are primarily responsible for the interaction of the antibody with antigen. Numbering of amino acid positions used herein is according to the EU Index, as in Kabat et al. (1991) Sequences of proteins of immunological interest. (U.S. Department of Health and Human Services, Washington, D.C.) 5thed. A variable region can be a human variable region.

A CDR refers to one of three hypervariable regions (H1, H2 or H3) within the non-framework region of the immunoglobulin (Ig or antibody) VH β-sheet framework, or one of three hypervariable regions (L1, L2 or L3) within the non- framework region of the antibody VL β-sheet framework. Accordingly, CDRs are variable region sequences interspersed within the framework region sequences. CDR regions are well known to those skilled in the art and have been defined by a variety of methods/systems. These systems and/or definitions have been developed and refined over years and include Kabat, Chothia, IMGT, AbM, and Contact. For example, Kabat defines the regions of most hypervariability within the antibody variable (V) domains (Kabat et al, J. Biol. Chem. 252:6609-6616 (1977); Kabat, Adv. Prot. Chem. 32: 1-75 (1978)). The Chothia definition is based on the location of the structural loop regions, which defines CDR region sequences as those residues that are not part of the conserved β-sheet framework, and thus are able to adapt different conformations (Chothia and Lesk, J. Mol. Biol. 196:901-917 (1987)). Both terminologies are well recognized in the art. Additionally, the IMGT system is based on sequence variability and location within the structure of the variable regions. The AbM definition is a compromise between Kabat and Chothia. The Contact definition is based on analyses of the available antibody crystal structures. Software programs (e.g., abYsis) are available and known to those of skill in the art for analysis of antibody sequence and determination of CDRs. The positions of CDRs within a canonical antibody variable domain have been determined by comparison of numerous structures (Al-Lazikani et al, J. Mol. Biol. 273:927-948 (1997); Morea et al, Methods 20:267-279 (2000)). Because the number of residues within a hypervariable region varies in different antibodies, additional residues relative to the canonical positions are conventionally numbered with a, b, c and so forth next to the residue number in the canonical variable domain numbering scheme (Al-Lazikani et al., supra (1997)). Such nomenclature is similarly well known to those skilled in the art.

For example, CDRs defined according to either the Kabat (hypervariable) or Chothia (structural) designations, are set forth in the table below.

| | Kabat¹ | Chothia² | Loop Location |
|---|---|---|---|
| VHCDR1 | 31-35 | 26-32 | linking B and C strands |
| VHCDR2 | 50-65 | 53-55 | linking C′ and C″ strands |
| VHCDR3 | 95-102 | 96-101 | linking F and G strands |
| VLCDR1 | 24-34 | 26-32 | linking B and C strands |
| VLCDR2 | 50-56 | 50-52 | linking C′ and C″ strands |
| VLCDR3 | 89-97 | 91-96 | linking F and G strands |

¹Residue numbering follows the nomenclature of Kabat et al., supra
²Residue numbering follows the nomenclature of Chothia et al., supra

One or more CDRs also can be incorporated into a molecule either covalently or noncovalently to make it an immunoadhesin. An immunoadhesin can incorporate the CDR(s) as part of a larger polypeptide chain, can covalently link the CDR(s) to another polypeptide chain, or can incorporate the CDR(s) noncovalently. The CDRs permit the immunoadhesin to bind to a particular antigen of interest. The CDR regions can be analyzed by, for example, abysis website (http://abysis.org/).

The term “monoclonal antibody” as used herein refers to a substantially homogenous antibody population involved in the highly specific recognition and binding of a single antigenic determinant or epitope. The term “monoclonal antibody” encompasses intact and full-length monoclonal antibodies as well as antibody fragments (e.g., Fab, Fab′, F(ab′)2, Fv), single chain antibodies (e.g., scFv), fusion proteins comprising an antibody fragment, and any other modified immunoglobulin molecule comprising at least one antigen-binding site. Furthermore, “monoclonal antibody” refers to such antibodies made by any number of techniques, including but not limited to, hybridoma production, phage library display, recombinant expression, and transgenic animals.

The term “chimeric antibody” as used herein refers to an antibody wherein the amino acid sequence of the immunoglobulin molecule is derived from two or more species. Typically, the variable region of both light and heavy chains corresponds to the variable region of antibodies derived from one species of mammals (e.g., mouse, rat, rabbit, etc.) with the desired specificity, affinity, and/or binding capability, while the constant regions are homologous to the sequences in antibodies derived from another species (usually human) to avoid eliciting an immune response in that species.

The term “humanized antibody” as used herein refers to forms of non-human (e.g., murine) antibodies that are specific immunoglobulin chains, chimeric immunoglobulins, or fragments thereof that contain minimal non-human sequences. Typically, humanized antibodies are human immunoglobulin. In some instances, the Fv framework region residues of a human immunoglobulin are replaced with the corresponding residues in an antibody from a non-human species. In some instances, residues of the CDRs are replaced by residues from the CDRs of a non-human species (e.g., mouse, rat, hamster, camel) that have the desired specificity, affinity, and/or binding capability. The humanized antibody can be further modified by the substitution of additional residues either in the Fv framework region and/or within the replaced non-human residues to refine and optimize antibody specificity, affinity, and/or binding capability. The humanized antibody can comprise variable regions containing all or substantially all of the CDRs that correspond to the non-human immunoglobulin whereas all or substantially all of the framework regions are those of a human immunoglobulin sequence. In some embodiments, the variable regions comprise the framework regions of a human immunoglobulin sequence. In some embodiments, the variable regions comprise the framework regions of a human immunoglobulin consensus sequence. The humanized antibody can also comprise at least a portion of an immunoglobulin constant region or domain (Fc), typically that of a human immunoglobulin. A humanized antibody is usually considered distinct from a chimeric antibody.

The term “human antibody” as used herein refers to an antibody produced by a human or an antibody having an amino acid sequence corresponding to an antibody produced by a human made using any of the techniques known in the art. These techniques include, but not limited to, phage display libraries, yeast display libraries, transgenic animals, recombinant protein production, and B-cell hybridoma technology.

The terms “epitope” and “antigenic determinant” are used interchangeably herein refer to the site on the surface of a target molecule to which a binding moiety binds, such as a localized region on the surface of an antigen. The target molecule can comprise, a protein, a peptide, a nucleic acid, a carbohydrate, or a lipid. An epitope having immunogenic activity is a portion of a target molecule that elicits an immune response in an animal. An epitope of a target molecule having antigenic activity is a portion of the target molecule to which an antibody binds, as determined by any method well known in the art, including, for example, by an immunoassay. Antigenic epitopes need not necessarily be immunogenic. Epitopes often consist of chemically active surface groupings of molecules such as amino acids or sugar side chains and have specific three-dimensional structural characteristics as well as specific charge characteristics. The term, “epitope” includes linear epitopes and conformational epitopes. A region of a target molecule (e.g., a polypeptide) contributing to an epitope may be contiguous amino acids of the polypeptide or the epitope may come together from two or more non-contiguous regions of the target molecule. The epitope may or may not be a three-dimensional surface feature of the target molecule. Epitopes formed from contiguous amino acids (also referred to as linear epitopes) are typically retained upon protein denaturing, whereas epitopes formed by tertiary folding (also referred to as conformational epitopes) are typically lost upon protein denaturing. An epitope typically includes at least 3, and more usually, at least 5, 6, 7, or 8-10 amino acids in a unique spatial conformation. Epitopes can be predicted using a number of approaches, including software bioinformatic tools available on the internet. X-ray crystallography may be used to characterize an epitope on a target protein by analyzing the amino acid residue interactions of an antigen/antibody complex.

The terms “polypeptide,” “peptide,” and “protein” as used interchangeably herein refer to polymers of amino acids of any length, which can be linear or branched. It can include unnatural or modified amino acids or be interrupted by non-amino acids. A polypeptide, peptide, or protein can also be modified with, for example, disulfide bond formation, glycosylation, lipidation, acetylation, phosphorylation, or any other manipulation or modification.

The terms “polynucleotide” and “nucleic acid” as used interchangeably herein refer to polymers of nucleotides of any length and include DNA and RNA. The nucleotides can be deoxyribonucleotides, ribonucleotides, modified nucleotides or bases, and/or their analogs, or any substrate that can be incorporated into a polymer by DNA or RNA polymerase.

A polypeptide, peptide, protein, antibody, polynucleotide, vector, cell, or composition which is “isolated” is a polypeptide, peptide, protein, antibody, polynucleotide, vector, cell, or composition which is in a form not found in nature. Isolated polypeptides, peptides, proteins, antibodies, polynucleotides, vectors, cells, or compositions include those which have been purified to a degree that they are no longer in a form in which they are found in nature. A polypeptide, soluble protein, antibody, polynucleotide, vector, cell, or composition can be isolated from a natural source (e.g., tissue) or from a source such as an engineered cell line. In some embodiments, a polypeptide, peptide, protein, antibody, polynucleotide, vector, cell, or composition which is isolated is substantially pure.

The terms “identical” or percent “identity” as used herein in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software that can be used to obtain alignments of amino acid or nucleotide sequences are well-known in the art. These include, but are not limited to, BLAST, ALIGN, Megalign, BestFit, GCG Wisconsin Package, and variants thereof. In some embodiments, two nucleic acids or polypeptides provided herein are substantially identical, meaning they have at least 70%, at least 75%, at least 80%, at least 85%, at least 90%, and in some embodiments at least 95%, 96%, 97%, 98%, 99% nucleotide or amino acid residue identity, when compared and aligned for maximum correspondence, as measured using a sequence comparison algorithm or by visual inspection. In some embodiments, identity exists over a region of the amino acid sequences that is at least about 10 residues, at least about 20 residues, at least about 30 residues, at least about 40-60 residues, at least about 60-80 residues in length or any integral value there between. In some embodiments, identity exists over a longer region than 60-80 residues, such as at least about 80-100 residues, and in some embodiments the sequences are substantially identical over the full length of the sequences being compared, such as the coding region of a nucleotide sequence or an amino acid sequence. In some embodiments, identity exists over a region of the nucleotide sequences that is at least about 10 bases, at least about 20 bases, at least about 30 bases, at least about 40-60 bases, at least about 60-80 bases in length or any integral value there between. In some embodiments, identity exists over a longer region than 60-80 bases, such as at least about 80-1000 bases or more, and in some embodiments the sequences are substantially identical over the full length of the sequences being compared, such as a nucleotide sequence encoding a protein of interest.

The term “amino acid substitution,” as used herein, refers to the replacement of one amino acid residue with another in a polypeptide sequence. A “conservative amino acid substitution” is one in which one amino acid residue is replaced with another amino acid residue having a side chain with similar chemical characteristics. Families of amino acid residues having similar side chains have been generally defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. Generally, conservative substitutions in the sequences of the polypeptides, soluble proteins, and/or antibodies of the disclosure do not abrogate the binding of the polypeptide, soluble protein, or antibody containing the amino acid sequence, to the target binding site. Methods of identifying amino acid conservative substitutions which do not eliminate binding are well-known in the art.

The term “variant” as used herein in relation to a molecule (e.g., an antibody) having a polypeptide with particular sequence features (the “reference molecule”) refers to a different molecule having a polypeptide comprising one or more (for example, about 1 to about 80, about 1 to about 70, about 1 to about 60, about 1 to about 50, about 1 to about 40, about 1 to about 30, about 1 to about 25, about 1 to about 20, about 1 to about 15, about 1 to about 10, or about 1 to about 5) amino acid sequence substitutions, deletions, and/or additions as compared to the reference molecule, which still retains the main property, activity, and/or function of the reference molecule. For example, an anti-PD-L1-antibody variant at least retains binding to PD-L1, and can result from one or more (such as, for example, about 1 to about 25, about 1 to about 20, about 1 to about 15, about 1 to about 10, or about 1 to about 5) changes (substitutions, additions, and/or deletions) to an amino acid sequence of a reference anti-PD-L1 antibody. The changes to an amino acid sequence can be amino acid substitutions. In some embodiments, the changes to an amino acid sequence can be conservative amino acid substitutions. In some embodiments, an anti-PD-L1 antibody variant can have one or more (such as, for example, about 1 to about 25, about 1 to about 20, about 1 to about 15, about 1 to about 10, or about 1 to about 5) amino acid substitutions in the VH or VL regions or subregions, such as one or more CDRs, as compared to the reference antibody. In some embodiments, an anti-PD-L1 antibody variant can have one, up to two, up to three, up to four, up to five, up to six, up to seven, up to eight, up to nine, or up to ten amino acid substitutions in each of the VH or VL region as compared to the reference antibody. In some embodiments, an anti-PD-L1 antibody variant can have one, up to two, up to three, up to four, or up to five, up to six, up to seven, up to eight, up to nine, or up to ten amino acid substitutions in each of the CDRs as compared to the reference antibody. For another example, a variant of a TGFβ RII fragment that binds TGFβ retains its ability to bind TGFβ, and con have one or more (for example, about 1 to about 80, about 1 to about 70, about 1 to about 60, about 1 to about 50, about 1 to about 40, about 1 to about 30, about 1 to about 25, about 1 to about 20, about 1 to about 15, about 1 to about 10, or about 1 to about 5) amino acid sequence substitutions, deletions, and/or additions as compared to the reference TGFβ RII fragment.

The term “vector” refers to a construct that is used to carry or include a polynucleotide, which can be used to, for example, introduce a polynucleotide into a host cell. An expression vector is capable of not only delivering a polynucleotide into a host cell, but also expressing the polynucleotide in the cell. Vectors applicable for use include, for example, expression vectors, plasmids, cosmid, phage vectors, viral vectors, episomes and artificial chromosomes, which can include selection sequences or markers operable for stable integration into a host cell’s chromosome. Examples of vectors also include naked DNA or RNA expression vectors, DNA or RNA expression vectors associated with cationic condensing agents, and DNA or RNA expression vectors encapsulated in liposomes. Additionally, the vectors can include one or more selectable marker genes and appropriate expression control sequences. Selectable marker genes that can be included, for example, provide resistance to antibiotics or toxins, complement auxotrophic deficiencies, or supply critical nutrients not in the culture media. Expression control sequences can include constitutive and inducible promoters, transcription enhancers, transcription terminators, and the like which are well known in the art. When two or more polynucleotides are to be co-expressed (e.g., both an antibody heavy and light chain or an antibody VH and VL) both polynucleotides can be inserted, for example, into a single expression vector or in separate expression vectors. For single vector expression, the encoding polynucleotides can be operationally linked to one common expression control sequence or linked to different expression control sequences, such as one inducible promoter and one constitutive promoter. The introduction of polynucleotides into a host cell can be confirmed using methods well known in the art. It is understood by those skilled in the art that the polynucleotides are expressed in a sufficient amount to produce a desired product (e.g., a fusion protein as described herein), and it is further understood that expression levels can be optimized to obtain sufficient expression using methods well known in the art.

The term “subject” refers to any animal (e.g., a mammal), including, but not limited to, humans, non-human primates, canines, felines, rodents, and the like, which is to be the recipient of a particular treatment. In some embodiments, a subject is a human. A “subject” can be a patient with a disease. In some embodiments, a subject is a patient having a cancer or tumor.

The term “treat” as used herein in connection with a disease or a condition, or a subject having a disease or a condition refers to an action that suppresses, eliminates, reduces, and/or ameliorates a symptom, the severity of the symptom, and/or the frequency of the symptom associated with the disease or disorder being treated. When used in reference to a cancer or tumor, the term “treat” refers to an action that reduces the severity of the cancer or tumor, or retards or slows the progression of the cancer or tumor, including (a) inhibiting the growth, or arresting development of the cancer or tumor, or (b) causing regression of the cancer or tumor, or (c) delaying, ameliorating or minimizing one or more symptoms associated with the presence of the cancer or tumor.

The term “administer,” “administering,” or “administration” as used herein refers to the act of delivering, or causing to be delivered, a therapeutic or a pharmaceutical composition to the body of a subject by a method described herein or otherwise known in the art. The therapeutic can be a compound, a polypeptide, a cell, or a population of cells. Administering a therapeutic or a pharmaceutical composition includes prescribing a therapeutic or a pharmaceutical composition to be delivered into the body of a patient. Exemplary forms of administration include oral dosage forms, such as tablets, capsules, syrups, suspensions; injectable dosage forms, such as intravenous (IV), intramuscular (IM), or intraperitoneal (IP); transdermal dosage forms, including creams, jellies, powders, or patches; buccal dosage forms; inhalation powders, sprays, suspensions, and rectal suppositories.

The term “therapeutically effective amount” as used herein refers to an amount of a compound, polypeptide, cell, formulation, material, or composition, as described herein sufficient to provide a therapeutic benefit in the treatment of the disease or disorder or to delay or minimize one or more symptoms associated with the disease or disorder. The disease or disorder can be a cancer or tumor.

As used herein, the term “carrier” includes “pharmaceutically acceptable carriers,” excipients, or stabilizers that are nontoxic to the cell or mammal being exposed thereto at the dosages and concentrations employed. The term “carrier” can also refer to a diluent, adjuvant (e.g., Freund’s adjuvant (complete or incomplete)), excipient, or vehicle with which therapeutic is administered. Examples of suitable pharmaceutical carriers are described in Remington’s Pharmaceutical Sciences (1990) Mack Publishing Co., Easton, PA. Compositions, including pharmaceutical compositions, can contain a therapeutically effective amount of an fusion protein or an anti-PD-L1 antibody, for example, in isolated or purified form, together with a suitable amount of carrier so as to provide the form for proper administration to the subject (e.g., patient). The formulation should suit the mode of administration.

### 2. Fusion Proteins

Provided herein are fusion proteins having two domains linked by a rigid linker (e.g., a transferrin linker), wherein the two domains bind two different targets, at least one of which is on cell surface. Short amino acid linkers such as GGGGS (SEQ ID NO: 182) are commonly and preferably used in the art to tether different functional domains in fusion proteins because they are flexible and can help assure the proper three-dimensional positioning of the functional domains. This is particularly important for fusion proteins that function by binding target molecules on cell surface. However, it was surprisingly found by inventors of the present disclosure that fusion proteins having rigid linkers such as the transferrin linkers had superior stability and activity compared to those with flexible linkers. The “rigid linkers” disclosed herein refer to linkers that are substantially non-helical and can physically separate the two domains of a fusion protein. A linker that is substantially non-helical exhibits little or limited helical or spiral shape or secondary structure. Linkers that connect two functional domains in the fusion proteins disclosed herein can have at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 34, or more amino acids. In some embodiments, linkers that connect two functional domains in the fusion proteins disclosed herein can have 5 to 35 amino acids, 5 to 30 amino acids, 10 to 25, or 10 to 20 amino acids. The linkers can be rigid or substantially non-helical. In some embodiments, provided herein are fusion proteins having two domains linked by a rigid linker (e.g., a transferrin linker), wherein one or two domains of the fusion proteins bind a target molecule on cell surface, and the linker has 10 to 20 amino acids and is substantially non-helical.

In some embodiments, provided herein are fusion proteins wherein the first domain and the second domain are linked by a transferrin linker. In some embodiments, the transferrin linker is (PEAPTD)n, n=1, 2, 3, 4, or 5 (SEQ ID NO: 18). In some embodiments, the transferrin linker is (PEAPTDE)n, n=1, 2, 3, 4, or 5 (SEQ ID NO: 19). The transferrin linker can be PEAPTD (SEQ ID NO: 145). The transferrin linker can be (PEAPTD)₂ (SEQ ID NO: 146). The transferrin linker can be (PEAPTD)₃ (SEQ ID NO:147). The transferrin linker can be (PEAPTD)₄ (SEQ ID NO: 148). The transferrin linker can be (PEAPTD)₅ (SEQ ID NO: 149). The transferrin linker can be PEAPTDE (SEQ ID NO: 150). The transferrin linker can be (PEAPTDE)₂ (SEQ ID NO: 151). The transferrin linker can be (PEAPTDE)₃ (SEQ ID NO: 152). The transferrin linker can be (PEAPTDE)₄ (SEQ ID NO: 153). The transferrin linker can be (PEAPTDE)₅ (SEQ ID NO: 154).

In some embodiments, the transferrin linker is (PEAPTD)nP, n=1, 2, 3, 4, or 5 (SEQ ID NO:220). In some embodiments, the transferrin linker is (PEAPTD)nPE, n=1, 2, 3, 4, or 5 (SEQ ID NO:221). In some embodiments, the transferrin linker is (PEAPTD)nPEA, n=1, 2, 3, 4, or 5 (SEQ ID NO:222). In some embodiments, the transferrin linker is (PEAPTD)nPEAP, n=1, 2, 3, 4, or 5 (SEQ ID NO:223). In some embodiments, the transferrin linker is (PEAPTD)nPEAPT, n=1, 2, 3, 4, or 5 (SEQ ID NO:224). In some embodiments, the transferrin linker is (PEAPTDE)nP, n=1, 2, 3, 4, or 5 (SEQ ID NO:225). In some embodiments, the transferrin linker is (PEAPTDE)nPE, n=1, 2, 3, 4, or 5 (SEQ ID NO:226). In some embodiments, the transferrin linker is (PEAPTDE)nPEA, n=1, 2, 3, 4, or 5 (SEQ ID NO:227). In some embodiments, the transferrin linker is (PEAPTDE)nPEAP, n=1, 2, 3, 4, or 5 (SEQ ID NO:228). In some embodiments, the transferrin linker is (PEAPTDE)nPEAPT, n=1, 2, 3, 4, or 5 (SEQ ID NO:229). In some embodiments, the transferrin linker is (PEAPTDE)nPEAPTD, n=1, 2, 3, 4, or 5 (SEQ ID NO:230). For example, in some embodiments, the transferrin linker is (PEAPTD)2PEA, or PEAPTDPEAPTDPEA (SEQ ID NO:231).

The rigid linkers can be used to construct a variety of fusion proteins. For example, antibody-cytokine fusion proteins (sometimes referred to as immunocytokines) comprise cytokines fused to an antibody, which can localize immunomodulatory cytokine payloads to the tumor, thereby improving antibody-targeted cancer immunotherapy. These molecules have the capacity to enhance the tumoricidal activity of the antibodies and/or activate a secondary antitumor immune response. In some embodiments, the antibody-cytokine fusion proteins can include a rigid linker disclosed herein. In some embodiments, the antibody-cytokine fusion proteins provided herein comprise (a) an antibody or antigen-binding fragment, (b) a rigid linker, and (c) a cytokine payload. The rigid linker can be any rigid linker provided herein. The antibody or antigen-binding fragment of the antibody-cytokine fusion targets specific antigens that are abundantly expressed in neoplastic tissues but absent from normal tissue. In some embodiments, the antibody or antigen-binding fragment of the antibody-cytokine fusions targets an antigen located on new blood vessels or in surrounding extracellular matrix (ECM) structures of the neoplastic tissues. In some embodiments, the antibody or antigen-binding fragment of the antibody-cytokine fusions targets the alternatively-spliced extra domains A (EDA) or B (EDB) of fibronectin, the A1 domain of tenascin-C (TnC A1), or ECM components (such as F8, L19 or F16). In some embodiments, the antibody or antigen-binding fragment of the antibody-cytokine fusion proteins provided herein targets a tumor antigen such as integrins (αvβ3), annexin A1, prostate-specific membrane antigen (PSMA), vascular endothelia growth factors (VEGF) and their receptors, endoglin (CD105), CD44 isoforms, alanyl aminopeptidase (CD13), A33, carcinoembryonic (CEA) antigen, carbonic anhydrase IX, epithelial cell adhesion molecule (EpCAM), disialoganglioside 2 (GD2), the fibroblast activation protein (FAP), human epidermal growth factor receptor 2 (HER2), maj or histocompatibility complex class II (MHCII), phosphatidylserine (PS), epidermal growth factor (EGF), CD20, EGFR (epidermal growth factor receptor), αFR (alpha folate receptor), Histone, mesothelin (MSLN), CD30, human leukocyte antigen DR (HLA-DR), carbonic anhydrase IX (CAIX), gp240, or Lewis Y antigen (LeY).

The antibody-cytokine fusion proteins provided herein comprise (a) an antibody or antigen-binding fragment, (b) a rigid linker, and (c) a cytokine payload. In some embodiments, the cytokine payload is Granulocyte colony-stimulating factor (G-CSF), Granulocyte-macrophage colony-stimulating factor (GM-CSF), interleukin 1β (IL-1β), interleukin 2 (IL-2), interleukin 3 (IL-3), interleukin 4 (IL-4), interleukin 6 (IL-6), interleukin 7 (IL-7), interleukin 9 (IL-9), interleukin 12 (IL-12), interleukin 13 (IL-13), interleukin 15 (IL-15), interleukin 17 (IL-17), interleukin 18 (IL-18), interleukin 21 (IL-21), interferon α (IFN- α), interferon γ (IFN- γ), TNF, or heterodimeric cytokines such as IL2/IL12, TNF/IL2, IL4/GM-CSF, or IL12-GMCSF. In some embodiments, the cytokine payload is a chemokine, such as, CCL5, CCL17, CCL19, CCL20, CCL21, CXCL4, CXCL9, CXCL10, CXCL11a, or ITIP. In some embodiments, the cytokine payload is a TNF superfamily member, such as TRAIL, CD40L, OX40L, FasL, LiGHT, or VEGI.

In some embodiments, the cytokine payload in the antibody-cytokine fusion proteins can be IL-1, IL-2, IL-3, IL-4, IL-5, IL-6, IL-7, IL-8, IL-9, IL-10, IL-11, IL-12, IL-13, IL-14, IL-15, IL-16, IL-17A, IL-17B, IL-17C, IL-17D, IL-17E, IL-17F, IL-18, IL-19, IL-20, IL-21, IL-22, IL-23, IL-24, IL-25, IL-26, IL-27, IL-28A, IL-28B, IL-29, IL-30, IL-31, IL-32, IL-33, IL-34, IL-35, CNTF, CLC, CT-1, OSM, CXCL1, CXCL2, CXCL3, CXCL4, CXCL5, CXCL6, CXCL7, CXCL8, IFN-α, IFN-β, IFN-ω, IFN-γ, IFN-λ, TNF-α, TNF-β, LTα, BAFF, APRIL, OX40L, GITRL, CD70, 4-1BBL, CD40L, TL1A, LIGHT, RANKL, TWEAK, FasL, TRAIL, TGF-β1, TGF-β2, TGF-β3, Myostain, Nodal, GDF, Inhibin, Lefty, BMP, EGF, PDGF, FGF, IGF-I, IGF-II, LIF, NGF, OSM, TGF-α, VEGF, or GM-CSF.

Fusion proteins provided herein also include cytokine traps, namely, fusion proteins comprising the constant region of IgG and the extracellular domains of two cytokine receptor components involved in binding the cytokine, linked by a rigid linker disclosed herein. In some embodiments, the cytokine traps disclosed herein can have a cytokine-binding domain of a cytokine receptor that is IL-1R1, IL-1R2, IL-1R3, IL-1R3b, IL-1R4, IL-1R5, IL-1R6, IL-1R7, IL-1R8, IL-1R9 IL-2Rβ, IL-4Rα, IL-5Rα, IL-7Rα, IL-9Rα, IL-21R, GMRα, IL-3Rα, IL-6R, IL-11R, CNTF-R, CT-1R, LIF-R, OSM-R1, OSM-R2, IL-27R, IL-31R, IL-35R, CXCR1, CXCR2, IL-10R1, IL-20R1, IL-22R, IL-28R, IL-10R2, IL-20R2, IL-23R, IL-12Rβ1, IL-12Rβ2, IL-13R, IL-17RA, IL-17RB, IL-17RC, IL-17RD, IL-17RE, IL-31RA, CSF-1R, IFNAR1, IFNAR2, IFN-λr1, TNFR1, TNFR2, BAFFR, BCMA, TACI, OX40, GITR, CD27, 4-1BB, CD40, DR3, HVEM, RANK, Fn14, Fas, TRAILR1, TRAILR2, IL10R, IL11R, IL12R, IL13R, IL14R, IL15R, IL17R, IL18R, IL19R, IL20R, IL21R, IL22R, IL23R, IL24R, IL25R, IL26R, IL27R, IL28R, IL29R, IL30R, IL31R, IL32R, IL33R, IL34R, IL35R, TGFβR, ActR, BMPR, or any combination thereof. The constant region of IgG can be the Fc region (e.g., the Fc region of human IgG1). In some embodiments, the fusion proteins provided herein comprise a cytokine-binding region of a cytokine receptor linked to the Fc region of human IgG1 via a rigid linker provided herein.

In some embodiments, provided herein are fusion proteins having two domains linked by a rigid linker (e.g., a transferrin linker), wherein the first domain comprises an antibody that binds PD-L1 (e.g., human PD-L1), or an antigen-binding fragment thereof, and the second domain comprises a fragment of TGFβRII that binds TGFβ, or a variant thereof. The first domain of the fusion protein (or the “anti-PD-L1 domain”) targets the PD-1/PD-L1 immune checkpoint pathway and promotes T cell activity and proliferation by counteracting the suppression from the PD-1/PD-L1 signaling. The second domain of the fusion protein (or the “TGFβ trap domain”) binds and traps TGFβ, reducing its level in the tumor microenvironment by promoting its internalization and degradation. In some embodiments, the linker connects the C-terminus of the first domain to the N-terminus of the second domain. In some embodiments, the linker connects the C-terminus of the second domain to the N-terminus of the first domain.

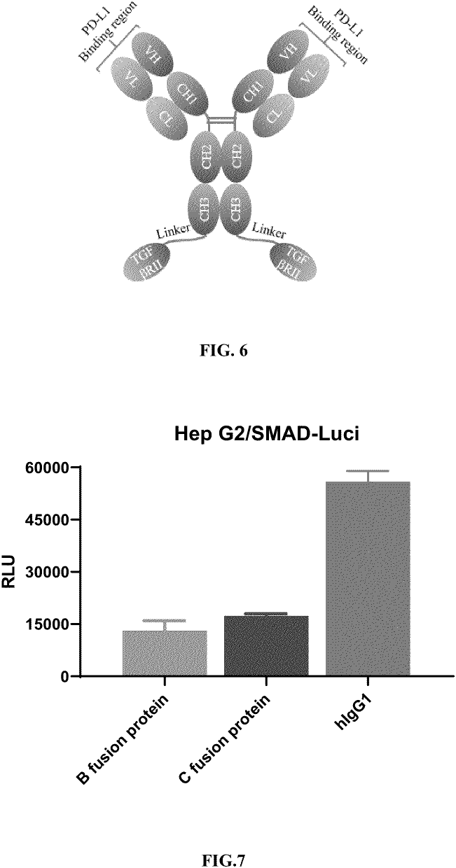

In some embodiments, the anti-PD-L1 domain consists of a single polypeptide, and the fusion protein also consists of a single polypeptide comprising the anti-PD-L1 domain linked to a TGFβ trap domain. For example, in some embodiments, the fusion protein consists of an anti-PD-L1 scFv linked to a TGFβ trap domain. In some embodiments, the fusion protein consists of an anti-PD-L1 single domain antibody linked to a TGFβ trap domain. In some embodiments, the anti-PD-L1 domain comprises a first polypeptide and a second polypeptide, and the TGFβ trap domain can be linked to either the first polypeptide or the second polypeptide to form the fusion protein. In some embodiments, the fusion protein also comprises (1) a first polypeptide comprising the first polypeptide of the anti-PD-L1 domain linked to a TGFβ trap domain, and (2) a second polypeptide comprising the second polypeptide of the anti-PD-L1 domain. In some embodiments, the anti-PD-L1 domain comprises a first polypeptide and a second polypeptide, and the fusion protein also comprises (1) a first polypeptide comprising the first polypeptide of the anti-PD-L1 domain linked to a TGFβ trap domain, and (2) a second polypeptide comprising the second polypeptide of the anti-PD-L1 domain. For example, in some embodiments, the anti-PD-L1 domain comprises an antibody heavy chain and an antibody light chain, and the fusion protein comprises a heavy chain comprising the antibody heavy chain linked to a TGFβ trap domain, and a light chain comprising the antibody light chain (e.g., FIG. 6). The antibody heavy chain can be directly connected to the TGFβ trap domain, or linked to the TGFβ trap domain via a linker. In some embodiments, the linker is a rigid linker disclosed herein. In some embodiments, the fusion protein comprises a heavy chain comprising the antibody heavy chain, and a light chain comprising the antibody light chain linked to a TGFβ trap domain.

Provided herein are bifunctional fusion proteins that targets the immunosuppressive signal from both TGFβ and PD-1/PD-L1 signaling, with the first domain being a TGFβ trap domain and the second domain being an anti-PD-L1 domain. TGFβRII is a natural receptor for TGFβ and can serve as the trap domain. TGFβRII has two isoforms, isoform 1 and isoform 2, both capable of binding TGFβ and serving as the TGFβ trap. The amino acid sequence of TGFβRII isoform 1 is shown below (SEQ ID NO:7). The underlined region (amino acid resides 24-159) represents the extracellular domain (ECD) of TGFβRII isoform 1 (SEQ ID NO:8).

MGRGLLRGLWPLHIVLWTRIASTIPPHVQKSVNNDMIVTDNNGAVKFPQL CKFCDVRFSTCDNQKSCMSNCSITSICEKPQEVCVAVWRKNDENITLETV CHDPKLPYHDFILEDAASPKCIMKEKKKPGETFFMCSCSSDECNDNIIFS EEYNTSNPDLLLVIFQVTGISLLPPLGVAISVIIIFYCYRVNRQQKLSST
WETGKTRKLMEFSEHCAIILEDDRSDISSTCANNINHNTELLPIELDTLV
GKGRFAEVYKAKLKQNTSEQFETVAVKIFPYEEYASWKTEKDIFSDINLK
HENILQFLTAEERKTELGKQYWLITAFHAKGNLQEYLTRHVISWEDLRKL
GSSLARGIAHLHSDHTPCGRPKMPIVHRDLKSSNILVKNDLTCCLCDFGL
SLRLDPTLSVDDLANSGQVGTARYMAPEVLESRMNLENVESFKQTDVYSM
ALVLWEMTSRCNAVGEVKDYEPPFGSKVREHPCVESMKDNVLRDRGRPEI
PSFWLNHQGIQMVCETLTECWDHDPEARLTAQCVAERFSELEHLDRLSGR
SCSEEKIPEDGSLNTTK (SEQ ID NO:7)

The amino acid sequence of TGFβRII isoform 2 is shown below (SEQ ID NO:8). The underlined region (amino acid resides 24-184) represents the ECD of TGFβRII isoform 2 (SEQ ID NO: 14).

MGRGLLRGLWPLHIVLWTRIASTIPPHVQKSDVEMEAQKDEIICPSCNRT AHPLRHINNDMIVTDNNGAVKFPQLCKFCDVRFSTCDNQKSCMSNCSITS ICEKPQEVCVAVWRKNDENITLETVCHDPKLPYHDFILEDAASPKCIMKE KKKPGETFFMCSCSSDECNDNIIFSEEYNTSNPDLLLVIFQVTGISLLPP
LGVAISVIIIFYCYRVNRQQKLSSTWETGKTRKLMEFSEHCAIILEDDRS
DISSTCANNINHNTELLPIELDTLVGKGRFAEVYKAKLKQNTSEQFETVA
VKIFPYEEYASWKTEKDIFSDINLKHENILQFLTAEERKTELGKQYWLIT
AFHAKGNLQEYLTRHVISWEDLRKLGSSLARGIAHLHSDHTPCGRPKMPI
VHRDLKSSNILVKNDLTCCLCDFGLSLRLDPTLSVDDLANSGQVGTARYM
APEVLESRMNLENVESFKQTDVYSMALVLWEMTSRCNAVGEVKDYEPPFG
SKVREHPCVESMKDNVLRDRGRPEIPSFWLNHQGIQMVCETLTECWDHDP
EARLTAQCVAERFSELEHLDRLSGRSCSEEKIPEDGSLNTTK (SEQ ID
NO: 8)

Accordingly, provided herein are fusion proteins having two domains, wherein the first domain comprises an antibody that binds PD-L1 (e.g., human PD-L1), or an antigen-binding fragment thereof, and the second domain comprises a fragment of TGFβRII that binds TGFβ, or a variant thereof. The two domains can be directly connected or linked by a linker. In some embodiments, the linker can be a rigid linker (e.g., a transferrin linker). The variant of TGFβRII fragment that binds TGFβ retains the binding to TGFβ. In some embodiments, the second domain comprises the ECD of TGFβRII isoform 1 (SEQ ID NO:8), or a variant thereof. The variant of the ECD of TGFβRII isoform 1 retains the binding to TGFβ. In some embodiments, the second domain comprises the ECD of TGFβRII isoform 2 (SEQ ID NO: 14), or a variant thereof. The variant of the ECD of TGFβRII isoform 2 retains the binding to TGFβ.

In some embodiments, the second domain comprises the ECD of TGFβRII isoform 1 (SEQ ID NO: 8). In some embodiments, the second domain consists of the ECD of TGFβRII isoform 1 (SEQ ID NO:8). In some embodiments, the second domain comprises a variant of the ECD of TGFβRII isoform 1. In some embodiments, the second domain of the fusion proteins provided herein comprises a variant of the TGFβRII ECD isoform 1 having at least 85%, at least 90%, at least 95%, or at least 98% sequence identity to SEQ ID NO:8. In some embodiments, the second domain of the fusion proteins provided herein has at least 85% sequence identity to SEQ ID NO:8. In some embodiments, the second domain of the fusion proteins provided herein has at least 90% sequence identity to SEQ ID NO:8. In some embodiments, the second domain of the fusion proteins provided herein has at least 95% sequence identity to SEQ ID NO:8. In some embodiments, the second domain of the fusion proteins provided herein has at least 98% sequence identity to SEQ ID NO:8. In some embodiments, the second domain has the amino acid sequence of SEQ ID NO:9. In some embodiments, the second domain comprises a variant of the ECD of TGFβRII isoform 1 (SEQ ID NO:8), wherein the variant comprises an amino acid mutation at Q6, K7, N19 or G20 of SEQ ID NO: 8, or any combination thereof. In some embodiments, the variant comprises an amino acid mutation at Q6. In some embodiments, the variant comprises an amino acid mutation at K7. In some embodiments, the variant comprises an amino acid mutation at N19. In some embodiments, the variant comprises an amino acid mutation at G20. In some embodiments, the variant comprises amino acid mutations at K7 and N19. In some embodiments, the variant comprises amino acid mutations at K7 and G20. In some embodiments, the variant comprises amino acid mutations at N19 and G20. In some embodiments, the variant comprises amino acid mutations at Q6 and K7. In some embodiments, the variant comprises amino acid mutations at Q6 and N19. In some embodiments, the variant comprises amino acid mutations at Q6 and G20. In some embodiments, the variant comprises amino acid mutations at K7, N19 and G20. In some embodiments, the variant comprises amino acid mutations at Q6, K7 and N19. In some embodiments, the variant comprises amino acid mutations at Q6, K7 and G20. In some embodiments, the variant comprises amino acid mutations at Q6, N19 and G20. In some embodiments, the variant comprises amino acid mutations at Q6, K7, N19 and G20. In some embodiments, the amino acid mutation at Q6 is an amino acid substitution at Q6. In some embodiments, the amino acid mutation at K7 is an amino acid substitution at K7. In some embodiments, the amino acid mutation at N19 is an amino acid substitution at N19. In some embodiments, the amino acid mutation at G20 is an amino acid substitution at G20. The substitution can change the original amino acid to any other amino acid that is different from the original amino acid. For example, the K7 substitution can change the K residue to any amino acid that is not K. In some embodiments, the variant comprises a N19T substitution. In some embodiments, the variant comprises a N19A substitution. In some embodiments, the variant comprises a Q6G substitution. In some embodiments, the variant comprises a K7G substitution. In some embodiments, the variant comprises a K7G substitution and a N19T substitution. In some embodiments, the variant comprises a K7G substitution and a N19A substitution. In some embodiments, the variant comprises a Q6G substitution, a K7G substitution and a N19A substitution. In some embodiments, the second domain has the amino acid sequence of SEQ ID NO:201. In some embodiments, the second domain has the amino acid sequence of SEQ ID NO:202. In some embodiments, the second domain has the amino acid sequence of SEQ ID NO:203.

In some embodiments, the second domain of the fusion proteins provided herein comprises a variant of the TGFβRII ECD isoform 1 that is either a N-terminal truncated form (i.e., lacks a N-terminal fragment) or a C-terminal truncated form (i.e., lacks C-terminal fragment) of TGFβRII ECD isoform 1. In some embodiments, the second domain of the fusion proteins provided herein comprises a truncated form of the TGFβRII ECD isoform 1 (SEQ ID NO:8) that lacks a N-terminal fragment. In some embodiments, the truncated form of the TGFβRII ECD isoform 1 lacks amino acid residues 1 to n of SEQ ID NO:8, wherein n ranges from 2 to 30, or a variant thereof. In other words, the second domain of the fusion proteins provided herein can comprise a truncated form of the TGFβRII ECD isoform 1 (SEQ ID NO:8) lacking a N-terminal fragment that ranges from 2 to 30 amino acids. In some embodiments, the truncated form of the TGFβRII ECD isoform 1 lacks amino acid residues 1 to 2, 1 to 3, 1 to 4, 1 to 5, 1 to 6, 1 to 7, 1 to 8, 1 to 9, 1 to 10, 1 to 11, 1 to 12, 1 to 13, 1 to 14, 1 to 15, 1 to 16, 1 to 17, 1 to 18, 1 to 19, 1 to 20, 1 to 21, 1 to 22, 1 to 23, 1 to 24, 1 to 25, 1 to 26, 1 to 27, 1 to 28, 1 to 29, or 1 to 30 of SEQ ID NO:8. In some embodiments, n is 19, and the second domain of the fusion proteins provided herein comprises a truncated form of the TGFβRII ECD isoform 1 (SEQ ID NO:8) that lacks amino acid residues 1 to 19 of SEQ ID NO:8, or a variant thereof. In some embodiments, the second domain has the amino acid sequence of SEQ ID NO:10. In some embodiments, the second domain is a truncated form of the TGFβRII ECD isoform 1 (SEQ ID NO:8) that lacks amino acid residues 1 to 24 of SEQ ID NO:8.

In some embodiments, the second domain of the fusion proteins provided herein comprises a truncated form of the TGFβRII ECD isoform 1 (SEQ ID NO:8) that lacks a C-terminal fragment, or a variant thereof. In some embodiments, the truncated form of the TGFβRII ECD isoform 1 lacks amino acid residues m to 136 of SEQ ID NO:8, wherein m ranges from 80 to 135. In other words, the second domain of the fusion proteins provided herein can comprise a truncated form of the TGFβRII ECD isoform 1 (SEQ ID NO:8) lacking a C-terminal fragment that ranges from 1 to 56 amino acids. In some embodiments, the truncated form of the TGFβRII ECD isoform 1 lacks amino acid residues 80 to 136, 81 to 136, 82 to 136, 83 to 136, 84 to 136, 85 to 136, 86 to 136, 87 to 136, 88 to 136, 89 to 136, 90 to 136, 91 to 136, 92 to 136, 93 to 136, 94 to 136, 95 to 136, 96 to 136, 97 to 136, 98 to 136, 99 to 136, 100 to 136, 101 to 136, 102 to 136, 103 to 136, 104 to 136, 105 to 136, 106 to 136, 107 to 136, 108 to 136, 109 to 136, 110 to 136, 111 to 136, 112 to 136, 113 to 136, 114 to 136, 115 to 136, 116 to 136, 117 to 136, 118 to 136, 119 to 136, 120 to 136, 121 to 136, 122 to 136, 123 to 136, 124 to 136, 125 to 136, 126 to 136, 127 to 136, 128 to 136, 129 to 136, 130 to 136, 131 to 136, 132 to 136, 133 to 136, 134 to 136, or 135 to 136 of SEQ ID NO:8. In some embodiments, the truncated form of the TGFβRII ECD isoform 1 (SEQ ID NO:8) lacks a C-terminal fragment that ranges from 15 to 25 amino acids. In some embodiments, the truncated form of the TGFβRII ECD isoform 1 (SEQ ID NO:8) lacks a C-terminal fragment that ranges from 5 to 15 amino acids. In some embodiments, m is 131, and the second domain of the fusion proteins provided herein comprises a truncated form of the TGFβRII ECD isoform 1 (SEQ ID NO:8) that lacks amino acid residues 131 to 136 of SEQ ID NO:8. In some embodiments, the second domain has the amino acid sequence of SEQ ID NO:11. In some embodiments, m is 128, and the second domain of the fusion proteins provided herein comprises a truncated form of the TGFβRII ECD isoform 1 (SEQ ID NO:8) that lacks amino acid residues 128 to 136 of SEQ ID NO:8. In some embodiments, the second domain has the amino acid sequence of SEQ ID NO:12. In some embodiments, the second domain of the fusion proteins provided herein comprises a truncated form of the TGFβRII ECD isoform 1 (SEQ ID NO:8) that lacks amino acid residues 83 to 136 of SEQ ID NO:8.

In some embodiments, the second domain comprises a C-terminal truncated form of the ECD of TGFβRII isoform 1 (SEQ ID NO:8) that further comprises an amino acid mutation at K7, N19 or G20, or any combination thereof. In some embodiments, the truncated form further comprises an amino acid mutation at Q6. In some embodiments, the truncated form further comprises an amino acid mutation at K7. In some embodiments, the truncated form further comprises an amino acid mutation at N19. In some embodiments, the truncated form further comprises an amino acid mutation at G20. In some embodiments, the truncated form further comprises amino acid mutations at Q6 and K7. In some embodiments, the truncated form further comprises amino acid mutations at K7 and N19. In some embodiments, the truncated form further comprises amino acid mutations at K7 and G20. In some embodiments, the truncated form further comprises amino acid mutations at N19 and G20. In some embodiments, the truncated form further comprises amino acid mutations at Q6 and N19. In some embodiments, the truncated form further comprises amino acid mutations at Q6 and G20. In some embodiments, the truncated form further comprises amino acid mutations at Q6, K7, and N19. In some embodiments, the truncated form further comprises amino acid mutations at Q6, K7, and G20. In some embodiments, the truncated form further comprises amino acid mutations at Q6, N19 and G20.In some embodiments, the truncated form further comprises amino acid mutations at K7, N19 and G20. In some embodiments, the truncated form further comprises amino acid mutations at Q6, K7, N19 and G20. In some embodiments, the amino acid mutation at Q6 is an amino acid substitution at Q6. In some embodiments, the amino acid mutation at K7 is an amino acid substitution at K7. In some embodiments, the amino acid mutation at N19 is an amino acid substitution at N19. In some embodiments, the amino acid mutation at G20 is an amino acid substitution at G20. The substitution can change the original amino acid to any other amino acid that is different from the original amino acid. For example, the K7 substitution can change the K residue to any amino acid that is not K. In some embodiments, the truncated form further comprises a K7G substitution. In some embodiments, the truncated form further comprises a N19T substitution. In some embodiments, the truncated form further comprises a N19A substitution. In some embodiments, the truncated form further comprises a Q6G substitution. In some embodiments, the truncated form further comprises a K7G substitution and a N19T substitution. In some embodiments, the truncated form further comprises a K7G substitution and a N19A substitution. In some embodiments, the truncated form further comprises a Q6G substitution, a K7G substitution and a N19A substitution. In some embodiments, the second domain has the amino acid sequence of SEQ ID NO:204. In some embodiments, the second domain has the amino acid sequence of SEQ ID NO:205. In some embodiments, the second domain has the amino acid sequence of SEQ ID NO:232.

In some embodiments, the second domain of the fusion proteins provided herein comprises a variant of the TGFβRII ECD isoform 2 having at least 85%, at least 90%, at least 95%, or at least 98% sequence identity to SEQ ID NO:14. In some embodiments, the second domain of the fusion proteins provided herein has at least 85% sequence identity to SEQ ID NO:14. In some embodiments, the second domain of the fusion proteins provided herein has at least 90% sequence identity to SEQ ID NO:14. In some embodiments, the second domain of the fusion proteins provided herein has at least 95% sequence identity to SEQ ID NO:14. In some embodiments, the second domain of the fusion proteins provided herein has at least 98% sequence identity to SEQ ID NO:14.

In some embodiments, the second domain of the fusion proteins provided herein comprises a variant of the TGFβRII ECD isoform 2 that is either a N-terminal truncated form (i.e., lacks a N-terminal fragment) or a C-terminal truncated form (i.e., lacks C-terminal fragment) of TGFβRII ECD isoform 2. In some embodiments, the second domain of the fusion proteins provided herein comprises a truncated form of the TGFβRII ECD isoform 2 (SEQ ID NO:14) lacking a N-terminal fragment. In some embodiments, the truncated form of the TGFβRII ECD isoform 1 lacks amino acid residues 1 to n of SEQ ID NO:14, wherein n ranges from 2 to 60. In some embodiments, the truncated form of the TGFβRII ECD isoform 2 lacks amino acid residues 1 to 2, 1 to 3, 1 to 4, 1 to 5, 1 to 6, 1 to 7, 1 to 8, 1 to 9, 1 to 10, 1 to 11, 1 to 12, 1 to 13, 1 to 14, 1 to 15, 1 to 16, 1 to 17, 1 to 18, 1 to 19, 1 to 20, 1 to 21, 1 to 22, 1 to 23, 1 to 24, 1 to 25, 1 to 26, 1 to 27, 1 to 28, 1 to 29, 1 to 30, 1 to 31, 1 to 32, 1 to 33, 1 to 34, 1 to 35, 1 to 36, 1 to 37, 1 to 38, 1 to 39, 1 to 40, 1 to 41, 1 to 42, 1 to 43, 1 to 44, 1 to 45, 1 to 46, 1 to 47, 1 to 48, 1 to 49, 1 to 50, 1 to 51, 1 to 52, 1 to 53, 1 to 54, 1 to 55, 1 to 56, 1 to 57, 1 to 58, 1 to 59, or 1 to 60 of SEQ ID NO:14. In some embodiments, the second domain of the fusion proteins provided herein comprises a truncated form of the TGFβRII ECD isoform 2 (SEQ ID NO:14) lacking amino acid residues 1 to 44 of SEQ ID NO:14. In some embodiments, the second domain has the amino acid sequence of SEQ ID NO:15. In some embodiments, the second domain of the fusion proteins provided herein comprises a truncated form of the TGFβRII ECD isoform 2 (SEQ ID NO:14) lacking amino acid residues 1 to 49 of SEQ ID NO:14.

In some embodiments, the second domain of the fusion proteins provided herein comprises a variant of the TGFβRII ECD isoform 2 (SEQ ID NO:14) lacking a C-terminal fragment. In some embodiments, the truncated form of the TGFβRII ECD isoform 2 lacks amino acid residues m to 161 of SEQ ID NO:14, wherein m ranges from 100 to 160. In some embodiments, the truncated form of the TGFβRII ECD isoform 1 lacks amino acid residues 100 to 161, 101 to 161, 102 to 161, 103 to 161, 104 to 161, 105 to 161, 106 to 161, 107 to 161, 108 to 161, 109 to 161, 110 to 161, 111 to 161, 112 to 161, 113 to 161, 114 to 161, 115 to 161, 116 to 161, 117 to 161, 118 to 161, 119 to 161, 120 to 161, 121 to 161, 122 to 161, 123 to 161, 124 to 161, 125 to 161, 126 to 161, 127 to 161, 128 to 161, 129 to 161, 130 to 161, 131 to 161, 132 to 161, 133 to 161, 134 to 161, 135 to 161, 136 to 161, 137 to 161, 138 to 161, 139 to 161, 140 to 161, 141 to 161, 142 to 161, 143 to 161, 144 to 161, 145 to 161, 146 to 161, 147 to 161, 148 to 161, 149 to 161, 150 to 161, 151 to 161, 152 to 161, 153 to 161, 154 to 161, 155 to 161, 156 to 161, 157 to 161, 158 to 161, 159 to 161, or 160 to 161 of SEQ ID NO: 14. In some embodiments, the second domain of the fusion proteins provided herein comprises a truncated form of the TGFβRII ECD isoform 2 (SEQ ID NO: 14) that lacks amino acid residues 128 to 161 of SEQ ID NO:14. In some embodiments, the second domain has the amino acid sequence of SEQ ID NO:16.

Provided herein are fusion proteins having an anti-PD-L1 domain and a TGFβ trap domain. In some embodiments, the two domains are linked via a linker. The linker can be a rigid linker (e.g., a transferrin linker). The TGFβ trap domain can be a domain that comprises any of the TGFβRII fragments that bind TGFβ disclosed above or otherwise known in the art. The anti-PD-L1 domain can also be an anti-PD-L1 antibody disclosed in the section below. The anti-PD-L1 domain can also be any anti-PD-L1 antibody known in the art (e.g., Akinleye & Zoaib Rasool, Journal of Hematology & Oncology volume 12:92 (2019)). For example, in some embodiments, the anti-PD-L1 domain of the fusion proteins provided herein comprise the anti-PD-L1 antibody selected from the group consisting of durvalumab (AstraZeneca), avelumab (MercK KGaA/Pfizer), atezolizumab (Roche/Genentech), envafolimab (Alphamab Oncology), BMS-936559 (Bristol-Myers Squibb), CK-301(Checkpoint Therapeutics), CS-1001 (CStone Pharmaceuticals), SHR-1316 (HTI-1088; Hengrui Therapeutics), and BGB-A333 (BeiGene). In some embodiments, the anti-PD-L1is durvalumab. In some embodiments, the anti-PD-L1 is avelumab. In some embodiments, the anti-PD-L1is atezolizumab. In some embodiments, the fusion protein comprises avelumab and a TGFβ trap domain linked by a rigid linker. In some embodiments, the fusion protein has a heavy chain and a light chain, wherein the amino acid sequence of the heavy chain is SEQ ID NO:242 and the amino acid sequence of the light chain is SEQ ID NO:243.

Provided herein are fusion proteins having an anti-PD-L1 domain and a TGFβ trap domain. In some embodiments, the two domains are linked via a linker. The linker can be a rigid linker (e.g., a transferrin linker). Different combinations and permutations of the anti-PD-L1 domain, the linker, and the TGFβ trap domain are contemplated herein. For illustrative purposes, in some embodiments, provided herein are fusions proteins having an anti-PD-L1 antibody, a transferrin linker (SEQ ID NO:18 or 19), and an ECD of TGFβRII, or a variant thereof. In some embodiments, the transferrin linker has the amino acid sequence of SEQ ID NO: 147. In some embodiments, the TGFβ trap domain is a variant of the ECD of TGFβRII isoform 1 that lacks a C-terminal fragment of the ECD. In some embodiments, the TGFβ trap domain has the amino acid sequence of SEQ ID NO:11. In some embodiments, the TGFβ trap domain has the amino acid sequence of SEQ ID NO:12.

In some embodiments, the first domain of the fusion proteins provided herein comprises an anti-PD-L1 antibody or antigen-binding fragment which is a bispecific antibody that binds a second target molecule. In some embodiments, the second target molecule is a membrane protein. In some embodiments, the second target molecule is an immune checkpoint protein. In some embodiments, the second target molecule is selected from the group consisting of HER2, HER3, CD33, VEGF, VEGFR, VEGFR-2, CD152, TNF, IL-1, IL-5, IL-17, IL-6R, IL-2R, BLYS, PCSK9, EGFR, c-Met, CD2, CD3, CDlla, CD19, CD30, CD38, CD20, CD52, CD60, CD80, CD86, TNF-α, IL-12, IL-17, IL-23, IL-6, RSVF, IgE, RANK, BLyS, α4β7, PD-1, CCR4, SLAMF7, GD2, CD21, CD79b, IL20Ra, CD22, CD79a, CD72, and IGF-IRRANKL. In some embodiments, the first domain of the fusion proteins provided herein comprises a bispecific antibody that binds PD-L1 and a second target molecule selected from the group consisting of HER2, HER3, CD33, VEGF, VEGFR, VEGFR-2, CD152, TNF, IL-1, IL-5, IL-17, IL-6R, IL-2R, BLYS, PCSK9, EGFR, c-Met, CD2, CD3, CDlla, CD19, CD30, CD38, CD20, CD52, CD60, CD80, CD86, TNF-α, IL-12, IL-17, IL-23, IL-6, RSVF, IgE, RANK, BLyS, α4β7, PD-1, CCR4, SLAMF7, GD2, CD21, CD79b, IL20Ra, CD22, CD79a, CD72, and IGF-IRRANKL.

### 3. Anti-PD-L1

Human PD-L1 (also known as CD274, PDCD1L1, or B7-H1) is a cell membrane immune checkpoint protein expressed by many cancers, which can upon binding to PD-1, promotes evasion of T-cell immunity and induces cancer cells progression. The amino acid sequence of human PD-L1 is shown below.

MRIFAVFIFM TYWHLLNAFT VTVPKDLYVV EYGSNMTIEC KFPVEK
QLDL AALIVYWEMEDKNIIQFVHG EEDLKVQHSS YRQRARLLKD QL
SLGNAALQ ITDVKLQDAG VYRCMISYGGADYKRITVKV NAPYNKINQ
R ILVVDPVTSE HELTCQAEGY PKAEVIWTSS DHQVLSGKTTTTNSK
REEKL FNVTSTLRIN TTTNEIFYCT FRRLDPEENH TAELVIPELP
LAHPPNERTHLVILGAILLC LGVALTFIFR LRKGRMMDVK KCGIQDT
NSK KQSDTHLEET (SEQ ID NO:1)

Also provided herein are antibodies or antigen-binding fragments thereof that bind PD-L1 (e.g., human PD-L1). The anti-PD-L1 antibodies or antigen-binding fragments thereof provided herein can reduce or block the PD-1/PD-L1 checkpoint signaling and thereby promote T cell proliferation and activation. Glycosylation of PD-L1 expressed on tumor cells can promote or enhance binding to PD-1 expressed on immune effector cells, such as T cells, and increase the suppression of T cell activity against the tumor cells. Glycosylation of PD-L1 can also stabilize PD-L1 expression on the cell surface, thus reducing the rate of internalization and intracellular degradation of the PD-L1.

In some embodiments, antibodies or antigen-binding fragments thereof provided herein can bind glycosylated PD-L1. In some embodiments, the antibodies or antigen-binding fragments thereof provided herein inhibit PD-1/PD-L1 binding and promote PD-L1 internalization and degradation by targeting glycosylated PD-L1. In some embodiments, the antibodies or antigen-binding fragments thereof provided herein can bind heavily glycosylated PD-L1. In some embodiments, the antibodies or antigen-binding fragments thereof provided herein can bind modestly glycosylated PD-L1. In some embodiments, the antibodies or antigen-binding fragments thereof provided herein can bind both heavily glycosylated PD-L1 and modestly glycosylated PD-L1. PD-L1 is glycosylated at four sites in the extracellular domain at amino acid positions N35, N192, N200 and/or N219 of the human PD-L1 protein. In some embodiments, the antibodies or antigen-binding fragments thereof provided herein can bind human PD-L1 glycosylated at one, two, three or four of the four residues (i.e., N35, N192, N200 and/or N219). For example, in some embodiments, antibodies or antigen-binding fragments thereof provided herein can bind human PD-L1 glycosylated at N35; N192; N200; or N219. In some embodiments, antibodies or antigen-binding fragments thereof provided herein can bind human PD-L1 glycosylated at N35 and N192; N35 and N200; N35 and N219; N192 and N200; N192 and N219; N200 and N219. In some embodiments, antibodies or antigen-binding fragments thereof provided herein can bind human PD-L1 glycosylated at N35, N192, and N200; N35, N192, and N219; N35, N200, and N219; or N192, N200, and N219. In some embodiments, antibodies or antigen-binding fragments thereof provided herein can bind human PD-L1 glycosylated at N35, N192, N200, and N219.

In some embodiments, antibodies or antigen-binding fragments provided herein can be a single domain antibody (sdAb), a heavy chain antibody (HCAb), a Fab, a Fab′, a F(ab′)₂, a Fv, a scFv,or a (scFv)₂. In some embodiments, the antibodies provided herein can be an IgG1 antibody, an IgG2 antibody, an IgG3 antibody, or an IgG4 antibody.

In some embodiments, provided herein are anti-PD-L1 antibodies. In some embodiments, the antibody is an IgA, IgD, IgE, IgG, or IgM antibody. In some embodiments, the antibody is an IgA antibody. In some embodiments, the antibody is an IgD antibody. In some embodiments, the antibody is an IgE antibody. In some embodiments, the antibody is an IgG antibody. In some embodiments, the antibody is an IgM antibody. In some embodiments, the antibody is an IgG1 antibody. In some embodiments, the antibody is an IgG2 antibody. In some embodiments, the antibody is an IgG3 antibody. In some embodiments, the antibody is an IgG4 antibody.

In some embodiments, provided herein are antigen-binding fragments of an anti-PD-L1 antibody. In some embodiments, the antigen-binding fragment of an anti-PD-L1 antibody is a single domain antibody (sdAb). In some embodiments, the antigen-binding fragment of an anti-PD-L1 antibody is a heavy chain antibody (HCAb). In some embodiments, the antigen-binding fragment of an anti-PD-L1 antibody is a Fab. In some embodiments, the antigen-binding fragment of an anti-PD-L1 antibody is a Fab′. In some embodiments, the antigen-binding fragment of an anti-PD-L1 antibody is a F(ab′)₂. In some embodiments, the antigen-binding fragment of an anti-PD-L1 antibody is a Fv. In some embodiments, the antigen-binding fragment of an anti-PD-L1 antibody is a scFv. In some embodiments, the antigen-binding fragment of an anti-PD-L1 antibody is a disulfide-linked scFv [(scFv)₂]. In some embodiments, the antigen-binding fragment of an anti-PD-L1 antibody is a diabody (dAb).

In some embodiments, the anti-PD-L1 antibodies or antigen-binding fragments provided herein comprise recombinant antibodies or antigen-binding fragments. In some embodiments, the anti-PD-L1 antibodies or antigen-binding fragments provided herein comprise monoclonal antibodies or antigen-binding fragments. In some embodiments, the anti-PD-L1 antibodies or antigen-binding fragments provided herein comprise polyclonal antibodies or antigen-binding fragments. In some embodiments, the anti-PD-L1 antibodies or antigen-binding fragments provided herein comprise camelid (e.g., camels, dromedary and llamas) antibodies or antigen-binding fragments. In some embodiments, the anti-PD-L1 antibodies or antigen-binding fragments provided herein comprise chimeric antibodies or antigen-binding fragments. In some embodiments, the anti-PD-L1 antibodies or antigen-binding fragments provided herein comprise humanized antibodies or antigen-binding fragments. In some embodiments, the anti-PD-L1 antibodies or antigen-binding fragments provided herein comprise human antibodies or antigen-binding fragments.

In some embodiments, the anti-PD-L1 antibodies or antigen-binding fragments provided herein are isolated. In some embodiments, the anti-PD-L1 antibodies or antigen-binding fragments provided herein are substantially pure.

In some embodiments, the anti-PD-L1 antibody or antigen-binding fragment provided herein comprises a bispecific antibody or antigen-binding fragment. In some embodiments, the anti-PD-L1 antibody or antigen-binding fragment provided herein comprises a multispecific antibody or antigen-binding fragment.

In some embodiments, the anti-PD-L1 antibody or antigen-binding fragment provided herein comprises a monovalent antigen-binding site. In some embodiments, an anti-PD-L1 antibody or antigen-binding fragment comprises a monospecific binding site. In some embodiments, an anti-PD-L1 antibody or antigen-binding fragment comprises a bivalent binding site.

In some embodiments, an anti-PD-L1 antibody or antigen-binding fragment is a monoclonal antibody or antigen-binding fragment. Monoclonal antibodies can be prepared by any method known to those of skill in the art. One exemplary approach is screening protein expression libraries, e.g., phage or ribosome display libraries. Phage display is described, for example, in Ladner et al., U.S. Pat. No. 5,223,409; Smith (1985) Science 228:1315-1317; and WO 92/18619. In some embodiments, recombinant monoclonal antibodies are isolated from phage display libraries expressing variable regions or CDRs of a desired species. Screening of phage libraries can be accomplished by various techniques known in the art.

In some embodiments, monoclonal antibodies are prepared using hybridoma methods known to one of skill in the art. For example, using a hybridoma method, a mouse, rat, rabbit, hamster, or other appropriate host animal, is immunized as described above. In some embodiments, lymphocytes are immunized in vitro. In some embodiments, the immunizing antigen is a human protein or a fragment thereof. In some embodiments, the immunizing antigen is a human protein or a fragment thereof.

Following immunization, lymphocytes are isolated and fused with a suitable myeloma cell line using, for example, polyethylene glycol. The hybridoma cells are selected using specialized media as known in the art and unfused lymphocytes and myeloma cells do not survive the selection process. Hybridomas that produce monoclonal antibodies directed to a chosen antigen can be identified by a variety of methods including, but not limited to, immunoprecipitation, immunoblotting, and in vitro binding assays (e.g., flow cytometry, FACS, ELISA, SPR (e.g., Biacore), and radioimmunoassay). Once hybridoma cells that produce antibodies of the desired specificity, affinity, and/or activity are identified, the clones may be subcloned by limiting dilution or other techniques. The hybridomas can be propagated either in in vitro culture using standard methods or in vivo as ascites tumors in an animal. The monoclonal antibodies can be purified from the culture medium or ascites fluid according to standard methods in the art including, but not limited to, affinity chromatography, ion-exchange chromatography, gel electrophoresis, and dialysis.

In some embodiments, monoclonal antibodies are made using recombinant DNA techniques as known to one skilled in the art. For example, the polynucleotides encoding an antibody are isolated from mature B-cells or hybridoma cells, such as by RT-PCR using oligonucleotide primers that specifically amplify the genes encoding the heavy and light chains of the antibody, and their sequence is determined using standard techniques. The isolated polynucleotides encoding the heavy and light chains are then cloned into suitable expression vectors which produce the monoclonal antibodies when transfected into host cells such as E. coli, simian COS cells, Chinese hamster ovary (CHO) cells, or myeloma cells that do not otherwise produce immunoglobulin proteins.

In some embodiments, recombinant monoclonal antibodies are isolated from phage display libraries expressing variable regions or CDRs of a desired species. Screening of phage libraries can be accomplished by various techniques known in the art.

In some embodiments, a monoclonal antibody is modified by using recombinant DNA technology to generate alternative antibodies. In some embodiments, the constant domains of the light chain and heavy chain of a mouse monoclonal antibody are replaced with the constant regions of a human antibody to generate a chimeric antibody. In some embodiments, the constant regions are truncated or removed to generate a desired antibody fragment of a monoclonal antibody. In some embodiments, site-directed or high-density mutagenesis of the variable region(s) is used to optimize specificity and/or affinity of a monoclonal antibody.

In some embodiments, an anti-PD-L1 antibody or antigen-binding fragment is a humanized antibody or antigen-binding fragment. Various methods for generating humanized antibodies are known in the art. In some embodiments, a humanized antibody comprises one or more amino acid residues that have been introduced into its sequence from a source that is non-human. In some embodiments, humanization is performed by substituting one or more non-human CDR sequences for the corresponding CDR sequences of a human antibody. In some embodiments, the humanized antibodies are constructed by substituting all three CDRs of a non-human antibody (e.g., a heavy chain or light chain antibody) for the corresponding CDRs of a human antibody. In some embodiments, the humanized antibodies are constructed by substituting all six CDRs of a non-human antibody (e.g., a mouse antibody) for the corresponding CDRs of a human antibody.

The choice of which human heavy chain variable region and/or light chain variable region are used for generating humanized antibodies can be made based on a variety of factors and by a variety of methods known in the art. In some embodiments, a particular variable region framework derived from a consensus sequence of all human antibodies of a particular subgroup of light or heavy chains is selected as the variable region framework. In some embodiments, the variable region framework sequence is derived from the consensus sequences of the most abundant human subclasses. In some embodiments, human germline genes are used as the source of the variable region framework sequences.

Methods are known in the art for achieving high affinity binding with humanized antibodies. A non-limiting example of such a method is hypermutation of the variable region and selection of the cells expressing such high affinity antibodies (affinity maturation). In addition to the use of display libraries, the specified antigen (e.g., recombinant PD-L1 or an epitope thereof) can be used to immunize a non-human animal, e.g., a rodent. In certain embodiments, rodent antigen-binding fragments (e.g., mouse antigen-binding fragments) can be generated and isolated using methods known in the art and/or disclosed herein. In some embodiments, a mouse can be immunized with an antigen (e.g., recombinant PD-L1 or an epitope thereof).

In some embodiments, an anti-PD-L1 antibody or antigen-binding fragment is a human antibody or antigen-binding fragment. Human antibodies can be prepared using various techniques known in the art. In some embodiments, human antibodies are generated from immortalized human B lymphocytes immunized in vitro. In some embodiments, human antibodies are generated from lymphocytes isolated from an immunized individual. In any case, cells that produce an antibody directed against a target antigen can be generated and isolated. In some embodiments, a human antibody is selected from a phage library, where that phage library expresses human antibodies. Alternatively, phage display technology may be used to produce human antibodies and antibody fragments in vitro, from immunoglobulin variable region gene repertoires from unimmunized donors. Techniques for the generation and use of antibody phage libraries are well-known in the art. Once antibodies are identified, affinity maturation strategies known in the art, including but not limited to, chain shuffling and site-directed mutagenesis, may be employed to generate higher affinity human antibodies. In some embodiments, human antibodies are produced in transgenic mice that contain human immunoglobulin loci. Upon immunization these mice are capable of producing the full repertoire of human antibodies in the absence of endogenous immunoglobulin production.

CDRs of an antibody are defined by those skilled in the art using a variety of methods/systems. These systems and/or definitions have been developed and refined over years and include Kabat, Chothia, IMGT, AbM, and Contact. The Kabat definition is based on sequence variability and is commonly used. The Chothia definition is based on the location of the structural loop regions. The IMGT system is based on sequence variability and location within the structure of the variable regions. The AbM definition is a compromise between Kabat and Chothia. The Contact definition is based on analyses of the available antibody crystal structures. An Exemplary system is a combination of Kabat and Chothia. Software programs (e.g., abYsis) are available and known to those of skill in the art for analysis of antibody sequence and determination of CDRs.

The specific CDR sequences defined herein are generally based on a combination of Kabat and Chothia definitions (exemplary system). However, it will be understood that reference to a heavy chain CDR or CDRs and/or a light chain CDR or CDRs of a specific antibody will encompass all CDR definitions as known to those of skill in the art.

Anti-PD-L1 antibodies or antigen-binding fragments provided herein include Clone A, Clone B, Clone C, Clone D, Clone F, Clone G, Clone H, Clone J, Clone K, Clone L, Clone N, Clone P, and Clone Y as disclosed below, and humanized versions thereof.

In some embodiments, anti-PD-L1 antibodies or antigen-binding fragments provided herein comprise one, two, three, four, five, and/or six CDRs of any one of the antibodies described herein. In some embodiments, anti-PD-L1 antibodies or antigen-binding fragments provided herein comprise one, two, and/or three, VH CDRs from Table 1. In some embodiments, anti-PD-L1 antibodies or antigen-binding fragments provided herein comprise one, two, and/or three VL CDRs from Table 2. In some embodiments, anti-PD-L1 antibodies or antigen-binding fragments provided herein comprise one, two, and/or three VH CDRs from Table 1 and one, two, and/or three VL CDRs from Table 2.

TABLE 1

Amino acid sequences of heavy chain variable region CDRs (VH CDRs) of anti-PD-L1 antibodies

| | | | |
|---|---|---|---|
| Antibody | VH CDR1 | VH CDR2 | VH CDR3 |
| Clone A | DTYMH (SEQ ID NO:20) | RIDPANGDTKYDPKFQG (SEQ ID NO:21) | PRLVRFPYVMDY (SEQ ID NO:22) |
| Clone B | SSYIS (SEQ ID NO:26) | WIYAGTGGTSYNQKFTG (SEQ ID NO:27) | HEGKYWYFDV (SEQ ID NO:28) |
| Clone C | DTYMH (SEQ ID NO:32) | RIDPANGNTKYDPKFQG (SEQ ID NO:33) | GLGRWFAY (SEQ ID NO:34) |
| Clone D | GYTMN (SEQ ID NO:38) | LIIPYNGGISYNQKFKD (SEQ ID NO:39) | LITTAPRDSMDY (SEQ ID NO:40) |
| Clone F | SGYWN (SEQ ID NO:44) | YISYSGSTYCIPSLKS (SEQ ID NO:45) | GNWGREAWFAY (SEQ ID NO:46) |
| Clone G | STYIS (SEQ ID NO:50) | WIYAGTGGTSYNQKFTG (SEQ ID NO:51) | HYGTYWYFDV (SEQ ID NO:52) |
| Clone H | SSTYIS (SEQ ID NO:56) | WIYAGTGGTSYNQKFTG (SEQ ID NO:57) | HIGNYWYFDI (SEQ ID NO:58) |
| Clone J | DYWMH (SEQ ID NO:62) | AVDTSDRYTTYNQKFRG (SEQ ID NO:63) | GLTGPYYYPMDF (SEQ ID NO:64) |
| Clone K | GYTMN (SEQ ID NO:68) | LIIPHNGGTSYNQKFKD (SEQ ID NO:69) | LMTTAPRDSMDY (SEQ ID NO:70) |
| Clone L | GYTMN (SEQ ID NO:74) | LIIPSNGGTNYNQRFKD (SEQ ID NO:75) | LITTAPRDSMDY (SEQ ID NO:76) |
| Clone N | GYTMN (SEQ ID NO:80) | LIIPHNGGTSYNQKFKD (SEQ ID NO:81) | LMTTAPRDSMDY (SEQ ID NO:82) |
| Clone P | SGYWN (SEQ ID NO:86) | YISYTGSTYHTPSLKG (SEQ ID NO:87) | GNWGREAWFPY (SEQ ID NO:88) |
| Clone Y | SYWMH (SEQ ID NO:92) | MIDPSDSETKLNQKFRD (SEQ ID NO:93) | DYRYDVGAMDY (SEQ ID NO:94) |

TABLE 2

Amino acid sequences of light chain variable region CDRs ((VL CDRs) of anti-PD-L1 antibodies

| | | | |
|---|---|---|---|
| Antibody | VL CDR1 | VL CDR2 | VL CDR3 |
| Clone A | RASENVYSYLA (SEQ ID NO:23) | NAKTLAE (SEQ ID NO:24) | QHHYGFPYT (SEQ ID NO:25) |
| Clone B | SASSSVSYVH (SEQ ID NO:29) | DTSNLAS (SEQ ID NO:30) | HQRSSYPWT (SEQ ID NO:31) |
| Clone C | KASQDVSNAVA (SEQ ID NO:35) | SASNRYT (SEQ ID NO:36) | QQHDSTPLT (SEQ ID NO:37) |
| Clone D | RASQDISNYLN (SEQ ID NO:41) | YTSKLHS (SEQ ID NO:42) | QQGDALPWT (SEQ ID NO:43) |
| Clone F | RASENIYSYLA (SEQ ID NO:47) | NVKILAE (SEQ ID NO:48) | QHHYGLPYT (SEQ ID NO:49) |
| Clone G | KASQDVSTAVA (SEQ ID NO:53) | SASYRYT (SEQ ID NO:54) | LQHYSTPWT (SEQ ID NO:55) |
| Clone H | SASSSVSYIH (SEQ ID NO:59) | DTSNLAS (SEQ ID NO:60) | HQRSSYPWT (SEQ ID NO:61) |
| Clone J | LASQTIGTWLA (SEQ ID NO:65) | AATSLAD (SEQ ID NO:66) | QQVFSSPYT (SEQ ID NO:67) |
| Clone K | LASQTIGTWLA (SEQ ID NO:71) | AATSLAD (SEQ ID NO:72) | QQLFSTPWT (SEQ ID NO:73) |
| Clone L | RASQDISNYLN (SEQ ID NO:77) | YTSKLHS (SEQ ID NO:78) | QQGDALPWT (SEQ ID NO:79) |
| Clone N | RASQDISNYLN (SEQ ID NO:83) | YTSRLHS (SEQ ID NO:84) | QQGATLPWT (SEQ ID NO:85) |
| Clone P | RASESIYSYLA (SEQ ID NO:89) | NAKTLAE (SEQ ID NO:90) | QHHYGFPYT (SEQ ID NO:91) |
| Clone Y | RASKSISKFLA (SEQ ID NO:95) | SGSTLQS (SEQ ID NO:96) | QQHHEYPWT (SEQ ID NO:97) |

In some embodiments, an anti-PD-L1 antibody or antigen-binding fragment thereof comprises a humanized antibody or antigen-binding fragment. In some embodiments, an anti-PD-L1 antibody or antigen-binding fragment thereof comprises an antibody or antigen-binding fragment having a VH CDR1, VH CDR2, VH CDR3, VL CDR1, VL CDR2, and/or VL CDR3 from an antibody or antigen-binding fragment described herein. In some embodiments, an anti-PD-L1 antibody or antigen-binding fragment thereof comprises a humanized version of an antibody described herein. In some embodiments, an anti-PD-L1 antibody or antigen-binding fragment thereof comprises a variant of an anti-PD-L1 antibody or antigen-binding fragment described herein. In some embodiments, a variant of the anti-PD-L1 antibody or antigen-binding fragment comprises one to 30 amino acid substitutions, additions, and/or deletions. In some embodiments, a variant of the anti-PD-L1 antibody or antigen-binding fragment comprises one to 25 amino acid substitutions, additions, and/or deletions. In some embodiments, a variant of the anti-PD-L1 antibody or antigen-binding fragment comprises one to 20 substitutions, additions, and/or deletions. In some embodiments, a variant of the anti-PD-L1 antibody or antigen-binding fragment comprises one to 15 substitutions, additions, and/or deletions. In some embodiments, a variant of the anti-PD-L1 antibody or antigen-binding fragment comprises one to 10 substitutions, additions, and/or deletions. In some embodiments, a variant of the anti-PD-L1 antibody or antigen-binding fragment comprises one to five conservative amino acid substitutions, additions, and/or deletions. In some embodiments, a variant of the anti-PD-L1 antibody or antigen-binding fragment comprises one to three amino acid substitutions, additions, and/or deletions. In some embodiments, the amino acid substitutions, additions, and/or deletions are conservative amino acid substitutions. In some embodiments, the conservative amino acid substitution(s) is in a CDR of the antibody or antigen-binding fragment. In some embodiments, the conservative amino acid substitution(s) is not in a CDR of the antibody or antigen-binding fragment. In some embodiments, the conservative amino acid substitution(s) is in a framework region of the antibody or antigen-binding fragment.

In some embodiments, provided herein are antibodies or antigen-binding fragments thereof that bind PD-L1, comprising a heavy chain variable region (VH) comprising (1) a heavy chain CDR1 (VH CDR1) having an amino acid sequence selected from the group consisting of SEQ ID NOs:20, 26, 32, 38, 44, 50, 56, 62, 68, 74, 80, 86, and 92; (2) a heavy chain CDR2 (VH CDR2) having an amino acid sequence selected from the group consisting of SEQ ID NOs:21, 27, 33, 39, 45, 51, 57, 63, 69, 75, 81, 87 and 93; or (3) a heavy chain CDR3 (VH CDR3) having an amino acid sequence selected from the group consisting of SEQ ID NOs:22, 28, 34, 40, 46, 52, 58, 64, 70, 76, 82, 89 and 94; or a variant thereof having up to about 3, about 5, about 8, about 10, about 12, or about 15 amino acid substitutions, additions, and/or deletions in the VH CDRs. In some embodiments, the variant has about 5 amino acid substitutions, additions, and/or deletions in the VH CDRs.

In some embodiments, provided herein are antibodies or antigen-binding fragments thereof that bind PD-L1, comprising a VH comprising (1) a VH CDR1 having an amino acid sequence selected from the group consisting of SEQ ID NOs:20, 26, 32, 38, 44, 50, 56, 62, 68, 74, 80, 86, and 92; (2) a VH CDR2 having an amino acid sequence selected from the group consisting of SEQ ID NOs:21, 27, 33, 39, 45, 51, 57, 63, 69, 75, 81, 87 and 93; and (3) a VH CDR3 having an amino acid sequence selected from the group consisting of SEQ ID NOs:22, 28, 34, 40, 46, 52, 58, 64, 70, 76, 82, 89 and 94; or a variant thereof having up to about 3, about 5, about 8, about 10, about 12, or about 15 amino acid substitutions, additions, and/or deletions in the VH CDRs. In some embodiments, the variant has up to about 5 amino acid substitutions, additions, and/or deletions in the VH CDRs.

In some embodiments, provided herein are antibodies or antigen-binding fragments thereof that bind PD-L1 comprising a light chain variable region (VL) comprising (1) a light chain CDR1 (VL CDR1) having an amino acid sequence selected from the group consisting of SEQ ID NOs:23, 29, 35, 41, 47, 53, 59, 65, 71, 77, 83, 89, and 95; (2) a light chain CDR2 (VL CDR2) having an amino acid sequence selected from the group consisting of SEQ ID NOs:24, 30, 36, 42, 48, 54, 60, 66, 72, 78, 84, 90, and 96; or (3) a light chain CDR3 (VL CDR3) having an amino acid sequence selected from the group consisting of SEQ ID NOs:25, 31, 37, 43, 49, 55, 61, 67, 63, 79, 85, 91, and 97; or a variant thereof having up to about 3, about 5, about 8, about 10, about 12, or about 15 amino acid substitutions, additions, and/or deletions in the VL CDRs. In some embodiments, the variant has up about 5 amino acid substitutions, additions, and/or deletions in the VL CDRs.

In some embodiments, provided herein are antibodies or antigen-binding fragments thereof that bind PD-L1 comprising a VL comprising (1) a VL CDR1 having an amino acid sequence selected from the group consisting of SEQ ID NOs:23, 29, 35, 41, 47, 53, 59, 65, 71, 77, 83, 89, and 95; (2) a VL CDR2 having an amino acid sequence selected from the group consisting of SEQ ID NOs:24, 30, 36, 42, 48, 54, 60, 66, 72, 78, 84, 90, and 96; and (3) a VL CDR3 having an amino acid sequence selected from the group consisting of SEQ ID NOs:25, 31, 37, 43, 49, 55, 61, 67, 63, 79, 85, 91, and 97; or a variant thereof having up to about 3, about 5, about 8, about 10, about 12, or about 15 amino acid substitutions, additions, and/or deletions in the VL CDRs. In some embodiments, the variant has up about 5 amino acid substitutions, additions, and/or deletions in the VL CDRs.

In some embodiments, provided herein are antibodies or antigen-binding fragments thereof that bind PD-L1, comprising (a) a VH comprising (1) a VH CDR1 having an amino acid sequence selected from the group consisting of SEQ ID NOs:20, 26, 32, 38, 44, 50, 56, 62, 68, 74, 80, 86, and 92; (2) a VH CDR2 having an amino acid sequence selected from the group consisting of SEQ ID NOs:21, 27, 33, 39, 45, 51, 57, 63, 69, 75, 81, 87 and 93; and (3) a VH CDR3 having an amino acid sequence selected from the group consisting of SEQ ID NOs:22, 28, 34, 40, 46, 52, 58, 64, 70, 76, 82, 89 and 94; or a variant thereof having up to about 5 amino acid substitutions, additions, and/or deletions in the VH CDRs; and (b) a VL comprising (1) a VL CDR1 having an amino acid sequence selected from the group consisting of SEQ ID NOs:23, 29, 35, 41, 47, 53, 59, 65, 71, 77, 83, 89, and 95; (2) a VL CDR2 having an amino acid sequence selected from the group consisting of SEQ ID NOs:24, 30, 36, 42, 48, 54, 60, 66, 72, 78, 84, 90, and 96; and (3) a VL CDR3 having an amino acid sequence selected from the group consisting of SEQ ID NOs:25, 31, 37, 43, 49, 55, 61, 67, 63, 79, 85, 91, and 97; or a variant thereof having up to about 5 amino acid substitutions, additions, and/or deletions in the VL CDRs.

In some embodiments, provided herein are antibodies or antigen-binding fragments thereof that bind PD-L1 having a VH, wherein the VH comprises VH CDR1, CDR2 and CDR3 having (1) the amino acid sequences of SEQ ID NOs:20, 21, and 22, respectively; (2) the amino acid sequences of SEQ ID NOs:26, 27, and 28, respectively; (3) the amino acid sequences of SEQ ID NOs:32, 33, and 34, respectively; (4) the amino acid sequences of SEQ ID NOs:38, 39, and 40, respectively; (5) the amino acid sequences of SEQ ID NOs:44, 45 and 46, respectively; (6) the amino acid sequences of SEQ ID NOs: 50, 51, and 52, respectively; (7) the amino acid sequences of SEQ ID NOs: 56, 57, and 58, respectively; (8) the amino acid sequences of SEQ ID NOs:62, 63, and 64, respectively; (9) the amino acid sequences of SEQ ID NOs:68, 69, and 70, respectively; (10) the amino acid sequences of SEQ ID NOs:74, 75, and 76, respectively; (11) the amino acid sequences of SEQ ID NOs:80, 81, and 82, respectively; (12) the amino acid sequences of SEQ ID NOs:86, 87, and 88, respectively; or (13) the amino acid sequences of SEQ ID NOs:92, 93 and 94, respectively; or a variant thereof having up to about 3, about 5, about 8, about 10, about 12, or about 15 amino acid substitutions, additions, and/or deletions in the VH CDRs. In some embodiments, the variant has up about 5 amino acid substitutions, additions, and/or deletions in the VH CDRs.

In some embodiments, provided herein are antibodies or antigen-binding fragments thereof that bind PD-L1 having a VL, wherein the VL comprises VL CDR1, CDR2 and CDR3 having the amino acid sequences of SEQ ID NOs:23, 24 and 25, respectively; (2) the amino acid sequences of SEQ ID NOs:29, 30, and 31, respectively; (3) the amino acid sequences of SEQ ID NOs:35, 36 and 37, respectively; (4) the amino acid sequences of SEQ ID NOs:41, 42 and 43, respectively; (5) the amino acid sequences of SEQ ID NOs:47, 48 and 49, respectively; (6) the amino acid sequences of SEQ ID NOs:53, 54, and 55, respectively; (7) the amino acid sequences of SEQ ID NOs:59, 60, and 61, respectively; (8) the amino acid sequences of SEQ ID NOs:65, 66, and 67, respectively; (9) the amino acid sequences of SEQ ID NOs:71, 72 and 73, respectively; (10) the amino acid sequences of SEQ ID NOs:77, 78 and 79, respectively; (11) the amino acid sequences of SEQ ID NOs:83, 84, and 85, respectively; (12) the amino acid sequences of SEQ ID NOs:89, 90 and 91, respectively; or (13) the amino acid sequences of SEQ ID NOs:95, 96 and 97, respectively; or a variant thereof having up to about 3, about 5, about 8, about 10, about 12, or about 15 amino acid substitutions, additions, and/or deletions in the VL CDRs. In some embodiments, the variant has up about 5 amino acid substitutions, additions, and/or deletions in the VL CDRs.

In some embodiments, provided herein are antibodies or antigen-binding fragments thereof that bind PD-L1 having a VH and a VL, wherein (a) the VH comprises VH CDR1, CDR2 and CDR3 having (1) the amino acid sequences of SEQ ID NOs:20, 21, and 22, respectively; (2) the amino acid sequences of SEQ ID NOs:26, 27, and 28, respectively; (3) the amino acid sequences of SEQ ID NOs:32, 33, and 34, respectively; (4) the amino acid sequences of SEQ ID NOs:38, 39, and 40, respectively; (5) the amino acid sequences of SEQ ID NOs:44, 45 and 46, respectively; (6) the amino acid sequences of SEQ ID NOs: 50, 51, and 52, respectively; (7) the amino acid sequences of SEQ ID NOs:56, 57, and 58, respectively; (8) the amino acid sequences of SEQ ID NOs:62, 63, and 64, respectively; (9) the amino acid sequences of SEQ ID NOs:68, 69, and 70, respectively; (10) the amino acid sequences of SEQ ID NOs:74, 75, and 76, respectively; (11) the amino acid sequences of SEQ ID NOs:80, 81, and 82, respectively; (12) the amino acid sequences of SEQ ID NOs:86, 87, and 88, respectively; or (13) the amino acid sequences of SEQ ID NOs:92, 93 and 94, respectively; or a variant thereof having up to about 5 amino acid substitutions, additions, and/or deletions in the VH CDRs; and (b) the VL comprises VL CDR1, CDR2 and CDR3 having the amino acid sequences of SEQ ID NOs:23, 24 and 25, respectively; (2) the amino acid sequences of SEQ ID NOs:29, 30, and 31, respectively; (3) the amino acid sequences of SEQ ID NOs:35, 36 and 37, respectively; (4) the amino acid sequences of SEQ ID NOs:41, 42 and 43, respectively; (5) the amino acid sequences of SEQ ID NOs:47, 48 and 49, respectively; (6) the amino acid sequences of SEQ ID NOs:53, 54, and 55, respectively; (7) the amino acid sequences of SEQ ID NOs:59, 60, and 61, respectively; (8) the amino acid sequences of SEQ ID NOs:65, 66, and 67, respectively; (9) the amino acid sequences of SEQ ID NOs:71, 72 and 73, respectively; (10) the amino acid sequences of SEQ ID NOs:77, 78 and 79, respectively; (11) the amino acid sequences of SEQ ID NOs:83, 84, and 85, respectively; (12) the amino acid sequences of SEQ ID NOs:89, 90 and 91, respectively; or (13) the amino acid sequences of SEQ ID NOs:95, 96 and 97, respectively; or a variant thereof having up to about 5 amino acid substitutions, additions, and/or deletions in the VL CDRs.

In some embodiments, provided herein are antibodies or antigen-binding fragments thereof that bind PD-L1 having a VH, comprising (1) a VH CDR1 having the amino acid sequence of SEQ ID NO:20, (2) a VH CDR2 having the amino acid sequence of SEQ ID NO:21, or (3) a VH CDR3 having the amino acid sequence of SEQ ID NO:22. In some embodiments, provided herein are antibodies or antigen-binding fragments thereof that bind PD-L1 having a VH, comprising (1) a VH CDR1 having the amino acid sequence of SEQ ID NO:20, (2) a VH CDR2 having the amino acid sequence of SEQ ID NO:21, and (3) a VH CDR3 having the amino acid sequence of SEQ ID NO:22.

In some embodiments, provided herein are antibodies or antigen-binding fragments thereof that bind PD-L1 having a VL, comprising (1) a VL CDR1 having the amino acid sequence of SEQ ID NO:23, (2) a VL CDR2 having the amino acid sequence of SEQ ID NO:24, or (3) a VL CDR3 having the amino acid sequence of SEQ ID NO:25. In some embodiments, provided herein are antibodies or antigen-binding fragments thereof that bind PD-L1 having a VL, comprising (1) a VL CDR1 having the amino acid sequence of SEQ ID NO:23, (2) a VL CDR2 having the amino acid sequence of SEQ ID NO:24, and (3) a VL CDR3 having the amino acid sequence of SEQ ID NO:25.

In some embodiments, provided herein are antibodies or antigen-binding fragments thereof that bind PD-L1 having (a) a VH that comprises (1) a VH CDR1 having the amino acid sequence of SEQ ID NO:20, (2) a VH CDR2 having the amino acid sequence of SEQ ID NO:21, and (3) a VH CDR3 having the amino acid sequence of SEQ ID NO:22, and (b) a VL that comprises (1) a VL CDR1 having the amino acid sequence of SEQ ID NO:23, (2) a VL CDR2 having the amino acid sequence of SEQ ID NO:24, and (3) a VL CDR3 having the amino acid sequence of SEQ ID NO:25.

In some embodiments, provided herein are antibodies or antigen-binding fragments thereof that bind PD-L1 having a VH, comprising (1) a VH CDR1 having the amino acid sequence of SEQ ID NO:26, (2) a VH CDR2 having the amino acid sequence of SEQ ID NO:27, or (3) a VH CDR3 having the amino acid sequence of SEQ ID NO:28. In some embodiments, provided herein are antibodies or antigen-binding fragments thereof that bind PD-L1 having a VH, comprising (1) a VH CDR1 having the amino acid sequence of SEQ ID NO:26, (2) a VH CDR2 having the amino acid sequence of SEQ ID NO:27, and (3) a VH CDR3 having the amino acid sequence of SEQ ID NO:28.

In some embodiments, provided herein are antibodies or antigen-binding fragments thereof that bind PD-L1 having a VL, comprising (1) a VL CDR1 having the amino acid sequence of SEQ ID NO:29, (2) a VL CDR2 having the amino acid sequence of SEQ ID NO:30, or (3) a VL CDR3 having the amino acid sequence of SEQ ID NO:31. In some embodiments, provided herein are antibodies or antigen-binding fragments thereof that bind PD-L1 having a VL, comprising (1) a VL CDR1 having the amino acid sequence of SEQ ID NO:29, (2) a VL CDR2 having the amino acid sequence of SEQ ID NO:30, and (3) a VL CDR3 having the amino acid sequence of SEQ ID NO:31.

In some embodiments, provided herein are antibodies or antigen-binding fragments thereof that bind PD-L1 having (a) a VH that comprises (1) a VH CDR1 having the amino acid sequence of SEQ ID NO:26, (2) a VH CDR2 having the amino acid sequence of SEQ ID NO:27, and (3) a VH CDR3 having the amino acid sequence of SEQ ID NO:28, and (b) a VL that comprises (1) a VL CDR1 having the amino acid sequence of SEQ ID NO:29, (2) a VL CDR2 having the amino acid sequence of SEQ ID NO:30, and (3) a VL CDR3 having the amino acid sequence of SEQ ID NO:31.

In some embodiments, provided herein are antibodies or antigen-binding fragments thereof that bind PD-L1 having a VH, comprising (1) a VH CDR1 having the amino acid sequence of SEQ ID NO:32, (2) a VH CDR2 having the amino acid sequence of SEQ ID NO:33, or (3) a VH CDR3 having the amino acid sequence of SEQ ID NO:34. In some embodiments, provided herein are antibodies or antigen-binding fragments thereof that bind PD-L1 having a VH, comprising (1) a VH CDR1 having the amino acid sequence of SEQ ID NO:32, (2) a VH CDR2 having the amino acid sequence of SEQ ID NO:33, and (3) a VH CDR3 having the amino acid sequence of SEQ ID NO:34.

In some embodiments, provided herein are antibodies or antigen-binding fragments thereof that bind PD-L1 having a VL, comprising (1) a VL CDR1 having the amino acid sequence of SEQ ID NO:35, (2) a VL CDR2 having the amino acid sequence of SEQ ID NO:36, or (3) a VL CDR3 having the amino acid sequence of SEQ ID NO:37. In some embodiments, provided herein are antibodies or antigen-binding fragments thereof that bind PD-L1 having a VL, comprising (1) a VL CDR1 having the amino acid sequence of SEQ ID NO:35, (2) a VL CDR2 having the amino acid sequence of SEQ ID NO:36, and (3) a VL CDR3 having the amino acid sequence of SEQ ID NO:37.

In some embodiments, provided herein are antibodies or antigen-binding fragments thereof that bind PD-L1 having (a) a VH that comprises (1) a VH CDR1 having the amino acid sequence of SEQ ID NO:32, (2) a VH CDR2 having the amino acid sequence of SEQ ID NO:33, and (3) a VH CDR3 having the amino acid sequence of SEQ ID NO:34, and (b) a VL that comprises (1) a VL CDR1 having the amino acid sequence of SEQ ID NO:35, (2) a VL CDR2 having the amino acid sequence of SEQ ID NO:36, and (3) a VL CDR3 having the amino acid sequence of SEQ ID NO:37.

In some embodiments, provided herein are antibodies or antigen-binding fragments thereof that bind PD-L1 having a VH, comprising (1) a VH CDR1 having the amino acid sequence of SEQ ID NO:38, (2) a VH CDR2 having the amino acid sequence of SEQ ID NO:39, or (3) a VH CDR3 having the amino acid sequence of SEQ ID NO:40. In some embodiments, provided herein are antibodies or antigen-binding fragments thereof that bind PD-L1 having a VH, comprising (1) a VH CDR1 having the amino acid sequence of SEQ ID NO:38, (2) a VH CDR2 having the amino acid sequence of SEQ ID NO:39, and (3) a VH CDR3 having the amino acid sequence of SEQ ID NO:40.

In some embodiments, provided herein are antibodies or antigen-binding fragments thereof that bind PD-L1 having a VL, comprising (1) a VL CDR1 having the amino acid sequence of SEQ ID NO:41, (2) a VL CDR2 having the amino acid sequence of SEQ ID NO:42, or (3) a VL CDR3 having the amino acid sequence of SEQ ID NO:43. In some embodiments, provided herein are antibodies or antigen-binding fragments thereof that bind PD-L1 having a VL, comprising (1) a VL CDR1 having the amino acid sequence of SEQ ID NO:41, (2) a VL CDR2 having the amino acid sequence of SEQ ID NO:42, and (3) a VL CDR3 having the amino acid sequence of SEQ ID NO:43.

In some embodiments, provided herein are antibodies or antigen-binding fragments thereof that bind PD-L1 having (a) a VH that comprises (1) a VH CDR1 having the amino acid sequence of SEQ ID NO:38, (2) a VH CDR2 having the amino acid sequence of SEQ ID NO:39, and (3) a VH CDR3 having the amino acid sequence of SEQ ID NO:40, and (b) a VL that comprises (1) a VL CDR1 having the amino acid sequence of SEQ ID NO:41, (2) a VL CDR2 having the amino acid sequence of SEQ ID NO:42, and (3) a VL CDR3 having the amino acid sequence of SEQ ID NO:43.

In some embodiments, provided herein are antibodies or antigen-binding fragments thereof that bind PD-L1 having a VH, comprising (1) a VH CDR1 having the amino acid sequence of SEQ ID NO:44, (2) a VH CDR2 having the amino acid sequence of SEQ ID NO:45, or (3) a VH CDR3 having the amino acid sequence of SEQ ID NO:46. In some embodiments, provided herein are antibodies or antigen-binding fragments thereof that bind PD-L1 having a VH, comprising (1) a VH CDR1 having the amino acid sequence of SEQ ID NO:44, (2) a VH CDR2 having the amino acid sequence of SEQ ID NO:45, and (3) a VH CDR3 having the amino acid sequence of SEQ ID NO:46.

In some embodiments, provided herein are antibodies or antigen-binding fragments thereof that bind PD-L1 having a VL, comprising (1) a VL CDR1 having the amino acid sequence of SEQ ID NO:47, (2) a VL CDR2 having the amino acid sequence of SEQ ID NO:48, or (3) a VL CDR3 having the amino acid sequence of SEQ ID NO:49. In some embodiments, provided herein are antibodies or antigen-binding fragments thereof that bind PD-L1 having a VL, comprising (1) a VL CDR1 having the amino acid sequence of SEQ ID NO:47, (2) a VL CDR2 having the amino acid sequence of SEQ ID NO:48, and (3) a VL CDR3 having the amino acid sequence of SEQ ID NO:49.

In some embodiments, provided herein are antibodies or antigen-binding fragments thereof that bind PD-L1 having (a) a VH that comprises (1) a VH CDR1 having the amino acid sequence of SEQ ID NO:44, (2) a VH CDR2 having the amino acid sequence of SEQ ID NO:45, and (3) a VH CDR3 having the amino acid sequence of SEQ ID NO:46, and (b) a VL that comprises (1) a VL CDR1 having the amino acid sequence of SEQ ID NO:47, (2) a VL CDR2 having the amino acid sequence of SEQ ID NO:48, and (3) a VL CDR3 having the amino acid sequence of SEQ ID NO:49.

In some embodiments, provided herein are antibodies or antigen-binding fragments thereof that bind PD-L1 having a VH, comprising (1) a VH CDR1 having the amino acid sequence of SEQ ID NO:50, (2) a VH CDR2 having the amino acid sequence of SEQ ID NO:51, or (3) a VH CDR3 having the amino acid sequence of SEQ ID NO:52. In some embodiments, provided herein are antibodies or antigen-binding fragments thereof that bind PD-L1 having a VH, comprising (1) a VH CDR1 having the amino acid sequence of SEQ ID NO:50, (2) a VH CDR2 having the amino acid sequence of SEQ ID NO:51, and (3) a VH CDR3 having the amino acid sequence of SEQ ID NO:52.

In some embodiments, provided herein are antibodies or antigen-binding fragments thereof that bind PD-L1 having a VL, comprising (1) a VL CDR1 having the amino acid sequence of SEQ ID NO:53, (2) a VL CDR2 having the amino acid sequence of SEQ ID NO:54, or (3) a VL CDR3 having the amino acid sequence of SEQ ID NO:55. In some embodiments, provided herein are antibodies or antigen-binding fragments thereof that bind PD-L1 having a VL, comprising (1) a VL CDR1 having the amino acid sequence of SEQ ID NO:53, (2) a VL CDR2 having the amino acid sequence of SEQ ID NO:54, and (3) a VL CDR3 having the amino acid sequence of SEQ ID NO:55.

In some embodiments, provided herein are antibodies or antigen-binding fragments thereof that bind PD-L1 having (a) a VH that comprises (1) a VH CDR1 having the amino acid sequence of SEQ ID NO:50, (2) a VH CDR2 having the amino acid sequence of SEQ ID NO:51, and (3) a VH CDR3 having the amino acid sequence of SEQ ID NO:52, and (b) a VL that comprises (1) a VL CDR1 having the amino acid sequence of SEQ ID NO:53, (2) a VL CDR2 having the amino acid sequence of SEQ ID NO:54, and (3) a VL CDR3 having the amino acid sequence of SEQ ID NO:55.

In some embodiments, provided herein are antibodies or antigen-binding fragments thereof that bind PD-L1 having a VH, comprising (1) a VH CDR1 having the amino acid sequence of SEQ ID NO:56, (2) a VH CDR2 having the amino acid sequence of SEQ ID NO:57, or (3) a VH CDR3 having the amino acid sequence of SEQ ID NO:58. In some embodiments, provided herein are antibodies or antigen-binding fragments thereof that bind PD-L1 having a VH, comprising (1) a VH CDR1 having the amino acid sequence of SEQ ID NO:56, (2) a VH CDR2 having the amino acid sequence of SEQ ID NO:57, and (3) a VH CDR3 having the amino acid sequence of SEQ ID NO:58.

In some embodiments, provided herein are antibodies or antigen-binding fragments thereof that bind PD-L1 having a VL, comprising (1) a VL CDR1 having the amino acid sequence of SEQ ID NO:59, (2) a VL CDR2 having the amino acid sequence of SEQ ID NO:60, or (3) a VL CDR3 having the amino acid sequence of SEQ ID NO:61. In some embodiments, provided herein are antibodies or antigen-binding fragments thereof that bind PD-L1 having a VL, comprising (1) a VL CDR1 having the amino acid sequence of SEQ ID NO:59, (2) a VL CDR2 having the amino acid sequence of SEQ ID NO:60, and (3) a VL CDR3 having the amino acid sequence of SEQ ID NO:61.

In some embodiments, provided herein are antibodies or antigen-binding fragments thereof that bind PD-L1 having (a) a VH that comprises (1) a VH CDR1 having the amino acid sequence of SEQ ID NO:56, (2) a VH CDR2 having the amino acid sequence of SEQ ID NO:57, and (3) a VH CDR3 having the amino acid sequence of SEQ ID NO:58, and (b) a VL that comprises (1) a VL CDR1 having the amino acid sequence of SEQ ID NO:59, (2) a VL CDR2 having the amino acid sequence of SEQ ID NO:60, and (3) a VL CDR3 having the amino acid sequence of SEQ ID NO:61.

In some embodiments, provided herein are antibodies or antigen-binding fragments thereof that bind PD-L1 having a VH, comprising (1) a VH CDR1 having the amino acid sequence of SEQ ID NO:62, (2) a VH CDR2 having the amino acid sequence of SEQ ID NO:63, or (3) a VH CDR3 having the amino acid sequence of SEQ ID NO:64. In some embodiments, provided herein are antibodies or antigen-binding fragments thereof that bind PD-L1 having a VH, comprising (1) a VH CDR1 having the amino acid sequence of SEQ ID NO:62, (2) a VH CDR2 having the amino acid sequence of SEQ ID NO:63, and (3) a VH CDR3 having the amino acid sequence of SEQ ID NO:64.

In some embodiments, provided herein are antibodies or antigen-binding fragments thereof that bind PD-L1 having a VL, comprising (1) a VL CDR1 having the amino acid sequence of SEQ ID NO:65, (2) a VL CDR2 having the amino acid sequence of SEQ ID NO:66, or (3) a VL CDR3 having the amino acid sequence of SEQ ID NO:67. In some embodiments, provided herein are antibodies or antigen-binding fragments thereof that bind PD-L1 having a VL, comprising (1) a VL CDR1 having the amino acid sequence of SEQ ID NO:65, (2) a VL CDR2 having the amino acid sequence of SEQ ID NO:66, and (3) a VL CDR3 having the amino acid sequence of SEQ ID NO:67.

In some embodiments, provided herein are antibodies or antigen-binding fragments thereof that bind PD-L1 having (a) a VH that comprises (1) a VH CDR1 having the amino acid sequence of SEQ ID NO:62, (2) a VH CDR2 having the amino acid sequence of SEQ ID NO:63, and (3) a VH CDR3 having the amino acid sequence of SEQ ID NO:64, and (b) a VL that comprises (1) a VL CDR1 having the amino acid sequence of SEQ ID NO:65, (2) a VL CDR2 having the amino acid sequence of SEQ ID NO:66, and (3) a VL CDR3 having the amino acid sequence of SEQ ID NO:67.

In some embodiments, provided herein are antibodies or antigen-binding fragments thereof that bind PD-L1 having a VH, comprising (1) a VH CDR1 having the amino acid sequence of SEQ ID NO:68, (2) a VH CDR2 having the amino acid sequence of SEQ ID NO:69, or (3) a VH CDR3 having the amino acid sequence of SEQ ID NO:70. In some embodiments, provided herein are antibodies or antigen-binding fragments thereof that bind PD-L1 having a VH, comprising (1) a VH CDR1 having the amino acid sequence of SEQ ID NO:68, (2) a VH CDR2 having the amino acid sequence of SEQ ID NO:69, and (3) a VH CDR3 having the amino acid sequence of SEQ ID NO:70.

In some embodiments, provided herein are antibodies or antigen-binding fragments thereof that bind PD-L1 having a VL, comprising (1) a VL CDR1 having the amino acid sequence of SEQ ID NO:71, (2) a VL CDR2 having the amino acid sequence of SEQ ID NO:72, or (3) a VL CDR3 having the amino acid sequence of SEQ ID NO:73. In some embodiments, provided herein are antibodies or antigen-binding fragments thereof that bind PD-L1 having a VL, comprising (1) a VL CDR1 having the amino acid sequence of SEQ ID NO:71, (2) a VL CDR2 having the amino acid sequence of SEQ ID NO:72, and (3) a VL CDR3 having the amino acid sequence of SEQ ID NO:73.

In some embodiments, provided herein are antibodies or antigen-binding fragments thereof that bind PD-L1 having (a) a VH that comprises (1) a VH CDR1 having the amino acid sequence of SEQ ID NO:68, (2) a VH CDR2 having the amino acid sequence of SEQ ID NO:69, and (3) a VH CDR3 having the amino acid sequence of SEQ ID NO:70, and (b) a VL that comprises (1) a VL CDR1 having the amino acid sequence of SEQ ID NO:71, (2) a VL CDR2 having the amino acid sequence of SEQ ID NO:72, and (3) a VL CDR3 having the amino acid sequence of SEQ ID NO:73.

In some embodiments, provided herein are antibodies or antigen-binding fragments thereof that bind PD-L1 having a VH, comprising (1) a VH CDR1 having the amino acid sequence of SEQ ID NO:74, (2) a VH CDR2 having the amino acid sequence of SEQ ID NO:75, or (3) a VH CDR3 having the amino acid sequence of SEQ ID NO:76. In some embodiments, provided herein are antibodies or antigen-binding fragments thereof that bind PD-L1 having a VH, comprising (1) a VH CDR1 having the amino acid sequence of SEQ ID NO:74, (2) a VH CDR2 having the amino acid sequence of SEQ ID NO:75, and (3) a VH CDR3 having the amino acid sequence of SEQ ID NO:76.

In some embodiments, provided herein are antibodies or antigen-binding fragments thereof that bind PD-L1 having a VL, comprising (1) a VL CDR1 having the amino acid sequence of SEQ ID NO:77, (2) a VL CDR2 having the amino acid sequence of SEQ ID NO:78, or (3) a VL CDR3 having the amino acid sequence of SEQ ID NO:79. In some embodiments, provided herein are antibodies or antigen-binding fragments thereof that bind PD-L1 having a VL, comprising (1) a VL CDR1 having the amino acid sequence of SEQ ID NO:77, (2) a VL CDR2 having the amino acid sequence of SEQ ID NO:78, and (3) a VL CDR3 having the amino acid sequence of SEQ ID NO:79.

In some embodiments, provided herein are antibodies or antigen-binding fragments thereof that bind PD-L1 having (a) a VH that comprises (1) a VH CDR1 having the amino acid sequence of SEQ ID NO:74, (2) a VH CDR2 having the amino acid sequence of SEQ ID NO:75, and (3) a VH CDR3 having the amino acid sequence of SEQ ID NO:76, and (b) a VL that comprises (1) a VL CDR1 having the amino acid sequence of SEQ ID NO:77, (2) a VL CDR2 having the amino acid sequence of SEQ ID NO:78, and (3) a VL CDR3 having the amino acid sequence of SEQ ID NO:79.

In some embodiments, provided herein are antibodies or antigen-binding fragments thereof that bind PD-L1 having a VH, comprising (1) a VH CDR1 having the amino acid sequence of SEQ ID NO:80, (2) a VH CDR2 having the amino acid sequence of SEQ ID NO:81, or (3) a VH CDR3 having the amino acid sequence of SEQ ID NO:82. In some embodiments, provided herein are antibodies or antigen-binding fragments thereof that bind PD-L1 having a VH, comprising (1) a VH CDR1 having the amino acid sequence of SEQ ID NO:80, (2) a VH CDR2 having the amino acid sequence of SEQ ID NO:81, and (3) a VH CDR3 having the amino acid sequence of SEQ ID NO:82.

In some embodiments, provided herein are antibodies or antigen-binding fragments thereof that bind PD-L1 having a VL, comprising (1) a VL CDR1 having the amino acid sequence of SEQ ID NO:83, (2) a VL CDR2 having the amino acid sequence of SEQ ID NO: 84, or (3) a VL CDR3 having the amino acid sequence of SEQ ID NO:85. In some embodiments, provided herein are antibodies or antigen-binding fragments thereof that bind PD-L1 having a VL, comprising (1) a VL CDR1 having the amino acid sequence of SEQ ID NO:83, (2) a VL CDR2 having the amino acid sequence of SEQ ID NO:84, and (3) a VL CDR3 having the amino acid sequence of SEQ ID NO:85.

In some embodiments, provided herein are antibodies or antigen-binding fragments thereof that bind PD-L1 having (a) a VH that comprises (1) a VH CDR1 having the amino acid sequence of SEQ ID NO:80, (2) a VH CDR2 having the amino acid sequence of SEQ ID NO:81, and (3) a VH CDR3 having the amino acid sequence of SEQ ID NO:82, and (b) a VL that comprises (1) a VL CDR1 having the amino acid sequence of SEQ ID NO:83, (2) a VL CDR2 having the amino acid sequence of SEQ ID NO:84, and (3) a VL CDR3 having the amino acid sequence of SEQ ID NO:85.

In some embodiments, provided herein are antibodies or antigen-binding fragments thereof that bind PD-L1 having a VH, comprising (1) a VH CDR1 having the amino acid sequence of SEQ ID NO:86, (2) a VH CDR2 having the amino acid sequence of SEQ ID NO:87, or (3) a VH CDR3 having the amino acid sequence of SEQ ID NO:88. In some embodiments, provided herein are antibodies or antigen-binding fragments thereof that bind PD-L1 having a VH, comprising (1) a VH CDR1 having the amino acid sequence of SEQ ID NO:86, (2) a VH CDR2 having the amino acid sequence of SEQ ID NO:87, and (3) a VH CDR3 having the amino acid sequence of SEQ ID NO:88.

In some embodiments, provided herein are antibodies or antigen-binding fragments thereof that bind PD-L1 having a VL, comprising (1) a VL CDR1 having the amino acid sequence of SEQ ID NO:89, (2) a VL CDR2 having the amino acid sequence of SEQ ID NO:90, or (3) a VL CDR3 having the amino acid sequence of SEQ ID NO:91. In some embodiments, provided herein are antibodies or antigen-binding fragments thereof that bind PD-L1 having a VL, comprising (1) a VL CDR1 having the amino acid sequence of SEQ ID NO:89, (2) a VL CDR2 having the amino acid sequence of SEQ ID NO:90, and (3) a VL CDR3 having the amino acid sequence of SEQ ID NO:91.

In some embodiments, provided herein are antibodies or antigen-binding fragments thereof that bind PD-L1 having (a) a VH that comprises (1) a VH CDR1 having the amino acid sequence of SEQ ID NO:86, (2) a VH CDR2 having the amino acid sequence of SEQ ID NO:87, and (3) a VH CDR3 having the amino acid sequence of SEQ ID NO:88, and (b) a VL that comprises (1) a VL CDR1 having the amino acid sequence of SEQ ID NO:89, (2) a VL CDR2 having the amino acid sequence of SEQ ID NO:90, and (3) a VL CDR3 having the amino acid sequence of SEQ ID NO:91.

In some embodiments, provided herein are antibodies or antigen-binding fragments thereof that bind PD-L1 having a VH, comprising (1) a VH CDR1 having the amino acid sequence of SEQ ID NO:92, (2) a VH CDR2 having the amino acid sequence of SEQ ID NO:93, or (3) a VH CDR3 having the amino acid sequence of SEQ ID NO:94. In some embodiments, provided herein are antibodies or antigen-binding fragments thereof that bind PD-L1 having a VH, comprising (1) a VH CDR1 having the amino acid sequence of SEQ ID NO:92, (2) a VH CDR2 having the amino acid sequence of SEQ ID NO:93, and (3) a VH CDR3 having the amino acid sequence of SEQ ID NO:94.

In some embodiments, provided herein are antibodies or antigen-binding fragments thereof that bind PD-L1 having a VL, comprising (1) a VL CDR1 having the amino acid sequence of SEQ ID NO:95, (2) a VL CDR2 having the amino acid sequence of SEQ ID NO:96, or (3) a VL CDR3 having the amino acid sequence of SEQ ID NO:97. In some embodiments, provided herein are antibodies or antigen-binding fragments thereof that bind PD-L1 having a VL, comprising (1) a VL CDR1 having the amino acid sequence of SEQ ID NO:95, (2) a VL CDR2 having the amino acid sequence of SEQ ID NO:96, and (3) a VL CDR3 having the amino acid sequence of SEQ ID NO:97.

In some embodiments, provided herein are antibodies or antigen-binding fragments thereof that bind PD-L1 having (a) a VH that comprises (1) a VH CDR1 having the amino acid sequence of SEQ ID NO:92, (2) a VH CDR2 having the amino acid sequence of SEQ ID NO:93, and (3) a VH CDR3 having the amino acid sequence of SEQ ID NO:94, and (b) a VL that comprises (1) a VL CDR1 having the amino acid sequence of SEQ ID NO:95, (2) a VL CDR2 having the amino acid sequence of SEQ ID NO:96, and (3) a VL CDR3 having the amino acid sequence of SEQ ID NO:97.

In some embodiments, provided herein are antibodies or antigen-binding fragments thereof that bind PD-L1 comprising a VH having at least 85%, at least 90%, at least 95%, at least 98% or 100% sequence identity to an amino acid sequence selected from the group consisting of SEQ ID NOs:98-110, 124, 126-128, 131-136 and 174-178. In some embodiments, provided herein are antibodies or antigen-binding fragments thereof that bind PD-L1 comprising a VL having at least 85%, at least 90%, at least 95%, at least 98% or 100% sequence identity to an amino acid sequence selected from the group consisting of SEQ ID NOs:111-123, 125, 129-130, 137-144 and 179-181.

TABLE 3

Amino acid sequences of heavy chain variable regions (VHs) and light chain variable region (VLs) of anti-PD-L1 antibodies

| | | |
|---|---|---|
| Antibody | VH | VL |
| Clone A | EVQLQQSGAELVKPGASVKLSCTASGFN IKDTYMHWVKQRPEQGLEWIGRIDPAN GDTKYDPKFQGKATITADTSSNTAYLHL SSLTSEDTAVYYCASPRLVRFPYVMDYC GQGTSVTVSS (SEQ ID NO:98) | DIQMTQSPASLSASVGETVTITCRASENV YSYLAWYQQKQGKSPQLLVYNAKTLAE GVPSRFSGSGSGTQFSLKINSLQPEDFGS YYCQHHYGFPYTFGGGTKLEIK (SEQ ID NO:111) |
| Clone B | QGQMQQSGAELVKPGASVKLSCKTSGF TFSSSYISWLKQKPGQSLEWIAWIYAGT GGTSYNQKFTGKAQLTVDTPSSTAYMQ FSSLTTEDSAIYYCARHEGKYWYFDVW GAGTTVTVSS (SEQ ID NO:99) | QILLTQSPAIMSASPGEKVTMTCSASSSV SYVHWYQQKPGSSPKPWIYDTSNLASGF PARFSGSGSGTSYSLIISSMEAEDAATYY CHQRSSYPWTFGGGTKLEIK (SEQ ID NO:112) |
| Clone C | EVQLQQSGADLVKPGASVKLSCTASGFN IKDTYMHWVKQRPEQGLEWIGRIDPAN GNTKYDPKFQGKATITTDTSSNTAYLQL SSLTSEDTAVYYCARGLGRWFAYWGQG TLVTVSA (SEQ ID NO:100) | DIVMTQSHKFMSTSVGDRVSITCKASQD VSNAVAWYQQKPGQSPELLIYSASNRYT GVPDRFTGSGSGTDFTFTISSVQAEDLAV YYCQQHDSTPLTFGAGTKTELK (SEQ ID NO:113) |
| Clone D | EVQLQQSGPELVKPGASMKISCKASGYS FTGYTMNWVKQSHGKNLEWIGLIIPYNG GISYNQKFKDKATLTVDKSSSTAYMELL SLTSEDSAVYYCASLITTAPRDSMDYWG QGTSVTVSS (SEQ ID NO:101) | DIQMTQTTSSLSASLGDRVTISCRASQDI SNYLNWYQQKPDGSVKLLIYYTSKLHS GVPSRFSGSGSGTDYSLTISNLEQEDIAT YFCQQGDALPWTFGGGTKLEIK (SEQ ID NO:114) |
| Clone F | EVQLQESGPTLVKPSQTLSLTCSVTGDSI TSGYWNWIRKFPGNKVEYMGYISYSGS TYCIPSLKSRISITRDTSKNQYYLQLNSVT TADTATYYCARGNWGREAWFAYWGQ GTLVTVSA (SEQ ID NO:102) | DIQMTQSPASLSASVGETVTITCRASENI YSYLAWYQQKQGKSPQLLVYNVKILAE GVSSRFSGSGSGTQFSLKINSLQPEDFGS YYCQHHYGLPYTFGGGTQLEIK (SEQ ID NO:115) |
| Clone G | QGQMQQSGAELVKPGASVKLSCKTSGF TFSSTYISWLKQKPGQSLEWIAWIYAGT GGTSYNQKFTGKAQLTVDTSSSTAYMQ | DIVMTQSHKFMSTSVGDRVSITCKASQD VSTAVAWYQQKPGQSPKLLIYSASYRYT GVPDRFTGSGSGTDFTFTISSVQAGDLAV |
| | FSSLTTEDSAIYYCARHYGTYWYFDVW GAGTTVTVSS (SEQ ID NO:103) | YYCLQHYSTPWTFGGGTKLEIK (SEQ ID NO:116) |
| Clone H | QGQMQQSGAELVKPGASVKLSCKTSGF TFSSTYISWLKQKPGQSLEWIAWIYAGT GGTSYNQKFTGKAQLTVDTSSSTAYMQ FSSLTTEDSAIYYCARHIGNYWYFDIWG AGTTVTVSS (SEQ ID NO:104) | QILLTQSPAIMSASPGEKVTMTCSASSSV SYIHWYQQKPGSSPKPWIYDTSNLASGF PARFSGSGSGTSYFLIISSMEAEDAATYY CHQRSSYPWTFGGGTKLEIK (SEQ ID NO:117) |
| Clone J | QVQLQQPGAELVMPGPSVKMSCKASGY TFTDYWMHWVKQRPGQGLEWIGAVDT SDRYTTYNQKFRGKATMTVDESSSTAY MQLSNLTSEDSAVYYCARGLTGPYYYP MDFWGQGTSVTASS (SEQ ID NO:105) | DIQMTQSPASQSASLGESVTITCLASQTI GTWLAWYQQKPGKSPQLLIYAATSLAD GVPSRFSGGGSGTKFSFKISSLQAEDFGS YYCQQVFSSPYTFGGGTKLEIK (SEQ ID NO:118) |
| Clone K | EVQLQQSGPELVKPGASMKISCKASGYS FTGYTMNWVKQSHGKNLEWIGLIIPHNG GTSYNQKFKDKATLTVDKSSRTAYMEL LSLTSEDSAVYYCASLMTTAPRDSMDY WGQGTSVTVSS (SEQ ID NO:106) | DIQMTQSPASQSASLGESVTITCLASQTI GTWLAWYQQKPGKSPQLLIYAATSLAD GVPSRFSGSGSGTKFSFKISSLQAEDFVS YYCQQLFSTPWTFGGGTKLEIK (SEQ ID NO:119) |
| Clone L | EVHLQQSGPELVKPGASMKISCKASGYS FTGYTMNWVKQSHGKNLEWIGLIIPSNG GTNYNQRFKDKATLSVDKSSSTAYMEL LSLTSEDSAVYYCASLITTAPRDSMDYW GQGTSVTVSS (SEQ ID NO:107) | DIQMTQTPSSLSASLGDRVTISCRASQDIS NYLNWYQQKPDGSVKLLIYYTSKLHSG VPSRFSGSGSGTDYSLTINNLDQEDIATY FCQQGDALPWTFGGGTKLEIK (SEQ ID NO:120) |
| Clone N | EVQLQQSGPELVKPGASMKISCKASGYS FTGYTMNWVKQSHGKNLEWIGLIIPHNG GTSYNQKFKDKATLTVDKSSRTAYMEL LSLTSEDSAVYYCASLMTTAPRDSMDY WGQGTSVTVSS (SEQ ID NO:108) | DIQMTQTTSSLSASLGDRVTISCRASQDI SNYLNWYQQKPDGTVKLLIYYTSRLHS GVPSRFSGSGSGTDYSLTISNLDQEDIAT YFCQQGATLPWTFGGGTKLEIK (SEQ ID NO:121) |
| Clone P | EVQLQESGPSLVKYSQTLSLTCSVTGDSI TSGYWNWIRKFPGNKLEYMGYISYTGST YHTPSLKGRISITRDTSKNQYYMQLDSV TTEDTATYYCARGNWGREAWFPYWGQ GTLVTVSA (SEQ ID NO:109) | DIQMTQSPASLSASVGETVTITCRASESIY SYLAWYQQKQGKSPQLLVYNAKTLAEG VPSRFSGSGSGTQFSLKINSLQTEDFGTY YCQHHYGFPYTFGGGTKLEIK (SEQ ID NO:122) |
| Clone Y | QVQLQQSGPQLVRPGASVKISCKASGYS FTSYWMHWVKQRPGHGLEWIGMIDPSD SETKLNQKFRDKATLTVDTSSSTAYMQL SSPTSEDSAVYYCARDYRYDVGAMDY WGQGTSVTVSS (SEQ ID NO:110) | DVQITQSPSFLAASPGETITLNCRASKSIS KFLAWYQEKPGKTNKVLIYSGSTLQSGI PSRFSGSGSGTDFTLTISSLEPEDFAMYY CQQHHEYPWTFGGGTKLEIK (SEQ ID NO:122) |

In some embodiments, provided herein are antibodies or antigen-binding fragments thereof that bind PD-L1 comprising: (a) a VH having at least 85%, at least 90%, at least 95%, at least 98%, or 100% sequence identity to an amino acid sequence selected from the group consisting of SEQ ID NOs:98-110, 124, 126-128, 131-136 and 174-178; and (b) a VL having at least 85%, at least 90%, at least 95%, at least 98%, or 100% sequence identity to an amino acid sequence selected from the group consisting of SEQ ID NOs: 111-123, 125, 129-130, 137-144 and 179-181.

In some embodiments, provided herein are antibodies or antigen-binding fragments thereof that bind PD-L1 comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:98. In some embodiments, the anti-PD-L1 antibody or antigen-binding fragment thereof has a VH having at least 85% sequence identity to SEQ ID NO:98. In some embodiments, the anti-PD-L1 antibody or antigen-binding fragment thereof has a VH having at least 90% sequence identity to SEQ ID NO:98. In some embodiments, the anti-PD-L1 antibody or antigen-binding fragment thereof has a VH having at least 95% sequence identity to SEQ ID NO:98. In some embodiments, the anti-PD-L1 antibody or antigen-binding fragment thereof has a VH having at least 98% sequence identity to SEQ ID NO:98. In some embodiments, provided herein are antibodies or antigen-binding fragments thereof that bind PD-L1 comprising a VH having the amino acid sequence of SEQ ID NO:98.

In some embodiments, provided herein are antibodies or antigen-binding fragments thereof that bind PD-L1 comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:99. In some embodiments, the anti-PD-L1 antibody or antigen-binding fragment thereof has a VH having at least 85% sequence identity to SEQ ID NO:99. In some embodiments, the anti-PD-L1 antibody or antigen-binding fragment thereof has a VH having at least 90% sequence identity to SEQ ID NO:99. In some embodiments, the anti-PD-L1 antibody or antigen-binding fragment thereof has a VH having at least 95% sequence identity to SEQ ID NO:99. In some embodiments, the anti-PD-L1 antibody or antigen-binding fragment thereof has a VH having at least 98% sequence identity to SEQ ID NO:99. In some embodiments, provided herein are antibodies or antigen-binding fragments thereof that bind PD-L1 comprising a VH having the amino acid sequence of SEQ ID NO:99.

In some embodiments, provided herein are antibodies or antigen-binding fragments thereof that bind PD-L1 comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:100. In some embodiments, the anti-PD-L1 antibody or antigen-binding fragment thereof has a VH having at least 85% sequence identity to SEQ ID NO: 100. In some embodiments, the anti-PD-L1 antibody or antigen-binding fragment thereof has a VH having at least 90% sequence identity to SEQ ID NO:100. In some embodiments, the anti-PD-L1 antibody or antigen-binding fragment thereof has a VH having at least 95% sequence identity to SEQ ID NO:100. In some embodiments, the anti-PD-L1 antibody or antigen-binding fragment thereof has a VH having at least 98% sequence identity to SEQ ID NO:100. In some embodiments, provided herein are antibodies or antigen-binding fragments thereof that bind PD-L1 comprising a VH having the amino acid sequence of SEQ ID NO:100.

In some embodiments, provided herein are antibodies or antigen-binding fragments thereof that bind PD-L1 comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:101. In some embodiments, the anti-PD-L1 antibody or antigen-binding fragment thereof has a VH having at least 85% sequence identity to SEQ ID NO: 101. In some embodiments, the anti-PD-L1 antibody or antigen-binding fragment thereof has a VH having at least 90% sequence identity to SEQ ID NO: 101. In some embodiments, the anti-PD-L1 antibody or antigen-binding fragment thereof has a VH having at least 95% sequence identity to SEQ ID NO:101. In some embodiments, the anti-PD-L1 antibody or antigen-binding fragment thereof has a VH having at least 98% sequence identity to SEQ ID NO:101. In some embodiments, provided herein are antibodies or antigen-binding fragments thereof that bind PD-L1 comprising a VH having the amino acid sequence of SEQ ID NO:101.

In some embodiments, provided herein are antibodies or antigen-binding fragments thereof that bind PD-L1 comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:102. In some embodiments, the anti-PD-L1 antibody or antigen-binding fragment thereof has a VH having at least 85% sequence identity to SEQ ID NO: 102. In some embodiments, the anti-PD-L1 antibody or antigen-binding fragment thereof has a VH having at least 90% sequence identity to SEQ ID NO:102. In some embodiments, the anti-PD-L1 antibody or antigen-binding fragment thereof has a VH having at least 95% sequence identity to SEQ ID NO:102. In some embodiments, the anti-PD-L1 antibody or antigen-binding fragment thereof has a VH having at least 98% sequence identity to SEQ ID NO:102. In some embodiments, provided herein are antibodies or antigen-binding fragments thereof that bind PD-L1 comprising a VH having the amino acid sequence of SEQ ID NO:102.

In some embodiments, provided herein are antibodies or antigen-binding fragments thereof that bind PD-L1 comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:103. In some embodiments, the anti-PD-L1 antibody or antigen-binding fragment thereof has a VH having at least 85% sequence identity to SEQ ID NO:103. In some embodiments, the anti-PD-L1 antibody or antigen-binding fragment thereof has a VH having at least 90% sequence identity to SEQ ID NO:103. In some embodiments, the anti-PD-L1 antibody or antigen-binding fragment thereof has a VH having at least 95% sequence identity to SEQ ID NO:103. In some embodiments, the anti-PD-L1 antibody or antigen-binding fragment thereof has a VH having at least 98% sequence identity to SEQ ID NO:103. In some embodiments, provided herein are antibodies or antigen-binding fragments thereof that bind PD-L1 comprising a VH having the amino acid sequence of SEQ ID NO:103.

In some embodiments, provided herein are antibodies or antigen-binding fragments thereof that bind PD-L1 comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:104. In some embodiments, the anti-PD-L1 antibody or antigen-binding fragment thereof has a VH having at least 85% sequence identity to SEQ ID NO: 104. In some embodiments, the anti-PD-L1 antibody or antigen-binding fragment thereof has a VH having at least 90% sequence identity to SEQ ID NO:104. In some embodiments, the anti-PD-L1 antibody or antigen-binding fragment thereof has a VH having at least 95% sequence identity to SEQ ID NO:104. In some embodiments, the anti-PD-L1 antibody or antigen-binding fragment thereof has a VH having at least 98% sequence identity to SEQ ID NO:104. In some embodiments, provided herein are antibodies or antigen-binding fragments thereof that bind PD-L1 comprising a VH having the amino acid sequence of SEQ ID NO:104.

In some embodiments, provided herein are antibodies or antigen-binding fragments thereof that bind PD-L1 comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:105. In some embodiments, the anti-PD-L1 antibody or antigen-binding fragment thereof has a VH having at least 85% sequence identity to SEQ ID NO: 105. In some embodiments, the anti-PD-L1 antibody or antigen-binding fragment thereof has a VH having at least 90% sequence identity to SEQ ID NO: 105. In some embodiments, the anti-PD-L1 antibody or antigen-binding fragment thereof has a VH having at least 95% sequence identity to SEQ ID NO:105. In some embodiments, the anti-PD-L1 antibody or antigen-binding fragment thereof has a VH having at least 98% sequence identity to SEQ ID NO: 105. In some embodiments, provided herein are antibodies or antigen-binding fragments thereof that bind PD-L1 comprising a VH having the amino acid sequence of SEQ ID NO:105.

In some embodiments, provided herein are antibodies or antigen-binding fragments thereof that bind PD-L1 comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:106. In some embodiments, the anti-PD-L1 antibody or antigen-binding fragment thereof has a VH having at least 85% sequence identity to SEQ ID NO: 106. In some embodiments, the anti-PD-L1 antibody or antigen-binding fragment thereof has a VH having at least 90% sequence identity to SEQ ID NO:106. In some embodiments, the anti-PD-L1 antibody or antigen-binding fragment thereof has a VH having at least 95% sequence identity to SEQ ID NO:106. In some embodiments, the anti-PD-L1 antibody or antigen-binding fragment thereof has a VH having at least 98% sequence identity to SEQ ID NO:106. In some embodiments, provided herein are antibodies or antigen-binding fragments thereof that bind PD-L1 comprising a VH having the amino acid sequence of SEQ ID NO:106.

In some embodiments, provided herein are antibodies or antigen-binding fragments thereof that bind PD-L1 comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:107. In some embodiments, the anti-PD-L1 antibody or antigen-binding fragment thereof has a VH having at least 85% sequence identity to SEQ ID NO: 107. In some embodiments, the anti-PD-L1 antibody or antigen-binding fragment thereof has a VH having at least 90% sequence identity to SEQ ID NO:107. In some embodiments, the anti-PD-L1 antibody or antigen-binding fragment thereof has a VH having at least 95% sequence identity to SEQ ID NO:107. In some embodiments, the anti-PD-L1 antibody or antigen-binding fragment thereof has a VH having at least 98% sequence identity to SEQ ID NO:107. In some embodiments, provided herein are antibodies or antigen-binding fragments thereof that bind PD-L1 comprising a VH having the amino acid sequence of SEQ ID NO:107.

In some embodiments, provided herein are antibodies or antigen-binding fragments thereof that bind PD-L1 comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:108. In some embodiments, the anti-PD-L1 antibody or antigen-binding fragment thereof has a VH having at least 85% sequence identity to SEQ ID NO: 108. In some embodiments, the anti-PD-L1 antibody or antigen-binding fragment thereof has a VH having at least 90% sequence identity to SEQ ID NO:108. In some embodiments, the anti-PD-L1 antibody or antigen-binding fragment thereof has a VH having at least 95% sequence identity to SEQ ID NO:108. In some embodiments, the anti-PD-L1 antibody or antigen-binding fragment thereof has a VH having at least 98% sequence identity to SEQ ID NO:108. In some embodiments, provided herein are antibodies or antigen-binding fragments thereof that bind PD-L1 comprising a VH having the amino acid sequence of SEQ ID NO:108.

In some embodiments, provided herein are antibodies or antigen-binding fragments thereof that bind PD-L1 comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:109. In some embodiments, the anti-PD-L1 antibody or antigen-binding fragment thereof has a VH having at least 85% sequence identity to SEQ ID NO: 109. In some embodiments, the anti-PD-L1 antibody or antigen-binding fragment thereof has a VH having at least 90% sequence identity to SEQ ID NO: 109. In some embodiments, the anti-PD-L1 antibody or antigen-binding fragment thereof has a VH having at least 95% sequence identity to SEQ ID NO:109. In some embodiments, the anti-PD-L1 antibody or antigen-binding fragment thereof has a VH having at least 98% sequence identity to SEQ ID NO:109. In some embodiments, provided herein are antibodies or antigen-binding fragments thereof that bind PD-L1 comprising a VH having the amino acid sequence of SEQ ID NO:109.

In some embodiments, provided herein are antibodies or antigen-binding fragments thereof that bind PD-L1 comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:110. In some embodiments, the anti-PD-L1 antibody or antigen-binding fragment thereof has a VH having at least 85% sequence identity to SEQ ID NO: 110. In some embodiments, the anti-PD-L1 antibody or antigen-binding fragment thereof has a VH having at least 90% sequence identity to SEQ ID NO: 110. In some embodiments, the anti-PD-L1 antibody or antigen-binding fragment thereof has a VH having at least 95% sequence identity to SEQ ID NO:110. In some embodiments, the anti-PD-L1 antibody or antigen-binding fragment thereof has a VH having at least 98% sequence identity to SEQ ID NO:110. In some embodiments, provided herein are antibodies or antigen-binding fragments thereof that bind PD-L1 comprising a VH having the amino acid sequence of SEQ ID NO:110.

In some embodiments, provided herein are antibodies or antigen-binding fragments thereof that bind PD-L1 comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:124. In some embodiments, the anti-PD-L1 antibody or antigen-binding fragment thereof has a VH having at least 85% sequence identity to SEQ ID NO:124. In some embodiments, the anti-PD-L1 antibody or antigen-binding fragment thereof has a VH having at least 90% sequence identity to SEQ ID NO:124. In some embodiments, the anti-PD-L1 antibody or antigen-binding fragment thereof has a VH having at least 95% sequence identity to SEQ ID NO:124. In some embodiments, the anti-PD-L1 antibody or antigen-binding fragment thereof has a VH having at least 98% sequence identity to SEQ ID NO:124. In some embodiments, provided herein are antibodies or antigen-binding fragments thereof that bind PD-L1 comprising a VH having the amino acid sequence of SEQ ID NO:124.

In some embodiments, provided herein are antibodies or antigen-binding fragments thereof that bind PD-L1 comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:126. In some embodiments, the anti-PD-L1 antibody or antigen-binding fragment thereof has a VH having at least 85% sequence identity to SEQ ID NO:126. In some embodiments, the anti-PD-L1 antibody or antigen-binding fragment thereof has a VH having at least 90% sequence identity to SEQ ID NO:126. In some embodiments, the anti-PD-L1 antibody or antigen-binding fragment thereof has a VH having at least 95% sequence identity to SEQ ID NO:126. In some embodiments, the anti-PD-L1 antibody or antigen-binding fragment thereof has a VH having at least 98% sequence identity to SEQ ID NO:126. In some embodiments, provided herein are antibodies or antigen-binding fragments thereof that bind PD-L1 comprising a VH having the amino acid sequence of SEQ ID NO:126.

In some embodiments, provided herein are antibodies or antigen-binding fragments thereof that bind PD-L1 comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:127. In some embodiments, the anti-PD-L1 antibody or antigen-binding fragment thereof has a VH having at least 85% sequence identity to SEQ ID NO:127. In some embodiments, the anti-PD-L1 antibody or antigen-binding fragment thereof has a VH having at least 90% sequence identity to SEQ ID NO:127. In some embodiments, the anti-PD-L1 antibody or antigen-binding fragment thereof has a VH having at least 95% sequence identity to SEQ ID NO:127. In some embodiments, the anti-PD-L1 antibody or antigen-binding fragment thereof has a VH having at least 98% sequence identity to SEQ ID NO:127. In some embodiments, provided herein are antibodies or antigen-binding fragments thereof that bind PD-L1 comprising a VH having the amino acid sequence of SEQ ID NO:127.

In some embodiments, provided herein are antibodies or antigen-binding fragments thereof that bind PD-L1 comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:128. In some embodiments, the anti-PD-L1 antibody or antigen-binding fragment thereof has a VH having at least 85% sequence identity to SEQ ID NO:128. In some embodiments, the anti-PD-L1 antibody or antigen-binding fragment thereof has a VH having at least 90% sequence identity to SEQ ID NO:128. In some embodiments, the anti-PD-L1 antibody or antigen-binding fragment thereof has a VH having at least 95% sequence identity to SEQ ID NO:128. In some embodiments, the anti-PD-L1 antibody or antigen-binding fragment thereof has a VH having at least 98% sequence identity to SEQ ID NO:128. In some embodiments, provided herein are antibodies or antigen-binding fragments thereof that bind PD-L1 comprising a VH having the amino acid sequence of SEQ ID NO:128.

In some embodiments, provided herein are antibodies or antigen-binding fragments thereof that bind PD-L1 comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:131. In some embodiments, the anti-PD-L1 antibody or antigen-binding fragment thereof has a VH having at least 85% sequence identity to SEQ ID NO:131. In some embodiments, the anti-PD-L1 antibody or antigen-binding fragment thereof has a VH having at least 90% sequence identity to SEQ ID NO:131. In some embodiments, the anti-PD-L1 antibody or antigen-binding fragment thereof has a VH having at least 95% sequence identity to SEQ ID NO:131. In some embodiments, the anti-PD-L1 antibody or antigen-binding fragment thereof has a VH having at least 98% sequence identity to SEQ ID NO:131. In some embodiments, provided herein are antibodies or antigen-binding fragments thereof that bind PD-L1 comprising a VH having the amino acid sequence of SEQ ID NO:131.

In some embodiments, provided herein are antibodies or antigen-binding fragments thereof that bind PD-L1 comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:132. In some embodiments, the anti-PD-L1 antibody or antigen-binding fragment thereof has a VH having at least 85% sequence identity to SEQ ID NO:132. In some embodiments, the anti-PD-L1 antibody or antigen-binding fragment thereof has a VH having at least 90% sequence identity to SEQ ID NO:132. In some embodiments, the anti-PD-L1 antibody or antigen-binding fragment thereof has a VH having at least 95% sequence identity to SEQ ID NO:132. In some embodiments, the anti-PD-L1 antibody or antigen-binding fragment thereof has a VH having at least 98% sequence identity to SEQ ID NO:132. In some embodiments, provided herein are antibodies or antigen-binding fragments thereof that bind PD-L1 comprising a VH having the amino acid sequence of SEQ ID NO:132.

In some embodiments, provided herein are antibodies or antigen-binding fragments thereof that bind PD-L1 comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:133. In some embodiments, the anti-PD-L1 antibody or antigen-binding fragment thereof has a VH having at least 85% sequence identity to SEQ ID NO:133. In some embodiments, the anti-PD-L1 antibody or antigen-binding fragment thereof has a VH having at least 90% sequence identity to SEQ ID NO:133. In some embodiments, the anti-PD-L1 antibody or antigen-binding fragment thereof has a VH having at least 95% sequence identity to SEQ ID NO:133. In some embodiments, the anti-PD-L1 antibody or antigen-binding fragment thereof has a VH having at least 98% sequence identity to SEQ ID NO:133. In some embodiments, provided herein are antibodies or antigen-binding fragments thereof that bind PD-L1 comprising a VH having the amino acid sequence of SEQ ID NO:133.

In some embodiments, provided herein are antibodies or antigen-binding fragments thereof that bind PD-L1 comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:134. In some embodiments, the anti-PD-L1 antibody or antigen-binding fragment thereof has a VH having at least 85% sequence identity to SEQ ID NO:134. In some embodiments, the anti-PD-L1 antibody or antigen-binding fragment thereof has a VH having at least 90% sequence identity to SEQ ID NO: 134. In some embodiments, the anti-PD-L1 antibody or antigen-binding fragment thereof has a VH having at least 95% sequence identity to SEQ ID NO:134. In some embodiments, the anti-PD-L1 antibody or antigen-binding fragment thereof has a VH having at least 98% sequence identity to SEQ ID NO:134. In some embodiments, provided herein are antibodies or antigen-binding fragments thereof that bind PD-L1 comprising a VH having the amino acid sequence of SEQ ID NO:134.

In some embodiments, provided herein are antibodies or antigen-binding fragments thereof that bind PD-L1 comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:135. In some embodiments, the anti-PD-L1 antibody or antigen-binding fragment thereof has a VH having at least 85% sequence identity to SEQ ID NO:135. In some embodiments, the anti-PD-L1 antibody or antigen-binding fragment thereof has a VH having at least 90% sequence identity to SEQ ID NO:135. In some embodiments, the anti-PD-L1 antibody or antigen-binding fragment thereof has a VH having at least 95% sequence identity to SEQ ID NO:135. In some embodiments, the anti-PD-L1 antibody or antigen-binding fragment thereof has a VH having at least 98% sequence identity to SEQ ID NO:135. In some embodiments, provided herein are antibodies or antigen-binding fragments thereof that bind PD-L1 comprising a VH having the amino acid sequence of SEQ ID NO: 135.

In some embodiments, provided herein are antibodies or antigen-binding fragments thereof that bind PD-L1 comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:136. In some embodiments, the anti-PD-L1 antibody or antigen-binding fragment thereof has a VH having at least 85% sequence identity to SEQ ID NO:136. In some embodiments, the anti-PD-L1 antibody or antigen-binding fragment thereof has a VH having at least 90% sequence identity to SEQ ID NO: 136. In some embodiments, the anti-PD-L1 antibody or antigen-binding fragment thereof has a VH having at least 95% sequence identity to SEQ ID NO:136. In some embodiments, the anti-PD-L1 antibody or antigen-binding fragment thereof has a VH having at least 98% sequence identity to SEQ ID NO:136. In some embodiments, provided herein are antibodies or antigen-binding fragments thereof that bind PD-L1 comprising a VH having the amino acid sequence of SEQ ID NO:136.

In some embodiments, provided herein are antibodies or antigen-binding fragments thereof that bind PD-L1 comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:174. In some embodiments, the anti-PD-L1 antibody or antigen-binding fragment thereof has a VH having at least 85% sequence identity to SEQ ID NO: 174. In some embodiments, the anti-PD-L1 antibody or antigen-binding fragment thereof has a VH having at least 90% sequence identity to SEQ ID NO: 174. In some embodiments, the anti-PD-L1 antibody or antigen-binding fragment thereof has a VH having at least 95% sequence identity to SEQ ID NO:174. In some embodiments, the anti-PD-L1 antibody or antigen-binding fragment thereof has a VH having at least 98% sequence identity to SEQ ID NO:174. In some embodiments, provided herein are antibodies or antigen-binding fragments thereof that bind PD-L1 comprising a VH having the amino acid sequence of SEQ ID NO:174.

In some embodiments, provided herein are antibodies or antigen-binding fragments thereof that bind PD-L1 comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:175. In some embodiments, the anti-PD-L1 antibody or antigen-binding fragment thereof has a VH having at least 85% sequence identity to SEQ ID NO: 175. In some embodiments, the anti-PD-L1 antibody or antigen-binding fragment thereof has a VH having at least 90% sequence identity to SEQ ID NO: 175. In some embodiments, the anti-PD-L1 antibody or antigen-binding fragment thereof has a VH having at least 95% sequence identity to SEQ ID NO:175. In some embodiments, the anti-PD-L1 antibody or antigen-binding fragment thereof has a VH having at least 98% sequence identity to SEQ ID NO:175. In some embodiments, provided herein are antibodies or antigen-binding fragments thereof that bind PD-L1 comprising a VH having the amino acid sequence of SEQ ID NO: 175.

In some embodiments, provided herein are antibodies or antigen-binding fragments thereof that bind PD-L1 comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:176. In some embodiments, the anti-PD-L1 antibody or antigen-binding fragment thereof has a VH having at least 85% sequence identity to SEQ ID NO: 176. In some embodiments, the anti-PD-L1 antibody or antigen-binding fragment thereof has a VH having at least 90% sequence identity to SEQ ID NO: 176. In some embodiments, the anti-PD-L1 antibody or antigen-binding fragment thereof has a VH having at least 95% sequence identity to SEQ ID NO:176. In some embodiments, the anti-PD-L1 antibody or antigen-binding fragment thereof has a VH having at least 98% sequence identity to SEQ ID NO:176. In some embodiments, provided herein are antibodies or antigen-binding fragments thereof that bind PD-L1 comprising a VH having the amino acid sequence of SEQ ID NO: 176.

In some embodiments, provided herein are antibodies or antigen-binding fragments thereof that bind PD-L1 comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:177. In some embodiments, the anti-PD-L1 antibody or antigen-binding fragment thereof has a VH having at least 85% sequence identity to SEQ ID NO: 177. In some embodiments, the anti-PD-L1 antibody or antigen-binding fragment thereof has a VH having at least 90% sequence identity to SEQ ID NO:177. In some embodiments, the anti-PD-L1 antibody or antigen-binding fragment thereof has a VH having at least 95% sequence identity to SEQ ID NO:177. In some embodiments, the anti-PD-L1 antibody or antigen-binding fragment thereof has a VH having at least 98% sequence identity to SEQ ID NO:177. In some embodiments, provided herein are antibodies or antigen-binding fragments thereof that bind PD-L1 comprising a VH having the amino acid sequence of SEQ ID NO:177.

In some embodiments, provided herein are antibodies or antigen-binding fragments thereof that bind PD-L1 comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:178. In some embodiments, the anti-PD-L1 antibody or antigen-binding fragment thereof has a VH having at least 85% sequence identity to SEQ ID NO: 178. In some embodiments, the anti-PD-L1 antibody or antigen-binding fragment thereof has a VH having at least 90% sequence identity to SEQ ID NO:178. In some embodiments, the anti-PD-L1 antibody or antigen-binding fragment thereof has a VH having at least 95% sequence identity to SEQ ID NO:178. In some embodiments, the anti-PD-L1 antibody or antigen-binding fragment thereof has a VH having at least 98% sequence identity to SEQ ID NO:178. In some embodiments, provided herein are antibodies or antigen-binding fragments thereof that bind PD-L1 comprising a VH having the amino acid sequence of SEQ ID NO:178.

In some embodiments, provided herein are antibodies or antigen-binding fragments thereof that bind PD-L1 comprising a VL, wherein the VL has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:111. In some embodiments, the anti-PD-L1 antibody or antigen-binding fragment thereof has a VL having at least 85% sequence identity to SEQ ID NO:111. In some embodiments, the anti-PD-L1 antibody or antigen-binding fragment thereof has a VL having at least 90% sequence identity to SEQ ID NO:111. In some embodiments, the anti-PD-L1 antibody or antigen-binding fragment thereof has a VL having at least 95% sequence identity to SEQ ID NO:111. In some embodiments, the anti-PD-L1 antibody or antigen-binding fragment thereof has a VL having at least 98% sequence identity to SEQ ID NO:111. In some embodiments, provided herein are antibodies or antigen-binding fragments thereof that bind PD-L1 comprising a VL having the amino acid sequence of SEQ ID NO:111.

In some embodiments, provided herein are antibodies or antigen-binding fragments thereof that bind PD-L1 comprising a VL, wherein the VL has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:112. In some embodiments, the anti-PD-L1 antibody or antigen-binding fragment thereof has a VL having at least 85% sequence identity to SEQ ID NO:112. In some embodiments, the anti-PD-L1 antibody or antigen-binding fragment thereof has a VL having at least 90% sequence identity to SEQ ID NO:112. In some embodiments, the anti-PD-L1 antibody or antigen-binding fragment thereof has a VL having at least 95% sequence identity to SEQ ID NO:112. In some embodiments, the anti-PD-L1 antibody or antigen-binding fragment thereof has a VL having at least 98% sequence identity to SEQ ID NO:112. In some embodiments, provided herein are antibodies or antigen-binding fragments thereof that bind PD-L1 comprising a VL having the amino acid sequence of SEQ ID NO:112.

In some embodiments, provided herein are antibodies or antigen-binding fragments thereof that bind PD-L1 comprising a VL, wherein the VL has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:113. In some embodiments, the anti-PD-L1 antibody or antigen-binding fragment thereof has a VL having at least 85% sequence identity to SEQ ID NO:113. In some embodiments, the anti-PD-L1 antibody or antigen-binding fragment thereof has a VL having at least 90% sequence identity to SEQ ID NO:113. In some embodiments, the anti-PD-L1 antibody or antigen-binding fragment thereof has a VL having at least 95% sequence identity to SEQ ID NO:113. In some embodiments, the anti-PD-L1 antibody or antigen-binding fragment thereof has a VL having at least 98% sequence identity to SEQ ID NO:113. In some embodiments, provided herein are antibodies or antigen-binding fragments thereof that bind PD-L1 comprising a VL having the amino acid sequence of SEQ ID NO:113.

In some embodiments, provided herein are antibodies or antigen-binding fragments thereof that bind PD-L1 comprising a VL, wherein the VL has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:114. In some embodiments, the anti-PD-L1 antibody or antigen-binding fragment thereof has a VL having at least 85% sequence identity to SEQ ID NO:114. In some embodiments, the anti-PD-L1 antibody or antigen-binding fragment thereof has a VL having at least 90% sequence identity to SEQ ID NO:114. In some embodiments, the anti-PD-L1 antibody or antigen-binding fragment thereof has a VL having at least 95% sequence identity to SEQ ID NO:114. In some embodiments, the anti-PD-L1 antibody or antigen-binding fragment thereof has a VL having at least 98% sequence identity to SEQ ID NO:114. In some embodiments, provided herein are antibodies or antigen-binding fragments thereof that bind PD-L1 comprising a VL having the amino acid sequence of SEQ ID NO:114.

In some embodiments, provided herein are antibodies or antigen-binding fragments thereof that bind PD-L1 comprising a VL, wherein the VL has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:115. In some embodiments, the anti-PD-L1 antibody or antigen-binding fragment thereof has a VL having at least 85% sequence identity to SEQ ID NO:115. In some embodiments, the anti-PD-L1 antibody or antigen-binding fragment thereof has a VL having at least 90% sequence identity to SEQ ID NO:115. In some embodiments, the anti-PD-L1 antibody or antigen-binding fragment thereof has a VL having at least 95% sequence identity to SEQ ID NO:115. In some embodiments, the anti-PD-L1 antibody or antigen-binding fragment thereof has a VL having at least 98% sequence identity to SEQ ID NO:115. In some embodiments, provided herein are antibodies or antigen-binding fragments thereof that bind PD-L1 comprising a VL having the amino acid sequence of SEQ ID NO:115.

In some embodiments, provided herein are antibodies or antigen-binding fragments thereof that bind PD-L1 comprising a VL, wherein the VL has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:116. In some embodiments, the anti-PD-L1 antibody or antigen-binding fragment thereof has a VL having at least 85% sequence identity to SEQ ID NO:116. In some embodiments, the anti-PD-L1 antibody or antigen-binding fragment thereof has a VL having at least 90% sequence identity to SEQ ID NO:116. In some embodiments, the anti-PD-L1 antibody or antigen-binding fragment thereof has a VL having at least 95% sequence identity to SEQ ID NO:116. In some embodiments, the anti-PD-L1 antibody or antigen-binding fragment thereof has a VL having at least 98% sequence identity to SEQ ID NO:116. In some embodiments, provided herein are antibodies or antigen-binding fragments thereof that bind PD-L1 comprising a VL having the amino acid sequence of SEQ ID NO:116.

In some embodiments, provided herein are antibodies or antigen-binding fragments thereof that bind PD-L1 comprising a VL, wherein the VL has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:117. In some embodiments, the anti-PD-L1 antibody or antigen-binding fragment thereof has a VL having at least 85% sequence identity to SEQ ID NO:117. In some embodiments, the anti-PD-L1 antibody or antigen-binding fragment thereof has a VL having at least 90% sequence identity to SEQ ID NO:117. In some embodiments, the anti-PD-L1 antibody or antigen-binding fragment thereof has a VL having at least 95% sequence identity to SEQ ID NO:117. In some embodiments, the anti-PD-L1 antibody or antigen-binding fragment thereof has a VL having at least 98% sequence identity to SEQ ID NO:117. In some embodiments, provided herein are antibodies or antigen-binding fragments thereof that bind PD-L1 comprising a VL having the amino acid sequence of SEQ ID NO:117.

In some embodiments, provided herein are antibodies or antigen-binding fragments thereof that bind PD-L1 comprising a VL, wherein the VL has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:118. In some embodiments, the anti-PD-L1 antibody or antigen-binding fragment thereof has a VL having at least 85% sequence identity to SEQ ID NO:118. In some embodiments, the anti-PD-L1 antibody or antigen-binding fragment thereof has a VL having at least 90% sequence identity to SEQ ID NO:118. In some embodiments, the anti-PD-L1 antibody or antigen-binding fragment thereof has a VL having at least 95% sequence identity to SEQ ID NO:118. In some embodiments, the anti-PD-L1 antibody or antigen-binding fragment thereof has a VL having at least 98% sequence identity to SEQ ID NO:118. In some embodiments, provided herein are antibodies or antigen-binding fragments thereof that bind PD-L1 comprising a VL having the amino acid sequence of SEQ ID NO:118.

In some embodiments, provided herein are antibodies or antigen-binding fragments thereof that bind PD-L1 comprising a VL, wherein the VL has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:119. In some embodiments, the anti-PD-L1 antibody or antigen-binding fragment thereof has a VL having at least 85% sequence identity to SEQ ID NO:119. In some embodiments, the anti-PD-L1 antibody or antigen-binding fragment thereof has a VL having at least 90% sequence identity to SEQ ID NO:119. In some embodiments, the anti-PD-L1 antibody or antigen-binding fragment thereof has a VL having at least 95% sequence identity to SEQ ID NO:119. In some embodiments, the anti-PD-L1 antibody or antigen-binding fragment thereof has a VL having at least 98% sequence identity to SEQ ID NO:119. In some embodiments, provided herein are antibodies or antigen-binding fragments thereof that bind PD-L1 comprising a VL having the amino acid sequence of SEQ ID NO:119.

In some embodiments, provided herein are antibodies or antigen-binding fragments thereof that bind PD-L1 comprising a VL, wherein the VL has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:120. In some embodiments, the anti-PD-L1 antibody or antigen-binding fragment thereof has a VL having at least 85% sequence identity to SEQ ID NO:120. In some embodiments, the anti-PD-L1 antibody or antigen-binding fragment thereof has a VL having at least 90% sequence identity to SEQ ID NO:120. In some embodiments, the anti-PD-L1 antibody or antigen-binding fragment thereof has a VL having at least 95% sequence identity to SEQ ID NO:120. In some embodiments, the anti-PD-L1 antibody or antigen-binding fragment thereof has a VL having at least 98% sequence identity to SEQ ID NO:120. In some embodiments, provided herein are antibodies or antigen-binding fragments thereof that bind PD-L1 comprising a VL having the amino acid sequence of SEQ ID NO:120.

In some embodiments, provided herein are antibodies or antigen-binding fragments thereof that bind PD-L1 comprising a VL, wherein the VL has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:121. In some embodiments, the anti-PD-L1 antibody or antigen-binding fragment thereof has a VL having at least 85% sequence identity to SEQ ID NO:121. In some embodiments, the anti-PD-L1 antibody or antigen-binding fragment thereof has a VL having at least 90% sequence identity to SEQ ID NO: 121. In some embodiments, the anti-PD-L1 antibody or antigen-binding fragment thereof has a VL having at least 95% sequence identity to SEQ ID NO:121. In some embodiments, the anti-PD-L1 antibody or antigen-binding fragment thereof has a VL having at least 98% sequence identity to SEQ ID NO:121. In some embodiments, provided herein are antibodies or antigen-binding fragments thereof that bind PD-L1 comprising a VL having the amino acid sequence of SEQ ID NO:121.

In some embodiments, provided herein are antibodies or antigen-binding fragments thereof that bind PD-L1 comprising a VL, wherein the VL has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:122. In some embodiments, the anti-PD-L1 antibody or antigen-binding fragment thereof has a VL having at least 85% sequence identity to SEQ ID NO:122. In some embodiments, the anti-PD-L1 antibody or antigen-binding fragment thereof has a VL having at least 90% sequence identity to SEQ ID NO: 122. In some embodiments, the anti-PD-L1 antibody or antigen-binding fragment thereof has a VL having at least 95% sequence identity to SEQ ID NO:122. In some embodiments, the anti-PD-L1 antibody or antigen-binding fragment thereof has a VL having at least 98% sequence identity to SEQ ID NO:122. In some embodiments, provided herein are antibodies or antigen-binding fragments thereof that bind PD-L1 comprising a VL having the amino acid sequence of SEQ ID NO:122.

In some embodiments, provided herein are antibodies or antigen-binding fragments thereof that bind PD-L1 comprising a VL, wherein the VL has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:123. In some embodiments, the anti-PD-L1 antibody or antigen-binding fragment thereof has a VL having at least 85% sequence identity to SEQ ID NO:123. In some embodiments, the anti-PD-L1 antibody or antigen-binding fragment thereof has a VL having at least 90% sequence identity to SEQ ID NO:123. In some embodiments, the anti-PD-L1 antibody or antigen-binding fragment thereof has a VL having at least 95% sequence identity to SEQ ID NO:123. In some embodiments, the anti-PD-L1 antibody or antigen-binding fragment thereof has a VL having at least 98% sequence identity to SEQ ID NO:123. In some embodiments, provided herein are antibodies or antigen-binding fragments thereof that bind PD-L1 comprising a VL having the amino acid sequence of SEQ ID NO:123.

In some embodiments, provided herein are antibodies or antigen-binding fragments thereof that bind PD-L1 comprising a VL, wherein the VL has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:125. In some embodiments, the anti-PD-L1 antibody or antigen-binding fragment thereof has a VL having at least 85% sequence identity to SEQ ID NO:125. In some embodiments, the anti-PD-L1 antibody or antigen-binding fragment thereof has a VL having at least 90% sequence identity to SEQ ID NO: 125. In some embodiments, the anti-PD-L1 antibody or antigen-binding fragment thereof has a VL having at least 95% sequence identity to SEQ ID NO:125. In some embodiments, the anti-PD-L1 antibody or antigen-binding fragment thereof has a VL having at least 98% sequence identity to SEQ ID NO:125. In some embodiments, provided herein are antibodies or antigen-binding fragments thereof that bind PD-L1 comprising a VL having the amino acid sequence of SEQ ID NO: 125.

In some embodiments, provided herein are antibodies or antigen-binding fragments thereof that bind PD-L1 comprising a VL, wherein the VL has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:129. In some embodiments, the anti-PD-L1 antibody or antigen-binding fragment thereof has a VL having at least 85% sequence identity to SEQ ID NO:129. In some embodiments, the anti-PD-L1 antibody or antigen-binding fragment thereof has a VL having at least 90% sequence identity to SEQ ID NO:129. In some embodiments, the anti-PD-L1 antibody or antigen-binding fragment thereof has a VL having at least 95% sequence identity to SEQ ID NO:129. In some embodiments, the anti-PD-L1 antibody or antigen-binding fragment thereof has a VL having at least 98% sequence identity to SEQ ID NO:129. In some embodiments, provided herein are antibodies or antigen-binding fragments thereof that bind PD-L1 comprising a VL having the amino acid sequence of SEQ ID NO:129.

In some embodiments, provided herein are antibodies or antigen-binding fragments thereof that bind PD-L1 comprising a VL, wherein the VL has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:130. In some embodiments, the anti-PD-L1 antibody or antigen-binding fragment thereof has a VL having at least 85% sequence identity to SEQ ID NO:130. In some embodiments, the anti-PD-L1 antibody or antigen-binding fragment thereof has a VL having at least 90% sequence identity to SEQ ID NO:130. In some embodiments, the anti-PD-L1 antibody or antigen-binding fragment thereof has a VL having at least 95% sequence identity to SEQ ID NO:130. In some embodiments, the anti-PD-L1 antibody or antigen-binding fragment thereof has a VL having at least 98% sequence identity to SEQ ID NO:130. In some embodiments, provided herein are antibodies or antigen-binding fragments thereof that bind PD-L1 comprising a VL having the amino acid sequence of SEQ ID NO:130.

In some embodiments, provided herein are antibodies or antigen-binding fragments thereof that bind PD-L1 comprising a VL, wherein the VL has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:137. In some embodiments, the anti-PD-L1 antibody or antigen-binding fragment thereof has a VL having at least 85% sequence identity to SEQ ID NO:137. In some embodiments, the anti-PD-L1 antibody or antigen-binding fragment thereof has a VL having at least 90% sequence identity to SEQ ID NO:137. In some embodiments, the anti-PD-L1 antibody or antigen-binding fragment thereof has a VL having at least 95% sequence identity to SEQ ID NO:137. In some embodiments, the anti-PD-L1 antibody or antigen-binding fragment thereof has a VL having at least 98% sequence identity to SEQ ID NO:137. In some embodiments, provided herein are antibodies or antigen-binding fragments thereof that bind PD-L1 comprising a VL having the amino acid sequence of SEQ ID NO:137.

In some embodiments, provided herein are antibodies or antigen-binding fragments thereof that bind PD-L1 comprising a VL, wherein the VL has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:138. In some embodiments, the anti-PD-L1 antibody or antigen-binding fragment thereof has a VL having at least 85% sequence identity to SEQ ID NO:138. In some embodiments, the anti-PD-L1 antibody or antigen-binding fragment thereof has a VL having at least 90% sequence identity to SEQ ID NO:138. In some embodiments, the anti-PD-L1 antibody or antigen-binding fragment thereof has a VL having at least 95% sequence identity to SEQ ID NO:138. In some embodiments, the anti-PD-L1 antibody or antigen-binding fragment thereof has a VL having at least 98% sequence identity to SEQ ID NO:138. In some embodiments, provided herein are antibodies or antigen-binding fragments thereof that bind PD-L1 comprising a VL having the amino acid sequence of SEQ ID NO:138.

In some embodiments, provided herein are antibodies or antigen-binding fragments thereof that bind PD-L1 comprising a VL, wherein the VL has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:139. In some embodiments, the anti-PD-L1 antibody or antigen-binding fragment thereof has a VL having at least 85% sequence identity to SEQ ID NO:139. In some embodiments, the anti-PD-L1 antibody or antigen-binding fragment thereof has a VL having at least 90% sequence identity to SEQ ID NO:139. In some embodiments, the anti-PD-L1 antibody or antigen-binding fragment thereof has a VL having at least 95% sequence identity to SEQ ID NO:139. In some embodiments, the anti-PD-L1 antibody or antigen-binding fragment thereof has a VL having at least 98% sequence identity to SEQ ID NO:139. In some embodiments, provided herein are antibodies or antigen-binding fragments thereof that bind PD-L1 comprising a VL having the amino acid sequence of SEQ ID NO:139.

In some embodiments, provided herein are antibodies or antigen-binding fragments thereof that bind PD-L1 comprising a VL, wherein the VL has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:140. In some embodiments, the anti-PD-L1 antibody or antigen-binding fragment thereof has a VL having at least 85% sequence identity to SEQ ID NO:140. In some embodiments, the anti-PD-L1 antibody or antigen-binding fragment thereof has a VL having at least 90% sequence identity to SEQ ID NO:140. In some embodiments, the anti-PD-L1 antibody or antigen-binding fragment thereof has a VL having at least 95% sequence identity to SEQ ID NO:140. In some embodiments, the anti-PD-L1 antibody or antigen-binding fragment thereof has a VL having at least 98% sequence identity to SEQ ID NO:140. In some embodiments, provided herein are antibodies or antigen-binding fragments thereof that bind PD-L1 comprising a VL having the amino acid sequence of SEQ ID NO:140.

In some embodiments, provided herein are antibodies or antigen-binding fragments thereof that bind PD-L1 comprising a VL, wherein the VL has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:141. In some embodiments, the anti-PD-L1 antibody or antigen-binding fragment thereof has a VL having at least 85% sequence identity to SEQ ID NO:141. In some embodiments, the anti-PD-L1 antibody or antigen-binding fragment thereof has a VL having at least 90% sequence identity to SEQ ID NO:141. In some embodiments, the anti-PD-L1 antibody or antigen-binding fragment thereof has a VL having at least 95% sequence identity to SEQ ID NO:141. In some embodiments, the anti-PD-L1 antibody or antigen-binding fragment thereof has a VL having at least 98% sequence identity to SEQ ID NO:141. In some embodiments, provided herein are antibodies or antigen-binding fragments thereof that bind PD-L1 comprising a VL having the amino acid sequence of SEQ ID NO:141.

In some embodiments, provided herein are antibodies or antigen-binding fragments thereof that bind PD-L1 comprising a VL, wherein the VL has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:142. In some embodiments, the anti-PD-L1 antibody or antigen-binding fragment thereof has a VL having at least 85% sequence identity to SEQ ID NO:142. In some embodiments, the anti-PD-L1 antibody or antigen-binding fragment thereof has a VL having at least 90% sequence identity to SEQ ID NO:142. In some embodiments, the anti-PD-L1 antibody or antigen-binding fragment thereof has a VL having at least 95% sequence identity to SEQ ID NO:142. In some embodiments, the anti-PD-L1 antibody or antigen-binding fragment thereof has a VL having at least 98% sequence identity to SEQ ID NO:142. In some embodiments, provided herein are antibodies or antigen-binding fragments thereof that bind PD-L1 comprising a VL having the amino acid sequence of SEQ ID NO:142.

In some embodiments, provided herein are antibodies or antigen-binding fragments thereof that bind PD-L1 comprising a VL, wherein the VL has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:143. In some embodiments, the anti-PD-L1 antibody or antigen-binding fragment thereof has a VL having at least 85% sequence identity to SEQ ID NO:143. In some embodiments, the anti-PD-L1 antibody or antigen-binding fragment thereof has a VL having at least 90% sequence identity to SEQ ID NO:143. In some embodiments, the anti-PD-L1 antibody or antigen-binding fragment thereof has a VL having at least 95% sequence identity to SEQ ID NO:143. In some embodiments, the anti-PD-L1 antibody or antigen-binding fragment thereof has a VL having at least 98% sequence identity to SEQ ID NO:143. In some embodiments, provided herein are antibodies or antigen-binding fragments thereof that bind PD-L1 comprising a VL having the amino acid sequence of SEQ ID NO:143.

In some embodiments, provided herein are antibodies or antigen-binding fragments thereof that bind PD-L1 comprising a VL, wherein the VL has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:144. In some embodiments, the anti-PD-L1 antibody or antigen-binding fragment thereof has a VL having at least 85% sequence identity to SEQ ID NO:144. In some embodiments, the anti-PD-L1 antibody or antigen-binding fragment thereof has a VL having at least 90% sequence identity to SEQ ID NO:144. In some embodiments, the anti-PD-L1 antibody or antigen-binding fragment thereof has a VL having at least 95% sequence identity to SEQ ID NO:144. In some embodiments, the anti-PD-L1 antibody or antigen-binding fragment thereof has a VL having at least 98% sequence identity to SEQ ID NO:144. In some embodiments, provided herein are antibodies or antigen-binding fragments thereof that bind PD-L1 comprising a VL having the amino acid sequence of SEQ ID NO: 144.

In some embodiments, provided herein are antibodies or antigen-binding fragments thereof that bind PD-L1 comprising a VL, wherein the VL has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:179. In some embodiments, the anti-PD-L1 antibody or antigen-binding fragment thereof has a VL having at least 85% sequence identity to SEQ ID NO:179. In some embodiments, the anti-PD-L1 antibody or antigen-binding fragment thereof has a VL having at least 90% sequence identity to SEQ ID NO: 179. In some embodiments, the anti-PD-L1 antibody or antigen-binding fragment thereof has a VL having at least 95% sequence identity to SEQ ID NO:179. In some embodiments, the anti-PD-L1 antibody or antigen-binding fragment thereof has a VL having at least 98% sequence identity to SEQ ID NO:179. In some embodiments, provided herein are antibodies or antigen-binding fragments thereof that bind PD-L1 comprising a VL having the amino acid sequence of SEQ ID NO: 179.

In some embodiments, provided herein are antibodies or antigen-binding fragments thereof that bind PD-L1 comprising a VL, wherein the VL has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:180. In some embodiments, the anti-PD-L1 antibody or antigen-binding fragment thereof has a VL having at least 85% sequence identity to SEQ ID NO:180. In some embodiments, the anti-PD-L1 antibody or antigen-binding fragment thereof has a VL having at least 90% sequence identity to SEQ ID NO:180. In some embodiments, the anti-PD-L1 antibody or antigen-binding fragment thereof has a VL having at least 95% sequence identity to SEQ ID NO:180. In some embodiments, the anti-PD-L1 antibody or antigen-binding fragment thereof has a VL having at least 98% sequence identity to SEQ ID NO:180. In some embodiments, provided herein are antibodies or antigen-binding fragments thereof that bind PD-L1 comprising a VL having the amino acid sequence of SEQ ID NO:180.

In some embodiments, provided herein are antibodies or antigen-binding fragments thereof that bind PD-L1 comprising a VL, wherein the VL has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:181. In some embodiments, the anti-PD-L1 antibody or antigen-binding fragment thereof has a VL having at least 85% sequence identity to SEQ ID NO:181. In some embodiments, the anti-PD-L1 antibody or antigen-binding fragment thereof has a VL having at least 90% sequence identity to SEQ ID NO:181. In some embodiments, the anti-PD-L1 antibody or antigen-binding fragment thereof has a VL having at least 95% sequence identity to SEQ ID NO:181. In some embodiments, the anti-PD-L1 antibody or antigen-binding fragment thereof has a VL having at least 98% sequence identity to SEQ ID NO: 181. In some embodiments, provided herein are antibodies or antigen-binding fragments thereof that bind PD-L1 comprising a VL having the amino acid sequence of SEQ ID NO:181.

The anti-PD-L1 antibodies or antigen-binding fragments thereof can comprise a combination of any VH disclosed herein and any VL disclosed herein.

In some embodiments, provided herein are antibodies or antigen-binding fragments thereof that bind PD-L1 comprising a VH and a VL, wherein the VH and VL have the amino acid sequences of (1) SEQ ID NOs:98 and 111, respectively; (2) SEQ ID NOs:99 and 112, respectively; (3) SEQ ID NOs: 100 and 113, respectively; (4) SEQ ID NOs: 101 and 114, respectively; (5) SEQ ID NOs: 102 and 115, respectively; (6) SEQ ID NOs:103 and 116, respectively; (7) SEQ ID NOs: 104 and 117, respectively; (8) SEQ ID NOs:105 and 118, respectively; (9) SEQ ID NOs: 106 and 119, respectively; (10) SEQ ID NOs: 107 and 120, respectively; (11) SEQ ID NOs: 108 and 121, respectively;(12) SEQ ID NOs:109 and 122, respectively; (13) SEQ ID NOs: 110 and 123, respectively; or (14) SEQ ID NOs:124 and 125, respectively.

In some embodiments, provided herein are antibodies or antigen-binding fragments thereof that bind PD-L1 comprising a VH and a VL, wherein the VH has an amino acid sequence selected from the group consisting of SEQ ID NOs: 126-128, and/or wherein the VL has the amino acid sequence of SEQ ID NO: 129 or 130. In some embodiments, the VH and the VL have the amino acid sequences of (1) SEQ ID NOs:126 and 129, respectively; (2) SEQ ID NOs:126 and 130, respectively; (3) SEQ ID NOs: 127 and 129, respectively; (4) SEQ ID NOs: 127 and 130, respectively; (5) SEQ ID NOs: 128 and 129, respectively; or (6) SEQ ID NOs: 128 and 130, respectively.

In some embodiments, provided herein are antibodies or antigen-binding fragments thereof that bind PD-L1 comprising a VH and a VL, wherein the VH has an amino acid sequence selected from the group consisting of SEQ ID NOs: 131-136, and/or wherein the VL has an amino acid sequence selected from the group consisting of SEQ ID NOs:137-144. In some embodiments, the VH and the VL have the amino acid sequences of (1) SEQ ID NOs: 131 and 137, respectively; (2) SEQ ID NOs: 131 and 138, respectively; (3) SEQ ID NOs: 131 and 139, respectively; (4) SEQ ID NOs: 131 and 140, respectively; (5) SEQ ID NOs: 131 and 141, respectively; (6) SEQ ID NOs: 131 and 142, respectively; (7) SEQ ID NOs: 131 and 143, respectively; (8) SEQ ID NOs: 131 and 144, respectively; (9) SEQ ID NOs: 132 and 137, respectively; (10) SEQ ID NOs: 132 and 138, respectively; (11) SEQ ID NOs:132 and 139, respectively; (12) SEQ ID NOs: 132 and 140, respectively; (13) SEQ ID NOs:132 and 141, respectively; (14) SEQ ID NOs: 132 and 142, respectively; (15) SEQ ID NOs:132 and 143, respectively; (16) SEQ ID NOs: 132 and 144, respectively; (17) SEQ ID NOs: 133 and 137, respectively; (18) SEQ ID NOs: 133 and 138, respectively; (19) SEQ ID NOs: 133 and 139, respectively; (20) SEQ ID NOs: 133 and 140, respectively; (21) SEQ ID NOs: 133 and 141, respectively; (22) SEQ ID NOs:133 and 142, respectively; (23) SEQ ID NOs:133 and 143, respectively; (24) SEQ ID NOs: 133 and 144, respectively; (25) SEQ ID NOs: 134 and 137, respectively; (26) SEQ ID NOs: 134 and 138, respectively; (27) SEQ ID NOs: 134 and 139, respectively; (28) SEQ ID NOs:134 and 140, respectively; (29) SEQ ID NOs:134 and 141, respectively; (30) SEQ ID NOs: 134 and 142, respectively; (31) SEQ ID NOs: 134 and 143, respectively; (32) SEQ ID NOs: 134 and 144, respectively; (33) SEQ ID NOs: 135 and 137, respectively; (34) SEQ ID NOs:135 and 138, respectively; (35) SEQ ID NOs:135 and 139, respectively; (36) SEQ ID NOs: 135 and 140, respectively; (37) SEQ ID NOs: 135 and 141, respectively; (38) SEQ ID NOs: 135 and 142, respectively; (39) SEQ ID NOs: 135 and 143, respectively; (40) SEQ ID NOs:135 and 144, respectively; (41) SEQ ID NOs:136 and 137, respectively; (42) SEQ ID NOs: 136 and 138, respectively; (43) SEQ ID NOs: 136 and 139, respectively; (44) SEQ ID NOs: 136 and 140, respectively; (45) SEQ ID NOs: 136 and 141, respectively; (46) SEQ ID NOs:136 and 142, respectively; (47) SEQ ID NOs:136 and 143, respectively; or (48) SEQ ID NOs:136 and 144, respectively.

In some embodiments, provided herein are antibodies or antigen-binding fragments thereof that bind PD-L1 comprising a VH and a VL, wherein the VH has an amino acid sequence selected from the group consisting of SEQ ID NOs: 174-178, and/or wherein the VL has the amino acid sequence of SEQ ID NOs: 179-181. In some embodiments, the VH and the VL have the amino acid sequences of (1) SEQ ID NOs: 174 and 179, respectively; (2) SEQ ID NOs: 175 and 179, respectively; (3) SEQ ID NOs:176 and 179, respectively; (4) SEQ ID NOs: 177 and 179, respectively; (5) SEQ ID NOs: 178 and 179, respectively; (6) SEQ ID NOs: 174 and 180, respectively; (7) SEQ ID NOs: 175 and 180, respectively; (8) SEQ ID NOs: 176 and 180, respectively; (9) SEQ ID NOs: 177 and 180, respectively; (10) SEQ ID NOs: 178 and 180, respectively; (11) SEQ ID NOs: 174 and 181, respectively; (12) SEQ ID NOs: 175 and 181, respectively; (13) SEQ ID NOs: 176 and 181, respectively; (14) SEQ ID NOs: 177 and 181, respectively; or (15) SEQ ID NOs: 178 and 181, respectively.

In some embodiments, provided herein are antibodies or antigen-binding fragments thereof that bind PD-L1 comprising (a) a VH comprising VH CDRs 1, 2, and 3 from a VH having an amino acid sequence selected from the group consisting of SEQ ID NOs:98-110, 124, 126-128, 131-136 and 174-178; and/or (b) a VL comprising VL CDRs 1, 2, and 3 from a VL having an amino acid sequence selected from the group consisting of SEQ ID NOs: 111-123, 125, 129-130, 137-144 and 179-181.

In some embodiments, the anti-PD-L1 antibody or antigen-binding fragment thereof provided herein is the antibody designated as Clone A. In some embodiments, the anti-PD-L1 antibody or antigen-binding fragment thereof provided herein has a VH from Clone A. In some embodiments, the anti-PD-L1 antibody or antigen-binding fragment thereof provided herein has a VL from Clone A. In some embodiments, the anti-PD-L1 antibody or antigen-binding fragment thereof provided herein has a VH and a VL from Clone A. In some embodiments, the anti-PD-L1 antibody or antigen-binding fragment thereof provided herein has a VH that comprises VH CDRs 1, 2, and 3 from a VH from Clone A. In some embodiments, the anti-PD-L1 antibody or antigen-binding fragment thereof provided herein has a VL that comprises VL CDRs 1, 2, and 3 from a VL from Clone A. In some embodiments, the anti-PD-L1 antibody or antigen-binding fragment thereof provided herein has a VH comprising VH CDRs 1, 2, and 3 and a VL comprising VL CDRs 1, 2, and 3 from the VH and VL of Clone A, respectively. In some embodiments, the VH from Clone A has the amino acid sequence of SEQ ID NO:98. In some embodiments, the VL from Clone A has the amino acid sequence of SEQ ID NO:111. In some embodiments, the anti-PD-L1 antibody or antigen-binding fragment thereof provided herein has a VH that is a variant of the VH of Clone A having up to about 5 amino acid substitutions, additions, and/or deletions in SEQ ID NO:98. In some embodiments, the amino acid substitutions, additions, and/or deletions are in the VH CDRs. In some embodiments, the amino acid substitutions, additions, and/or deletions are not in the VH CDRs. In some embodiments, the variant of the VH of Clone A has up to about 5 conservative amino acid substitutions. In some embodiments, the variant of the VH of Clone A has up to 3 conservative amino acid substitutions. In some embodiments, the anti-PD-L1 antibody or antigen-binding fragment thereof provided herein has a VL that is a variant of the VL of Clone A having up to about 5 amino acid substitutions, additions, and/or deletions in SEQ ID NO:111. In some embodiments, the amino acid substitutions, additions, and/or deletions are in the VL CDRs. In some embodiments, the amino acid substitutions, additions, and/or deletions are not in the VL CDRs. In some embodiments, the variant of the VL of Clone A has up to about 5 conservative amino acid substitutions. In some embodiments, the variant of the VL of Clone A has up to about 3 conservative amino acid substitutions. In some embodiments, the anti-PD-L1 antibody or antigen-binding fragment thereof provided herein is a chimeric antibody or antigen-binding fragment derived from Clone A. In some embodiments, the anti-PD-L1 antibody or antigen-binding fragment thereof provided herein is a humanized antibody or antigen-binding fragment derived from Clone A. In some embodiments, the anti-PD-L1 antibody or antigen-binding fragment thereof provided herein is a human antibody or antigen-binding fragment derived from Clone A.

In some embodiments, the anti-PD-L1 antibody or antigen-binding fragment thereof provided herein is the antibody designated as Clone B. In some embodiments, the anti-PD-L1 antibody or antigen-binding fragment thereof provided herein has a VH from Clone B. In some embodiments, the anti-PD-L1 antibody or antigen-binding fragment thereof provided herein has a VL from Clone B. In some embodiments, the anti-PD-L1 antibody or antigen-binding fragment thereof provided herein has a VH and a VL from Clone B. In some embodiments, the anti-PD-L1 antibody or antigen-binding fragment thereof provided herein has a VH that comprises VH CDRs 1, 2, and 3 from a VH from Clone B. In some embodiments, the anti-PD-L1 antibody or antigen-binding fragment thereof provided herein has a VL that comprises VL CDRs 1, 2, and 3 from a VL from Clone B. In some embodiments, the anti-PD-L1 antibody or antigen-binding fragment thereof provided herein has a VH comprising VH CDRs 1, 2, and 3 and a VL comprising VL CDRs 1, 2, and 3 from the VH and VL of Clone B, respectively. In some embodiments, the VH from Clone B has the amino acid sequence of SEQ ID NO:99. In some embodiments, the VL from Clone B has the amino acid sequence of SEQ ID NO: 112. In some embodiments, the anti-PD-L1 antibody or antigen-binding fragment thereof provided herein has a VH that is a variant of the VH of Clone B having up to about 5 amino acid substitutions, additions, and/or deletions in SEQ ID NO:99. In some embodiments, the amino acid substitutions, additions, and/or deletions are in the VH CDRs. In some embodiments, the amino acid substitutions, additions, and/or deletions are not in the VH CDRs. In some embodiments, the variant of the VH of Clone B has up to about 5 conservative amino acid substitutions. In some embodiments, the variant of the VH of Clone B has up to 3 conservative amino acid substitutions. In some embodiments, the anti-PD-L1 antibody or antigen-binding fragment thereof provided herein has a VL that is a variant of the VL of Clone B having up to about 5 amino acid substitutions, additions, and/or deletions in SEQ ID NO: 112. In some embodiments, the amino acid substitutions, additions, and/or deletions are in the VL CDRs. In some embodiments, the amino acid substitutions, additions, and/or deletions are not in the VL CDRs. In some embodiments, the variant of the VL of Clone B has up to about 5 conservative amino acid substitutions. In some embodiments, the variant of the VL of Clone B has up to about 3 conservative amino acid substitutions. In some embodiments, the anti-PD-L1 antibody or antigen-binding fragment thereof provided herein is a chimeric antibody or antigen-binding fragment derived from Clone B. In some embodiments, the anti-PD-L1 antibody or antigen-binding fragment thereof provided herein is a humanized antibody or antigen-binding fragment derived from Clone B. In some embodiments, the anti-PD-L1 antibody or antigen-binding fragment thereof provided herein is a human antibody or antigen-binding fragment derived from Clone B.

TABLE 4

Amino acid sequences of heavy chain variable regions (VHs) and light chain variable region (VLs) of humanized Clone B

| | |
|---|---|
| Domain | Sequences |
| Bh VH1 | QVQLVQSGAEVKKPGASVKVSCKASGFTFSSSYISWVRQAPGQGLEWMGWIYAGTG GTSYNQKFTGRVTMTTDTSTSTAYMELRSLRSDDTAVYYCARHEGKYWYFDVWGQ GTTVTVSS (SEQ ID NO:131) |
| Bh VH2 | QGQLVQSGAEVKKPGASVKVSCKTSGFTFSSSYISWVRQAPGQGLEWMGWIYAGTG GTSYNQKFTGRVTMTVDTSTSTAYMELRSLRSDDTAVYYCARHEGKYWYFDVWGQ GTTVTVSS (SEQ ID NO:132) |
| Bh VH3 | QVQLVQSGAEVKKPGASVKVSCKTSGFTFSSSYISWVRQAPGQGLEWMGWIYAGTG GTSYNQKFTGRVTMTVDTSTSTAYMELRSLRSDDTAVYYCARHEGKYWYFDVWGQ GTTVTVSS (SEQ ID NO:133) |
| Bh VH4 | QVQLVQSGAEVKKPGSSVKVSCKASGFTFSSSYISWVRQAPGQGLEWMGWIYAGTG GTSYNQKFTGRVTITADKSTSTAYMELSSLRSEDTAVYYCARHEGKYWYFDVWGQG TTVTVSS (SEQ ID NO:134) |
| Bh VH5 | QGQLVQSGAEVKKPGSSVKVSCKTSGFTFSSSYISWVRQAPGQGLEWMGWIYAGTG GTSYNQKFTGRVTITVDTSTSTAYMELSSLRSEDTAVYYCARHEGKYWYFDVWGQG TTVTVSS (SEQ ID NO:135) |
| Bh VH6 | QVQLVQSGAEVKKPGSSVKVSCKTSGFTFSSSYISWVRQAPGQGLEWMGWIYAGTG GTSYNQKFTGRVTITVDTSTSTAYMELSSLRSEDTAVYYCARHEGKYWYFDVWGQG TTVTVSS (SEQ ID NO:136) |
| Bh VL1 | DIQMTQSPSSLSASVGDRVTITCSASSSVSYVHWYQQKPGKAPKPWIYDTSNLASGFP SRFSGSGSGTDYTLTISSLQPEDFATYYCHQRSSYPWTFGGGTKVEIK (SEQ ID NO:137) |
| Bh VL2 | EIVLTQSPATLSLSPGERATLSCSASSSVSYVHWYQQKPGQAPRPWIYDTSNLASGFPA RFSGSGSGTDYTLTISSLEPEDAAVYYCHQRSSYPWTFGGGTKVEIK (SEQ ID NO: 138) |
| Bh VL3 | DIQMTQSPSTLSASVGDRVTITCSASSSVSYVHWYQQKPGKAPKLLIYDTSNLASGVP SRFSGSGSGTEFTLTISSLQPDDFATYYCHQRSSYPWTFGGGTKVEIK (SEQ ID NO:139) |
| Bh VL4 | DIQMTQSPSTLSASVGDRVTITCSASSSVSYVHWYQQKPGKAPKPWIYDTSNLASGFP SRFSGSGSGTEYTLTISSLQPDDFATYYCHQRSSYPWTFGGGTKVEIK (SEQ ID NO:140) |
| Bh VL5 | DIVMTQSPLSLPVTPGEPASISCSASSSVSYVHWYLQKPGQSPQLLIYDTSNLASGVPD RFSGSGSGTDFTLKISRVEAEDVGVYYCHQRSSYPWTFGGGTKVEIK (SEQ ID NO:141) |
| Bh VL6 | DIVMTQSPLSLPVTPGEPASISCSASSSVSYVIIWYLQKPGQSPQPWIYDTSNLASGFPD RFSGSGSGTDYTLKISRVEAEDVGVYYCHQRSSYPWTFGGGTKVEIK (SEQ ID NO:142) |
| Bh VL7 | EIVMTQSPATLSVSPGERATLSCSASSSVSYVHWYQQKPGQAPRLLIYDTSNLASGIPA RFSGSGSGTEFTLTISSLQSEDFAVYYCHQRSSYPWTFGGGTKVEIK (SEQ ID NO: 143) |
| Bh VL8 | EIVMTQSPATLSVSPGERATLSCSASSSVSYVHWYQQKPGQAPRPWIYDTSNLASGFP ARFSGSGSGTEYTLTISSLQSEDAAVYYCHQRSSYPWTFGGGTKVEIK (SEQ ID NO:144) |

In some embodiments, the anti-PD-L1 antibody or antigen-binding fragment thereof provided herein is derived from Clone B. In some embodiments, provided herein are humanized Clone B. In some embodiments, the humanized anti-PD-L1 antibody or antigen-binding fragment thereof provided herein comprises a VH having an amino acid sequence selected from SEQ ID NOs: 131-136. In some embodiments, the humanized anti-PD-L1 antibody or antigen-binding fragment thereof provided herein comprises a VL having an amino acid sequence selected from SEQ ID NOs: 137-144. In some embodiments, the humanized anti-PD-L1 antibody or antigen-binding fragment thereof provided herein comprises a VH and a VL, wherein the VH has the amino acid sequence of SEQ ID NOs: 131, and the VL has an amino acid sequence selected from SEQ ID NOs: 137-144. In some embodiments, the VH has the amino acid sequence of SEQ ID NOs: 132, and the VL has an amino acid sequence selected from SEQ ID NOs: 137-144. In some embodiments, the VH has the amino acid sequence of SEQ ID NOs: 133, and the VL has an amino acid sequence selected from SEQ ID NOs: 137-144. In some embodiments, the VH has the amino acid sequence of SEQ ID NOs: 134, and the VL has an amino acid sequence selected from SEQ ID NOs: 137-144. In some embodiments, the VH has the amino acid sequence of SEQ ID NOs: 135, and the VL has an amino acid sequence selected from SEQ ID NOs:137-144. In some embodiments, the VH has the amino acid sequence of SEQ ID NOs: 136, and the VL has an amino acid sequence selected from SEQ ID NOs: 137-144. In some embodiments, the VH has an amino acid sequence selected from SEQ ID NOs: 131-136, and the VL has the amino acid sequence of SEQ ID NO: 137. In some embodiments, the VH has an amino acid sequence selected from SEQ ID NOs:131-136, and the VL has the amino acid sequence of SEQ ID NO:138. In some embodiments, the VH has an amino acid sequence selected from SEQ ID NOs:131-136, and the VL has the amino acid sequence of SEQ ID NO: 139. In some embodiments, the VH has an amino acid sequence selected from SEQ ID NOs: 131-136, and the VL has the amino acid sequence of SEQ ID NO: 140. In some embodiments, the VH has an amino acid sequence selected from SEQ ID NOs: 131-136, and the VL has the amino acid sequence of SEQ ID NO:141. In some embodiments, the VH has an amino acid sequence selected from SEQ ID NOs:131-136, and the VL has the amino acid sequence of SEQ ID NO: 142. In some embodiments, the VH has an amino acid sequence selected from SEQ ID NOs: 131-136, and the VL has the amino acid sequence of SEQ ID NO: 143. In some embodiments, the VH has an amino acid sequence selected from SEQ ID NOs: 131-136, and the VL has the amino acid sequence of SEQ ID NO:144.

In some embodiments, the anti-PD-L1 antibody or antigen-binding fragment thereof provided herein is the antibody designated as Clone C. In some embodiments, the anti-PD-L1 antibody or antigen-binding fragment thereof provided herein has a VH from Clone C. In some embodiments, the anti-PD-L1 antibody or antigen-binding fragment thereof provided herein has a VL from Clone C. In some embodiments, the anti-PD-L1 antibody or antigen-binding fragment thereof provided herein has a VH and a VL from Clone C. In some embodiments, the anti-PD-L1 antibody or antigen-binding fragment thereof provided herein has a VH that comprises VH CDRs 1, 2, and 3 from a VH from Clone C. In some embodiments, the anti-PD-L1 antibody or antigen-binding fragment thereof provided herein has a VL that comprises VL CDRs 1, 2, and 3 from a VL from Clone C. In some embodiments, the anti-PD-L1 antibody or antigen-binding fragment thereof provided herein has a VH comprising VH CDRs 1, 2, and 3 and a VL comprising VL CDRs 1, 2, and 3 from the VH and VL of Clone C, respectively. In some embodiments, the VH from Clone C has the amino acid sequence of SEQ ID NO: 100. In some embodiments, the VL from Clone C has the amino acid sequence of SEQ ID NO: 113. In some embodiments, the anti-PD-L1 antibody or antigen-binding fragment thereof provided herein has a VH that is a variant of the VH of Clone C having up to about 5 amino acid substitutions, additions, and/or deletions in SEQ ID NO: 100. In some embodiments, the amino acid substitutions, additions, and/or deletions are in the VH CDRs. In some embodiments, the amino acid substitutions, additions, and/or deletions are not in the VH CDRs. In some embodiments, the variant of the VH of Clone C has up to about 5 conservative amino acid substitutions. In some embodiments, the variant of the VH of Clone C has up to 3 conservative amino acid substitutions. In some embodiments, the anti-PD-L1 antibody or antigen-binding fragment thereof provided herein has a VL that is a variant of the VL of Clone C having up to about 5 amino acid substitutions, additions, and/or deletions in SEQ ID NO: 113. In some embodiments, the amino acid substitutions, additions, and/or deletions are in the VL CDRs. In some embodiments, the amino acid substitutions, additions, and/or deletions are not in the VL CDRs. In some embodiments, the variant of the VL of Clone C has up to about 5 conservative amino acid substitutions. In some embodiments, the variant of the VL of Clone C has up to about 3 conservative amino acid substitutions. In some embodiments, the anti-PD-L1 antibody or antigen-binding fragment thereof provided herein is a chimeric antibody or antigen-binding fragment derived from Clone C. In some embodiments, the anti-PD-L1 antibody or antigen-binding fragment thereof provided herein is a humanized antibody or antigen-binding fragment derived from Clone C. In some embodiments, the anti-PD-L1 antibody or antigen-binding fragment thereof provided herein is a human antibody or antigen-binding fragment derived from Clone C.

TABLE 5

Amino acid sequences of heavy chain variable regions (VHs) and light chain variable region (VLs) of humanized Clone C

| | |
|---|---|
| Domain | Sequences |
| Ch VH1 | QVQLVQSGAEVKKPGASVKLSCTASGFNIKDTYMHWVKQRPGQGLEWIGRIDPANG NTKYDPKFQGKATITTDTSTNTAYLELSSLRSEDTAVYYCARGLGRWFAYWGQGTT VTVSS (SEQ ID NO:174) |
| Ch VH2 | QVQLVQSGAEVKKPGASVKVSCKASGFNIKDTYMHWVRQAPGQGLEWIGRIDPANG NTKYDPKFQGRATITTDTSTNTAYLELSSLRSEDTAVYYCARGLGRWFAYWGQGTT VTVSS (SEQ ID NO:175) |
| Ch VH3 | QVQLVQSGAEVKKPGASVKVSCKASGFNIKDTYMHWVRQAPGQGLEWIGRIDPANG NTKYDPKFQGRVTITTDTSANTAYMELSSLRSEDTAVYYCARGLGRWFAYWGQGTT VTVSS (SEQ ID NO:176) |
| Ch VH4 | QVQLVQSGAEVKKPGASVKVSCKASGFNIKDTYMHWVRQAPGQGLEWIGRIDPANG NTKYDPKFQGRVTITTDTSASTAYLELSSLRSEDTAVYYCARGLGRWFAYWGQGTT VTVSS (SEQ ID NO:177) |
| Ch VH5 | QVQLVQSGAEVKKPGASVKVSCKASGFNIKDTYMHWVRQAPGQRLEWIGRIDPANG NTKYSQKFQGRVTITRDTSASTAYMELSSLRSEDTAVYYCARGLGRWFAYWGQGTT VTVSS (SEQ ID NO:178) |
| Ch VL1 | DIQMTQSPSSLSASVGDRVTITCKASQDVSNAVAWYQQKPGKAPKLLIYSASNRYTG VPDRFSGSGSGTDFTFTISSLQPEDIATYYCQQHDSTPLTFGQGTKLELK (SEQ ID NO:179) |
| Ch VL2 | DIQMTQSPSSLSASVGDRVTITCKASQDVSNAVAWYQQKPGKAPKLLIYSASNRYTG VPSRFSGSGSGTDFTFTISSLQPEDIATYYCQQHDSTPLTFGQGTKLELK (SEQ ID NO:180) |
| Ch VL3 | DIQMTQSPSSLSASVGDRVTITCQASQDVSNAVAWYQQKPGKAPKLLIYSASNLYTG VPSRFSGSGSGTDFTFTISSLQPEDIATYYCQQHDSTPLTFGQGTKLELK (SEQ ID NO:181) |

In some embodiments, the anti-PD-L1 antibody or antigen-binding fragment thereof provided herein is derived from Clone C. In some embodiments, provided herein are humanized Clone C. In some embodiments, the humanized anti-PD-L1 antibody or antigen-binding fragment thereof provided herein comprises a VH having an amino acid sequence selected from SEQ ID NOs: 174-178. In some embodiments, the humanized anti-PD-L1 antibody or antigen-binding fragment thereof provided herein comprises a VL having an amino acid sequence selected from SEQ ID NOs: 179-181. In some embodiments, the humanized anti-PD-L1 antibody or antigen-binding fragment thereof provided herein comprises a VH and a VL, wherein the VH and VL have the amino acid sequences of SEQ ID NOs: 174 and 179, respectively. In some embodiments, the VH and VL have the amino acid sequences of SEQ ID NOs: 175 and 179, respectively. In some embodiments, the VH and VL have the amino acid sequences of SEQ ID NOs: 176 and 179, respectively. In some embodiments, the VH and VL have the amino acid sequences of SEQ ID NOs: 177 and 179, respectively. In some embodiments, the VH and VL have the amino acid sequences of SEQ ID NOs: 178 and 179, respectively. In some embodiments, the VH and VL have the amino acid sequences of SEQ ID NOs: 174 and 180. In some embodiments, the VH and VL have the amino acid sequences of SEQ ID NOs: 175 and 180, respectively. In some embodiments, the VH and VL have the amino acid sequences of SEQ ID NOs: 176 and 180, respectively. In some embodiments, the VH and VL have the amino acid sequences of SEQ ID NOs: 177 and 180, respectively. In some embodiments, the VH and VL have the amino acid sequences of SEQ ID NOs: 178 and 180, respectively. In some embodiments, the VH and VL have the amino acid sequences of SEQ ID NOs: 174 and 181. In some embodiments, the VH and VL have the amino acid sequences of SEQ ID NOs: 175 and 181, respectively. In some embodiments, the VH and VL have the amino acid sequences of SEQ ID NOs: 176 and 181, respectively. In some embodiments, the VH and VL have the amino acid sequences of SEQ ID NOs: 177 and 181, respectively. In some embodiments, the VH and VL have the amino acid sequences of SEQ ID NOs: 178 and 181, respectively.

In some embodiments, the anti-PD-L1 antibody or antigen-binding fragment thereof provided herein is the antibody designated as Clone D. In some embodiments, the anti-PD-L1 antibody or antigen-binding fragment thereof provided herein has a VH from Clone D. In some embodiments, the anti-PD-L1 antibody or antigen-binding fragment thereof provided herein has a VL from Clone D. In some embodiments, the anti-PD-L1 antibody or antigen-binding fragment thereof provided herein has a VH and a VL from Clone D. In some embodiments, the anti-PD-L1 antibody or antigen-binding fragment thereof provided herein has a VH that comprises VH CDRs 1, 2, and 3 from a VH from Clone D. In some embodiments, the anti-PD-L1 antibody or antigen-binding fragment thereof provided herein has a VL that comprises VL CDRs 1, 2, and 3 from a VL from Clone D. In some embodiments, the anti-PD-L1 antibody or antigen-binding fragment thereof provided herein has a VH comprising VH CDRs 1, 2, and 3 and a VL comprising VL CDRs 1, 2, and 3 from the VH and VL of Clone D, respectively. In some embodiments, the VH from Clone D has the amino acid sequence of SEQ ID NO: 101. In some embodiments, the VL from Clone D has the amino acid sequence of SEQ ID NO: 114. In some embodiments, the anti-PD-L1 antibody or antigen-binding fragment thereof provided herein has a VH that is a variant of the VH of Clone D having up to about 5 amino acid substitutions, additions, and/or deletions in SEQ ID NO: 101. In some embodiments, the amino acid substitutions, additions, and/or deletions are in the VH CDRs. In some embodiments, the amino acid substitutions, additions, and/or deletions are not in the VH CDRs. In some embodiments, the variant of the VH of Clone D has up to about 5 conservative amino acid substitutions. In some embodiments, the variant of the VH of Clone D has up to 3 conservative amino acid substitutions. In some embodiments, the anti-PD-L1 antibody or antigen-binding fragment thereof provided herein has a VL that is a variant of the VL of Clone D having up to about 5 amino acid substitutions, additions, and/or deletions in SEQ ID NO: 114. In some embodiments, the amino acid substitutions, additions, and/or deletions are in the VL CDRs. In some embodiments, the amino acid substitutions, additions, and/or deletions are not in the VL CDRs. In some embodiments, the variant of the VL of Clone D has up to about 5 conservative amino acid substitutions. In some embodiments, the variant of the VL of Clone D has up to about 3 conservative amino acid substitutions. In some embodiments, the anti-PD-L1 antibody or antigen-binding fragment thereof provided herein is a chimeric antibody or antigen-binding fragment derived from Clone D. In some embodiments, the anti-PD-L1 antibody or antigen-binding fragment thereof provided herein is a humanized antibody or antigen-binding fragment derived from Clone D. In some embodiments, the anti-PD-L1 antibody or antigen-binding fragment thereof provided herein is a human antibody or antigen-binding fragment derived from Clone D.

TABLE 6

Amino acid sequences of heavy chain variable regions (VHs) and light chain variable regions (VLs) of humanized Clone D

| | |
|---|---|
| Domain | Sequences |
| Dh VH1 | QVQLVQSGAEVKKPGASVKVSCKASGYSFTGYTMNWVKQSIIGQGLEWIGLIIPYNG GISYNQKFKDRATLTVDTSSSTAYMELSRLRSDDTAVYYCASLITTAPRDSMDYWGQ GTLVTVSS (SEQ ID NO:126) |
| Dh VH2 | QGQLVQSGAEVKKPGASVKVSCKTSGFTFSSSYISWVRQAPGQGLEWMGWIYAGTG GTSYNQKFTGRVTMTVDTSTSTAYMELRSLRSDDTAVYYCARHEGKYWYFDVWGQ GTTVTVSS (SEQ ID NO:127) |
| Dh VH3 | QVQLVQSGAEVKKPGASVKVSCKTSGFTFSSSYISWVRQAPGQGLEWMGWIYAGTG GTSYNQKFTGRVTMTVDTSTSTAYMELRSLRSDDTAVYYCARHEGKYWYFDVWGQ GTTVTVSS (SEQ ID NO:128) |
| Dh VL1 | DIQMTQSPSSLSASVGDRVTITCSASSSVSYVHWYQQKPGKAPKPWIYDTSNLASGFP SRFSGSGSGTDYTLTISSLQPEDFATYYCHQRSSYPWTFGGGTKVEIK (SEQ ID NO:129) |
| Dh VL2 | EIVLTQSPATLSLSPGERATLSCSASSSVSYVHWYQQKPGQAPRPWIYDTSNLASGFPA RFSGSGSGTDYTLTISSLEPEDAAVYYCHQRSSYPWTFGGGTKVEIK (SEQ ID NO:130) |

In some embodiments, the anti-PD-L1 antibody or antigen-binding fragment thereof provided herein is derived from Clone D. In some embodiments, the anti-PD-L1 antibody or antigen-binding fragment thereof provided herein comprises a VH having the amino acid sequence of SEQ ID NO: 124. In some embodiments, the anti-PD-L1 antibody or antigen-binding fragment thereof provided comprises has a VL having the amino acid sequence of SEQ ID NO:125. In some embodiments, the anti-PD-L1 antibody or antigen-binding fragment thereof provided herein has a VH and a VL having the amino acid sequences of SEQ ID NOs: 124 and 125, respectively. In some embodiments, provided herein are humanized Clone D. In some embodiments, the humanized anti-PD-L1 antibody or antigen-binding fragment thereof provided herein comprises a VH having an amino acid sequence selected from SEQ ID NOs: 126-128. In some embodiments, the humanized anti-PD-L1 antibody or antigen-binding fragment thereof provided herein comprises a VL having an amino acid sequence selected from SEQ ID NOs: 129 and 130. In some embodiments, the humanized anti-PD-L1 antibody or antigen-binding fragment thereof provided herein comprises a VH and a VL, wherein the VH and VL have the amino acid sequences of SEQ ID NOs: 126 and 129, respectively. In some embodiments, the VH and VL have the amino acid sequences of SEQ ID NOs: 127 and 129, respectively. In some embodiments, the VH and VL have the amino acid sequences of SEQ ID NOs: 128 and 129, respectively. In some embodiments, the VH and VL have the amino acid sequences of SEQ ID NOs: 126 and 130, respectively. In some embodiments, the VH and VL have the amino acid sequences of SEQ ID NOs: 127 and 130, respectively. In some embodiments, the VH and VL have the amino acid sequences of SEQ ID NOs: 128 and 130, respectively.

In some embodiments, the anti-PD-L1 antibody or antigen-binding fragment thereof provided herein is the antibody designated as Clone F. In some embodiments, the anti-PD-L1 antibody or antigen-binding fragment thereof provided herein has a VH from Clone F. In some embodiments, the anti-PD-L1 antibody or antigen-binding fragment thereof provided herein has a VL from Clone F. In some embodiments, the anti-PD-L1 antibody or antigen-binding fragment thereof provided herein has a VH and a VL from Clone F. In some embodiments, the anti-PD-L1 antibody or antigen-binding fragment thereof provided herein has a VH that comprises VH CDRs 1, 2, and 3 from a VH from Clone F. In some embodiments, the anti-PD-L1 antibody or antigen-binding fragment thereof provided herein has a VL that comprises VL CDRs 1, 2, and 3 from a VL from Clone F. In some embodiments, the anti-PD-L1 antibody or antigen-binding fragment thereof provided herein has a VH comprising VH CDRs 1, 2, and 3 and a VL comprising VL CDRs 1, 2, and 3 from the VH and VL of Clone F, respectively. In some embodiments, the VH from Clone F has the amino acid sequence of SEQ ID NO: 102. In some embodiments, the VL from Clone F has the amino acid sequence of SEQ ID NO: 115. In some embodiments, the anti-PD-L1 antibody or antigen-binding fragment thereof provided herein has a VH that is a variant of the VH of Clone F having up to about 5 amino acid substitutions, additions, and/or deletions in SEQ ID NO: 102. In some embodiments, the amino acid substitutions, additions, and/or deletions are in the VH CDRs. In some embodiments, the amino acid substitutions, additions, and/or deletions are not in the VH CDRs. In some embodiments, the variant of the VH of Clone F has up to about 5 conservative amino acid substitutions. In some embodiments, the variant of the VH of Clone F has up to 3 conservative amino acid substitutions. In some embodiments, the anti-PD-L1 antibody or antigen-binding fragment thereof provided herein has a VL that is a variant of the VL of Clone F having up to about 5 amino acid substitutions, additions, and/or deletions in SEQ ID NO: 115. In some embodiments, the amino acid substitutions, additions, and/or deletions are in the VL CDRs. In some embodiments, the amino acid substitutions, additions, and/or deletions are not in the VL CDRs. In some embodiments, the variant of the VL of Clone F has up to about 5 conservative amino acid substitutions. In some embodiments, the variant of the VL of Clone F has up to about 3 conservative amino acid substitutions. In some embodiments, the anti-PD-L1 antibody or antigen-binding fragment thereof provided herein is a chimeric antibody or antigen-binding fragment derived from Clone F. In some embodiments, the anti-PD-L1 antibody or antigen-binding fragment thereof provided herein is a humanized antibody or antigen-binding fragment derived from Clone F. In some embodiments, the anti-PD-L1 antibody or antigen-binding fragment thereof provided herein is a human antibody or antigen-binding fragment derived from Clone F.

In some embodiments, the anti-PD-L1 antibody or antigen-binding fragment thereof provided herein is the antibody designated as Clone G. In some embodiments, the anti-PD-L1 antibody or antigen-binding fragment thereof provided herein has a VH from Clone G. In some embodiments, the anti-PD-L1 antibody or antigen-binding fragment thereof provided herein has a VL from Clone G. In some embodiments, the anti-PD-L1 antibody or antigen-binding fragment thereof provided herein has a VH and a VL from Clone G. In some embodiments, the anti-PD-L1 antibody or antigen-binding fragment thereof provided herein has a VH that comprises VH CDRs 1, 2, and 3 from a VH from Clone G. In some embodiments, the anti-PD-L1 antibody or antigen-binding fragment thereof provided herein has a VL that comprises VL CDRs 1, 2, and 3 from a VL from Clone G. In some embodiments, the anti-PD-L1 antibody or antigen-binding fragment thereof provided herein has a VH comprising VH CDRs 1, 2, and 3 and a VL comprising VL CDRs 1, 2, and 3 from the VH and VL of Clone G, respectively. In some embodiments, the VH from Clone G has the amino acid sequence of SEQ ID NO: 103. In some embodiments, the VL from Clone G has the amino acid sequence of SEQ ID NO: 116. In some embodiments, the anti-PD-L1 antibody or antigen-binding fragment thereof provided herein has a VH that is a variant of the VH of Clone G having up to about 5 amino acid substitutions, additions, and/or deletions in SEQ ID NO: 103. In some embodiments, the amino acid substitutions, additions, and/or deletions are in the VH CDRs. In some embodiments, the amino acid substitutions, additions, and/or deletions are not in the VH CDRs. In some embodiments, the variant of the VH of Clone G has up to about 5 conservative amino acid substitutions. In some embodiments, the variant of the VH of Clone G has up to 3 conservative amino acid substitutions. In some embodiments, the anti-PD-L1 antibody or antigen-binding fragment thereof provided herein has a VL that is a variant of the VL of Clone G having up to about 5 amino acid substitutions, additions, and/or deletions in SEQ ID NO: 116. In some embodiments, the amino acid substitutions, additions, and/or deletions are in the VL CDRs. In some embodiments, the amino acid substitutions, additions, and/or deletions are not in the VL CDRs. In some embodiments, the variant of the VL of Clone G has up to about 5 conservative amino acid substitutions. In some embodiments, the variant of the VL of Clone G has up to about 3 conservative amino acid substitutions. In some embodiments, the anti-PD-L1 antibody or antigen-binding fragment thereof provided herein is a chimeric antibody or antigen-binding fragment derived from Clone G. In some embodiments, the anti-PD-L1 antibody or antigen-binding fragment thereof provided herein is a humanized antibody or antigen-binding fragment derived from Clone G. In some embodiments, the anti-PD-L1 antibody or antigen-binding fragment thereof provided herein is a human antibody or antigen-binding fragment derived from Clone G.

In some embodiments, the anti-PD-L1 antibody or antigen-binding fragment thereof provided herein is the antibody designated as Clone H. In some embodiments, the anti-PD-L1 antibody or antigen-binding fragment thereof provided herein has a VH from Clone H. In some embodiments, the anti-PD-L1 antibody or antigen-binding fragment thereof provided herein has a VL from Clone H. In some embodiments, the anti-PD-L1 antibody or antigen-binding fragment thereof provided herein has a VH and a VL from Clone H. In some embodiments, the anti-PD-L1 antibody or antigen-binding fragment thereof provided herein has a VH that comprises VH CDRs 1, 2, and 3 from a VH from Clone H. In some embodiments, the anti-PD-L1 antibody or antigen-binding fragment thereof provided herein has a VL that comprises VL CDRs 1, 2, and 3 from a VL from Clone H. In some embodiments, the anti-PD-L1 antibody or antigen-binding fragment thereof provided herein has a VH comprising VH CDRs 1, 2, and 3 and a VL comprising VL CDRs 1, 2, and 3 from the VH and VL of Clone H, respectively. In some embodiments, the VH from Clone H has the amino acid sequence of SEQ ID NO: 104. In some embodiments, the VL from Clone H has the amino acid sequence of SEQ ID NO: 117. In some embodiments, the anti-PD-L1 antibody or antigen-binding fragment thereof provided herein has a VH that is a variant of the VH of Clone H having up to about 5 amino acid substitutions, additions, and/or deletions in SEQ ID NO: 104. In some embodiments, the amino acid substitutions, additions, and/or deletions are in the VH CDRs. In some embodiments, the amino acid substitutions, additions, and/or deletions are not in the VH CDRs. In some embodiments, the variant of the VH of Clone H has up to about 5 conservative amino acid substitutions. In some embodiments, the variant of the VH of Clone H has up to 3 conservative amino acid substitutions. In some embodiments, the anti-PD-L1 antibody or antigen-binding fragment thereof provided herein has a VL that is a variant of the VL of Clone H having up to about 5 amino acid substitutions, additions, and/or deletions in SEQ ID NO: 117. In some embodiments, the amino acid substitutions, additions, and/or deletions are in the VL CDRs. In some embodiments, the amino acid substitutions, additions, and/or deletions are not in the VL CDRs. In some embodiments, the variant of the VL of Clone H has up to about 5 conservative amino acid substitutions. In some embodiments, the variant of the VL of Clone H has up to about 3 conservative amino acid substitutions. In some embodiments, the anti-PD-L1 antibody or antigen-binding fragment thereof provided herein is a chimeric antibody or antigen-binding fragment derived from Clone H. In some embodiments, the anti-PD-L1 antibody or antigen-binding fragment thereof provided herein is a humanized antibody or antigen-binding fragment derived from Clone H. In some embodiments, the anti-PD-L1 antibody or antigen-binding fragment thereof provided herein is a human antibody or antigen-binding fragment derived from Clone H.

In some embodiments, the anti-PD-L1 antibody or antigen-binding fragment thereof provided herein is the antibody designated as Clone J. In some embodiments, the anti-PD-L1 antibody or antigen-binding fragment thereof provided herein has a VH from Clone J. In some embodiments, the anti-PD-L1 antibody or antigen-binding fragment thereof provided herein has a VL from Clone J. In some embodiments, the anti-PD-L1 antibody or antigen-binding fragment thereof provided herein has a VH and a VL from Clone J. In some embodiments, the anti-PD-L1 antibody or antigen-binding fragment thereof provided herein has a VH that comprises VH CDRs 1, 2, and 3 from a VH from Clone J. In some embodiments, the anti-PD-L1 antibody or antigen-binding fragment thereof provided herein has a VL that comprises VL CDRs 1, 2, and 3 from a VL from Clone J. In some embodiments, the anti-PD-L1 antibody or antigen-binding fragment thereof provided herein has a VH comprising VH CDRs 1, 2, and 3 and a VL comprising VL CDRs 1, 2, and 3 from the VH and VL of Clone J, respectively. In some embodiments, the VH from Clone J has the amino acid sequence of SEQ ID NO: 105. In some embodiments, the VL from Clone J has the amino acid sequence of SEQ ID NO: 118. In some embodiments, the anti-PD-L1 antibody or antigen-binding fragment thereof provided herein has a VH that is a variant of the VH of Clone J having up to about 5 amino acid substitutions, additions, and/or deletions in SEQ ID NO: 105. In some embodiments, the amino acid substitutions, additions, and/or deletions are in the VH CDRs. In some embodiments, the amino acid substitutions, additions, and/or deletions are not in the VH CDRs. In some embodiments, the variant of the VH of Clone J has up to about 5 conservative amino acid substitutions. In some embodiments, the variant of the VH of Clone J has up to 3 conservative amino acid substitutions. In some embodiments, the anti-PD-L1 antibody or antigen-binding fragment thereof provided herein has a VL that is a variant of the VL of Clone J having up to about 5 amino acid substitutions, additions, and/or deletions in SEQ ID NO: 118. In some embodiments, the amino acid substitutions, additions, and/or deletions are in the VL CDRs. In some embodiments, the amino acid substitutions, additions, and/or deletions are not in the VL CDRs. In some embodiments, the variant of the VL of Clone J has up to about 5 conservative amino acid substitutions. In some embodiments, the variant of the VL of Clone J has up to about 3 conservative amino acid substitutions. In some embodiments, the anti-PD-L1 antibody or antigen-binding fragment thereof provided herein is a chimeric antibody or antigen-binding fragment derived from Clone J. In some embodiments, the anti-PD-L1 antibody or antigen-binding fragment thereof provided herein is a humanized antibody or antigen-binding fragment derived from Clone J. In some embodiments, the anti-PD-L1 antibody or antigen-binding fragment thereof provided herein is a human antibody or antigen-binding fragment derived from Clone J.

In some embodiments, the anti-PD-L1 antibody or antigen-binding fragment thereof provided herein is the antibody designated as Clone K. In some embodiments, the anti-PD-L1 antibody or antigen-binding fragment thereof provided herein has a VH from Clone K. In some embodiments, the anti-PD-L1 antibody or antigen-binding fragment thereof provided herein has a VL from Clone K. In some embodiments, the anti-PD-L1 antibody or antigen-binding fragment thereof provided herein has a VH and a VL from Clone K. In some embodiments, the anti-PD-L1 antibody or antigen-binding fragment thereof provided herein has a VH that comprises VH CDRs 1, 2, and 3 from a VH from Clone K. In some embodiments, the anti-PD-L1 antibody or antigen-binding fragment thereof provided herein has a VL that comprises VL CDRs 1, 2, and 3 from a VL from Clone K. In some embodiments, the anti-PD-L1 antibody or antigen-binding fragment thereof provided herein has a VH comprising VH CDRs 1, 2, and 3 and a VL comprising VL CDRs 1, 2, and 3 from the VH and VL of Clone K, respectively. In some embodiments, the VH from Clone K has the amino acid sequence of SEQ ID NO: 106. In some embodiments, the VL from Clone K has the amino acid sequence of SEQ ID NO: 119. In some embodiments, the anti-PD-L1 antibody or antigen-binding fragment thereof provided herein has a VH that is a variant of the VH of Clone K having up to about 5 amino acid substitutions, additions, and/or deletions in SEQ ID NO: 106. In some embodiments, the amino acid substitutions, additions, and/or deletions are in the VH CDRs. In some embodiments, the amino acid substitutions, additions, and/or deletions are not in the VH CDRs. In some embodiments, the variant of the VH of Clone K has up to about 5 conservative amino acid substitutions. In some embodiments, the variant of the VH of Clone K has up to 3 conservative amino acid substitutions. In some embodiments, the anti-PD-L1 antibody or antigen-binding fragment thereof provided herein has a VL that is a variant of the VL of Clone K having up to about 5 amino acid substitutions, additions, and/or deletions in SEQ ID NO: 119. In some embodiments, the amino acid substitutions, additions, and/or deletions are in the VL CDRs. In some embodiments, the amino acid substitutions, additions, and/or deletions are not in the VL CDRs. In some embodiments, the variant of the VL of Clone K has up to about 5 conservative amino acid substitutions. In some embodiments, the variant of the VL of Clone K has up to about 3 conservative amino acid substitutions. In some embodiments, the anti-PD-L1 antibody or antigen-binding fragment thereof provided herein is a chimeric antibody or antigen-binding fragment derived from Clone K. In some embodiments, the anti-PD-L1 antibody or antigen-binding fragment thereof provided herein is a humanized antibody or antigen-binding fragment derived from Clone K. In some embodiments, the anti-PD-L1 antibody or antigen-binding fragment thereof provided herein is a human antibody or antigen-binding fragment derived from Clone K.

In some embodiments, the anti-PD-L1 antibody or antigen-binding fragment thereof provided herein is the antibody designated as Clone L. In some embodiments, the anti-PD-L1 antibody or antigen-binding fragment thereof provided herein has a VH from Clone L. In some embodiments, the anti-PD-L1 antibody or antigen-binding fragment thereof provided herein has a VL from Clone L. In some embodiments, the anti-PD-L1 antibody or antigen-binding fragment thereof provided herein has a VH and a VL from Clone L. In some embodiments, the anti-PD-L1 antibody or antigen-binding fragment thereof provided herein has a VH that comprises VH CDRs 1, 2, and 3 from a VH from Clone L. In some embodiments, the anti-PD-L1 antibody or antigen-binding fragment thereof provided herein has a VL that comprises VL CDRs 1, 2, and 3 from a VL from Clone L. In some embodiments, the anti-PD-L1 antibody or antigen-binding fragment thereof provided herein has a VH comprising VH CDRs 1, 2, and 3 and a VL comprising VL CDRs 1, 2, and 3 from the VH and VL of Clone L, respectively. In some embodiments, the VH from Clone L has the amino acid sequence of SEQ ID NO: 107. In some embodiments, the VL from Clone L has the amino acid sequence of SEQ ID NO: 120. In some embodiments, the anti-PD-L1 antibody or antigen-binding fragment thereof provided herein has a VH that is a variant of the VH of Clone L having up to about 5 amino acid substitutions, additions, and/or deletions in SEQ ID NO: 107. In some embodiments, the amino acid substitutions, additions, and/or deletions are in the VH CDRs. In some embodiments, the amino acid substitutions, additions, and/or deletions are not in the VH CDRs. In some embodiments, the variant of the VH of Clone L has up to about 5 conservative amino acid substitutions. In some embodiments, the variant of the VH of Clone L has up to 3 conservative amino acid substitutions. In some embodiments, the anti-PD-L1 antibody or antigen-binding fragment thereof provided herein has a VL that is a variant of the VL of Clone L having up to about 5 amino acid substitutions, additions, and/or deletions in SEQ ID NO: 120. In some embodiments, the amino acid substitutions, additions, and/or deletions are in the VL CDRs. In some embodiments, the amino acid substitutions, additions, and/or deletions are not in the VL CDRs. In some embodiments, the variant of the VL of Clone L has up to about 5 conservative amino acid substitutions. In some embodiments, the variant of the VL of Clone L has up to about 3 conservative amino acid substitutions. In some embodiments, the anti-PD-L1 antibody or antigen-binding fragment thereof provided herein is a chimeric antibody or antigen-binding fragment derived from Clone L. In some embodiments, the anti-PD-L1 antibody or antigen-binding fragment thereof provided herein is a humanized antibody or antigen-binding fragment derived from Clone L. In some embodiments, the anti-PD-L1 antibody or antigen-binding fragment thereof provided herein is a human antibody or antigen-binding fragment derived from Clone L.

In some embodiments, the anti-PD-L1 antibody or antigen-binding fragment thereof provided herein is the antibody designated as Clone N. In some embodiments, the anti-PD-L1 antibody or antigen-binding fragment thereof provided herein has a VH from Clone N. In some embodiments, the anti-PD-L1 antibody or antigen-binding fragment thereof provided herein has a VL from Clone N. In some embodiments, the anti-PD-L1 antibody or antigen-binding fragment thereof provided herein has a VH and a VL from Clone N. In some embodiments, the anti-PD-L1 antibody or antigen-binding fragment thereof provided herein has a VH that comprises VH CDRs 1, 2, and 3 from a VH from Clone N. In some embodiments, the anti-PD-L1 antibody or antigen-binding fragment thereof provided herein has a VL that comprises VL CDRs 1, 2, and 3 from a VL from Clone N. In some embodiments, the anti-PD-L1 antibody or antigen-binding fragment thereof provided herein has a VH comprising VH CDRs 1, 2, and 3 and a VL comprising VL CDRs 1, 2, and 3 from the VH and VL of Clone N, respectively. In some embodiments, the VH from Clone N has the amino acid sequence of SEQ ID NO: 108. In some embodiments, the VL from Clone N has the amino acid sequence of SEQ ID NO: 121. In some embodiments, the anti-PD-L1 antibody or antigen-binding fragment thereof provided herein has a VH that is a variant of the VH of Clone N having up to about 5 amino acid substitutions, additions, and/or deletions in SEQ ID NO: 108. In some embodiments, the amino acid substitutions, additions, and/or deletions are in the VH CDRs. In some embodiments, the amino acid substitutions, additions, and/or deletions are not in the VH CDRs. In some embodiments, the variant of the VH of Clone N has up to about 5 conservative amino acid substitutions. In some embodiments, the variant of the VH of Clone N has up to 3 conservative amino acid substitutions. In some embodiments, the anti-PD-L1 antibody or antigen-binding fragment thereof provided herein has a VL that is a variant of the VL of Clone N having up to about 5 amino acid substitutions, additions, and/or deletions in SEQ ID NO: 121. In some embodiments, the amino acid substitutions, additions, and/or deletions are in the VL CDRs. In some embodiments, the amino acid substitutions, additions, and/or deletions are not in the VL CDRs. In some embodiments, the variant of the VL of Clone N has up to about 5 conservative amino acid substitutions. In some embodiments, the variant of the VL of Clone N has up to about 3 conservative amino acid substitutions. In some embodiments, the anti-PD-L1 antibody or antigen-binding fragment thereof provided herein is a chimeric antibody or antigen-binding fragment derived from Clone N. In some embodiments, the anti-PD-L1 antibody or antigen-binding fragment thereof provided herein is a humanized antibody or antigen-binding fragment derived from Clone N. In some embodiments, the anti-PD-L1 antibody or antigen-binding fragment thereof provided herein is a human antibody or antigen-binding fragment derived from Clone N.

In some embodiments, the anti-PD-L1 antibody or antigen-binding fragment thereof provided herein is the antibody designated as Clone P. In some embodiments, the anti-PD-L1 antibody or antigen-binding fragment thereof provided herein has a VH from Clone P. In some embodiments, the anti-PD-L1 antibody or antigen-binding fragment thereof provided herein has a VL from Clone P. In some embodiments, the anti-PD-L1 antibody or antigen-binding fragment thereof provided herein has a VH and a VL from Clone P. In some embodiments, the anti-PD-L1 antibody or antigen-binding fragment thereof provided herein has a VH that comprises VH CDRs 1, 2, and 3 from a VH from Clone P. In some embodiments, the anti-PD-L1 antibody or antigen-binding fragment thereof provided herein has a VL that comprises VL CDRs 1, 2, and 3 from a VL from Clone P. In some embodiments, the anti-PD-L1 antibody or antigen-binding fragment thereof provided herein has a VH comprising VH CDRs 1, 2, and 3 and a VL comprising VL CDRs 1, 2, and 3 from the VH and VL of Clone P, respectively. In some embodiments, the VH from Clone P has the amino acid sequence of SEQ ID NO: 109. In some embodiments, the VL from Clone P has the amino acid sequence of SEQ ID NO: 122. In some embodiments, the anti-PD-L1 antibody or antigen-binding fragment thereof provided herein has a VH that is a variant of the VH of Clone P having up to about 5 amino acid substitutions, additions, and/or deletions in SEQ ID NO: 109. In some embodiments, the amino acid substitutions, additions, and/or deletions are in the VH CDRs. In some embodiments, the amino acid substitutions, additions, and/or deletions are not in the VH CDRs. In some embodiments, the variant of the VH of Clone P has up to about 5 conservative amino acid substitutions. In some embodiments, the variant of the VH of Clone P has up to 3 conservative amino acid substitutions. In some embodiments, the anti-PD-L1 antibody or antigen-binding fragment thereof provided herein has a VL that is a variant of the VL of Clone P having up to about 5 amino acid substitutions, additions, and/or deletions in SEQ ID NO: 122. In some embodiments, the amino acid substitutions, additions, and/or deletions are in the VL CDRs. In some embodiments, the amino acid substitutions, additions, and/or deletions are not in the VL CDRs. In some embodiments, the variant of the VL of Clone P has up to about 5 conservative amino acid substitutions. In some embodiments, the variant of the VL of Clone P has up to about 3 conservative amino acid substitutions. In some embodiments, the anti-PD-L1 antibody or antigen-binding fragment thereof provided herein is a chimeric antibody or antigen-binding fragment derived from Clone P. In some embodiments, the anti-PD-L1 antibody or antigen-binding fragment thereof provided herein is a humanized antibody or antigen-binding fragment derived from Clone P. In some embodiments, the anti-PD-L1 antibody or antigen-binding fragment thereof provided herein is a human antibody or antigen-binding fragment derived from Clone P.

In some embodiments, the anti-PD-L1 antibody or antigen-binding fragment thereof provided herein is the antibody designated as Clone Y. In some embodiments, the anti-PD-L1 antibody or antigen-binding fragment thereof provided herein has a VH from Clone Y. In some embodiments, the anti-PD-L1 antibody or antigen-binding fragment thereof provided herein has a VL from Clone Y. In some embodiments, the anti-PD-L1 antibody or antigen-binding fragment thereof provided herein has a VH and a VL from Clone Y. In some embodiments, the anti-PD-L1 antibody or antigen-binding fragment thereof provided herein has a VH that comprises VH CDRs 1, 2, and 3 from a VH from Clone Y. In some embodiments, the anti-PD-L1 antibody or antigen-binding fragment thereof provided herein has a VL that comprises VL CDRs 1, 2, and 3 from a VL from Clone Y. In some embodiments, the anti-PD-L1 antibody or antigen-binding fragment thereof provided herein has a VH comprising VH CDRs 1, 2, and 3 and a VL comprising VL CDRs 1, 2, and 3 from the VH and VL of Clone Y, respectively. In some embodiments, the VH from Clone Y has the amino acid sequence of SEQ ID NO:110. In some embodiments, the VL from Clone Y has the amino acid sequence of SEQ ID NO:123. In some embodiments, the anti-PD-L1 antibody or antigen-binding fragment thereof provided herein has a VH that is a variant of the VH of Clone Y having up to about 5 amino acid substitutions, additions, and/or deletions in SEQ ID NO:110. In some embodiments, the amino acid substitutions, additions, and/or deletions are in the VH CDRs. In some embodiments, the amino acid substitutions, additions, and/or deletions are not in the VH CDRs. In some embodiments, the variant of the VH of Clone Y has up to about 5 conservative amino acid substitutions. In some embodiments, the variant of the VH of Clone Y has up to 3 conservative amino acid substitutions. In some embodiments, the anti-PD-L1 antibody or antigen-binding fragment thereof provided herein has a VL that is a variant of the VL of Clone Y having up to about 5 amino acid substitutions, additions, and/or deletions in SEQ ID NO: 123. In some embodiments, the amino acid substitutions, additions, and/or deletions are in the VL CDRs. In some embodiments, the amino acid substitutions, additions, and/or deletions are not in the VL CDRs. In some embodiments, the variant of the VL of Clone Y has up to about 5 conservative amino acid substitutions. In some embodiments, the variant of the VL of Clone Y has up to about 3 conservative amino acid substitutions. In some embodiments, the anti-PD-L1 antibody or antigen-binding fragment thereof provided herein is a chimeric antibody or antigen-binding fragment derived from Clone Y. In some embodiments, the anti-PD-L1 antibody or antigen-binding fragment thereof provided herein is a humanized antibody or antigen-binding fragment derived from Clone Y. In some embodiments, the anti-PD-L1 antibody or antigen-binding fragment thereof provided herein is a human antibody or antigen-binding fragment derived from Clone Y.

In some embodiments, provided herein are also antibodies or antigen-binding fragments that compete with the antibody or antigen-binding fragment provided above for binding to PD-L1 (e.g., human PD-L1). Antibodies that “compete with another antibody for binding to a target” refer to antibodies that inhibit (partially or completely) the binding of the other antibody to the target. Whether two antibodies compete with each other for binding to a target, i.e., whether and to what extent one antibody inhibits the binding of the other antibody to a target, can be determined using known competition experiments, e.g., BIACORE® surface plasmon resonance (SPR) analysis. In some embodiments, an anti-PD-L1 antibody or antigen-binding fragment competes with, and inhibits binding of another antibody or antigen-binding fragment to PD-L1 by at least 50%, 60%, 70%, 80%, 90% or 100%. Competition assays can be conducted as described, for example, in Ed Harlow and David Lane, Cold Spring Harb Protoc ; 2006; doi: 10.H01/pdb.prot4277 or in Chapter 11 of “Using Antibodies” by Ed Harlow and David Lane, Cold Spring Harbor Laboratory Press, Cold Spring Harbor, NY, USA 1999.

In some embodiments, provided herein are antibodies or antigen-binding fragments that compete with Clone A for binding to PD-L1. In some embodiments, provided herein are antibodies or antigen-binding fragments that compete with Clone B for binding to PD-L1. In some embodiments, provided herein are antibodies or antigen-binding fragments that compete with Clone C for binding to PD-L1. In some embodiments, provided herein are antibodies or antigen-binding fragments that compete with Clone D for binding to PD-L1. In some embodiments, provided herein are antibodies or antigen-binding fragments that compete with Clone F for binding to PD-L1. In some embodiments, provided herein are antibodies or antigen-binding fragments that compete with Clone G for binding to PD-L1. In some embodiments, provided herein are antibodies or antigen-binding fragments that compete with Clone H for binding to PD-L1. In some embodiments, provided herein are antibodies or antigen-binding fragments that compete with Clone J for binding to PD-L1. In some embodiments, provided herein are antibodies or antigen-binding fragments that compete with Clone K for binding to PD-L1. In some embodiments, provided herein are antibodies or antigen-binding fragments that compete with Clone L for binding to PD-L1. In some embodiments, provided herein are antibodies or antigen-binding fragments that compete with Clone N for binding to PD-L1. In some embodiments, provided herein are antibodies or antigen-binding fragments that compete with Clone P for binding to PD-L1. In some embodiments, provided herein are antibodies or antigen-binding fragments that compete with Clone Y for binding to PD-L1.

The present disclosure further contemplates additional variants and equivalents that are substantially homologous to the recombinant, monoclonal, chimeric, humanized, and human antibodies, or antibody fragments thereof, described herein. In some embodiments, it is desirable to improve the binding affinity of the antibody. In some embodiments, it is desirable to modulate biological properties of the antibody, including but not limited to, specificity, thermostability, expression level, effector function(s), glycosylation, immunogenicity, and/or solubility. Those skilled in the art will appreciate that amino acid changes may alter post-translational processes of an antibody, such as changing the number or position of glycosylation sites or altering membrane anchoring characteristics.

Variations may be a substitution, deletion, or insertion of one or more nucleotides encoding the antibody or polypeptide that results in a change in the amino acid sequence as compared with the native antibody or polypeptide sequence. In some embodiments, amino acid substitutions are the result of replacing one amino acid with another amino acid having similar structural and/or chemical properties, such as the replacement of a leucine with a serine, e.g., conservative amino acid replacements. Insertions or deletions can be in the range of about 1 to 5 amino acids. In some embodiments, the substitution, deletion, or insertion includes (epsilon receptors), IgA (alpha receptors) and IgM (mu receptors). Binding of antibody to Fc receptors on cell surfaces triggers a number of important and diverse biological responses including engulfment and destruction of antibody-coated particles, clearance of immune complexes, lysis of antibody-coated target cells by killer cells (called antibody-dependent cell cytotoxicity or ADCC), release of inflammatory mediators, placental transfer, and control of immunoglobulin production.

In some embodiments, at least one or more of the constant regions has been modified or deleted in the anti-PD-L1 antibody or antigen-binding fragment described herein. In some embodiments, the antibodies comprise modifications to one or more of the three heavy chain constant regions (CH1, CH2 or CH3) and/or to the light chain constant region (CL). In some embodiments, the heavy chain constant region of the modified antibodies comprises at least one human constant region. In some embodiments, the heavy chain constant region of the modified antibodies comprises more than one human constant region. In some embodiments, modifications to the constant region comprise additions, deletions, or substitutions of one or more amino acids in one or more regions. In some embodiments, one or more regions are partially or entirely deleted from the constant regions of the modified antibodies. In some embodiments, the entire CH2 domain has been removed from an antibody (ΔCH2 constructs). In some embodiments, a deleted constant region is replaced by a short amino acid spacer that provides some of the molecular flexibility typically imparted by the absent constant region. In some embodiments, a modified antibody comprises a CH3 domain directly fused to the hinge region of the antibody. In some embodiments, a modified antibody comprises a peptide spacer inserted between the hinge region and modified CH2 and/or CH3 domains.

It is known in the art that the constant region(s) of an antibody mediates several effector functions and these effector functions can vary depending on the isotype of the antibody. For example, binding of the C1 component of complement to the Fc region of IgG or IgM antibodies (bound to antigen) activates the complement system. Activation of complement is important in the opsonization and lysis of cell pathogens. The activation of complement also stimulates the inflammatory response and can be involved in autoimmune hypersensitivity. In addition, the Fc region of an antibody can bind a cell expressing a Fc receptor (FcR). There are a number of Fc receptors which are specific for different classes of antibody, including IgG (gamma receptors), IgE (epsilon receptors), IgA (alpha receptors) and IgM (mu receptors). Binding of antibody to Fc receptors on cell surfaces triggers a number of important and diverse biological responses including engulfment and destruction of antibody-coated particles, clearance of immune complexes, lysis of antibody-coated target cells by killer cells (called antibody-dependent cell cytotoxicity or ADCC), release of inflammatory mediators, placental transfer, and control of immunoglobulin production.

In some embodiments, an anti-PD-L1 antibody or antigen-binding fragment comprises a Fc region. In some embodiments, the Fc region is fused via a hinge. The hinge can be an IgG1 hinge, an IgG2 hinge, or an IgG3 hinge. The amino acid sequences of the Fc region of human IgG1, IgG2, IgG3, and IgG4 are known to those of ordinary skill in the art. In some cases, Fc regions with amino acid variations have been identified in native antibodies. In some embodiments, the modified antibodies (e.g., modified Fc region) provide for altered effector functions that, in turn, affect the biological profile of the antibody. For example, in some embodiments, the deletion or inactivation (through point mutations or other means) of a constant region reduces Fc receptor binding of the modified antibody as it circulates. In some embodiments, the constant region modifications reduce the immunogenicity of the antibody. In some embodiments, the constant region modifications increase the serum half-life of the antibody. In some embodiments, the constant region modifications reduce the serum half-life of the antibody. In some embodiments, the constant region modifications decrease or remove ADCC and/or complement dependent cytotoxicity (CDC) of the antibody. In some embodiments, specific amino acid substitutions in a human IgG1 Fc region with corresponding IgG2 or IgG4 residues reduce effector functions (e.g., ADCC and CDC) in the modified antibody. In some embodiments, an antibody does not have one or more effector functions (e.g., “effectorless” antibodies). In some embodiments, the antibody has no ADCC activity and/or no CDC activity. In some embodiments, the antibody does not bind an Fc receptor and/or complement factors. In some embodiments, the antibody has no effector function(s). In some embodiments, the constant region modifications increase or enhance ADCC and/or CDC of the antibody. In some embodiments, the constant region is modified to eliminate disulfide linkages or oligosaccharide moieties. In some embodiments, the constant region is modified to add/substitute one or more amino acids to provide one or more cytotoxin, oligosaccharide, or carbohydrate attachment sites. In some embodiments, an anti-PD-L1 antibody or antigen-binding fragment comprises a variant Fc region that is engineered with substitutions at specific amino acid positions as compared to a native Fc region. In some embodiments, an anti-PD-L1 antibody or antigen-binding fragment described herein comprises an IgG1 heavy chain constant region that comprises one or more amino acid substitutions selected from the group consisting of K214R, L234A, L235E, G237A, D356E, and L358M, per EU numbering. In some embodiments, the IgG1 heavy chain constant region comprises one or more amino acid substitutions selected from the group consisting of K214R, L234A, L235E, G237A, A330S, P331S, D356E, and L358M, per EU numbering. In some embodiments, the IgG1 heavy chain constant region comprises one or more amino acid substitutions selected from the group consisting of K214R, C226S, C229S, and P238S, per EU numbering. In some embodiments, the IgG1 heavy chain constant region comprises one or more amino acid substitutions selected from the group consisting of K214R, D356E, and L358M, per EU numbering. In some embodiments, the IgG1 heavy chain constant region comprises one or more amino acid substitutions selected from the group consisting of S131C, K133R, G137E, G138S, Q196K, I199T, N203D, K214R, C226S, C229S, and P238S, per EU numbering.

In some embodiments, variants can include addition of amino acid residues at the amino-and/or carboxyl-terminal end of the antibody or polypeptide. The length of additional amino acids residues can range from one residue to a hundred or more residues. In some embodiments, a variant comprises an N-terminal methionyl residue. In some embodiments, the variant comprises an additional polypeptide/protein (e.g., Fc region) to create a fusion protein. In some embodiments, a variant is engineered to be detectable and may comprise a detectable label and/or protein (e.g., a fluorescent tag or an enzyme).

The variant antibodies or antigen-binding fragments described herein can be generated using methods known in the art, including but not limited to, site-directed mutagenesis, alanine scanning mutagenesis, and PCR mutagenesis.

In some embodiments, a variant of an anti-PD-L1 antibody or antigen-binding fragment disclosed herein can retain the ability to recognize a target (e.g., PD-L1) to a similar extent, the same extent, or to a higher extent, as the parent binding moiety. In some embodiments, the variant can be at least about 80%, about 85%, about 90%, about 91 %, about 92%, about 93%, about 94%, about 95%, about 96%, about 97%, about 98%, about 99% or more identical in amino acid sequence to the parent antibody or antigen-binding fragment.

In certain embodiments, a variant of an anti-PD-L1 antibody or antigen-binding fragment comprises the amino acid sequence of the parent anti-PD-L1 antibody or antigen-binding fragment with one or more conservative amino acid substitution. Conservative amino acid substitutions are known in the art and include amino acid substitutions in which one amino acid having certain physical and/or chemical properties is exchanged for another amino acid that has the same or similar chemical or physical properties.

In some embodiments, a variant of an anti-PD-L1 antibody or antigen-binding fragment comprises the amino acid sequence of the parent antibody or antigen-binding fragment with one or more non-conservative amino acid substitutions. In some embodiments, a variant of an anti-PD-L1 antibody or antigen-binding fragment comprises the amino acid sequence of the parent binding antibody or antigen-binding fragment with one or more non-conservative amino acid substitution, wherein the one or more non-conservative amino acid substitutions do not interfere with or inhibit one or more biological activities of the variant (e.g., PD-L1 binding). In certain embodiments, the one or more conservative amino acid substitutions and/or the one or more non-conservative amino acid substitutions can enhance a biological activity of the variant, such that the biological activity of the functional variant is increased as compared to the parent binding moiety.

In some embodiments, the variant can have 1, 2, 3, 4, or 5 amino acid substitutions in the CDRs (e.g., VH CDR1, VH CDR2, VH CDR3, VL CDR1, VL CDR2 and VL CDR3) of the binding moiety.

In some embodiments, anti-PD-L1 antibodies or antigen-binding fragments described herein are chemically modified naturally or by intervention. In some embodiments, the anti-PD-L1 antibodies or antigen-binding fragments have been chemically modified by glycosylation, acetylation, pegylation, phosphorylation, amidation, derivatization by known protecting/blocking groups, proteolytic cleavage, and/or linkage to a cellular ligand or other protein. Any of numerous chemical modifications can be carried out by known techniques. The anti-PD-L1 antibodies or antigen-binding fragments can comprise one or more analogs of an amino acid (including, for example, unnatural amino acids), as well as other modifications known in the art.

In some embodiments, an anti-PD-L1 antibody or antigen-binding fragment (e.g., an antibody) binds PD-L1 (e.g., human PD-L1) with a dissociation constant (K_{D}) of about 1 µM or less, about 100 nM or less, about 40 nM or less, about 20 nM or less, about 10 nM or less, about 1 nM or less, about 0.1 nM or less, 50 pM or less, 10 pM or less, or 1 pM or less. In some embodiments, an anti-PD-L1 antibody or antigen-binding fragment binds PD-L1 (e.g., human PD-L1) with a K_{D} of about 20 nM or less. In some embodiments, an anti-PD-L1 antibody or antigen-binding fragment binds PD-L1 (e.g., human PD-L1) with a K_{D} of about 10 nM or less. In some embodiments, an anti-PD-L1 antibody or antigen-binding fragment binds PD-L1 (e.g., human PD-L1) with a K_{D} of about 1 nM or less. In some embodiments, an anti-PD-L1 antibody or antigen-binding fragment binds PD-L1 (e.g., human PD-L1) with a K_{D} of about 0.5 nM or less. In some embodiments, an anti-PD-L1 antibody or antigen-binding fragment binds PD-L1 (e.g., human PD-L1) with a K_{D} of about 0.1 nM or less. In some embodiments, an anti-PD-L1 antibody or antigen-binding fragment binds PD-L1 (e.g., human PD-L1) with a K_{D} of about 50 pM or less. In some embodiments, an anti-PD-L1 antibody or antigen-binding fragment binds PD-L1 (e.g., human PD-L1) with a K_{D} of about 25 pM or less. In some embodiments, an anti-PD-L1 antibody or antigen-binding fragment binds PD-L1 (e.g., human PD-L1) with a K_{D} of about 10 pM or less. In some embodiments, an anti-PD-L1 antibody or antigen-binding fragment binds PD-L1 (e.g., human PD-L1) with a K_{D} of about 1 pM or less. In some embodiments, the dissociation constant of the binding agent (e.g., an antibody) for PD-L1 is the dissociation constant determined using a PD-L1 protein immobilized on a Biacore chip and the binding agent flowed over the chip. In some embodiments, the dissociation constant of the binding agent (e.g., an antibody) for PD-L1 is the dissociation constant determined using the binding agent captured by an anti-human IgG antibody on a Biacore chip and soluble PD-L1 flowed over the chip.

In some embodiments, an anti-PD-L1 antibody or antigen-binding fragment (e.g., an antibody) binds PD-L1 (e.g., human PD-L1) with a half maximal effective concentration (EC₅₀) of about 1 µM or less, about 100 nM or less, about 40 nM or less, about 20 nM or less, about 10 nM or less, about 1 nM or less, or about 0.1 nM or less. In some embodiments, an anti-PD-L1 antibody or antigen-binding fragment binds to PD-L1 with an EC₅₀ of about 1 µM or less, about 100 nM or less, about 40 nM or less, about 20 nM or less, about 10 nM or less, about 1 nM or less, or about 0.1 nM or less. In some embodiments, an anti-PD-L1 antibody or antigen-binding fragment binds PD-L1 with an EC₅₀ of about 40 nM or less. In some embodiments, an anti-PD-L1 antibody or antigen-binding fragment binds PD-L1 with an EC₅₀ of about 20 nM or less. In some embodiments, an anti-PD-L1 antibody or antigen-binding fragment binds PD-L1 with an EC₅₀ of about 10 nM or less. In some embodiments, an anti-PD-L1 antibody or antigen-binding fragment binds PD-L1 with an EC₅₀ of about 1 nM or less. In some embodiments, an anti-PD-L1 antibody or antigen-binding fragment binds PD-L1 with an EC₅₀ of about 0.1 nM or less.

The anti-PD-L1 antibodies or antigen-binding fragments of the present disclosure can be analyzed for their physical, chemical and/or biological properties by various methods known in the art. In some embodiments, an anti-PD-L1 antibody is tested for its ability to bind PD-L1 (e.g., human PD-L1). Binding assays include, but are not limited to, SPR (e.g., Biacore), ELISA, and FACS. In addition, antibodies can be evaluated for solubility, stability, thermostability, viscosity, expression levels, expression quality, and/or purification efficiency.

Epitope mapping is a method of identifying the binding site, region, or epitope on a target protein where an antibody binds. A variety of methods are known in the art for mapping epitopes on target proteins. These methods include mutagenesis, including but not limited to, shotgun mutagenesis, site-directed mutagenesis, and alanine scanning; domain or fragment scanning; peptide scanning (e.g., Pepscan technology); display methods (e.g., phage display, microbial display, and ribosome/mRNA display); methods involving proteolysis and mass spectroscopy; and structural determination (e.g., X-ray crystallography and NMR). In some embodiments, anti-PD-L1 antibodies or antigen-binding fragments described herein are characterized by assays including, but not limited to, N-terminal sequencing, amino acid analysis, HPLC, mass spectrometry, ion exchange chromatography, and papain digestion.

In some embodiments, an anti-PD-L1 antibody or antigen-binding fragment is conjugated to a cytotoxic agent or moiety. In some embodiments, an anti-PD-L1 antibody or antigen-binding fragment is conjugated to a cytotoxic agent to form an ADC (antibody-drug conjugate). In some embodiments, the cytotoxic moiety is a chemotherapeutic agent including, but not limited to, methotrexate, adriamycin/doxorubicin, melphalan, mitomycin C, chlorambucil, duocarmycin, daunorubicin, pyrrolobenzodiazepines (PBDs), or other intercalating agents. In some embodiments, the cytotoxic moiety is a microtubule inhibitor including, but not limited to, auristatins, maytansinoids (e.g., DM1 and DM4), and tubulysins. In some embodiments, the cytotoxic moiety is an enzymatically active toxin of bacterial, fungal, plant, or animal origin, or fragments thereof, including, but not limited to, diphtheria A chain, non-binding active fragments of diphtheria toxin, exotoxin A chain, ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes. In some embodiments, an antibody is conjugated to one or more small molecule toxins, such as calicheamicins, maytansinoids, trichothenes, and CC1065.

In some embodiments, an anti-PD-L1 antibody or antigen-binding fragment described herein is conjugated to a detectable substance or molecule that allows the agent to be used for diagnosis and/or detection. A detectable substance can include, but is not limited to, enzymes, such as horseradish peroxidase, alkaline phosphatase, beta-galactosidase, and acetylcholinesterase; prosthetic groups, such as biotin and flavine(s); fluorescent materials, such as, umbelliferone, fluorescein, fluorescein isothiocyanate (FITC), rhodamine, tetramethylrhodamine isothiocyanate (TRITC), dichlorotriazinylamine fluorescein, dansyl chloride, cyanine (Cy3), and phycoerythrin; bioluminescent materials, such as luciferase; radioactive materials, such as ²¹²Bi, ¹⁴C, ⁵⁷Co, ⁵¹Cr, ⁶⁷Cu, ¹⁸F, ⁶⁸Ga, ⁶⁷Ga, ¹⁵³Gd, ¹⁵⁹Gd, ⁶⁸Ge, ³H, ¹⁶⁶Ho, ¹³¹I, ¹²⁵I, ¹²³I, ¹²¹I, ¹¹⁵In, ¹¹³In, ¹¹²In, ¹¹¹In, ¹⁴⁰La, ¹⁷⁷Lu, ⁵⁴Mn, ⁹⁹Mo, ³²P, ¹⁰³Pd, ¹⁴⁹Pm, ¹⁴²Pr, ¹⁸⁶Re, ¹⁸⁸Re, ¹⁰⁵Rh, ⁹⁷Ru, ³⁵S, ⁴⁷Sc, ⁷⁵Se, ¹⁵³Sm, ¹¹³Sn, ¹¹⁷Sn, ⁸⁵Sr, ^{99m}Tc, ²⁰¹Ti, ¹³³Xe, ⁹⁰Y, ⁶⁹Yb, ¹⁷⁵Yb, ⁶⁵Zn; positron emitting metals; and magnetic metal ions positron emitting metals; and magnetic metal ions.

An anti-PD-L1 antibody or antigen-binding fragment described herein can be attached to a solid support. Such solid supports include, but are not limited to, glass, cellulose, polyacrylamide, nylon, polystyrene, polyvinyl chloride, or polypropylene. In some embodiments, an immobilized anti-PD-L1 antibody or antigen-binding fragment is used in an immunoassay. In some embodiments, an immobilized anti-PD-L1 antibody or antigen-binding fragment is used in purification of the target antigen (e.g., human PD-L1).

The anti-PD-L1 antibodies or antigen-binding fragments provided herein can be linked with a TGFβRII or a fragment thereof that binds TGFβ to form a fusion protein. Accordingly, provided herein are fusion proteins having a first domain comprising an antibody or an antigen-binding fragment thereof disclosed herein that binds PD-L1 (e.g., human PD-L1), and a second domain that comprises TGFβRII or a fragment thereof that binds TGFβ. The second domain can comprise any TGFβRII fragment disclosed in the section above that binds TGFβ. In some embodiments, the first and second domains are directly connected without a linker. In some embodiments, the first and second domains are linked via a linker. In some embodiments, the first and second domains are linked via a flexible linker. In some embodiments, the first and second domains are linked via a rigid linker. In some embodiments, the first and second domains are linked via a transferrin linker. In some embodiments, the C-terminus of the first domain is linked to the N-terminus of the second domain. In some embodiments, the C-terminus of the second domain is linked to the N-terminus of the first domain.

In some embodiments, the linker comprises the amino acid sequence of (GGGGS)n, n=1, 2, 3,4, or 5 (SEQ ID NO:245). In some embodiments, the linker is a rigid linker. In some embodiments, the linker has the amino acid sequence of (EAAAK)n, n=1,2, 3, 4, or 5 (SEQ ID NO:246).

In some embodiments, provided herein are fusion proteins wherein the first domain and the second domain are linked by a transferrin linker. In some embodiments, the transferrin linker is (PEAPTD)n, n=1, 2, 3, 4, or 5 (SEQ ID NO: 18). In some embodiments, the transferrin linker is (PEAPTDE)n, n=1, 2, 3, 4, or 5 (SEQ ID NO:19). The transferrin linker can be PEAPTD (SEQ ID NO: 145). The transferrin linker can be (PEAPTD)₂ (SEQ ID NO: 146). The transferrin linker can be (PEAPTD)₃ (SEQ ID NO: 147). The transferrin linker can be (PEAPTD)₄ (SEQ ID NO: 148). The transferrin linker can be (PEAPTD)₅ (SEQ ID NO: 149). The transferrin linker can be PEAPTDE (SEQ ID NO: 150). The transferrin linker can be (PEAPTDE)₂ (SEQ ID NO:151). The transferrin linker can be (PEAPTDE)₃ (SEQ ID NO: 152). The transferrin linker can be (PEAPTDE)₄ (SEQ ID NO: 153). The transferrin linker can be (PEAPTDE)₅ (SEQ ID NO: 154). In some embodiments, the transferrin linker is (PEAPTD)nP, n=1, 2, 3, 4, or 5 (SEQ ID NO:220). In some embodiments, the transferrin linker is (PEAPTD)nPE, n=1, 2, 3, 4, or 5 (SEQ ID NO:221). In some embodiments, the transferrin linker is (PEAPTD)nPEA, n=1, 2, 3, 4, or 5 (SEQ ID NO:222). In some embodiments, the transferrin linker is (PEAPTD)nPEAP, n=1, 2, 3, 4, or 5 (SEQ ID NO:223). In some embodiments, the transferrin linker is (PEAPTD)nPEAPT, n=1, 2, 3, 4, or 5 (SEQ ID NO:224). In some embodiments, the transferrin linker is (PEAPTDE)nP, n=1, 2, 3, 4, or 5 (SEQ ID NO:225). In some embodiments, the transferrin linker is (PEAPTDE)nPE, n=1, 2, 3, 4, or 5 (SEQ ID NO:226). In some embodiments, the transferrin linker is (PEAPTDE)nPEA, n=1, 2, 3, 4, or 5 (SEQ ID NO:227). In some embodiments, the transferrin linker is (PEAPTDE)nPEAP, n=1, 2, 3, 4, or 5 (SEQ ID NO:228). In some embodiments, the transferrin linker is (PEAPTDE)nPEAPT, n=1, 2, 3, 4, or 5 (SEQ ID NO:229). In some embodiments, the transferrin linker is (PEAPTDE)nPEAPTD, n=1, 2, 3, 4, or 5 (SEQ ID NO:230). For example, in some embodiments, the transferrin linker is (PEAPTD)2PEA, or PEAPTDPEAPTDPEA (SEQ ID NO:231).

In some embodiments, provided herein are fusion proteins having two domains, wherein the first domain comprises an antibody or an antigen-binding fragment disclosed herein that binds PD-L1, and the second domain comprises a fragment of TGFβRII that binds TGFβ, or a variant thereof. In some embodiments, the second domain comprises the ECD of TGFβRII isoform 1 (SEQ ID NO: 8), or a variant thereof that retains the binding to TGFβ. In some embodiments, the second domain comprises the ECD of TGFβRII isoform 2 (SEQ ID NO:14), or a variant thereof that retains the binding to TGFβ.

In some embodiments, the second domain of the fusion proteins provided herein comprises a variant of the TGFβRII ECD isoform 1 having at least 85%, at least 90%, at least 95%, or at least 98% sequence identity to SEQ ID NO:8. In some embodiments, the second domain has the amino acid sequence of SEQ ID NO:8. In some embodiments, the second domain has the amino acid sequence of SEQ ID NO:9. In some embodiments, the second domain comprises a variant of the ECD of TGFβRII isoform 1 (SEQ ID NO:8), wherein the variant comprises an amino acid mutation at Q6, K7, N19 or G20 of SEQ ID NO:8, or any combination thereof. In some embodiments, the variant comprises an amino acid mutation at Q6. In some embodiments, the variant comprises an amino acid mutation at K7. In some embodiments, the variant comprises an amino acid mutation at N19. In some embodiments, the variant comprises an amino acid mutation at G20. In some embodiments, the variant comprises amino acid mutations at K7 and N19. In some embodiments, the variant comprises amino acid mutations at K7 and G20. In some embodiments, the variant comprises amino acid mutations at N19 and G20. In some embodiments, the variant comprises amino acid mutations at Q6 and K7. In some embodiments, the variant comprises amino acid mutations at Q6 and N19. In some embodiments, the variant comprises amino acid mutations at Q6 and G20. In some embodiments, the variant comprises amino acid mutations at K7, N19 and G20. In some embodiments, the variant comprises amino acid mutations at Q6, K7 and N19. In some embodiments, the variant comprises amino acid mutations at Q6, K7 and G20. In some embodiments, the variant comprises amino acid mutations at Q6, N19 and G20. In some embodiments, the variant comprises amino acid mutations at Q6, K7, N19 and G20. In some embodiments, the amino acid mutation at Q6 is an amino acid substitution at Q6. In some embodiments, the amino acid mutation at K7 is an amino acid substitution at K7. In some embodiments, the amino acid mutation at N19 is an amino acid substitution at N19. In some embodiments, the amino acid mutation at G20 is an amino acid substitution at G20. The substitution can change the original amino acid to any other amino acid that is different from the original amino acid. For example, the K7 substitution can change the K residue to any amino acid that is not K. In some embodiments, the variant comprises a N19T substitution. In some embodiments, the variant comprises a N19A substitution. In some embodiments, the variant comprises a Q6G substitution. In some embodiments, the variant comprises a K7G substitution. In some embodiments, the variant comprises a K7G substitution and a N19T substitution. In some embodiments, the variant comprises a K7G substitution and a N19A substitution. In some embodiments, the variant comprises a Q6G substitution, a K7G substitution and a N19A substitution. In some embodiments, the second domain has the amino acid sequence of SEQ ID NO:201. In some embodiments, the second domain has the amino acid sequence of SEQ ID NO:202. In some embodiments, the second domain has the amino acid sequence of SEQ ID NO:203.

In some embodiments, the second domain of the fusion proteins provided herein comprises a variant of the TGFβRII ECD isoform 1 that is either a N-terminal truncation (i.e., lacks a N-terminal fragment) or a C-terminal truncation (i.e., lacks C-terminal fragment) of TGFβRII ECD isoform 1. In some embodiments, the second domain of the fusion proteins provided herein comprises a truncated form of the ECD of TGFβRII isoform 1 (SEQ ID NO:8), which lacks amino acid residues 1 to n of SEQ ID NO:8, wherein n ranges from 2 to 30, or a variant thereof. In other words, the second domain of the fusion proteins provided herein can comprise a truncated form of the TGFβRII ECD isoform 1 (SEQ ID NO:8) lacking a N-terminal fragment that ranges from 2 to 30 amino acids. In some embodiments, the truncated form of the TGFβRII ECD isoform 1 lacks amino acid residues 1 to 2, 1 to 3, 1 to 4, 1 to 5, 1 to 6, 1 to 7, 1 to 8, 1 to 9, 1 to 10, 1 to 11, 1 to 12, 1 to 13, 1 to 14, 1 to 15, 1 to 16, 1 to 17, 1 to 18, 1 to 19, 1 to 20, 1 to 21, 1 to 22, 1 to 23, 1 to 24, 1 to 25, 1 to 26, 1 to 27, 1 to 28, 1 to 29, or 1 to 30 of SEQ ID NO:8. In some embodiments, n is 19, and the second domain comprises amino acids 20-136 of the ECD of TGFβRII isoform 1 (SEQ ID NO: 8), or a variant thereof. In some embodiments, the second domain has the amino acid sequence of SEQ ID NO: 10. In some embodiments, the second domain is a truncated form of the TGFβRII ECD isoform 1 (SEQ ID NO: 8) that lacks amino acid residues 1 to 24 of SEQ ID NO:8.

In some embodiments, the second domain of the fusion proteins provided herein comprises a truncated form of the ECD of TGFβRII isoform 1 (SEQ ID NO: 8), which lacks amino acid residues m to 136 of SEQ ID NO: 8, wherein m ranges from 80 to 135, or a variant thereof. In other words, the second domain of the fusion proteins provided herein can comprise a truncated form of the TGFβRII ECD isoform 1 (SEQ ID NO:8) lacking a C-terminal fragment that ranges from 1 to 56 amino acids. In some embodiments, the truncated form of the TGFβRII ECD isoform 1 lacks amino acid residues 80 to 136, 81 to 136, 82 to 136, 83 to 136, 84 to 136, 85 to 136, 86 to 136, 87 to 136, 88 to 136, 89 to 136, 90 to 136, 91 to 136, 92 to 136, 93 to 136, 94 to 136, 95 to 136, 96 to 136, 97 to 136, 98 to 136, 99 to 136, 100 to 136, 101 to 136, 102 to 136, 103 to 136, 104 to 136, 105 to 136, 106 to 136, 107 to 136, 108 to 136, 109 to 136, 110 to 136, 111 to 136, 112 to 136, 113 to 136, 114 to 136, 115 to 136, 116 to 136, 117 to 136, 118 to 136, 119 to 136, 120 to 136, 121 to 136, 122 to 136, 123 to 136, 124 to 136, 125 to 136, 126 to 136, 127 to 136, 128 to 136, 129 to 136, 130 to 136, 131 to 136, 132 to 136, 133 to 136, 134 to 136, or 135 to 136 of SEQ ID NO:8. In some embodiments, the truncated form of the TGFβRII ECD isoform 1 (SEQ ID NO:8) lacks a C-terminal fragment that ranges from 15 to 25 amino acids. In some embodiments, the truncated form of the TGFβRII ECD isoform 1 (SEQ ID NO:8) lacks a C-terminal fragment that ranges from 5 to 15 amino acids. In some embodiments, m is 131, and the second domain comprises amino acids 1-130 of the ECD of TGFβRII isoform 1 (SEQ ID NO:8), or a variant thereof. In other words, the second domain can be a truncated form of the TGFβRII ECD isoform 1 (SEQ ID NO:8) lacking the 6 amino acids in the C-terminus. In some embodiments, m is 128, and the second domain comprises amino acids 1-127 of the ECD of TGFβRII isoform 1 (SEQ ID NO:8), or a variant thereof. In other words, the second domain can be a truncated form of the TGFβRII ECD isoform 1 (SEQ ID NO:8) lacking the 9 amino acids in the C-terminus. In some embodiments, the second domain has the amino acid sequence of SEQ ID NO:11. In some embodiments, the second domain has the amino acid sequence of SEQ ID NO:12. In some embodiments, the second domain of the fusion proteins provided herein comprises a truncated form of the TGFβRII ECD isoform 1 (SEQ ID NO:8) that lacks amino acid residues 83 to 136 of SEQ ID NO:8.

In some embodiments, the second domain comprises a C-terminal truncated form of the ECD of TGFβRII isoform 1 (SEQ ID NO:8) that further comprises an amino acid mutation at Q6, K7, N19 or G20, or any combination thereof. In some embodiments, the truncated form further comprises an amino acid mutation at Q6. In some embodiments, the truncated form further comprises an amino acid mutation at K7. In some embodiments, the truncated form further comprises an amino acid mutation at N19. In some embodiments, the truncated form further comprises an amino acid mutation at G20. In some embodiments, the truncated form further comprises amino acid mutations at Q6 and K7. In some embodiments, the truncated form further comprises amino acid mutations at Q6 and N19. In some embodiments, the truncated form further comprises amino acid mutations at Q6 and G20. In some embodiments, the truncated form further comprises amino acid mutations at K7 and N19. In some embodiments, the truncated form further comprises amino acid mutations at K7 and G20. In some embodiments, the truncated form further comprises amino acid mutations at N19 and G20. In some embodiments, the truncated form further comprises amino acid mutations at Q6, K7, and N19. In some embodiments, the truncated form further comprises amino acid mutations at Q6, K7, and G20. In some embodiments, the truncated form further comprises amino acid mutations at Q6, N19 and G20. In some embodiments, the truncated form further comprises amino acid mutations at K7, N19 and G20. In some embodiments, the truncated form further comprises amino acid mutations at Q6, K7, N19 and G20. In some embodiments, the amino acid mutation at Q6 is an amino acid substitution at Q6. In some embodiments, the amino acid mutation at K7 is an amino acid substitution at K7. In some embodiments, the amino acid mutation at N19 is an amino acid substitution at N19. In some embodiments, the amino acid mutation at G20 is an amino acid substitution at G20. The substitution can change the original amino acid to any other amino acid that is different from the original amino acid. For example, the K7 substitution can change the K residue to any amino acid that is not K. In some embodiments, the truncated form further comprises a Q6G substitution. In some embodiments, the truncated form further comprises a K7G substitution. In some embodiments, the truncated form further comprises a N19T substitution. In some embodiments, the truncated form further comprises a N19A substitution. In some embodiments, the truncated form further comprises a K7G substitution and a N19T substitution. In some embodiments, the truncated form further comprises a K7G substitution and a N19A substitution. In some embodiments, the truncated form further comprises a Q6G substitution, a K7G substitution and a N19A substitution. In some embodiments, the second domain has the amino acid sequence of SEQ ID NO:204. In some embodiments, the second domain has the amino acid sequence of SEQ ID NO:205. In some embodiments, the second domain has the amino acid sequence of SEQ ID NO:232.

In some embodiments, the second domain of the fusion proteins provided herein comprises a variant of the TGFβRII ECD isoform 2 having at least 85%, at least 90%, at least 95%, or at least 98% sequence identity to SEQ ID NO: 14. In some embodiments, the second domain of the fusion proteins provided herein has at least 85% sequence identity to SEQ ID NO:14. In some embodiments, the second domain of the fusion proteins provided herein has at least 90% sequence identity to SEQ ID NO: 14. In some embodiments, the second domain of the fusion proteins provided herein has at least 95% sequence identity to SEQ ID NO: 14. In some embodiments, the second domain of the fusion proteins provided herein has at least 98% sequence identity to SEQ ID NO:14. In some embodiments, the second domain has the amino acid sequence of SEQ ID NO:14. In some embodiments, the second domain of the fusion proteins provided herein comprises a variant of the TGFβRII ECD isoform 2 that is either a N-terminal truncation (i.e., lacks a N-terminal fragment) or a C-terminal truncation (i.e., lacks C-terminal fragment) of TGFβRII ECD isoform 2. In some embodiments, the second domain of the fusion proteins provided herein comprises a truncated form of the TGFβRII ECD isoform 2 (SEQ ID NO:14) lacking a N-terminal fragment. In some embodiments, the truncated form of the TGFβRII ECD isoform 1 lacks amino acid residues 1 to n of SEQ ID NO:14, wherein n ranges from 2 to 30. In some embodiments, the truncated form of the TGFβRII ECD isoform 2 lacks amino acid residues 1 to 2, 1 to 3, 1 to 4, 1 to 5, 1 to 6, 1 to 7, 1 to 8, 1 to 9, 1 to 10, 1 to 11, 1 to 12, 1 to 13, 1 to 14, 1 to 15, 1 to 16, 1 to 17, 1 to 18, 1 to 19, 1 to 20, 1 to 21, 1 to 22, 1 to 23, 1 to 24, 1 to 25, 1 to 26, 1 to 27, 1 to 28, 1 to 29, 1 to 30, 1 to 31, 1 to 32, 1 to 33, 1 to 34, 1 to 35, 1 to 36, 1 to 37, 1 to 38, 1 to 39, 1 to 40, 1 to 41, 1 to 42, 1 to 43, 1 to 44, 1 to 45, 1 to 46, 1 to 47, 1 to 48, 1 to 49, 1 to 50, 1 to 51, 1 to 52, 1 to 53, 1 to 54, 1 to 55, 1 to 56, 1 to 57, 1 to 58, 1 to 59, or 1 to 60 of SEQ ID NO:14. In some embodiments, the second domain of the fusion proteins provided herein comprises a variant of the TGFβRII ECD isoform 2 (SEQ ID NO:14) lacking a N-terminal fragment that ranges from 1 to 44 amino acids. In some embodiments, the second domain has the amino acid sequence of SEQ ID NO:15. In some embodiments, the second domain is a variant of the TGFβRII ECD isoform 2 (SEQ ID NO:14) lacking amino acid residues 1 to 49 in the N-terminus.

In some embodiments, the second domain of the fusion proteins provided herein comprises a variant of the TGFβRII ECD isoform 2 (SEQ ID NO:14) lacking a C-terminal fragment that ranges. In some embodiments, the truncated form of the TGFβRII ECD isoform 2 lacks amino acid residues m to 161 of SEQ ID NO:14, wherein m ranges from 100 to 160. In some embodiments, the truncated form of the TGFβRII ECD isoform 1 lacks amino acid residues 100 to 161, 101 to 161, 102 to 161, 103 to 161, 104 to 161, 105 to 161, 106 to 161, 107 to 161, 108 to 161, 109 to 161, 110 to 161, 111 to 161, 112 to 161, 113 to 161, 114 to 161, 115 to 161, 116 to 161, 117 to 161, 118 to 161, 119 to 161, 120 to 161, 121 to 161, 122 to 161, 123 to 161, 124 to 161, 125 to 161, 126 to 161, 127 to 161, 128 to 161, 129 to 161, 130 to 161, 131 to 161, 132 to 161, 133 to 161, 134 to 161, 135 to 161, 136 to 161, 137 to 161, 138 to 161, 139 to 161, 140 to 161, 141 to 161, 142 to 161, 143 to 161, 144 to 161, 145 to 161, 146 to 161, 147 to 161, 148 to 161, 149 to 161, 150 to 161, 151 to 161, 152 to 161, 153 to 161, 154 to 161, 155 to 161, 156 to 161, 157 to 161, 158 to 161, 159 to 161, or 160 to 161 of SEQ ID NO:14. In some embodiments, the second domain of the fusion proteins provided herein comprises a truncated form of the TGFβRII ECD isoform 2 (SEQ ID NO:14) lacking a C-terminal fragment that ranges from 5 to 15 amino acids. In some embodiments, the second domain is a variant of the TGFβRII ECD isoform 2 (SEQ ID NO:14) lacking the 9 amino acids in the C-terminus. In some embodiments, the second domain is a truncated form of the TGFβRII ECD isoform 2 (SEQ ID NO:14) lacking amino acid residues 128 to 161 of SEQ ID NO:14. In some embodiments, the second domain has the amino acid sequence of SEQ ID NO: 16.

As disclosed herein, the fusion proteins provided herein can comprise an anti-PD-L1 antibody or antigen-binding fragment (the anti-PD-L1 domain) provided herein linked with a TGFβRII or a fragment thereof that binds TGFβ (the TGFβ trap domain). In some embodiments, the anti-PD-L1 domain and the TGFβ trap domain are directly connected without a linker. In some embodiments, the anti-PD-L1 domain and the TGFβ trap domain are linked via a linker. Te linker can be a rigid linker provided herein. In some embodiments, the anti-PD-L1 domain consists of a single polypeptide, and the fusion protein also consists of a single polypeptide having the anti-PD-L1 domain linked to a TGFβ trap domain. For example, in some embodiments, the fusion protein consists of an anti-PD-L1 scFv disclosed herein linked to a TGFβ trap domain. In some embodiments, the fusion protein consists of an anti-PD-L1 single domain antibody linked to a TGFβ trap domain. In some embodiments, the anti-PD-L1 domain comprises a first polypeptide and a second polypeptide, and the TGFβ trap domain can be linked to either the first polypeptide or the polypeptide. In some embodiments, the fusion protein comprises (1) a first polypeptide comprising the first polypeptide of the anti-PD-L1 domain linked to a TGFβ trap domain, and (2) a second polypeptide comprising the second polypeptide of the anti-PD-L1 domain. For example, in some embodiments, the anti-PD-L1 domain comprises an antibody heavy chain and an antibody light chain, and the fusion protein comprises a heavy chain comprising the antibody heavy chain linked to a TGFβ trap domain, and a light chain comprising the antibody light chain (e.g., FIG. 6). In some embodiments, the anti-PD-L1 domain comprises an antibody heavy chain and an antibody light chain, and the fusion protein comprises a heavy chain comprising the antibody heavy chain, and a light chain comprising the antibody light chain linked to a TGFβ trap domain.

For example, in some embodiments, the fusion proteins provided herein comprise an anti-PD-L1 antibody that is an IgG1 antibody. Accordingly, the fusion proteins comprise (1) a heavy chain comprising an anti-PD-L1 IgG1 heavy chain having (a) an anti-PD-L1 VH disclosed herein and (b) IgG1 heavy chain constant regions, including an IgG1 Fc region, linked to a TGFβ trap domain and (2) an IgG1 light chain having (a) an anti-PD-L1 VL disclosed herein and (b) an IgG1 light chain constant region. For another example, in some embodiments, the fusion proteins provided herein comprise an anti-PD-L1 antibody that is an IgG4 antibody. Accordingly, the fusion proteins comprise (1) a heavy chain comprising an anti-PD-L1 IgG1 heavy chain having (a) an anti-PD-L1 VH disclosed herein and (b) an IgG4 heavy chain constant regions, including an IgG4 Fc region, linked to a TGFβ trap domain and (2) an IgG4 light chain having (a) an anti-PD-L1 VL disclosed herein and (b) an IgG1 light chain constant region. A person of ordinary skill in the art would understand that the anti-PD-L1 domain can have any configurations disclosed herein or otherwise known in the art, and the sequences of the constant regions or Fc regions of the different antibody configurations are also well known in the art.

In some embodiments, provided herein are fusion proteins comprising a heavy chain, wherein the heavy chain has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to an amino acid sequence selected from the group consisting of SEQ ID NOs:155-161, 166-172, 206-212 and 233-240. In some embodiments, provided herein are fusion proteins comprising a light chain, wherein the light chain has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to an amino acid sequence selected from the group consisting of SEQ ID NOs: 162-165.

TABLE 7

Amino acid sequences of heavy chains and light chains of fusion proteins

| | |
|---|---|
| Domain | Heavy Chain Sequences |
| Clone B-linker (PEAPTD)3-Trap 1-127 | QGQLVQSGAEVKKPGSSVKVSCKTSGFTFSSSYISWVRQAPGQGLEWMGWIYA GTGGTSYNQKFTGRVTITVDTSTSTAYMELSSLRSEDTAVYYCARHEGKYWYF DVWGQGTTVTVSSASTKGPSVFPLAPSSKSTSGGTAALGCLVKDYFPEPVTVSW NSGALTSGVHTFPAVLQSSGLYSLSSVVTVPSSSLGTQTYICNVNHKPSNTKVDK RVEPKSCDKTHTCPPCPAPELLGGPSVFLFPPKPKDTLMISRTPEVTCVVVDVSH EDPEVKFNWYVDGVEVHNAKTKPREEQYNSTYRVVSVLTVLHQDWLNGKEYK CKVSNKALPAPIEKTISKAKGQPREPQVYTLPPSREEMTKNQVSLTCLVKGFYPS DIAVEWESNGQPENNYKTTPPVLDSDGSFFLYSKLTVDKSRWQQGNVFSCSVM HEALHNHYTQKSLSLSPGAPEAPTDPEAPTDPEAPTDIPPHVQKSVNNDMIVTDN NGAVKFPQLCKFCDVRFSTCDNQKSCMSNCSITSICEKPQEVCVAVWRKNDENI TLETVCHDPKLPYHDFILEDAASPKCIMKEKKKPGETFFMCSCSSDECNDNIIFS (SEQ ID NO:155) |
| Clone B-linker (PEAPTD)3-Trap 1-136 | QGQLVQSGAEVKKPGSSVKVSCKTSGFTFSSSYISWVRQAPGQGLEWMGWIYA GTGGTSYNQKFTGRVTITVDTSTSTAYMELSSLRSEDTAVYYCARHEGKYWYF DVWGQGTTVTVSSASTKGPSVFPLAPSSKSTSGGTAALGCLVKDYFPEPVTVSW NSGALTSGVHTFPAVLQSSGLYSLSSVVTVPSSSLGTQTYICNVNHKPSNTKVDK RVEPKSCDKTHTCPPCPAPELLGGPSVFLFPPKPKDTLMISRTPEVTCVVVDVSH EDPEVKFNWYVDGVEVHNAKTKPREEQYNSTYRVVSVLTVLHQDWLNGKEYK CKVSNKALPAPIEKTISKAKGQPREPQVYTLPPSREEMTKNQVSLTCLVKGFYPS DIAVEWESNGQPENNYKTTPPVLDSDGSFFLYSKLTVDKSRWQQGNVFSCSVM HEALHNHYTQKSLSLSPGAPEAPTDPEAPTDPEAPTDIPPHVQKSVNNDMIVTDN NGAVKFPQLCKFCDVRFSTCDNQKSCMSNCSITSICEKPQEVCVAVWRKNDENI TLETVCHDPKLPYHDFILEDAASPKCIMKEKKKPGETFFMCSCSSDECNDNIIFSE EYNTSNPD (SEQ ID NO:156) |
| Clone B-linker (PEAPTD)2-Trap 1-127 | QGQLVQSGAEVKKPGSSVKVSCKTSGFTFSSSYISWVRQAPGQGLEWMGWIYA GTGGTSYNQKFTGRVTITVDTSTSTAYMELSSLRSEDTAVYYCARHEGKYWYF DVWGQGTTVTVSSASTKGPSVFPLAPSSKSTSGGTAALGCLVKDYFPEPVTVSW NSGALTSGVHTFPAVLQSSGLYSLSSVVTVPSSSLGTQTYICNVNHKPSNTKVDK |
| | RVEPKSCDKTHTCPPCPAPELLGGPSVFLFPPKPKDTLMISRTPEVTCVVVDVSH EDPEVKFNWYVDGVEVHNAKTKPREEQYNSTYRVVSVLTVLHQDWLNGKEYK CKVSNKALPAPIEKTISKAKGQPREPQVYTLPPSREEMTKNQVSLTCLVKGFYPS DIAVEWESNGQPENNYKTTPPVLDSDGSFFLYSKLTVDKSRWQQGNVFSCSVM HEALHNHYTQKSLSLSPGAPEAPTDPEAPTDIPPHVQKSVNNDMIVTDNNGAVK FPQLCKFCDVRFSTCDNQKSCMSNCSITSICEKPQEVCVAVWRKNDENITLETVC HDPKLPYHDFILEDAASPKCIMKEKKKPGETFFMCSCSSDECNDNIIFS (SEQ ID NO:157) |
| Clone B-linker (PEAPTD)4-Trap 1-127 | QGQLVQSGAEVKKPGSSVKVSCKTSGFTFSSSYISWVRQAPGQGLEWMGWIYA GTGGTSYNQKFTGRVTITVDTSTSTAYMELSSLRSEDTAVYYCARHEGKYWYF DVWGQGTTVTVSSASTKGPSVFPLAPSSKSTSGGTAALGCLVKDYFPEPVTVSW NSGALTSGVHTFPAVLQSSGLYSLSSVVTVPSSSLGTQTYICNVNHKPSNTKVDK RVEPKSCDKTHTCPPCPAPELLGGPSVFLFPPKPKDTLMISRTPEVTCVVVDVSH EDPEVKFNWYVDGVEVHNAKTKPREEQYNSTYRVVSVLTVLHQDWLNGKEYK CKVSNKALPAPIEKTISKAKGQPREPQVYTLPPSREEMTKNQVSLTCLVKGFYPS DIAVEWESNGQPENNYKTTPPVLDSDGSFFLYSKLTVDKSRWQQGNVFSCSVM HEALHNHYTQKSLSLSPGAPEAPTDPEAPTDPEAPTDPEAPTDIPPHVQKSVNND MIVTDNNGAVKFPQLCKFCDVRFSTCDNQKSCMSNCSITSICEKPQEVCVAVWR KNDENITLETVCHDPKLPYHDFILEDAASPKCIMKEKKKPGETFFMCSCSSDECN DNIIFS (SEQ ID NO:158) |
| Clone D-linker (PEAPTD)3-Trap 1-127 | QVQLVQSGAEVKKPGASVKVSCKASGYSFTGYTMNWVKQAHGQGLEWMGLII PYNGGISYNQKFKDRVTMTVDTSISTAYMELSRLRSDDTAVYYCASLITTAPRDS MDYWGQGTLVTVSSASTKGPSVFPLAPSSKSTSGGTAALGCLVKDYFPEPVTVS WNSGALTSGVHTFPAVLQSSGLYSLSSVVTVPSSSLGTQTYICNVNHKPSNTKV DKRVEPKSCDKTHTCPPCPAPELLGGPSVFLFPPKPKDTLMISRTPEVTCVVVDV SHEDPEVKFNWYVDGVEVHNAKTKPREEQYNSTYRVVSVLTVLHQDWLNGKE YKCKVSNKALPAPIEKTISKAKGQPREPQVYTLPPSREEMTKNQVSLTCLVKGFY PSDIAVEWESNGQPENNYKTTPPVLDSDGSFFLYSKLTVDKSRWQQGNVFSCSV MHEALHNHYTQKSLSLSPGAPEAPTDPEAPTDPEAPTDIPPHVQKSVNNDMIVT DNNGAVKFPQLCKFCDVRFSTCDNQKSCMSNCSITSICEKPQEVCVAVWRKND ENITLETVCHDPKLPYHDFILEDAASPKCIMKEKKKPGETFFMCSCSSDECNDNII FS (SEQ ID NO:159) |
| Clone C-linker (PEAPTD)3-Trap 1-127 | QVQLVQSGAEVKKPGASVKVSCKASGFNIKDTYMHWVRQAPGQGLEWIGRIDP ANGNTKYDPKFQGRVTITTDTSASTAYLELSSLRSEDTAVYYCARGLGRWFAY WGQGTTVTVSSASTKGPSVFPLAPSSKSTSGGTAALGCLVKDYFPEPVTVSWNS GALTSGVHTFPAVLQSSGLYSLSSVVTVPSSSLGTQTYICNVNHKPSNTKVDKRV EPKSCDKTHTCPPCPAPELLGGPSVFLFPPKPKDTLMISRTPEVTCVVVDVSHEDP EVKFNWYVDGVEVHNAKTKPREEQYNSTYRVVSVLTVLHQDWLNGKEYKCK VSNKALPAPIEKTISKAKGQPREPQVYTLPPSREEMTKNQVSLTCLVKGFYPSDI AVEWESNGQPENNYKTTPPVLDSDGSFFLYSKLTVDKSRWQQGNVFSCSVMHE ALHNHYTQKSLSLSPGAPEAPTDPEAPTDPEAPTDIPPHVQKSVNNDMIVTDNNG AVKFPQLCKFCDVRFSTCDNQKSCMSNCSITSICEKPQEVCVAVWRKNDENITL ETVCHDPKLPYHDFILEDAASPKCIMKEKKKPGETFFMCSCSSDECNDNIIFS (SEQ ID NO:160) |
| Clone N-linker (PEAPTD)3-Trap 1-127 | EVQLQQSGPELVKPGASMKISCKASGYSFTGYTMNWVKQSHGKNLEWIGLIIPH NGGTSYNQKFKDKATLTVDKSSRTAYMELLSLTSEDSAVYYCASLMTTAPRDS MDYWGQGTSVTVSSASTKGPSVFPLAPSSKSTSGGTAALGCLVKDYFPEPVTVS WNSGALTSGVHTFPAVLQSSGLYSLSSVVTVPSSSLGTQTYICNVNHKPSNTKV DKRVEPKSCDKTHTCPPCPAPELLGGPSVFLFPPKPKDTLMISRTPEVTCVVVDV |
| | SHEDPEVKFNWYVDGVEVHNAKTKPREEQYNSTYRVVSVLTVLHQDWLNGKE YKCKVSNKALPAPIEKTISKAKGQPREPQVYTLPPSREEMTKNQVSLTCLVKGFY PSDIAVEWESNGQPENNYKTTPPVLDSDGSFFLYSKLTVDKSRWQQGNVFSCSV MHEALHNHYTQKSLSLSPGAPEAPTDPEAPTDPEAPTDIPPHVQKSVNNDMIVT DNNGAVKFPQLCKFCDVRFSTCDNQKSCMSNCSITSICEKPQEVCVAVWRKND ENITLETVCHDPKLPYHDFILEDAASPKCIMKEKKKPGETFFMCSCSSDECNDNII FS (SEQ ID NO:161) |
| Clone B(IgG4)-linker (PEAPTD)3-Trap1-127 | QGQLVQSGAEVKKPGSSVKVSCKTSGFTFSSSYISWVRQAPGQGLEWMGWIYA GTGGTSYNQKFTGRVTITVDTSTSTAYMELSSLRSEDTAVYYCARHEGKYWYF DVWGQGTTVTVSSASTKGPSVFPLAPCSRSTSESTAALGCLVKDYFPEPVTVSW NSGALTSGVHTFPAVLQSSGLYSLSSVVTVPSSSLGTKTYTCNVDHKPSNTKVD KRVESKYGPPCPPCPAPEFLGGPSVFLFPPKPKDTLMISRTPEVTCVVVDVSQEDP EVQFNWYVDGVEVHNAKTKPREEQFNSTYRVVSVLTVLHQDWLNGKEYKCK VSNKGLPSSIEKTISKAKGQPREPQVYTLPPSQEEMTKNQVSLTCLVKGFYPSDIA VEWESNGQPENNYKTTPPVLDSDGSFFLYSRLTVDKSRWQEGNVFSCSVMHEA LHNHYTQKSLSLSLGAPEAPTDPEAPTDPEAPTDIPPHVQKSVNNDMIVTDNNG AVKFPQLCKFCDVRFSTCDNQKSCMSNCSITSICEKPQEVCVAVWRKNDENITL ETVCHDPKLPYHDFILEDAASPKCIMKEKKKPGETFFMCSCSSDECNDNIIFS (SEQ ID NO:166) |
| Clone B(IgG4)-linker (PEAPTD)3-Trap1-136 | QGQLVQSGAEVKKPGSSVKVSCKTSGFTFSSSYISWVRQAPGQGLEWMGWIYA GTGGTSYNQKFTGRVTITVDTSTSTAYMELSSLRSEDTAVYYCARHEGKYWYF DVWGQGTTVTVSSASTKGPSVFPLAPCSRSTSESTAALGCLVKDYFPEPVTVSW NSGALTSGVHTFPAVLQSSGLYSLSSVVTVPSSSLGTKTYTCNVDHKPSNTKVD KRVESKYGPPCPPCPAPEFLGGPSVFLFPPKPKDTLMISRTPEVTCVVVDVSQEDP EVQFNWYVDGVEVHNAKTKPREEQFNSTYRVVSVLTVLHQDWLNGKEYKCK VSNKGLPSSIEKTISKAKGQPREPQVYTLPPSQEEMTKNQVSLTCLVKGFYPSDIA VEWESNGQPENNYKTTPPVLDSDGSFFLYSRLTVDKSRWQEGNVFSCSVMHEA LHNHYTQKSLSLSLGAPEAPTDPEAPTDPEAPTDIPPHVQKSVNNDMIVTDNNG AVKFPQLCKFCDVRFSTCDNQKSCMSNCSITSICEKPQEVCVAVWRKNDENITL ETVCHDPKLPYHDFILEDAASPKCIMKEKKKPGETFFMCSCSSDECNDNIIFSEEY NTSNPD (SEQ ID NO:167) |
| Clone B(IgG4)-linker(PEAPT D)2-Trap1-127 | QGQLVQSGAEVKKPGSSVKVSCKTSGFTFSSSYISWVRQAPGQGLEWMGWIYA GTGGTSYNQKFTGRVTITVDTSTSTAYMELSSLRSEDTAVYYCARHEGKYWYF DVWGQGTTVTVSSASTKGPSVFPLAPCSRSTSESTAALGCLVKDYFPEPVTVSW NSGALTSGVHTFPAVLQSSGLYSLSSVVTVPSSSLGTKTYTCNVDHKPSNTKVD KRVESKYGPPCPPCPAPEFLGGPSVFLFPPKPKDTLMISRTPEVTCVVVDVSQEDP EVQFNWYVDGVEVHNAKTKPREEQFNSTYRVVSVLTVLHQDWLNGKEYKCK VSNKGLPSSIEKTISKAKGQPREPQVYTLPPSQEEMTKNQVSLTCLVKGFYPSDIA VEWESNGQPENNYKTTPPVLDSDGSFFLYSRLTVDKSRWQEGNVFSCSVMHEA LHNHYTQKSLSLSLGAPEAPTDPEAPTDIPPHVQKSVNNDMIVTDNNGAVKFPQ LCKFCDVRFSTCDNQKSCMSNCSITSICEKPQEVCVAVWRKNDENITLETVCHD PKLPYHDFILEDAASPKCIMKEKKKPGETFFMCSCSSDECNDNIIFS (SEQ ID NO:168) |
| Clone B(IgG4)-linker (PEAPTD)4-Trap1-127 | QGQLVQSGAEVKKPGSSVKVSCKTSGFTFSSSYISWVRQAPGQGLEWMGWIYA GTGGTSYNQKFTGRVTITVDTSTSTAYMELSSLRSEDTAVYYCARHEGKYWYF DVWGQGTTVTVSSASTKGPSVFPLAPCSRSTSESTAALGCLVKDYFPEPVTVSW NSGALTSGVHTFPAVLQSSGLYSLSSVVTVPSSSLGTKTYTCNVDHKPSNTKVD KRVESKYGPPCPPCPAPEFLGGPSVFLFPPKPKDTLMISRTPEVTCVVVDVSQEDP EVQFNWYVDGVEVHNAKTKPREEQFNSTYRVVSVLTVLHQDWLNGKEYKCK |
| | VSNKGLPSSIEKTISKAKGQPREPQVYTLPPSQEEMTKNQVSLTCLVKGFYPSDIA VEWESNGQPENNYKTTPPVLDSDGSFFLYSRLTVDKSRWQEGNVFSCSVMHEA LHNHYTQKSLSLSLGAPEAPTDPEAPTDPEAPTDPEAPTDIPPHVQKSVNNDMIV TDNNGAVKFPQLCKFCDVRFSTCDNQKSCMSNCSITSICEKPQEVCVAVWRKN DENITLETVCHDPKLPYHDFILEDAASPKCIMKEKKKPGETFFMCSCSSDECNDN IIFS (SEQ ID NO:169) |
| Clone D(IgG4)-linker (PEAPTD)3-Trap1-127 | QVQLVQSGAEVKKPGASVKVSCKASGYSFTGYTMNWVKQAHGQGLEWMGLII PYNGGISYNQKFKDRVTMTVDTSISTAYMELSRLRSDDTAVYYCASLITTAPRDS MDYWGQGTLVTVSSASTKGPSVFPLAPCSRSTSESTAALGCLVKDYFPEPVTVS WNSGALTSGVHTFPAVLQSSGLYSLSSVVTVPSSSLGTKTYTCNVDHKPSNTKV DKRVESKYGPPCPPCPAPEFLGGPSVFLFPPKPKDTLMISRTPEVTCVVVDVSQE DPEVQFNWYVDGVEVHNAKTKPREEQFNSTYRVVSVLTVLHQDWLNGKEYKC KVSNKGLPSSIEKTISKAKGQPREPQVYTLPPSQEEMTKNQVSLTCLVKGFYPSDI AVEWESNGQPENNYKTTPPVLDSDGSFFLYSRLTVDKSRWQEGNVFSCSVMHE ALHNHYTQKSLSLSLGAPEAPTDPEAPTDPEAPTDIPPHVQKSVNNDMIVTDNN GAVKFPQLCKFCDVRFSTCDNQKSCMSNCSITSICEKPQEVCVAVWRKNDENIT LETVCHDPKLPYHDFILEDAASPKCIMKEKKKPGETFFMCSCSSDECNDNIIFS (SEQ ID NO:170) |
| Clone C(IgG4)- linker (PEAPTD)3-Trap1-127 | QVQLVQSGAEVKKPGASVKVSCKASGFNIKDTYMHWVRQAPGQGLEWIGRIDP ANGNTKYDPKFQGRVTITTDTSASTAYLELSSLRSEDTAVYYCARGLGRWFAY WGQGTTVTVSSASTKGPSVFPLAPCSRSTSESTAALGCLVKDYFPEPVTVSWNS GALTSGVHTFPAVLQSSGLYSLSSVVTVPSSSLGTKTYTCNVDHKPSNTKVDKR VESKYGPPCPPCPAPEFLGGPSVFLFPPKPKDTLMISRTPEVTCVVVDVSQEDPEV QFNWYVDGVEVHNAKTKPREEQFNSTYRVVSVLTVLHQDWLNGKEYKCKVSN KGLPSSIEKTISKAKGQPREPQVYTLPPSQEEMTKNQVSLTCLVKGFYPSDIAVE WESNGQPENNYKTTPPVLDSDGSFFLYSRLTVDKSRWQEGNVFSCSVMHEALH NHYTQKSLSLSLGAPEAPTDPEAPTDPEAPTDIPPHVQKSVNNDMIVTDNNGAV KFPQLCKFCDVRFSTCDNQKSCMSNCSITSICEKPQEVCVAVWRKNDENITLETV CHDPKLPYHDFILEDAASPKCIMKFKKKPGETFFMCSCSSDECNDNIIFS (SEQ ID NO:171) |
| Clone N(IgG4) linker (PEAPTD)3-Trap1-127 | EVQLQQSGPELVKPGASMKISCKASGYSFTGYTMNWVKQSHGKNLEWIGLIIPH NGGTSYNQKFKDKATLTVDKSSRTAYMELLSLTSEDSAVYYCASLMTTAPRDS MDYWGQGTSVTVSSASTKGPSVFPLAPCSRSTSESTAALGCLVKDYFPEPVTVS WNSGALTSGVHTFPAVLQSSGLYSLSSVVTVPSSSLGTKTYTCNVDHKPSNTKV DKRVESKYGPPCPPCPAPEFLGGPSVFLFPPKPKDTLMISRTPEVTCVVVDVSQE DPEVQFNWYVDGVEVHNAKTKPREEQFNSTYRVVSVLTVLHQDWLNGKEYKC KVSNKGLPSSIEKTISKAKGQPREPQVYTLPPSQEEMTKNQVSLTCLVKGFYPSDI AVEWESNGQPENNYKTTPPVLDSDGSFFLYSRLTVDKSRWQEGNVFSCSVMHE ALHNHYTQKSLSLSLGAPEAPTDPEAPTDPEAPTDIPPHVQKSVNNDMIVTDNN GAVKFPQLCKFCDVRFSTCDNQKSCMSNCSITSICEKPQEVCVAVWRKNDENIT LETVCHDPKLPYHDFILEDAASPKCIMKEKKKPGETFFMCSCSSDECNDNIIFS (SEQ ID NO:172) |
| Clone B linker (PEAPTDE)2; Trap1-127 | QGQLVQSGAEVKKPGSSVKVSCKTSGFTFSSSYISWVRQAPGQGLEWMGWIYA GTGGTSYNQKFTGRVTITVDTSTSTAYMELSSLRSEDTAVYYCARHEGKYWYF DVWGQGTTVTVSSASTKGPSVFPLAPSSKSTSGGTAALGCLVKDYFPEPVTVSW NSGALTSGVHTFPAVLQSSGLYSLSSVVTVPSSSLGTQTYICNVNHKPSNTKVDK RVEPKSCDKTHTCPPCPAPELLGGPSVFLFPPKPKDTLMISRTPEVTCVVVDVSH EDPEVKFNWYVDGVEVHNAKTKPREEQYNSTYRVVSVLTVLHQDWLNGKEYK CKVSNKALPAPIEKTISKAKGQPREPQVYTLPPSREEMTKNQVSLTCLVKGFYPS |
| | DIAVEWESNGQPENNYKTTPPVLDSDGSFFLYSKLTVDKSRWQQGNVFSCSVM HEALHNHYTQKSLSLSPGAPEAPTDEPEAPTDEIPPHVQKSVNNDMIVTDNNGA VKFPQLCKFCDVRFSTCDNQKSCMSNCSITSICEKPQEVCVAVWRKNDENITLET VCHDPKLPYHDFILEDAASPKCIMKEKKKPGETFFMCSCSSDECNDNIIFS (SEQ ID NO:206) |
| Clone B linker (PEAPTDE)3; Trap1-127 | QGQLVQSGAEVKKPGSSVKVSCKTSGFTFSSSYISWVRQAPGQGLEWMGWIYA GTGGTSYNQKFTGRVTITVDTSTSTAYMELSSLRSEDTAVYYCARHEGKYWYF DVWGQGTTVTVSSASTKGPSVFPLAPSSKSTSGGTAALGCLVKDYFPEPVTVSW NSGALTSGVHTFPAVLQSSGLYSLSSVVTVPSSSLGTQTYICNVNHKPSNTKVDK RVEPKSCDKTHTCPPCPAPELLGGPSVFLFPPKPKDTLMISRTPEVTCVVVDVSH EDPEVKFNWYVDGVEVHNAKTKPREEQYNSTYRVVSVLTVLHQDWLNGKEYK CKVSNKALPAPIEKTISKAKGQPREPQVYTLPPSREEMTKNQVSLTCLVKGFYPS DIAVEWESNGQPENNYKTTPPVLDSDGSFFLYSKLTVDKSRWQQGNVFSCSVM HEALHNHYTQKSLSLSPGAPEAPTDEPEAPTDEPEAPTDEIPPHVQKSVNNDMIV TDNNGAVKFPQLCKFCDVRFSTCDNQKSCMSNCSITSICEKPQEVCVAVWRKN DENITLETVCHDPKLPYHDFILEDAASPKCIMKEKKKPGETFFMCSCSSDECNDN IIFS (SEQ ID NO:207) |
| Clone B; linker (PEAPTDE)4; Trap1-127 | QGQLVQSGAEVKKPGSSVKVSCKTSGFTFSSSYISWVRQAPGQGLEWMGWIYA GTGGTSYNQKFTGRVTITVDTSTSTAYMELSSLRSEDTAVYYCARHEGKYWYF DVWGQGTTVTVSSASTKGPSVFPLAPSSKSTSGGTAALGCLVKDYFPEPVTVSW NSGALTSGVHTFPAVLQSSGLYSLSSVVTVPSSSLGTQTYICNVNHKPSNTKVDK RVEPKSCDKTHTCPPCPAPELLGGPSVFLFPPKPKDTLMISRTPEVTCVVVDVSH EDPEVKFNWYVDGVEVHNAKTKPREEQYNSTYRVVSVLTVLHQDWLNGKEYK CKVSNKALPAPIEKTISKAKGQPREPQVYTLPPSREEMTKNQVSLTCLVKGFYPS DIAVEWESNGQPENNYKTTPPVLDSDGSFFLYSKLTVDKSRWQQGNVFSCSVM HEALHNHYTQKSLSLSPGAPEAPTDEPEAPTDEPEAPTDEPEAPTDEIPPHVQKSV NNDMIVTDNNGAVKFPQLCKFCDVRFSTCDNQKSCMSNCSITSICEKPQEVCVA VWRKNDENITLETVCHDPKLPYHDFILEDAASPKCIMKEKKKPGETFFMCSCSS DECNDNIIFS (SEQ ID NO:208) |
| Clone B; linker (PEAPTDE)3; Trap1-127, K7G | QGQLVQSGAEVKKPGSSVKVSCKTSGFTFSSSYISWVRQAPGQGLEWMGWIYA GTGGTSYNQKFTGRVTITVDTSTSTAYMELSSLRSEDTAVYYCARHEGKYWYF DVWGQGTTVTVSSASTKGPSVFPLAPSSKSTSGGTAALGCLVKDYFPEPVTVSW NSGALTSGVHTFPAVLQSSGLYSLSSVVTVPSSSLGTQTYICNVNHKPSNTKVDK RVEPKSCDKTHTCPPCPAPELLGGPSVFLFPPKPKDTLMISRTPEVTCVVVDVSH EDPEVKFNWYVDGVEVHNAKTKPREEQYNSTYRVVSVLTVLHQDWLNGKEYK CKVSNKALPAPIEKTISKAKGQPREPQVYTLPPSREEMTKNQVSLTCLVKGFYPS DIAVEWESNGQPENNYKTTPPVLDSDGSFFLYSKLTVDKSRWQQGNVFSCSVM HEALHNHYTQKSLSLSPGAPEAPTDEPEAPTDEPEAPTDEIPPHVQGSVNNDMIV TDNNGAVKFPQLCKFCDVRFSTCDNQKSCMSNCSITSICEKPQEVCVAVWRKN DENITLETVCHDPKLPYHDFILEDAASPKCIMKEKKKPGETFFMCSCSSDECNDN IIFS (SEQ ID NO:209) |
| Clone B; linker (PEAPTDE)3; Trap1-127, K7G, N19A: | QGQLVQSGAEVKKPGSSVKVSCKTSGFTFSSSYISWVRQAPGQGLEWMGWIYA GTGGTSYNQKFTGRVTITVDTSTSTAYMELSSLRSEDTAVYYCARHEGKYWYF DVWGQGTTVTVSSASTKGPSVFPLAPSSKSTSGGTAALGCLVKDYFPEPVTVSW NSGALTSGVHTFPAVLQSSGLYSLSSVVTVPSSSLGTQTYICNVNHKPSNTKVDK RVEPKSCDKTHTCPPCPAPELLGGPSVFLFPPKPKDTLMISRTPEVTCVVVDVSH EDPEVKFNWYVDGVEVHNAKTKPREEQYNSTYRVVSVLTVLHQDWLNGKEYK CKVSNKALPAPIEKTISKAKGQPREPQVYTLPPSREEMTKNQVSLTCLVKGFYPS DIAVEWESNGQPENNYKTTPPVLDSDGSFFLYSKLTVDKSRWQQGNVFSCSVM |
| | HEALHNHYTQKSLSLSPGAPEAPTDEPEAPTDEPEAPTDEIPPHVQGSVNNDMIV TDNAGAVKFPQLCKFCDVRFSTCDNQKSCMSNCSITSICEKPQEVCVAVWRKN DENITLETVCHDPKLPYHDFILEDAASPKCIMKEKKKPGETFFMCSCSSDECNDN IIFS (SEQ ID NO:210) |
| Clone B; linker (PEAPTDE)2; Trap1-127, K7G, N19A | QGQLVQSGAEVKKPGSSVKVSCKTSGFTFSSSYISWVRQAPGQGLEWMGWIYA GTGGTSYNQKFTGRVTITVDTSTSTAYMELSSLRSEDTAVYYCARHEGKYWYF DVWGQGTTVTVSSASTKGPSVFPLAPSSKSTSGGTAALGCLVKDYFPEPVTVSW NSGALTSGVHTFPAVLQSSGLYSLSSVVTVPSSSLGTQTYICNVNHKPSNTKVDK RVEPKSCDKTHTCPPCPAPELLGGPSVFLFPPKPKDTLMISRTPEVTCVVVDVSH EDPEVKFNWYVDGVEVHNAKTKPREEQYNSTYRVVSVLTVLHQDWLNGKEYK CKVSNKALPAPIEKTISKAKGQPREPQVYTLPPSREEMTKNQVSLTCLVKGFYPS DIAVEWESNGQPENNYKTTPPVLDSDGSFFLYSKLTVDKSRWQQGNVFSCSVM HEALHNHYTQKSLSLSPGAPEAPTDEPEAPTDEIPPHVQGSVNNDMIVTDNAGA VKFPQLCKFCDVRFSTCDNQKSCMSNCSITSICEKPQEVCVAVWRKNDENITLET VCHDPKLPYHDFILEDAASPKCIMKEKKKPGETFFMCSCSSDECNDNIIFS (SEQ ID NO:211) |
| Clone B; linker (PEAPTDE)2 PEA; Trap1-127, K7G, N19A | QGQLVQSGAEVKKPGSSVKVSCKTSGFTFSSSYISWVRQAPGQGLEWMGWIYA GTGGTSYNQKFTGRVTITVDTSTSTAYMELSSLRSEDTAVYYCARHEGKYWYF DVWGQGTTVTVSSASTKGPSVFPLAPSSKSTSGGTAALGCLVKDYFPEPVTVSW NSGALTSGVHTFPAVLQSSGLYSLSSVVTVPSSSLGTQTYICNVNHKPSNTKVDK RVEPKSCDKTHTCPPCPAPELLGGPSVFLFPPKPKDTLMISRTPEVTCVVVDVSH EDPEVKFNWYVDGVEVHNAKTKPREEQYNSTYRVVSVLTVLHQDWLNGKEYK CKVSNKALPAPIEKTISKAKGQPREPQVYTLPPSREEMTKNQVSLTCLVKGFYPS DIAVEWESNGQPENNYKTTPPVLDSDGSFFLYSKLTVDKSRWQQGNVFSCSVM HEALHNHYTQKSLSLSPGAPEAPTDEPEAPTDEPEAIPPHVQGSVNNDMIVTDNN GAVKFPQLCKFCDVRFSTCDNQKSCMSNCSITSICEKPQEVCVAVWRKNDENIT LETVCHDPKLPYHDFILEDAASPKCIMKEKKKPGETFFMCSCSSDECNDNIIFS (SEQ ID NO:212) |
| Clone B; linker (PEAPTD)3; Trap1-127, K7G, N19A | QGQLVQSGAEVKKPGSSVKVSCKTSGFTFSSSYISWVRQAPGQGLEWMGWIYA GTGGTSYNQKFTGRVTITVDTSTSTAYMELSSLRSEDTAVYYCARHEGKYWYF DVWGQGTTVTVSSASTKGPSVFPLAPSSKSTSGGTAALGCLVKDYFPEPVTVSW NSGALTSGVHTFPAVLQSSGLYSLSSVVTVPSSSLGTQTYICNVNHKPSNTKVDK RVEPKSCDKTHTCPPCPAPELLGGPSVFLFPPKPKDTLMISRTPEVTCVVVDVSH EDPEVKFNWYVDGVEVHNAKTKPREEQYNSTYRVVSVLTVLHQDWLNGKEYK CKVSNKALPAPIEKTISKAKGQPREPQVYTLPPSREEMTKNQVSLTCLVKGFYPS DIAVEWESNGQPENNYKTTPPVLDSDGSFFLYSKLTVDKSRWQQGNVFSCSVM HEALHNHYTQKSLSLSPGAPEAPTDPEAPTDPEAPTDIPPHVQGSVNNDMIVTDN AGAVKFPQLCKFCDVRFSTCDNQKSCMSNCSITSICEKPQEVCVAVWRKNDENI TLETVCHDPKLPYHDFILEDAASPKCIMKEKKKPGETFFMCSCSSDECNDNIIFS (SEQ ID NO:233) |
| Clone B; linker(PEAPT D)2PEA; Trap1-127, K7G, N19A | QGQLVQSGAEVKKPGSSVKVSCKTSGFTFSSSYISWVRQAPGQGLEWMGWIYA GTGGTSYNQKFTGRVTITVDTSTSTAYMELSSLRSEDTAVYYCARHEGKYWYF DVWGQGTTVTVSSASTKGPSVFPLAPSSKSTSGGTAALGCLVKDYFPEPVTVSW NSGALTSGVHTFPAVLQSSGLYSLSSVVTVPSSSLGTQTYICNVNHKPSNTKVDK RVEPKSCDKTHTCPPCPAPELLGGPSVFLFPPKPKDTLMISRTPEVTCVVVDVSH EDPEVKFNWYVDGVEVHNAKTKPREEQYNSTYRVVSVLTVLHQDWLNGKEYK CKVSNKALPAPIEKTISKAKGQPREPQVYTLPPSREEMTKNQVSLTCLVKGFYPS DIAVEWESNGQPENNYKTTPPVLDSDGSFFLYSKLTVDKSRWQQGNVFSCSVM HEALHNHYTQKSLSLSPGAPEAPTDPEAPTDPEAPTDPEAIPPHVQGSVNNDMIV |
| | TDNAGAVKFPQLCKFCDVRFSTCDNQKSCMSNCSITSICEKPQEVCVAVWRKN DENITLETVCHDPKLPYHDFILEDAASPKCIMKEKKKPGETFFMCSCSSDECNDN IIFS (SEQ ID NO:234) |
| Clone B; linker-none; Trap1-127, Q6G, K7G, N19A | QGQLVQSGAEVKKPGSSVKVSCKTSGFTFSSSYISWVRQAPGQGLEWMGWIYA GTGGTSYNQKFTGRVTITVDTSTSTAYMELSSLRSEDTAVYYCARHEGKYWYF DVWGQGTTVTVSSASTKGPSVFPLAPSSKSTSGGTAALGCLVKDYFPEPVTVSW NSGALTSGVHTFPAVLQSSGLYSLSSVVTVPSSSLGTQTYICNVNHKPSNTKVDK RVEPKSCDKTHTCPPCPAPELLGGPSVFLFPPKPKDTLMISRTPEVTCVVVDVSH EDPEVKFNWYVDGVEVHNAKTKPREEQYNSTYRVVSVLTVLHQDWLNGKEYK CKVSNKALPAPIEKTISKAKGQPREPQVYTLPPSREEMTKNQVSLTCLVKGFYPS DIAVEWESNGQPENNYKTTPPVLDSDGSFFLYSKLTVDKSRWQQGNVFSCSVM HEALHNHYTQKSLSLSPGAIPPHVGGSVNNDMIVTDNAGAVKFPQLCKFCDVRF STCDNQKSCMSNCSITSICEKPQEVCVAVWRKNDENITLETVCHDPKLPYHDFIL EDAASPKCIMKFKKKPGETFFMCSCSSDECNDNIIFS (SEQ ID NO:235) |
| Clone B; linker(PEAPT DE)2PEA; Trap1-127, Q6G, K7G, N19A | QGQLVQSGAEVKKPGSSVKVSCKTSGFTFSSSYISWVRQAPGQGLEWMGWIYA GTGGTSYNQKFTGRVTITVDTSTSTAYMELSSLRSEDTAVYYCARHEGKYWYF DVWGQGTTVTVSSASTKGPSVFPLAPSSKSTSGGTAALGCLVKDYFPEPVTVSW NSGALTSGVHTFPAVLQSSGLYSLSSVVTVPSSSLGTQTYICNVNHKPSNTKVDK RVEPKSCDKTHTCPPCPAPELLGGPSVFLFPPKPKDTLMISRTPEVTCVVVDVSH EDPEVKFNWYVDGVEVHNAKTKPREEQYNSTYRVVSVLTVLHQDWLNGKEYK CKVSNKALPAPIEKTISKAKGQPREPQVYTLPPSREEMTKNQVSLTCLVKGFYPS DIAVEWESNGQPENNYKTTPPVLDSDGSFFLYSKLTVDKSRWQQGNVFSCSVM HEALHNHYTQKSLSLSPGAPEAPTDEPEAPTDEPEAIPPHVGGSVNNDMIVTDNA GAVKFPQLCKFCDVRFSTCDNQKSCMSNCSITSICEKPQEVCVAVWRKNDENIT LETVCHDPKLPYHDFILEDAASPKCIMKEKKKPGETFFMCSCSSDECNDNIIFS (SEQ ID NO:236) |
| Clone B; linker(PEAPT DE)3; Trap1-127 | QGQLVQSGAEVKKPGSSVKVSCKTSGFTFSSSYISWVRQAPGQGLEWMGWIYA GTGGTSYNQKFTGRVTITVDTSTSTAYMELSSLRSEDTAVYYCARHEGKYWYF DVWGQGTTVTVSSASTKGPSVFPLAPSSKSTSGGTAALGCLVKDYFPEPVTVSW NSGALTSGVHTFPAVLQSSGLYSLSSVVTVPSSSLGTQTYICNVNHKPSNTKVDK RVEPKSCDKTHTCPPCPAPELLGGPSVFLFPPKPKDTLMISRTPEVTCVVVDVSH EDPEVKFNWYVDGVEVHNAKTKPREEQYNSTYRVVSVLTVLHQDWLNGKEYK CKVSNKALPAPIEKTISKAKGQPREPQVYTLPPSREEMTKNQVSLTCLVKGFYPS DIAVEWESNGQPENNYKTTPPVLDSDGSFFLYSKLTVDKSRWQQGNVFSCSVM HEALHNHYTQKSLSLSPGAPEAPTDEPEAPTDEPEAPTDEIPPHVQKSVNNDMIV TDNNGAVKFPQLCKFCDVRFSTCDNQKSCMSNCSITSICEKPQEVCVAVWRKN DENITLETVCHDPKLPYHDFILEDAASPKCIMKEKKKPGETFFMCSCSSDECNDN IIFS (SEQ ID NO:237) |
| Clone B; linker(PEAPT DE)2; Trap1-127, K7G, N19A | QGQLVQSGAEVKKPGSSVKVSCKTSGFTFSSSYISWVRQAPGQGLEWMGWIYA GTGGTSYNQKFTGRVTITVDTSTSTAYMELSSLRSEDTAVYYCARHEGKYWYF DVWGQGTTVTVSSASTKGPSVFPLAPSSKSTSGGTAALGCLVKDYFPEPVTVSW NSGALTSGVHTFPAVLQSSGLYSLSSVVTVPSSSLGTQTYICNVNHKPSNTKVDK RVEPKSCDKTHTCPPCPAPELLGGPSVFLFPPKPKDTLMISRTPEVTCVVVDVSH EDPEVKFNWYVDGVEVHNAKTKPREEQYNSTYRVVSVLTVLHQDWLNGKEYK CKVSNKALPAPIEKTISKAKGQPREPQVYTLPPSREEMTKNQVSLTCLVKGFYPS DIAVEWESNGQPENNYKTTPPVLDSDGSFFLYSKLTVDKSRWQQGNVFSCSVM HEALHNHYTQKSLSLSPGAPEAPTDEPEAPTDEIPPHVQGSVNNDMIVTDNAGA VKFPQLCKFCDVRFSTCDNQKSCMSNCSITSICEKPQEVCVAVWRKNDENITLET |
| | VCHDPKLPYHDFILEDAASPKCIMKEKKKPGETFFMCSCSSDECNDNIIFS (SEQ ID NO:238) |
| Clone B; linker(PEAPT DE)2; Trap1-127,Q6G, K7G, N19A | QGQLVQSGAEVKKPGSSVKVSCKTSGFTFSSSYISWVRQAPGQGLEWMGWIYA GTGGTSYNQKFTGRVTITVDTSTSTAYMELSSLRSEDTAVYYCARHEGKYWYF DVWGQGTTVTVSSASTKGPSVFPLAPSSKSTSGGTAALGCLVKDYFPEPVTVSW NSGALTSGVHTFPAVLQSSGLYSLSSVVTVPSSSLGTQTYICNVNHKPSNTKVDK RVEPKSCDKTHTCPPCPAPELLGGPSVFLFPPKPKDTLMISRTPEVTCVVVDVSH EDPEVKFNWYVDGVEVHNAKTKPREEQYNSTYRVVSVLTVLHQDWLNGKEYK CKVSNKALPAPIEKTISKAKGQPREPQVYTLPPSREEMTKNQVSLTCLVKGFYPS DIAVEWESNGQPENNYKTTPPVLDSDGSFFLYSKLTVDKSRWQQGNVFSCSVM HEALHNHYTQKSLSLSPGAPEAPTDEPEAPTDEIPPHVGGSVNNDMIVTDNAGA VKFPQLCKFCDVRFSTCDNQKSCMSNCSITSICEKPQEVCVAVWRKNDENITLET VCHDPKLPYHDFILEDAASPKCIMKEKKKPGETFFMCSCSSDECNDNIIFS (SEQ ID NO:239) |
| Clone B; linker(PEAPT DE)3; Trap1-127,Q6G, K7G, N19A | QGQLVQSGAEVKKPGSSVKVSCKTSGFTFSSSYISWVRQAPGQGLEWMGWIYA GTGGTSYNQKFTGRVTITVDTSTSTAYMELSSLRSEDTAVYYCARHEGKYWYF DVWGQGTTVTVSSASTKGPSVFPLAPSSKSTSGGTAALGCLVKDYFPEPVTVSW NSGALTSGVHTFPAVLQSSGLYSLSSVVTVPSSSLGTQTYICNVNHKPSNTKVDK RVEPKSCDKTHTCPPCPAPELLGGPSVFLFPPKPKDTLMISRTPEVTCVVVDVSH EDPEVKFNWYVDGVEVHNAKTKPREEQYNSTYRVVSVLTVLHQDWLNGKEYK CKVSNKALPAPIEKTISKAKGQPREPQVYTLPPSREEMTKNQVSLTCLVKGFYPS DIAVEWESNGQPENNYKTTPPVLDSDGSFFLYSKLTVDKSRWQQGNVFSCSVM HEALHNHYTQKSLSLSPGAPEAPTDEPEAPTDEPEAPTDEIPPHVGGSVNNDMIV TDNAGAVKFPQLCKFCDVRFSTCDNQKSCMSNCSITSICEKPQEVCVAVWRKN DENITLETVCHDPKLPYHDFILEDAASPKCIMKEKKKPGETFFMCSCSSDECNDN IIFS (SEQ ID NO:240) |
| Clone B | EIVMTQSPATLSVSPGERATLSCSASSSVSYVHWYQQKPGQAPRPWIYDTSNLAS GFPARFSGSGSGTEYTLTISSLQSEDAAVYYCHQRSSYPWTFGGGTKVEIKRTVA APSVFIFPPSDEQLKSGTASVVCLLNNFYPREAKVQWKVDNALQSGNSQESVTE QDSKDSTYSLSSTLTLSKADYEKHKVYACEVTHQGLSSPVTKSFNRGEC (SEQ ID NO:162) |
| Clone D | DIQMTQSPSSLSASVGDRVTITCRASQDISNYLNWYQQKPGKAPKLLIYYTSKLH SGVPSRFSGSGSGTDFTLTISSLQPEDIATYYCQQGDALPWTFGGGTKVEIKRTV AAPSVFIFPPSDEQLKSGTASVVCLLNNFYPREAKVQWKVDNALQSGNSQESVT EQDSKDSTYSLSSTLTLSKADYEKHKVYACEVTHQGLSSPVTKSFNRGEC (SEQ ID NO:163) |
| Clone C | DIQMTQSPSSLSASVGDRVTITCQASQDVSNAVAWYQQKPGKAPKLLIYSASNL YTGVPSRFSGSGSGTDFTFTISSLQPEDIATYYCQQHDSTPLTFGQGTKLELKRTV AAPSVFIFPPSDEQLKSGTASVVCLLNNFYPREAKVQWKVDNALQSGNSQESVT EQDSKDSTYSLSSTLTLSKADYEKHKVYACEVTHQGLSSPVTKSFNRGEC (SEQ ID NO:164) |
| Clone N | DIQMTQTTSSLSASLGDRVTISCRASQDISNYLNWYQQKPDGTVKLLIYYTSRLH SGVPSRFSGSGSGTDYSLTISNLDQEDIATYFCQQGATLPWTFGGGTKLEIKRTV AAPSVFIFPPSDEQLKSGTASVVCLLNNFYPREAKVQWKVDNALQSGNSQESVT |
| | EQDSKDSTYSLSSTLTLSKADYEKHKVYACEVTHQGLSSPVTKSFNRGEC (SEQ ID NO:165) |

In some embodiments, provided herein are fusion proteins comprising a heavy chain, wherein the heavy chain has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:155. In some embodiments, the heavy chain of the fusion protein has at least 85% sequence identity to SEQ ID NO: 155. In some embodiments, the heavy chain of the fusion protein has at least 90% sequence identity to SEQ ID NO:155. In some embodiments, the heavy chain of the fusion protein has at least 95% sequence identity to SEQ ID NO:155. In some embodiments, the heavy chain of the fusion protein has at least 98% sequence identity to SEQ ID NO: 155. In some embodiments, the heavy chain of the fusion protein has the amino acid sequence of SEQ ID NO:155.

In some embodiments, provided herein are fusion proteins comprising a heavy chain, wherein the heavy chain has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:156. In some embodiments, the heavy chain of the fusion protein has at least 85% sequence identity to SEQ ID NO:156. In some embodiments, the heavy chain of the fusion protein has at least 90% sequence identity to SEQ ID NO: 156. In some embodiments, the heavy chain of the fusion protein has at least 95% sequence identity to SEQ ID NO:156. In some embodiments, the heavy chain of the fusion protein has at least 98% sequence identity to SEQ ID NO:156. In some embodiments, the heavy chain of the fusion protein has the amino acid sequence of SEQ ID NO:156.

In some embodiments, provided herein are fusion proteins comprising a heavy chain, wherein the heavy chain has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:157. In some embodiments, the heavy chain of the fusion protein has at least 85% sequence identity to SEQ ID NO:157. In some embodiments, the heavy chain of the fusion protein has at least 90% sequence identity to SEQ ID NO:157. In some embodiments, the heavy chain of the fusion protein has at least 95% sequence identity to SEQ ID NO:157. In some embodiments, the heavy chain of the fusion protein has at least 98% sequence identity to SEQ ID NO:157. In some embodiments, the heavy chain of the fusion protein has the amino acid sequence of SEQ ID NO:157.

In some embodiments, provided herein are fusion proteins comprising a heavy chain, wherein the heavy chain has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:158. In some embodiments, the heavy chain of the fusion protein has at least 85% sequence identity to SEQ ID NO:158. In some embodiments, the heavy chain of the fusion protein has at least 90% sequence identity to SEQ ID NO:158. In some embodiments, the heavy chain of the fusion protein has at least 95% sequence identity to SEQ ID NO:158. In some embodiments, the heavy chain of the fusion protein has at least 98% sequence identity to SEQ ID NO:158. In some embodiments, the heavy chain of the fusion protein has the amino acid sequence of SEQ ID NO:158.

In some embodiments, provided herein are fusion proteins comprising a heavy chain, wherein the heavy chain has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:159. In some embodiments, the heavy chain of the fusion protein has at least 85% sequence identity to SEQ ID NO:159. In some embodiments, the heavy chain of the fusion protein has at least 90% sequence identity to SEQ ID NO:159. In some embodiments, the heavy chain of the fusion protein has at least 95% sequence identity to SEQ ID NO:159. In some embodiments, the heavy chain of the fusion protein has at least 98% sequence identity to SEQ ID NO:159. In some embodiments, the heavy chain of the fusion protein has the amino acid sequence of SEQ ID NO:159.

In some embodiments, provided herein are fusion proteins comprising a heavy chain, wherein the heavy chain has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:160. In some embodiments, the heavy chain of the fusion protein has at least 85% sequence identity to SEQ ID NO:160. In some embodiments, the heavy chain of the fusion protein has at least 90% sequence identity to SEQ ID NO:160. In some embodiments, the heavy chain of the fusion protein has at least 95% sequence identity to SEQ ID NO:160. In some embodiments, the heavy chain of the fusion protein has at least 98% sequence identity to SEQ ID NO:160. In some embodiments, the heavy chain of the fusion protein has the amino acid sequence of SEQ ID NO:160.

In some embodiments, provided herein are fusion proteins comprising a heavy chain, wherein the heavy chain has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:161. In some embodiments, the heavy chain of the fusion protein has at least 85% sequence identity to SEQ ID NO:161. In some embodiments, the heavy chain of the fusion protein has at least 90% sequence identity to SEQ ID NO:161. In some embodiments, the heavy chain of the fusion protein has at least 95% sequence identity to SEQ ID NO:161. In some embodiments, the heavy chain of the fusion protein has at least 98% sequence identity to SEQ ID NO:161. In some embodiments, the heavy chain of the fusion protein has the amino acid sequence of SEQ ID NO:161.

In some embodiments, provided herein are fusion proteins comprising a heavy chain, wherein the heavy chain has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:166. In some embodiments, the heavy chain of the fusion protein has at least 85% sequence identity to SEQ ID NO:166. In some embodiments, the heavy chain of the fusion protein has at least 90% sequence identity to SEQ ID NO:166. In some embodiments, the heavy chain of the fusion protein has at least 95% sequence identity to SEQ ID NO:166. In some embodiments, the heavy chain of the fusion protein has at least 98% sequence identity to SEQ ID NO: 166. In some embodiments, the heavy chain of the fusion protein has the amino acid sequence of SEQ ID NO:166.

In some embodiments, provided herein are fusion proteins comprising a heavy chain, wherein the heavy chain has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 167. In some embodiments, the heavy chain of the fusion protein has at least 85% sequence identity to SEQ ID NO: 167. In some embodiments, the heavy chain of the fusion protein has at least 90% sequence identity to SEQ ID NO: 167. In some embodiments, the heavy chain of the fusion protein has at least 95% sequence identity to SEQ ID NO: 167. In some embodiments, the heavy chain of the fusion protein has at least 98% sequence identity to SEQ ID NO: 167. In some embodiments, the heavy chain of the fusion protein has the amino acid sequence of SEQ ID NO: 167.

In some embodiments, provided herein are fusion proteins comprising a heavy chain, wherein the heavy chain has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:168. In some embodiments, the heavy chain of the fusion protein has at least 85% sequence identity to SEQ ID NO:168. In some embodiments, the heavy chain of the fusion protein has at least 90% sequence identity to SEQ ID NO:168. In some embodiments, the heavy chain of the fusion protein has at least 95% sequence identity to SEQ ID NO: 168. In some embodiments, the heavy chain of the fusion protein has at least 98% sequence identity to SEQ ID NO: 168. In some embodiments, the heavy chain of the fusion protein has the amino acid sequence of SEQ ID NO:168.

In some embodiments, provided herein are fusion proteins comprising a heavy chain, wherein the heavy chain has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:169. In some embodiments, the heavy chain of the fusion protein has at least 85% sequence identity to SEQ ID NO:169. In some embodiments, the heavy chain of the fusion protein has at least 90% sequence identity to SEQ ID NO:169. In some embodiments, the heavy chain of the fusion protein has at least 95% sequence identity to SEQ ID NO: 169. In some embodiments, the heavy chain of the fusion protein has at least 98% sequence identity to SEQ ID NO:169. In some embodiments, the heavy chain of the fusion protein has the amino acid sequence of SEQ ID NO:169.

In some embodiments, provided herein are fusion proteins comprising a heavy chain, wherein the heavy chain has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:170. In some embodiments, the heavy chain of the fusion protein has at least 85% sequence identity to SEQ ID NO:170. In some embodiments, the heavy chain of the fusion protein has at least 90% sequence identity to SEQ ID NO:170. In some embodiments, the heavy chain of the fusion protein has at least 95% sequence identity to SEQ ID NO: 170. In some embodiments, the heavy chain of the fusion protein has at least 98% sequence identity to SEQ ID NO:170. In some embodiments, the heavy chain of the fusion protein has the amino acid sequence of SEQ ID NO:170.

In some embodiments, provided herein are fusion proteins comprising a heavy chain, wherein the heavy chain has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:171. In some embodiments, the heavy chain of the fusion protein has at least 85% sequence identity to SEQ ID NO:171. In some embodiments, the heavy chain of the fusion protein has at least 90% sequence identity to SEQ ID NO:171. In some embodiments, the heavy chain of the fusion protein has at least 95% sequence identity to SEQ ID NO: 171. In some embodiments, the heavy chain of the fusion protein has at least 98% sequence identity to SEQ ID NO:171. In some embodiments, the heavy chain of the fusion protein has the amino acid sequence of SEQ ID NO:171.

In some embodiments, provided herein are fusion proteins comprising a heavy chain, wherein the heavy chain has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:172. In some embodiments, the heavy chain of the fusion protein has at least 85% sequence identity to SEQ ID NO:172. In some embodiments, the heavy chain of the fusion protein has at least 90% sequence identity to SEQ ID NO:172. In some embodiments, the heavy chain of the fusion protein has at least 95% sequence identity to SEQ ID NO:172. In some embodiments, the heavy chain of the fusion protein has at least 98% sequence identity to SEQ ID NO:172. In some embodiments, the heavy chain of the fusion protein has the amino acid sequence of SEQ ID NO:172.

In some embodiments, provided herein are fusion proteins comprising a heavy chain, wherein the heavy chain has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:206. In some embodiments, the heavy chain of the fusion protein has at least 85% sequence identity to SEQ ID NO:206. In some embodiments, the heavy chain of the fusion protein has at least 90% sequence identity to SEQ ID NO:206. In some embodiments, the heavy chain of the fusion protein has at least 95% sequence identity to SEQ ID NO:206. In some embodiments, the heavy chain of the fusion protein has at least 98% sequence identity to SEQ ID NO:206. In some embodiments, the heavy chain of the fusion protein has the amino acid sequence of SEQ ID NO:206.

In some embodiments, provided herein are fusion proteins comprising a heavy chain, wherein the heavy chain has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:207. In some embodiments, the heavy chain of the fusion protein has at least 85% sequence identity to SEQ ID NO:207. In some embodiments, the heavy chain of the fusion protein has at least 90% sequence identity to SEQ ID NO:207. In some embodiments, the heavy chain of the fusion protein has at least 95% sequence identity to SEQ ID NO:207. In some embodiments, the heavy chain of the fusion protein has at least 98% sequence identity to SEQ ID NO:207. In some embodiments, the heavy chain of the fusion protein has the amino acid sequence of SEQ ID NO:207.

In some embodiments, provided herein are fusion proteins comprising a heavy chain, wherein the heavy chain has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:208. In some embodiments, the heavy chain of the fusion protein has at least 85% sequence identity to SEQ ID NO:208. In some embodiments, the heavy chain of the fusion protein has at least 90% sequence identity to SEQ ID NO:208. In some embodiments, the heavy chain of the fusion protein has at least 95% sequence identity to SEQ ID NO:208. In some embodiments, the heavy chain of the fusion protein has at least 98% sequence identity to SEQ ID NO:208. In some embodiments, the heavy chain of the fusion protein has the amino acid sequence of SEQ ID NO:208.

In some embodiments, provided herein are fusion proteins comprising a heavy chain, wherein the heavy chain has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:209. In some embodiments, the heavy chain of the fusion protein has at least 85% sequence identity to SEQ ID NO:209. In some embodiments, the heavy chain of the fusion protein has at least 90% sequence identity to SEQ ID NO:209. In some embodiments, the heavy chain of the fusion protein has at least 95% sequence identity to SEQ ID NO:209. In some embodiments, the heavy chain of the fusion protein has at least 98% sequence identity to SEQ ID NO:209. In some embodiments, the heavy chain of the fusion protein has the amino acid sequence of SEQ ID NO:209.

In some embodiments, provided herein are fusion proteins comprising a heavy chain, wherein the heavy chain has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:210. In some embodiments, the heavy chain of the fusion protein has at least 85% sequence identity to SEQ ID NO:210. In some embodiments, the heavy chain of the fusion protein has at least 90% sequence identity to SEQ ID NO:210. In some embodiments, the heavy chain of the fusion protein has at least 95% sequence identity to SEQ ID NO:210. In some embodiments, the heavy chain of the fusion protein has at least 98% sequence identity to SEQ ID NO:210. In some embodiments, the heavy chain of the fusion protein has the amino acid sequence of SEQ ID NO:210.

In some embodiments, provided herein are fusion proteins comprising a heavy chain, wherein the heavy chain has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:211. In some embodiments, the heavy chain of the fusion protein has at least 85% sequence identity to SEQ ID NO:211. In some embodiments, the heavy chain of the fusion protein has at least 90% sequence identity to SEQ ID NO:211. In some embodiments, the heavy chain of the fusion protein has at least 95% sequence identity to SEQ ID NO:211. In some embodiments, the heavy chain of the fusion protein has at least 98% sequence identity to SEQ ID NO:211. In some embodiments, the heavy chain of the fusion protein has the amino acid sequence of SEQ ID NO:211.

In some embodiments, provided herein are fusion proteins comprising a heavy chain, wherein the heavy chain has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:212. In some embodiments, the heavy chain of the fusion protein has at least 85% sequence identity to SEQ ID NO:212. In some embodiments, the heavy chain of the fusion protein has at least 90% sequence identity to SEQ ID NO:212. In some embodiments, the heavy chain of the fusion protein has at least 95% sequence identity to SEQ ID NO:212. In some embodiments, the heavy chain of the fusion protein has at least 98% sequence identity to SEQ ID NO:212. In some embodiments, the heavy chain of the fusion protein has the amino acid sequence of SEQ ID NO:212.

In some embodiments, provided herein are fusion proteins comprising a heavy chain, wherein the heavy chain has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:233. In some embodiments, the heavy chain of the fusion protein has at least 85% sequence identity to SEQ ID NO:233. In some embodiments, the heavy chain of the fusion protein has at least 90% sequence identity to SEQ ID NO:233. In some embodiments, the heavy chain of the fusion protein has at least 95% sequence identity to SEQ ID NO:233. In some embodiments, the heavy chain of the fusion protein has at least 98% sequence identity to SEQ ID NO:233. In some embodiments, the heavy chain of the fusion protein has the amino acid sequence of SEQ ID NO:233.

In some embodiments, provided herein are fusion proteins comprising a heavy chain, wherein the heavy chain has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:234. In some embodiments, the heavy chain of the fusion protein has at least 85% sequence identity to SEQ ID NO:234. In some embodiments, the heavy chain of the fusion protein has at least 90% sequence identity to SEQ ID NO:234. In some embodiments, the heavy chain of the fusion protein has at least 95% sequence identity to SEQ ID NO:234. In some embodiments, the heavy chain of the fusion protein has at least 98% sequence identity to SEQ ID NO:234. In some embodiments, the heavy chain of the fusion protein has the amino acid sequence of SEQ ID NO:234.

In some embodiments, provided herein are fusion proteins comprising a heavy chain, wherein the heavy chain has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:235. In some embodiments, the heavy chain of the fusion protein has at least 85% sequence identity to SEQ ID NO:235. In some embodiments, the heavy chain of the fusion protein has at least 90% sequence identity to SEQ ID NO:235. In some embodiments, the heavy chain of the fusion protein has at least 95% sequence identity to SEQ ID NO:235. In some embodiments, the heavy chain of the fusion protein has at least 98% sequence identity to SEQ ID NO:235. In some embodiments, the heavy chain of the fusion protein has the amino acid sequence of SEQ ID NO:235.

In some embodiments, provided herein are fusion proteins comprising a heavy chain, wherein the heavy chain has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:236. In some embodiments, the heavy chain of the fusion protein has at least 85% sequence identity to SEQ ID NO:236. In some embodiments, the heavy chain of the fusion protein has at least 90% sequence identity to SEQ ID NO:236. In some embodiments, the heavy chain of the fusion protein has at least 95% sequence identity to SEQ ID NO:236. In some embodiments, the heavy chain of the fusion protein has at least 98% sequence identity to SEQ ID NO:236. In some embodiments, the heavy chain of the fusion protein has the amino acid sequence of SEQ ID NO:236.

In some embodiments, provided herein are fusion proteins comprising a heavy chain, wherein the heavy chain has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:237. In some embodiments, the heavy chain of the fusion protein has at least 85% sequence identity to SEQ ID NO:237. In some embodiments, the heavy chain of the fusion protein has at least 90% sequence identity to SEQ ID NO:237. In some embodiments, the heavy chain of the fusion protein has at least 95% sequence identity to SEQ ID NO:237. In some embodiments, the heavy chain of the fusion protein has at least 98% sequence identity to SEQ ID NO:237. In some embodiments, the heavy chain of the fusion protein has the amino acid sequence of SEQ ID NO:237.

In some embodiments, provided herein are fusion proteins comprising a heavy chain, wherein the heavy chain has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:238. In some embodiments, the heavy chain of the fusion protein has at least 85% sequence identity to SEQ ID NO:238. In some embodiments, the heavy chain of the fusion protein has at least 90% sequence identity to SEQ ID NO:238. In some embodiments, the heavy chain of the fusion protein has at least 95% sequence identity to SEQ ID NO:238. In some embodiments, the heavy chain of the fusion protein has at least 98% sequence identity to SEQ ID NO:238. In some embodiments, the heavy chain of the fusion protein has the amino acid sequence of SEQ ID NO:238.

In some embodiments, provided herein are fusion proteins comprising a heavy chain, wherein the heavy chain has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:239. In some embodiments, the heavy chain of the fusion protein has at least 85% sequence identity to SEQ ID NO:239. In some embodiments, the heavy chain of the fusion protein has at least 90% sequence identity to SEQ ID NO:239. In some embodiments, the heavy chain of the fusion protein has at least 95% sequence identity to SEQ ID NO:239. In some embodiments, the heavy chain of the fusion protein has at least 98% sequence identity to SEQ ID NO:239. In some embodiments, the heavy chain of the fusion protein has the amino acid sequence of SEQ ID NO:239.

In some embodiments, provided herein are fusion proteins comprising a heavy chain, wherein the heavy chain has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:240. In some embodiments, the heavy chain of the fusion protein has at least 85% sequence identity to SEQ ID NO:240. In some embodiments, the heavy chain of the fusion protein has at least 90% sequence identity to SEQ ID NO:240. In some embodiments, the heavy chain of the fusion protein has at least 95% sequence identity to SEQ ID NO:240. In some embodiments, the heavy chain of the fusion protein has at least 98% sequence identity to SEQ ID NO:240. In some embodiments, the heavy chain of the fusion protein has the amino acid sequence of SEQ ID NO:240.

In some embodiments, provided herein are fusion proteins comprising a light chain, wherein the light chain has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to an amino acid sequence selected from the group consisting of SEQ ID NOs:162-165. In some embodiments, provided herein are fusion proteins comprising a light chain, wherein the light chain has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:162. In some embodiments, the light chain of the fusion protein has at least 85% sequence identity to SEQ ID NO:162. In some embodiments, the light chain of the fusion protein has at least 90% sequence identity to SEQ ID NO:162. In some embodiments, the light chain of the fusion protein has at least 95% sequence identity to SEQ ID NO:162. In some embodiments, the light chain of the fusion protein has at least 98% sequence identity to SEQ ID NO:162. In some embodiments, the light chain of the fusion protein has the amino acid sequence of SEQ ID NO:162.

In some embodiments, provided herein are fusion proteins comprising a light chain, wherein the light chain has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:163. In some embodiments, the light chain of the fusion protein has at least 85% sequence identity to SEQ ID NO:163. In some embodiments, the light chain of the fusion protein has at least 90% sequence identity to SEQ ID NO:163. In some embodiments, the light chain of the fusion protein has at least 95% sequence identity to SEQ ID NO:163. In some embodiments, the light chain of the fusion protein has at least 98% sequence identity to SEQ ID NO:163. In some embodiments, the light chain of the fusion protein has the amino acid sequence of SEQ ID NO:163.

In some embodiments, provided herein are fusion proteins comprising a light chain, wherein the light chain has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:164. In some embodiments, the light chain of the fusion protein has at least 85% sequence identity to SEQ ID NO:164. In some embodiments, the light chain of the fusion protein has at least 90% sequence identity to SEQ ID NO:164. In some embodiments, the light chain of the fusion protein has at least 95% sequence identity to SEQ ID NO:164. In some embodiments, the light chain of the fusion protein has at least 98% sequence identity to SEQ ID NO:164. In some embodiments, the light chain of the fusion protein has the amino acid sequence of SEQ ID NO:164.

In some embodiments, provided herein are fusion proteins comprising a light chain, wherein the light chain has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:165. In some embodiments, the light chain of the fusion protein has at least 85% sequence identity to SEQ ID NO:165. In some embodiments, the light chain of the fusion protein has at least 90% sequence identity to SEQ ID NO:165. In some embodiments, the light chain of the fusion protein has at least 95% sequence identity to SEQ ID NO:165. In some embodiments, the light chain of the fusion protein has at least 98% sequence identity to SEQ ID NO:165. In some embodiments, the light chain of the fusion protein has the amino acid sequence of SEQ ID NO:165.

In some embodiments, provided herein are fusion proteins comprising a heavy chain and a light chain, wherein the heavy chain has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to an amino acid sequence selected from the group consisting of SEQ ID NOs:155-161, 166-172, 206-212 and 233-240; and the light chain has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to an amino acid sequence selected from the group consisting of SEQ ID NOs: 162-165.

In some embodiments, fusion proteins provided herein comprise a heavy chain and a light chain, wherein the heavy chain has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to an amino acid sequence selected from the group consisting of SEQ ID NOs: 155-158, 166-169, 206-212 and 233-240; and the light chain has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:162. In some embodiments, fusion proteins provided herein comprise a heavy chain and a light chain, wherein the heavy chain has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:159 or 170; and the light chain has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:163. In some embodiments, fusion proteins provided herein comprise a heavy chain and a light chain, wherein the heavy chain has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:160 or 171; and the light chain has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:164. In some embodiments, fusion proteins provided herein comprise a heavy chain and a light chain, wherein the heavy chain has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:161 or 172; and the light chain has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 165.

In some embodiments, the fusion protein provided herein comprises a heavy chain and a light chain having the amino acid sequences of SEQ ID NOs: 155 and 162, respectively. In some embodiments, the fusion protein comprises a heavy chain and a light chain having the amino acid sequences of SEQ ID NOs: 155 and 163, respectively. In some embodiments, the fusion protein comprises a heavy chain and a light chain having the amino acid sequences of SEQ ID NOs: 155 and 164, respectively. In some embodiments, the fusion protein comprises a heavy chain and a light chain having the amino acid sequences of SEQ ID NOs: 155 and 165, respectively.

In some embodiments, the fusion protein provided herein comprises a heavy chain and a light chain having the amino acid sequences of SEQ ID NOs: 156 and 162, respectively. In some embodiments, the fusion protein comprises a heavy chain and a light chain having the amino acid sequences of SEQ ID NOs: 156 and 163, respectively. In some embodiments, the fusion protein comprises a heavy chain and a light chain having the amino acid sequences of SEQ ID NOs:156 and 164, respectively. In some embodiments, the fusion protein comprises a heavy chain and a light chain having the amino acid sequences of SEQ ID NOs: 156 and 165, respectively.

In some embodiments, the fusion protein provided herein comprises a heavy chain and a light chain having the amino acid sequences of SEQ ID NOs: 157 and 162, respectively. In some embodiments, the fusion protein comprises a heavy chain and a light chain having the amino acid sequences of SEQ ID NOs:157 and 163, respectively. In some embodiments, the fusion protein comprises a heavy chain and a light chain having the amino acid sequences of SEQ ID NOs: 157 and 164, respectively. In some embodiments, the fusion protein comprises a heavy chain and a light chain having the amino acid sequences of SEQ ID NOs: 157 and 165, respectively.

In some embodiments, the fusion protein provided herein comprises a heavy chain and a light chain having the amino acid sequences of SEQ ID NOs: 158 and 162, respectively. In some embodiments, the fusion protein comprises a heavy chain and a light chain having the amino acid sequences of SEQ ID NOs: 158 and 163, respectively. In some embodiments, the fusion protein comprises a heavy chain and a light chain having the amino acid sequences of SEQ ID NOs: 158 and 164, respectively. In some embodiments, the fusion protein comprises a heavy chain and a light chain having the amino acid sequences of SEQ ID NOs: 158 and 165, respectively.

In some embodiments, the fusion protein provided herein comprises a heavy chain and a light chain having the amino acid sequences of SEQ ID NOs: 159 and 162, respectively. In some embodiments, the fusion protein comprises a heavy chain and a light chain having the amino acid sequences of SEQ ID NOs: 159 and 163, respectively. In some embodiments, the fusion protein comprises a heavy chain and a light chain having the amino acid sequences of SEQ ID NOs: 159 and 164, respectively. In some embodiments, the fusion protein comprises a heavy chain and a light chain having the amino acid sequences of SEQ ID NOs: 159 and 165, respectively.

In some embodiments, the fusion protein provided herein comprises a heavy chain and a light chain having the amino acid sequences of SEQ ID NOs: 160 and 162, respectively. In some embodiments, the fusion protein comprises a heavy chain and a light chain having the amino acid sequences of SEQ ID NOs: 160 and 163, respectively. In some embodiments, the fusion protein comprises a heavy chain and a light chain having the amino acid sequences of SEQ ID NOs: 160 and 164, respectively. In some embodiments, the fusion protein comprises a heavy chain and a light chain having the amino acid sequences of SEQ ID NOs: 160 and 165, respectively.

In some embodiments, the fusion protein provided herein comprises a heavy chain and a light chain having the amino acid sequences of SEQ ID NOs: 161 and 162, respectively. In some embodiments, the fusion protein comprises a heavy chain and a light chain having the amino acid sequences of SEQ ID NOs: 161 and 163, respectively. In some embodiments, the fusion protein comprises a heavy chain and a light chain having the amino acid sequences of SEQ ID NOs: 161 and 164, respectively. In some embodiments, the fusion protein comprises a heavy chain and a light chain having the amino acid sequences of SEQ ID NOs: 161 and 165, respectively.

In some embodiments, the fusion protein provided herein comprises a heavy chain and a light chain having the amino acid sequences of SEQ ID NOs: 166 and 162, respectively. In some embodiments, the fusion protein comprises a heavy chain and a light chain having the amino acid sequences of SEQ ID NOs: 166 and 163, respectively. In some embodiments, the fusion protein comprises a heavy chain and a light chain having the amino acid sequences of SEQ ID NOs: 166 and 164, respectively. In some embodiments, the fusion protein comprises a heavy chain and a light chain having the amino acid sequences of SEQ ID NOs: 166 and 165, respectively.

In some embodiments, the fusion protein provided herein comprises a heavy chain and a light chain having the amino acid sequences of SEQ ID NOs: 167 and 162, respectively. In some embodiments, the fusion protein comprises a heavy chain and a light chain having the amino acid sequences of SEQ ID NOs:167 and 163, respectively. In some embodiments, the fusion protein comprises a heavy chain and a light chain having the amino acid sequences of SEQ ID NOs: 167 and 164, respectively. In some embodiments, the fusion protein comprises a heavy chain and a light chain having the amino acid sequences of SEQ ID NOs:167 and 165, respectively.

In some embodiments, the fusion protein provided herein comprises a heavy chain and a light chain having the amino acid sequences of SEQ ID NOs: 168 and 162, respectively. In some embodiments, the fusion protein comprises a heavy chain and a light chain having the amino acid sequences of SEQ ID NOs:168 and 163, respectively. In some embodiments, the fusion protein comprises a heavy chain and a light chain having the amino acid sequences of SEQ ID NOs: 168 and 164, respectively. In some embodiments, the fusion protein comprises a heavy chain and a light chain having the amino acid sequences of SEQ ID NOs: 168 and 165, respectively.

In some embodiments, the fusion protein provided herein comprises a heavy chain and a light chain having the amino acid sequences of SEQ ID NOs: 169 and 162, respectively. In some embodiments, the fusion protein comprises a heavy chain and a light chain having the amino acid sequences of SEQ ID NOs:169 and 163, respectively. In some embodiments, the fusion protein comprises a heavy chain and a light chain having the amino acid sequences of SEQ ID NOs: 169 and 164, respectively. In some embodiments, the fusion protein comprises a heavy chain and a light chain having the amino acid sequences of SEQ ID NOs: 169 and 165, respectively.

In some embodiments, the fusion protein provided herein comprises a heavy chain and a light chain having the amino acid sequences of SEQ ID NOs: 170 and 162, respectively. In some embodiments, the fusion protein comprises a heavy chain and a light chain having the amino acid sequences of SEQ ID NOs: 170 and 163, respectively. In some embodiments, the fusion protein comprises a heavy chain and a light chain having the amino acid sequences of SEQ ID NOs: 170 and 164, respectively. In some embodiments, the fusion protein comprises a heavy chain and a light chain having the amino acid sequences of SEQ ID NOs: 170 and 165, respectively.

In some embodiments, the fusion protein provided herein comprises a heavy chain and a light chain having the amino acid sequences of SEQ ID NOs: 171 and 162, respectively. In some embodiments, the fusion protein comprises a heavy chain and a light chain having the amino acid sequences of SEQ ID NOs: 171 and 163, respectively. In some embodiments, the fusion protein comprises a heavy chain and a light chain having the amino acid sequences of SEQ ID NOs: 171 and 164, respectively. In some embodiments, the fusion protein comprises a heavy chain and a light chain having the amino acid sequences of SEQ ID NOs:171 and 165, respectively.

In some embodiments, the fusion protein provided herein comprises a heavy chain and a light chain having the amino acid sequences of SEQ ID NOs: 172 and 162, respectively. In some embodiments, the fusion protein comprises a heavy chain and a light chain having the amino acid sequences of SEQ ID NOs:172 and 163, respectively. In some embodiments, the fusion protein comprises a heavy chain and a light chain having the amino acid sequences of SEQ ID NOs: 172 and 164, respectively. In some embodiments, the fusion protein comprises a heavy chain and a light chain having the amino acid sequences of SEQ ID NOs: 172 and 165, respectively.

In some embodiments, the fusion protein provided herein comprises a heavy chain and a light chain having the amino acid sequences of SEQ ID NOs:206 and 162, respectively. In some embodiments, the fusion protein comprises a heavy chain and a light chain having the amino acid sequences of SEQ ID NOs:206 and 163, respectively. In some embodiments, the fusion protein comprises a heavy chain and a light chain having the amino acid sequences of SEQ ID NOs:206 and 164, respectively. In some embodiments, the fusion protein comprises a heavy chain and a light chain having the amino acid sequences of SEQ ID NOs:206 and 165, respectively.

In some embodiments, the fusion protein provided herein comprises a heavy chain and a light chain having the amino acid sequences of SEQ ID NOs:207 and 162, respectively. In some embodiments, the fusion protein comprises a heavy chain and a light chain having the amino acid sequences of SEQ ID NOs:207 and 163, respectively. In some embodiments, the fusion protein comprises a heavy chain and a light chain having the amino acid sequences of SEQ ID NOs:207 and 164, respectively. In some embodiments, the fusion protein comprises a heavy chain and a light chain having the amino acid sequences of SEQ ID NOs:207 and 165, respectively.

In some embodiments, the fusion protein provided herein comprises a heavy chain and a light chain having the amino acid sequences of SEQ ID NOs:208 and 162, respectively. In some embodiments, the fusion protein comprises a heavy chain and a light chain having the amino acid sequences of SEQ ID NOs:208 and 163, respectively. In some embodiments, the fusion protein comprises a heavy chain and a light chain having the amino acid sequences of SEQ ID NOs:208 and 164, respectively. In some embodiments, the fusion protein comprises a heavy chain and a light chain having the amino acid sequences of SEQ ID NOs:208 and 165, respectively.

In some embodiments, the fusion protein provided herein comprises a heavy chain and a light chain having the amino acid sequences of SEQ ID NOs:209 and 162, respectively. In some embodiments, the fusion protein comprises a heavy chain and a light chain having the amino acid sequences of SEQ ID NOs:209 and 163, respectively. In some embodiments, the fusion protein comprises a heavy chain and a light chain having the amino acid sequences of SEQ ID NOs:209 and 164, respectively. In some embodiments, the fusion protein comprises a heavy chain and a light chain having the amino acid sequences of SEQ ID NOs:209 and 165, respectively.

In some embodiments, the fusion protein provided herein comprises a heavy chain and a light chain having the amino acid sequences of SEQ ID NOs:210 and 162, respectively. In some embodiments, the fusion protein comprises a heavy chain and a light chain having the amino acid sequences of SEQ ID NOs:210 and 163, respectively. In some embodiments, the fusion protein comprises a heavy chain and a light chain having the amino acid sequences of SEQ ID NOs:210 and 164, respectively. In some embodiments, the fusion protein comprises a heavy chain and a light chain having the amino acid sequences of SEQ ID NOs:210 and 165, respectively.

In some embodiments, the fusion protein provided herein comprises a heavy chain and a light chain having the amino acid sequences of SEQ ID NOs:211 and 162, respectively. In some embodiments, the fusion protein comprises a heavy chain and a light chain having the amino acid sequences of SEQ ID NOs:211 and 163, respectively. In some embodiments, the fusion protein comprises a heavy chain and a light chain having the amino acid sequences of SEQ ID NOs:211 and 164, respectively. In some embodiments, the fusion protein comprises a heavy chain and a light chain having the amino acid sequences of SEQ ID NOs:211 and 165, respectively.

In some embodiments, the fusion protein provided herein comprises a heavy chain and a light chain having the amino acid sequences of SEQ ID NOs:212 and 162, respectively. In some embodiments, the fusion protein comprises a heavy chain and a light chain having the amino acid sequences of SEQ ID NOs:212 and 163, respectively. In some embodiments, the fusion protein comprises a heavy chain and a light chain having the amino acid sequences of SEQ ID NOs:212 and 164, respectively. In some embodiments, the fusion protein comprises a heavy chain and a light chain having the amino acid sequences of SEQ ID NOs:212 and 165, respectively.

In some embodiments, the fusion protein provided herein comprises a heavy chain and a light chain having the amino acid sequences of SEQ ID NOs:233 and 162, respectively. In some embodiments, the fusion protein comprises a heavy chain and a light chain having the amino acid sequences of SEQ ID NOs:233 and 163, respectively. In some embodiments, the fusion protein comprises a heavy chain and a light chain having the amino acid sequences of SEQ ID NOs:233 and 164, respectively. In some embodiments, the fusion protein comprises a heavy chain and a light chain having the amino acid sequences of SEQ ID NOs:233 and 165, respectively.

In some embodiments, the fusion protein provided herein comprises a heavy chain and a light chain having the amino acid sequences of SEQ ID NOs:234 and 162, respectively. In some embodiments, the fusion protein comprises a heavy chain and a light chain having the amino acid sequences of SEQ ID NOs:234 and 163, respectively. In some embodiments, the fusion protein comprises a heavy chain and a light chain having the amino acid sequences of SEQ ID NOs:234 and 164, respectively. In some embodiments, the fusion protein comprises a heavy chain and a light chain having the amino acid sequences of SEQ ID NOs:234 and 165, respectively.

In some embodiments, the fusion protein provided herein comprises a heavy chain and a light chain having the amino acid sequences of SEQ ID NOs:235 and 162, respectively. In some embodiments, the fusion protein comprises a heavy chain and a light chain having the amino acid sequences of SEQ ID NOs:235 and 163, respectively. In some embodiments, the fusion protein comprises a heavy chain and a light chain having the amino acid sequences of SEQ ID NOs:235 and 164, respectively. In some embodiments, the fusion protein comprises a heavy chain and a light chain having the amino acid sequences of SEQ ID NOs:235 and 165, respectively.

In some embodiments, the fusion protein provided herein comprises a heavy chain and a light chain having the amino acid sequences of SEQ ID NOs:236 and 162, respectively. In some embodiments, the fusion protein comprises a heavy chain and a light chain having the amino acid sequences of SEQ ID NOs:236 and 163, respectively. In some embodiments, the fusion protein comprises a heavy chain and a light chain having the amino acid sequences of SEQ ID NOs:236 and 164, respectively. In some embodiments, the fusion protein comprises a heavy chain and a light chain having the amino acid sequences of SEQ ID NOs:236 and 165, respectively.

In some embodiments, the fusion protein provided herein comprises a heavy chain and a light chain having the amino acid sequences of SEQ ID NOs:237 and 162, respectively. In some embodiments, the fusion protein comprises a heavy chain and a light chain having the amino acid sequences of SEQ ID NOs:237 and 163, respectively. In some embodiments, the fusion protein comprises a heavy chain and a light chain having the amino acid sequences of SEQ ID NOs:237 and 164, respectively. In some embodiments, the fusion protein comprises a heavy chain and a light chain having the amino acid sequences of SEQ ID NOs:237 and 165, respectively.

In some embodiments, the fusion protein provided herein comprises a heavy chain and a light chain having the amino acid sequences of SEQ ID NOs:238 and 162, respectively. In some embodiments, the fusion protein comprises a heavy chain and a light chain having the amino acid sequences of SEQ ID NOs:238 and 163, respectively. In some embodiments, the fusion protein comprises a heavy chain and a light chain having the amino acid sequences of SEQ ID NOs:238 and 164, respectively. In some embodiments, the fusion protein comprises a heavy chain and a light chain having the amino acid sequences of SEQ ID NOs:238 and 165, respectively.

In some embodiments, the fusion protein provided herein comprises a heavy chain and a light chain having the amino acid sequences of SEQ ID NOs:239 and 162, respectively. In some embodiments, the fusion protein comprises a heavy chain and a light chain having the amino acid sequences of SEQ ID NOs:239 and 163, respectively. In some embodiments, the fusion protein comprises a heavy chain and a light chain having the amino acid sequences of SEQ ID NOs:239 and 164, respectively. In some embodiments, the fusion protein comprises a heavy chain and a light chain having the amino acid sequences of SEQ ID NOs:239 and 165, respectively.

In some embodiments, the fusion protein provided herein comprises a heavy chain and a light chain having the amino acid sequences of SEQ ID NOs:240 and 162, respectively. In some embodiments, the fusion protein comprises a heavy chain and a light chain having the amino acid sequences of SEQ ID NOs:240 and 163, respectively. In some embodiments, the fusion protein comprises a heavy chain and a light chain having the amino acid sequences of SEQ ID NOs:240 and 164, respectively. In some embodiments, the fusion protein comprises a heavy chain and a light chain having the amino acid sequences of SEQ ID NOs:240 and 165, respectively.

### 4. Nucleic Acids, Vectors, and Cells

In some embodiments, provided herein are polynucleotides that encode a polypeptide (i.e., a fusion protein or an anti-PD-L1 antibody or antigen-binding fragment) described herein. The term “polynucleotide that encode a polypeptide” encompasses a polynucleotide which includes only coding sequences for the polypeptide as well as a polynucleotide which includes additional coding and/or non-coding sequences. The polynucleotides of the disclosure can be in the form of RNA or in the form of DNA. DNA includes cDNA, genomic DNA, and synthetic DNA; and can be double-stranded or single-stranded, and if single stranded can be the coding strand or non-coding (anti-sense) strand.

In some embodiments, the polynucleotides provided herein encode an anti-PD-L1 antibody or antigen-binding fragment disclosed herein. In some embodiments, the polynucleotide provided herein encode an anti-PD-L1 antibody or antigen-binding fragment disclosed herein comprising (a) VH comprising (1) a VH CDR1 having an amino acid sequence selected from the group consisting of SEQ ID NOs:20, 26, 32, 38, 44, 50, 56, 62, 68, 74, 80, 86, and 92; (2) a VH CDR2 having an amino acid sequence selected from the group consisting of SEQ ID NOs:21, 27, 33, 39, 45, 51, 57, 63, 69, 75, 81, 87 and 93; or (3) a VH CDR3 having an amino acid sequence selected from the group consisting of SEQ ID NOs:22, 28, 34, 40, 46, 52, 58, 64, 70, 76, 82, 89 and 94; or a variant thereof having up to about 5 amino acid substitutions, additions, and/or deletions in the VH CDRs; and (b) a VL comprising (1) a VL CDR1 having an amino acid sequence selected from the group consisting of SEQ ID NOs:23, 29, 35, 41, 47, 53, 59, 65, 71, 77, 83, 89, and 95; (2) a VL CDR2 having an amino acid sequence selected from the group consisting of SEQ ID NOs:24, 30, 36, 42, 48, 54, 60, 66, 72, 78, 84, 90, and 96; and (3) a VL CDR3 having an amino acid sequence selected from the group consisting of SEQ ID NOs:25, 31, 37, 43, 49, 55, 61, 67, 63, 79, 85, 91, and 97; or a variant thereof having up to about 5 amino acid substitutions, additions, and/or deletions in the VL CDRs.

In some embodiments, the polynucleotide provided herein encode an anti-PD-L1 antibody or antigen-binding fragment disclosed herein comprising provided herein are antibodies or antigen-binding fragments thereof that bind PD-L1 having a VH and a VL, wherein (a) the VH comprises VH CDR1, CDR2 and CDR3 having (1) the amino acid sequences of SEQ ID NOs:20, 21, and 22, respectively; (2) the amino acid sequences of SEQ ID NOs:26, 27, and 28, respectively; (3) the amino acid sequences of SEQ ID NOs:32, 33, and 34, respectively; (4) the amino acid sequences of SEQ ID NOs:38, 39, and 40, respectively; (5) the amino acid sequences of SEQ ID NOs:44, 45 and 46, respectively; (6) the amino acid sequences of SEQ ID NOs:50, 51, and 52, respectively; (7) the amino acid sequences of SEQ ID NOs:56, 57, and 58, respectively; (8) the amino acid sequences of SEQ ID NOs:62, 63, and 64, respectively; (9) the amino acid sequences of SEQ ID NOs:68, 69, and 70, respectively; (10) the amino acid sequences of SEQ ID NOs:74, 75, and 76, respectively; (11) the amino acid sequences of SEQ ID NOs:80, 81, and 82, respectively; (12) the amino acid sequences of SEQ ID NOs:86, 87, and 88, respectively; or (13) the amino acid sequences of SEQ ID NOs:92, 93 and 94, respectively; or a variant thereof having up to about 5 amino acid substitutions, additions, and/or deletions in the VH CDRs; and (b) the VL comprises VL CDR1, CDR2 and CDR3 having the amino acid sequences of SEQ ID NOs:23, 24 and 25, respectively; (2) the amino acid sequences of SEQ ID NOs:29, 30, and 31, respectively; (3) the amino acid sequences of SEQ ID NOs:35, 36 and 37, respectively; (4) the amino acid sequences of SEQ ID NOs:41, 42 and 43, respectively; (5) the amino acid sequences of SEQ ID NOs:47, 48 and 49, respectively; (6) the amino acid sequences of SEQ ID NOs:53, 54, and 55, respectively; (7) the amino acid sequences of SEQ ID NOs:59, 60, and 61, respectively; (8) the amino acid sequences of SEQ ID NOs:65, 66, and 67, respectively; (9) the amino acid sequences of SEQ ID NOs:71, 72 and 73, respectively; (10) the amino acid sequences of SEQ ID NOs:77, 78 and 79, respectively; (11) the amino acid sequences of SEQ ID NOs:83, 84, and 85, respectively; (12) the amino acid sequences of SEQ ID NOs:89, 90 and 91, respectively; or (13) the amino acid sequences of SEQ ID NOs:95, 96 and 97, respectively; or a variant thereof having up to about 5 amino acid substitutions, additions, and/or deletions in the VL CDRs.

In some embodiments, the polynucleotide provided herein encode an anti-PD-L1 antibody or antigen-binding fragment disclosed herein comprising a VH having at least 85%, at least 90%, at least 95%, at least 98%, or 100% sequence identity to an amino acid sequence selected from SEQ ID NOs:98-110, 124, 126-128, 131-136, and 174-178. In some embodiments, the polynucleotide provided herein encode an anti-PD-L1 antibody or antigen-binding fragment disclosed herein comprising a VL having at least 85%, at least 90%, at least 95%, at least 98%, or 100% sequence identity to an amino acid sequence selected from the group consisting of SEQ ID NOs:111-123, 125, 129-130, 137-144 and 179-181. Expressly contemplated herein are polynucleotides encode any anti-PD-L1 antibody or antigen-binding fragment disclosed herein.

In some embodiments, the polynucleotides provided herein encode a fusion protein disclosed herein. In some embodiments, the polynucleotides provided herein encode fusion proteins having two domains, wherein the first domain comprises an antibody that binds PD-L1or an antigen-binding fragment thereof, and the second domain comprises a fragment of TGFβRII that binds TGFβ, or a variant thereof. In some embodiments, the two domains are linked by a rigid linker (e.g., a transferrin linker). In some embodiments, polynucleotides provided herein encode fusion proteins comprising a heavy chain, wherein the heavy chain has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to an amino acid sequence selected from the group consisting of SEQ ID NOs: 155-161, 166-172, 206-212 and 233-240. In some embodiments, polynucleotides provided herein encode fusion proteins comprising a light chain, wherein the light chain has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to an amino acid sequence selected from the group consisting of SEQ ID NOs: 162-165. Expressly contemplated herein are polynucleotides encode any fusion protein disclosed herein.

In some embodiments, polynucleotides provided herein encode fusion proteins comprising a heavy chain. In some embodiments, the present disclosure provides a polynucleotide having a nucleotide sequence at least about 80% identical, at least about 85% identical, at least about 90% identical, at least about 95% identical, at least about 96% identical, at least about 97% identical, at least about 98% identical, or at least about 99% identical to a nucleotide sequence selected from the group consisting of SEQ ID NOs:183-189, 194-200 and 213-219. In some embodiments, provided herein are polynucleotides having a nucleotide sequence at least about 80% identical, at least about 85% identical, at least about 90% identical, at least about 95% identical, at least about 96% identical, at least about 97% identical, at least about 98% identical, or at least about 99% identical to SEQ ID NO:183. In some embodiments, provided herein are polynucleotides having a nucleotide sequence at least about 80% identical, at least about 85% identical, at least about 90% identical, at least about 95% identical, at least about 96% identical, at least about 97% identical, at least about 98% identical, or at least about 99% identical to SEQ ID NO:184. In some embodiments, provided herein are polynucleotides having a nucleotide sequence at least about 80% identical, at least about 85% identical, at least about 90% identical, at least about 95% identical, at least about 96% identical, at least about 97% identical, at least about 98% identical, or at least about 99% identical to SEQ ID NO:185. In some embodiments, provided herein are polynucleotides having a nucleotide sequence at least about 80% identical, at least about 85% identical, at least about 90% identical, at least about 95% identical, at least about 96% identical, at least about 97% identical, at least about 98% identical, or at least about 99% identical to SEQ ID NO:186. In some embodiments, provided herein are polynucleotides having a nucleotide sequence at least about 80% identical, at least about 85% identical, at least about 90% identical, at least about 95% identical, at least about 96% identical, at least about 97% identical, at least about 98% identical, or at least about 99% identical to SEQ ID NO:187. In some embodiments, provided herein are polynucleotides having a nucleotide sequence at least about 80% identical, at least about 85% identical, at least about 90% identical, at least about 95% identical, at least about 96% identical, at least about 97% identical, at least about 98% identical, or at least about 99% identical to SEQ ID NO: 188. In some embodiments, provided herein are polynucleotides having a nucleotide sequence at least about 80% identical, at least about 85% identical, at least about 90% identical, at least about 95% identical, at least about 96% identical, at least about 97% identical, at least about 98% identical, or at least about 99% identical to SEQ ID NO:189. In some embodiments, provided herein are polynucleotides having a nucleotide sequence at least about 80% identical, at least about 85% identical, at least about 90% identical, at least about 95% identical, at least about 96% identical, at least about 97% identical, at least about 98% identical, or at least about 99% identical to SEQ ID NO:194. In some embodiments, provided herein are polynucleotides having a nucleotide sequence at least about 80% identical, at least about 85% identical, at least about 90% identical, at least about 95% identical, at least about 96% identical, at least about 97% identical, at least about 98% identical, or at least about 99% identical to SEQ ID NO:195. In some embodiments, provided herein are polynucleotides having a nucleotide sequence at least about 80% identical, at least about 85% identical, at least about 90% identical, at least about 95% identical, at least about 96% identical, at least about 97% identical, at least about 98% identical, or at least about 99% identical to SEQ ID NO: 196. In some embodiments, provided herein are polynucleotides having a nucleotide sequence at least about 80% identical, at least about 85% identical, at least about 90% identical, at least about 95% identical, at least about 96% identical, at least about 97% identical, at least about 98% identical, or at least about 99% identical to SEQ ID NO:197. In some embodiments, provided herein are polynucleotides having a nucleotide sequence at least about 80% identical, at least about 85% identical, at least about 90% identical, at least about 95% identical, at least about 96% identical, at least about 97% identical, at least about 98% identical, or at least about 99% identical to SEQ ID NO:198. In some embodiments, provided herein are polynucleotides having a nucleotide sequence at least about 80% identical, at least about 85% identical, at least about 90% identical, at least about 95% identical, at least about 96% identical, at least about 97% identical, at least about 98% identical, or at least about 99% identical to SEQ ID NO:199. In some embodiments, provided herein are polynucleotides having a nucleotide sequence at least about 80% identical, at least about 85% identical, at least about 90% identical, at least about 95% identical, at least about 96% identical, at least about 97% identical, at least about 98% identical, or at least about 99% identical to SEQ ID NO:200. In some embodiments, provided herein are polynucleotides having a nucleotide sequence at least about 80% identical, at least about 85% identical, at least about 90% identical, at least about 95% identical, at least about 96% identical, at least about 97% identical, at least about 98% identical, or at least about 99% identical to SEQ ID NO:213. In some embodiments, provided herein are polynucleotides having a nucleotide sequence at least about 80% identical, at least about 85% identical, at least about 90% identical, at least about 95% identical, at least about 96% identical, at least about 97% identical, at least about 98% identical, or at least about 99% identical to SEQ ID NO:214. In some embodiments, provided herein are polynucleotides having a nucleotide sequence at least about 80% identical, at least about 85% identical, at least about 90% identical, at least about 95% identical, at least about 96% identical, at least about 97% identical, at least about 98% identical, or at least about 99% identical to SEQ ID NO:215. In some embodiments, provided herein are polynucleotides having a nucleotide sequence at least about 80% identical, at least about 85% identical, at least about 90% identical, at least about 95% identical, at least about 96% identical, at least about 97% identical, at least about 98% identical, or at least about 99% identical to SEQ ID NO:216. In some embodiments, provided herein are polynucleotides having a nucleotide sequence at least about 80% identical, at least about 85% identical, at least about 90% identical, at least about 95% identical, at least about 96% identical, at least about 97% identical, at least about 98% identical, or at least about 99% identical to SEQ ID NO:217. In some embodiments, provided herein are polynucleotides having a nucleotide sequence at least about 80% identical, at least about 85% identical, at least about 90% identical, at least about 95% identical, at least about 96% identical, at least about 97% identical, at least about 98% identical, or at least about 99% identical to SEQ ID NO:218. In some embodiments, provided herein are polynucleotides having a nucleotide sequence at least about 80% identical, at least about 85% identical, at least about 90% identical, at least about 95% identical, at least about 96% identical, at least about 97% identical, at least about 98% identical, or at least about 99% identical to SEQ ID NO:219. Also provided is a polynucleotide that hybridizes to a polynucleotide having a nucleotide sequence selected from the group consisting of SEQ ID NOs: 183-189, 194-200 and 213-219. In some embodiments, the hybridization is under conditions of high stringency as is known to those skilled in the art.

In some embodiments, polynucleotides provided herein encode fusion proteins comprising a light chain. In some embodiments, the present disclosure provides a polynucleotide having a nucleotide sequence at least about 80% identical, at least about 85% identical, at least about 90% identical, at least about 95% identical, at least about 96% identical, at least about 97% identical, at least about 98% identical, or at least about 99% identical to a nucleotide sequence selected from the group consisting of SEQ ID NOs: 190-193. In some embodiments, provided herein are polynucleotides having a nucleotide sequence at least about 80% identical, at least about 85% identical, at least about 90% identical, at least about 95% identical, at least about 96% identical, at least about 97% identical, at least about 98% identical, or at least about 99% identical to SEQ ID NO:190. In some embodiments, provided herein are polynucleotides having a nucleotide sequence at least about 80% identical, at least about 85% identical, at least about 90% identical, at least about 95% identical, at least about 96% identical, at least about 97% identical, at least about 98% identical, or at least about 99% identical to SEQ ID NO:191. In some embodiments, provided herein are polynucleotides having a nucleotide sequence at least about 80% identical, at least about 85% identical, at least about 90% identical, at least about 95% identical, at least about 96% identical, at least about 97% identical, at least about 98% identical, or at least about 99% identical to SEQ ID NO:192. In some embodiments, provided herein are polynucleotides having a nucleotide sequence at least about 80% identical, at least about 85% identical, at least about 90% identical, at least about 95% identical, at least about 96% identical, at least about 97% identical, at least about 98% identical, or at least about 99% identical to SEQ ID NO: 193. Also provided is a polynucleotide that hybridizes to a polynucleotide having a nucleotide sequence selected from the group consisting of SEQ ID NOs: 190-193. In some embodiments, the hybridization is under conditions of high stringency as is known to those skilled in the art.

The present disclosure also provides variants of the polynucleotides described herein, wherein the variant encodes, for example, fragments, analogs, and/or derivatives of a fusion protein or an anti-PD-L1 antibody or antigen-binding fragment described herein. In some embodiments, the present disclosure provides a polynucleotide having a nucleotide sequence at least about 80% identical, at least about 85% identical, at least about 90% identical, at least about 95% identical, at least about 96% identical, at least about 97% identical, at least about 98% identical, or at least about 99% identical to a polynucleotide sequence encoding a fusion protein described herein. In some embodiments, the present disclosure provides a polynucleotide having a nucleotide sequence at least about 80% identical, at least about 85% identical, at least about 90% identical, at least about 95% identical, at least about 96% identical, at least about 97% identical, at least about 98% identical, or at least about 99% identical to a polynucleotide sequence encoding an anti-PD-L1 antibody or antigen-binding fragment described herein.

As used herein, the phrase “a polynucleotide having a nucleotide sequence at least about 95% identical to a polynucleotide sequence” means that the nucleotide sequence of the polynucleotide is identical to a reference sequence except that the polynucleotide sequence can include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence can be deleted or substituted with another nucleotide, or a number of nucleotides up to 5% of the total nucleotides in the reference sequence can be inserted into the reference sequence. These mutations of the reference sequence can occur at the 5′ or 3′ terminal positions of the reference nucleotide sequence or anywhere between those terminal positions, interspersed either individually among nucleotides in the reference sequence or in one or more contiguous groups within the reference sequence.

The polynucleotide variants can contain alterations in the coding regions, non-coding regions, or both. In some embodiments, a polynucleotide variant contains alterations which produce silent substitutions, additions, or deletions, but does not alter the properties or activities of the encoded polypeptide. In some embodiments, a polynucleotide variant comprises silent substitutions that results in no change to the amino acid sequence of the polypeptide (due to the degeneracy of the genetic code). Polynucleotide variants can be produced for a variety of reasons, for example, to optimize codon expression for a particular host (e.g., change codons in the human mRNA to those preferred by a bacterial host such as E. coli). In some embodiments, a polynucleotide variant comprises at least one silent mutation in a non-coding or a coding region of the sequence.

In some embodiments, a polynucleotide variant is produced to modulate or alter expression (or expression levels) of the encoded polypeptide. In some embodiments, a polynucleotide variant is produced to increase expression of the encoded polypeptide. In some embodiments, a polynucleotide variant is produced to decrease expression of the encoded polypeptide. In some embodiments, a polynucleotide variant has increased expression of the encoded polypeptide as compared to a parental polynucleotide sequence. In some embodiments, a polynucleotide variant has decreased expression of the encoded polypeptide as compared to a parental polynucleotide sequence.

In some embodiments, a polynucleotide has a nucleotide sequence at least about 80% identical, at least about 85% identical, at least about 90% identical, at least about 95% identical, at least about 96% identical, at least about 97% identical, at least about 98% identical, or at least about 99% identical to a polynucleotide encoding an amino acid sequence selected from SEQ ID NOs:98-144, 155-172, 174-181, 201-212, and 233-240. Also provided is a polynucleotide that hybridizes to a polynucleotide encoding an amino acid sequence selected from SEQ ID NOs:98-144, 155-172, 174-181, 201-212, and 233-240. In some embodiments, the hybridization is under conditions of high stringency as is known to those skilled in the art.

In some embodiments, a polynucleotide comprises the coding sequence for a polypeptide (e.g., a fusion protein or an antibody) fused in the same reading frame to a polynucleotide which aids in expression and secretion of a polypeptide from a host cell (e.g., a leader sequence which functions as a secretory sequence for controlling transport of a polypeptide). The polypeptide can have the leader sequence cleaved by the host cell to form a “mature” form of the polypeptide.

In some embodiments, a polynucleotide comprises the coding sequence for a polypeptide (e.g., a fusion protein or an antibody) fused in the same reading frame to a marker or tag sequence. For example, in some embodiments, a marker sequence is a hexa-histidine tag (HIS-tag) that allows for efficient purification of the polypeptide fused to the marker. In some embodiments, a marker sequence is a hemagglutinin (HA) tag derived from the influenza hemagglutinin protein when a mammalian host (e.g., COS-7 cells) is used. In some embodiments, the marker sequence is a FLAG™ tag. In some embodiments, a marker may be used in conjunction with other markers or tags.

In some embodiments, a polynucleotide is isolated. In some embodiments, a polynucleotide is substantially pure.

Vectors and cells comprising the polynucleotides described herein are also provided. In some embodiments, an expression vector comprises a polynucleotide encoding an anti-PD-L1 antibody or antigen-binding fragment described herein. In some embodiments, an expression vector comprises a polynucleotide encoding a polypeptide that is part of an anti-PD-L1 antibody or antigen-binding fragment described herein. In some embodiments, a host cell comprises an expression vector comprising a polynucleotide encoding an anti-PD-L1 antibody or antigen-binding fragment described herein. In some embodiments, a host cell comprises an expression vector comprising a polynucleotide molecule encoding a polypeptide that is part of an anti-PD-L1 antibody or antigen-binding fragment described herein. In some embodiments, a host cell comprises a polynucleotide encoding an anti-PD-L1 antibody or antigen-binding fragment described herein. In some embodiments, an expression vector comprises a polynucleotide encoding a fusion protein described herein. In some embodiments, an expression vector comprises a polynucleotide encoding a polypeptide that is part of a fusion protein described herein. In some embodiments, a host cell comprises an expression vector comprising a polynucleotide encoding a fusion protein described herein. In some embodiments, a host cell comprises an expression vector comprising a polynucleotide molecule encoding a polypeptide that is part of a fusion protein described herein. In some embodiments, a host cell comprises a polynucleotide encoding a fusion protein described herein.

The fusion proteins described herein and anti-PD-L1 antibodies or antigen-binding fragments described herein can be produced by any method known in the art, including chemical synthesis and recombinant expression techniques. The practice of the invention employs, unless otherwise indicated, conventional techniques in molecular biology, microbiology, genetic analysis, recombinant DNA, organic chemistry, biochemistry, PCR, oligonucleotide synthesis and modification, nucleic acid hybridization, and related fields within the skill of the art. These techniques are described in the references cited herein and are fully explained in the literature. See, e.g., Maniatis et al. (1982) Molecular Cloning: A Laboratory Manual, Cold Spring Harbor Laboratory Press; Sambrook et al. (1989), Molecular Cloning: A Laboratory Manual, Second Edition, Cold Spring Harbor Laboratory Press; Sambrook et al. (2001) Molecular Cloning: A Laboratory Manual, Cold Spring Harbor Laboratory Press, Cold Spring Harbor, NY; Ausubel et al., Current Protocols in Molecular Biology, John Wiley & Sons (1987 and annual updates); Current Protocols in Immunology, John Wiley & Sons (1987 and annual updates) Gait (ed.) (1984) Oligonucleotide Synthesis: A Practical Approach, IRL Press; Eckstein (ed.) (1991) Oligonucleotides and Analogues: A Practical Approach, IRL Press; Birren et al. (eds.) (1999) Genome Analysis: A Laboratory Manual, Cold Spring Harbor Laboratory Press; Borrebaeck (ed.) (1995) Antibody Engineering, Second Edition, Oxford University Press; Lo (ed.) (2006) Antibody Engineering: Methods and Protocols (Methods in Molecular Biology); Vol. 248, Humana Press, Inc; each of which is incorporated herein by reference in its entirety.

The fusion proteins described herein and the anti-PD-L1 antibodies or antigen-binding fragments described herein can be produced and isolated using methods known in the art. Peptides can be synthesized, in whole or in part, using chemical methods (see, e.g., Caruthers (1980). Nucleic Acids Res. Symp. Ser. 215; Horn (1980); and Banga, A.K., Therapeutic Peptides and Proteins, Formulation, Processing and Delivery Systems (1995) Technomic Publishing Co., Lancaster, PA). Peptide synthesis can be performed using various solid-phase techniques (see, e.g., Roberge Science 269:202 (1995); Merrifield, Methods. Enzymol. 289:3 (1997)) and automated synthesis may be achieved, e.g., using the ABI 431A Peptide Synthesizer (Perkin Elmer) in accordance with the manufacturer’s instructions. Peptides can also be synthesized using combinatorial methodologies. Synthetic residues and polypeptides can be synthesized using a variety of procedures and methodologies known in the art (see, e.g., Organic Syntheses Collective Volumes, Gilman, et al. (Eds) John Wiley & Sons, Inc., NY). Modified peptides can be produced by chemical modification methods (see, for example, Belousov, Nucleic Acids Res. 25:3440 (1997); Frenkel, Free Radic. Biol. Med. 19:373 (1995); and Blommers, Biochemistry 33:7886 (1994)). Peptide sequence variations, derivatives, substitutions and modifications can also be made using methods such as oligonucleotide-mediated (site-directed) mutagenesis, alanine scanning, and PCR based mutagenesis. Site-directed mutagenesis (Carter et al., Nucl. Acids Res., 13:4331 (1986); Zoller et al., Nucl. Acids Res. 10:6487 (1987)), cassette mutagenesis (Wells et al., Gene 34:315 (1985)), restriction selection mutagenesis (Wells et al., Philos. Trans. R. Soc. London SerA 317:415 (1986)) and other techniques can be performed on cloned DNA to produce invention peptide sequences, variants, fusions and chimeras, and variations, derivatives, substitutions and modifications thereof.

Antibodies of the present disclosure can be produced by any techniques known in the art, including conventional monoclonal antibody methodology e.g., the standard somatic cell hybridization technique (See e.g., Kohler and Milstein, Nature 256:495 (1975)), viral or oncogenic transformation of B lymphocytes, or recombinant antibody technologies.

Hybridoma production is a well-established procedure. The common animal system for preparing hybridomas is the murine system. Immunization protocols and techniques for isolation of immunized splenocytes for fusion are known in the art. Fusion partners (e.g., murine myeloma cells) and fusion procedures are also known. The animal can be immunized with a PD-L1 antigen, which can be a purified PD-L1, preferably human PD-L1. It can be a fragment of PD-L1, such as the extracellular domain of PD-L1, particularly a human PD-L1 extracellular domain fragment comprising amino acid resides (Phe19-Arg238). Immunization of animals can be carried out by any method known in the art (See, e.g., Harlow and Lane, ANTIBODIES: A LABORATORY MANUAL, New York: Cold Spring Harbor Press, 1990). Methods for immunizing non-human animals such as mice, rats, sheep, goats, pigs, cattle and horses are well-known in the art (See, e.g., Harlow and Lane, supra, and U.S. Pat. No. 5,994,619). The PD-L1 antigen can be administered with an adjuvant to stimulate the immune response. Exemplary adjuvants include, for example, complete or incomplete Freund’s adjuvant, RIBI (muramyl dipeptides) or ISCOM (immunostimulating complexes). After immunization of an animal with a PD-L1 antigen, antibody-producing immortalized cell lines are prepared from cells isolated from the immunized animal. After immunization, the animal is sacrificed and lymph node and/or splenic B cells are immortalized. Methods of immortalizing cells include, but are not limited to, transferring them with oncogenes, inflecting them with the oncogenic virus cultivating them under conditions that select for immortalized cells, subjecting them to carcinogenic or mutating compounds, fusing them with an immortalized cell, e.g., a myeloma cell, and inactivating a tumor suppressor gene (See, e.g., Harlow and Lane, supra). If fusion with myeloma cells is used, the myeloma cells preferably do not secrete immunoglobulin polypeptides (a non-secretory cell line). Immortalized cells are screened using PD-L1, a portion thereof, or a cell expressing PD-L1. PD-L1 antibody-producing cells, e.g., hybridomas, are selected, cloned and further screened for desirable characteristics, including robust growth, high antibody production and desirable antibody characteristics. Hybridomas can be expanded in vivo in syngeneic animals, in animals that lack an immune system, e.g., nude mice, or in cell culture in vitro. Methods of selecting, cloning and expanding hybridomas are well known to those of ordinary skill in the art.

The fusion proteins described herein and anti-PD-L1 antibodies or antigen-binding fragments described herein can be prepared using a wide variety of techniques known in the art including the use of hybridoma and recombinant technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed. 1988); Hammerling et al., in: Monoclonal Antibodies and T-Cell Hybridomas 563 681 (Elsevier, N.Y., 1981), each of which is incorporated herein by reference in its entirety. Other methods of producing the polypeptides are also known in the art.

In some embodiments, a recombinant expression vector is used to amplify and express a polynucleotide encoding a fusion protein described herein or an anti-PD-L1 antibody or antigen-binding fragment described herein. For example, a recombinant expression vector can be a replicable DNA construct that includes synthetic or cDNA-derived DNA fragments encoding a fusion protein or a polypeptide chain of an anti-PD-L1 antibody, operatively linked to suitable transcriptional and/or translational regulatory elements derived from mammalian, microbial, viral or insect genes. In some embodiments, a viral vector is used. DNA regions are “operatively linked” when they are functionally related to each other. For example, a promoter is operatively linked to a coding sequence if it controls the transcription of the sequence; or a ribosome binding site is operatively linked to a coding sequence if it is positioned so as to permit translation. In some embodiments, structural elements intended for use in yeast expression systems include a leader sequence enabling extracellular secretion of translated protein by a host cell. In some embodiments, in situations where recombinant protein is expressed without a leader or transport sequence, a polypeptide may include an N-terminal methionine residue.

A wide variety of expression host/vector combinations can be employed. Useful expression vectors for eukaryotic hosts include, for example, vectors comprising expression control sequences from SV40, bovine papilloma virus, adenovirus, and cytomegalovirus. Useful expression vectors for bacterial hosts include known bacterial plasmids, such as plasmids from E. coli, including pCR1, pBR322, pMB9 and their derivatives, and wider host range plasmids, such as M13 and other filamentous single-stranded DNA phages.

In some embodiments, a fusion protein described herein or an anti-PD-L1 antibody or antigen-binding fragment described herein is expressed from one or more vectors. Suitable host cells for expression include prokaryotes, yeast cells, insect cells, or higher eukaryotic cells under the control of appropriate promoters. Appropriate cloning and expression vectors for use with bacterial, fungal, yeast, and mammalian cellular hosts, as well as methods of protein production, including antibody production are well-known in the art.

Examples of suitable mammalian host cell lines include, but are not limited to, COS-7 (monkey kidney-derived), L-929 (murine fibroblast-derived), C127 (murine mammary tumor-derived), 3T3 (murine fibroblast-derived), CHO (Chinese hamster ovary-derived), HeLa (human cervical cancer-derived), BHK (hamster kidney fibroblast-derived), HEK-293 (human embryonic kidney-derived) cell lines and variants thereof. Mammalian expression vectors can comprise non-transcribed elements such as an origin of replication, a suitable promoter and enhancer linked to the gene to be expressed, and other 5′ or 3′ flanking non-transcribed sequences, and 5′ or 3′ non-translated sequences, such as necessary ribosome binding sites, a polyadenylation site, splice donor and acceptor sites, and transcriptional termination sequences. Expression of recombinant proteins in insect cell culture systems (e.g., baculovirus) also offers a robust method for producing correctly folded and biologically functional proteins. Baculovirus systems for production of heterologous proteins in insect cells are well-known to those of skill in the art.

Thus, the present disclosure provides cells comprising the polypeptides described herein. In some embodiments, the present disclosure provides cells comprising the fusion proteins described herein. In some embodiments, the cells produce the fusion proteins described herein. In some embodiments, the present disclosure provides cells comprising the anti-PD-L1 antibodies or antigen-binding fragments described herein. In some embodiments, the cells produce the anti-PD-L1 antibodies or antigen-binding fragments described herein.

In some embodiments, the cells produce antibody Clone A or a variant thereof. In some embodiments, the cells produce chimeric version of antibody Clone A. In some embodiments, the cells produce a humanized version of antibody Clone A. In some embodiments, the cells produce antibody Clone B or a variant thereof. In some embodiments, the cells produce chimeric version of antibody Clone B. In some embodiments, the cells produce a humanized version of antibody Clone B. In some embodiments, the cells produce antibody Clone C or a variant thereof. In some embodiments, the cells produce chimeric version of antibody Clone C. In some embodiments, the cells produce a humanized version of antibody Clone C. In some embodiments, the cells produce antibody Clone D or a variant thereof. In some embodiments, the cells produce chimeric version of antibody Clone D. In some embodiments, the cells produce a humanized version of antibody Clone D. In some embodiments, the cells produce antibody Clone F or a variant thereof. In some embodiments, the cells produce chimeric version of antibody Clone F. In some embodiments, the cells produce a humanized version of antibody Clone F. In some embodiments, the cells produce antibody Clone H or a variant thereof. In some embodiments, the cells produce chimeric version of antibody Clone H. In some embodiments, the cells produce a humanized version of antibody Clone H. In some embodiments, the cells produce antibody Clone J or a variant thereof. In some embodiments, the cells produce chimeric version of antibody Clone J. In some embodiments, the cells produce a humanized version of antibody Clone J. In some embodiments, the cells produce antibody Clone K or a variant thereof. In some embodiments, the cells produce chimeric version of antibody Clone K. In some embodiments, the cells produce a humanized version of antibody Clone K. In some embodiments, the cells produce antibody Clone L or a variant thereof. In some embodiments, the cells produce chimeric version of antibody Clone L. In some embodiments, the cells produce a humanized version of antibody Clone L. In some embodiments, the cells produce antibody Clone N or a variant thereof. In some embodiments, the cells produce chimeric version of antibody Clone N. In some embodiments, the cells produce a humanized version of antibody Clone N. In some embodiments, the cells produce antibody Clone P or a variant thereof. In some embodiments, the cells produce chimeric version of antibody Clone P. In some embodiments, the cells produce a humanized version of antibody Clone P. In some embodiments, the cells produce antibody Clone Y or a variant thereof. In some embodiments, the cells produce chimeric version of antibody Clone Y. In some embodiments, the cells produce a humanized version of antibody Clone Y. In some embodiments, the cell is a prokaryotic cell (e.g., E. coli). In some embodiments, the cell is a eukaryotic cell. In some embodiments, the cell is a mammalian cell. In some embodiments, the cell is a hybridoma cell.

### 5. Compositions

Provided herein are pharmaceutical compositions having a fusion protein described herein. Further provided herein are compositions (e.g., pharmaceutical compositions) comprising an anti-PD-L1 antibody or antigen-binding fragment described herein. In some embodiments, provided herein are pharmaceutical compositions comprising a fusion protein described herein and a pharmaceutically acceptable carrier or vehicle. In some embodiments, provided herein are pharmaceutical compositions comprising an anti-PD-L1 antibody or antigen-binding fragment described herein and a pharmaceutically acceptable carrier or vehicle. In some embodiments, the pharmaceutical compositions are useful in immunotherapy. In some embodiments, the pharmaceutical compositions are useful in immune-oncology. In some embodiments, the compositions are useful in inhibiting tumor growth. In some embodiments, the pharmaceutical compositions are useful in inhibiting tumor growth in a subject (e.g., a human patient). In some embodiments, the compositions are useful in treating cancer. In some embodiments, the pharmaceutical compositions are useful in treating cancer in a subject (e.g., a human patient).

Pharmaceutically acceptable carriers that can be used in compositions or formulations provided herein include any and all solvents, dispersion media, coatings, antibacterial and antifungal agents, isotonic and absorption delaying agents, and the like that are physiologically compatible. In some embodiments, the carrier is suitable for intravenous, intramuscular, subcutaneous, parenteral, spinal or epidermal administration (e.g., by injection or infusion). Depending on the route of administration, the active ingredient, i.e., antibody or antigen binding fragment, or fusion protein, can be coated in a material to protect the active ingredient from the action of acids and other natural conditions that can inactivate the active ingredient.

Provided herein are also pharmaceutical compositions or formulations that improve the stability of the fusion proteins or anti-PD-L1 antibodies and antigen-binding fragments to allows for their long-term storage. In some embodiments, the pharmaceutical composition or formulation disclosed herein comprises: (a) a fusion protein described herein; (b) a buffering agent; (c) a stabilizing agent; (d) a salt; (e) a bulking agent; and/or (f) a surfactant. In some embodiments, the pharmaceutical composition or formulation disclosed herein comprises: (a) an anti-PD-L1 antibody or antigen-binding fragment; (b) a buffering agent; (c) a stabilizing agent; (d) a salt; (e) a bulking agent; and/or (f) a surfactant. In some embodiments, the pharmaceutical composition or formulation is stable for at least 1 month, at least 2 months, at least 3 months, at least 6 months, at least 1 year, at least 2 years, at least 3 years, at least 5 years or more. In some embodiments, the pharmaceutical composition or formulation is stable when stored at 4° C., 25° C., or 40° C.

Buffering agents useful in the pharmaceutical compositions or formulations disclosed herein can be a weak acid or base used to maintain the acidity (pH) of a solution near a chosen value after the addition of another acid or base. Suitable buffering agents can maximize the stability of the pharmaceutical formulations by maintaining pH control of the formulation. Suitable buffering agents can also ensure physiological compatibility or optimize solubility. Rheology, viscosity and other properties can also dependent on the pH of the formulation. Common buffering agents include, but are not limited to, histidine, citrate, succinate, acetate and phosphate. In some embodiments, a buffering agent comprises histidine (e.g., L-histidine) with isotonicity agents and potentially pH adjustment with an acid or a base known in the art. In certain embodiments, the buffering agent is L- histidine. In certain embodiments, the pH of the formulation is maintained between about 2 and about 10, or between about 4 and about 8.

Stabilizing agents are added to a pharmaceutical product in order to stabilize that product. Such agents can stabilize proteins in a number of different ways. Common stabilizing agents include, but are not limited to, amino acids such as glycine, alanine, lysine, arginine, or threonine, carbohydrates such as glucose, sucrose, trehalose, rafftnose, or maltose, polyols such as glycerol, mannitol, sorbitol, cyclodextrins or destrans of any kind and molecular weight, or PEG. In one aspect of the invention, the stabilizing agent is chosen in order to maximize the stability of FIX polypeptide in lyophilized preparations. In certain embodiments, the stabilizing agent is sucrose and/or arginine.

Bulking agents can be added to a pharmaceutical composition or formulation in order to add volume and mass to the product, thereby facilitating precise metering and handling thereof. Common bulking agents include, but are not limited to, lactose, sucrose, glucose, mannitol, sorbitol, calcium carbonate, or magnesium stearate.

Surfactants are amphipathic substances with lyophilic and lyophobic groups. A surfactant can be anionic, cationic, zwitterionic, or nonionic. Examples of nonionic surfactants include, but are not limited to, alkyl ethoxylate, nonylphenol ethoxylate, amine ethoxylate, polyethylene oxide, polypropylene oxide, fatty alcohols such as cetyl alcohol or oleyl alcohol, cocamide MEA, cocamide DEA, polysorbates, or dodecyl dimethylamine oxide. In some embodiments, the surfactant is polysorbate 20 or polysorbate 80.

The pharmaceutical compositions or formulations disclosed herein can further comprise one or more of a buffer system, a preservative, a tonicity agent, a chelating agent, a stabilizer and/or a surfactant, as well as various combinations thereof. The use of preservatives, isotonic agents, chelating agents, stabilizers and surfactants in pharmaceutical compositions or formulations is well-known to the skilled person. Reference may be made to Remington: The Science and Practice of Pharmacy, 19th edition, 1995.

In some embodiments, the pharmaceutical composition or formulation is an aqueous formulation. Such a formulation is typically a solution or a suspension, but may also include colloids, dispersions, emulsions, and multi-phase materials. The term “aqueous formulation” is defined as a formulation comprising at least 50% w/w water. Likewise, the term “aqueous solution” is defined as a solution comprising at least 50 % w/w water, and the term “aqueous suspension” is defined as a suspension comprising at least 50 % w/w water.

In some embodiments, the pharmaceutical composition or formulation disclosed herein is freeze-dried, to which the physician or the patient adds solvents and/or diluents prior to use.

Pharmaceutical compositions or formulations disclosed herein can also include a pharmaceutically acceptable antioxidant. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha- tocopherol, and the like; and (3) metal chelating agents, such as citric acid, ethylenediamine tetraacetic acid (EDTA), sorbitol, tartaric acid, phosphoric acid, and the like.

Examples of suitable aqueous and nonaqueous carriers that can be employed in the pharmaceutical compositions or formulations described herein include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions can also contain adjuvants such as preservatives, wetting agents, emulsifying agents and dispersing agents. Prevention of presence of microorganisms can be ensured both by sterilization procedures, supra, and by the inclusion of various antibacterial and antifungal agents, for example, paraben, chlorobutanol, phenol sorbic acid, and the like. It can also be desirable to include isotonic agents, such as sugars, sodium chloride, and the like into the compositions. In addition, prolonged absorption of the injectable pharmaceutical form can be brought about by the inclusion of agents which delay absorption such as aluminum monostearate and gelatin.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions described herein is contemplated. A pharmaceutical composition or formulation can comprise a preservative or can be devoid of a preservative. Supplementary active compounds can be incorporated into the compositions.

Pharmaceutical compositions or formulations typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, the compositions can include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated herein. In the case of sterile powders for the preparation of sterile injectable solutions, some methods of preparation are vacuum drying and freeze- drying (lyophilization) that yield a powder of the active ingredient plus any additional desired ingredient from a previously sterile-filtered solution thereof.

The amount of active ingredient which can be combined with a carrier material in the pharmaceutical compositions or formulations disclosed herein can vary. In some embodiments, the amount of active ingredient which can be combined with a carrier material is the amount that produces a therapeutic effect. Generally, out of one hundred percent, this amount will range from about 0.01 percent to about ninety-nine percent of active ingredient, from about 0.1 percent to about 70 percent, or from about 1 percent to about 30 percent of active ingredient in combination with a pharmaceutically acceptable carrier.

The pharmaceutical composition or formulation disclosed herein can be prepared with carriers that protect the active ingredient against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and poly lactic acid. Many methods for the preparation of such formulations are patented or generally known to those skilled in the art. See. e.g., Sustained and Controlled Release Drug Delivery Systems, J.R. Robinson, ed., Marcel Dekker, Inc., New York, 1978.

In some embodiments, the fusion proteins or anti-PD-L1 antibodies or antigen-binding fragments described herein can be formulated to ensure proper distribution in vivo. For example, the blood-brain barrier (BBB) excludes many highly hydrophilic compounds. To ensure that the activate ingredient described herein cross the BBB (if desired, e.g., for brain cancers), they can be formulated, for example, in liposomes. For methods of manufacturing liposomes, see, e.g., U.S. Pat. 4,522,811; 5,374,548; and 5,399,331. The liposomes can comprise one or more moieties which are selectively transported into specific cells or organs, thus enhance targeted drug delivery (see, e.g., V.V. Ranade (1989) J. Clin. Pharmacol. 29:685). Exemplary targeting moieties include folate or biotin (see, e.g., U.S. Pat. 5,416,016 to Low et al) mannosides (Umezawa et al, (1988) Biochem. Biophys. Res. Commun. 153: 1038); antibodies (P.G. Bloeman et al. (1995) FEBS Lett. 357: 140; M. Owais et al. (1995) Antimicrob. Agents Chemother. 39: 180); surfactant protein A receptor (Briscoe et al. (1995) Am. J. Physiol. 1233: 134); pl20 (Schreier et al. (1994) J. Biol. Chem. 269:9090); see also K. Keinanen; M.L. Laukkanen (1994) FEBS Lett. 346: 123; J.J. Killion; I.J. Fidler (1994) Immunomethods 4:273.

### 6. Methods and Uses

The present disclosure also provides methods of use of the fusion proteins, anti-PD-L1 antibodies or antigen-binding fragments thereof, or pharmaceutical compositions disclosed herein in treating cancer or tumor. In some embodiments, the tumor or cancer is a PD-L1 expressing tumor or cancer. In some embodiments, provided herein are methods of treating tumor or cancer in a subject in need thereof, comprising administering to the subject a therapeutically effective amount of the fusion protein disclosed herein. In some embodiments, provided herein are uses of the fusion protein disclosed herein in treatment of tumor or cancer. In some embodiments, provided herein are uses of the fusion protein provided herein for the preparation of a medicament for the treatment of tumor or cancer. In some embodiments, provided herein are methods of treating tumor or cancer in a subject in need thereof, comprising administering to the subject a therapeutically effective amount of the anti-PD-L1 antibody or antigen-binding fragment disclosed herein. In some embodiments, provided herein are uses of the anti-PD-L1 antibody or antigen-binding fragment disclosed herein in treatment of tumor or cancer. In some embodiments, provided herein are uses of the anti-PD-L1 antibody or antigen-binding fragment provided herein for the preparation of a medicament for the treatment of tumor or cancer. In some embodiments, provided herein are methods of treating tumor or cancer in a subject in need thereof, comprising administering to the subject a therapeutically effective amount of the therapeutic composition disclosed herein. In some embodiments, provided herein are uses of the therapeutic composition disclosed herein in treatment of tumor or cancer. In some embodiments, provided herein are uses of the therapeutic composition provided herein for the preparation of a medicament for the treatment of tumor or cancer.

In some embodiments, cancers or tumors that can be treated with the fusion proteins, anti-PD-L1 antibodies or antigen-binding fragments thereof, or pharmaceutical compositions disclosed herein are solid tumors. In some embodiments, the cancer or tumor can be gastric cancer, esophageal cancer, liver cancer, lung cancer (e.g., small cell lung cancer, or non-small cell lung cancer), head and neck cancer, skin cancer, nasopharyngeal cancer, thyroid cancer, kidney cancer, colorectal cancer, endometrial cancer, breast cancer, pancreatic cancer, testicular cancer, cervical cancer, ovarian cancer, endometrial cancer, uterine cancer, prostate cancer, bladder cancer, endocrine cancer, basal cell cancer, squamous cell cancer, dermatofibrosarcoma protuberans, mesothelioma, Merkel cell carcinoma, glioma, glioblastoma, or sarcoma. In some embodiments, cancers or tumors that can be treated with the fusion proteins, anti-PD-L1 antibodies or antigen-binding fragments thereof, or pharmaceutical compositions disclosed herein are hematological cancer. In some embodiments, the hematological cancer can be lymphoma, or leukemia, or myelodysplastic syndrome (MDS). In some embodiments, the hematological cancer can be acute myeloid leukemia (AML), chronic myelomonocytic leukemia (CMML), natural killer cell lymphoma (NK lymphoma), natural killer cell leukemia (NK leukemia), cutaneous T-Cell lymphoma (CTCL), peripheral T-cell lymphoma (PTCL), or chronic myeloid leukemia (CML).

In cancer treatment, eliminating cancer or tumor cells in a subject can occur, but any clinical improvement constitutes a benefit. Clinical improvement comprises decreased risk or rate of progression or reduction in pathological consequences of the cancer or tumor. It is also understood that a method of treating cancer can include any effect that ameliorates a sign or symptom associated with cancer. Such signs or symptoms include, but are not limited to, reducing tumor burden, including inhibiting growth of a tumor, slowing the growth rate of a tumor, reducing the size of a tumor, reducing the number of tumors, eliminating a tumor, all of which can be measured using routine tumor imaging techniques well known in the art. Other signs or symptoms associated with cancer include, but are not limited to, fatigue, pain, weight loss, and other signs or symptoms associated with various cancers.

In some embodiments, the methods or uses provided herein can reduce tumor burden. Thus, administration of the pharmaceutical composition disclosed herein can reduce the number of tumor cells, reduce tumor size, and/or eradicate the tumor in the subject. In some embodiments, the methods disclosed herein can decrease the number of PD-L1 positive tumor cells. In some embodiments, the methods disclosed herein can decrease tumor burden in the subject. Methods for monitoring patient response to administration of a pharmaceutical composition disclosed herein are known in the art and can be employed in accordance with methods disclosed herein. In some embodiments, methods known in the art can be employed to monitor the patient for response to administration of a pharmaceutical composition disclosed herein. In some embodiments, methods known in the art can be used to monitor size of lesions, and/or size of lymph nodes. As a non-limiting example, in some embodiments, contrast-enhanced CT scans can detect and/or monitor lesions and/or lymph nodes in a patient. In some embodiments, administration of a pharmaceutical composition disclosed herein can reduce the size of lesions detected by CT scans in a patient. In some embodiments, administration of a pharmaceutical composition disclosed herein can cause shrinkage of abnormal lymph nodes. In some embodiments, the methods or uses provided herein can provide for increased or lengthened survival of a subject having cancer. In some embodiments, the methods or uses provided herein can provide for an increased immune response in the subject against the cancer.

In the methods disclosed herein, a therapeutically effective amount of the fusion proteins, anti-PD-L1 antibodies or antigen-binding fragments thereof, or pharmaceutical compositions disclosed herein is administered to a subject in need of cancer treatment. The subject can be a mammal. In some embodiments, the subject is a human. Another group of suitable subjects can be a subject who has a history of cancer, but has been responsive to another mode of therapy. The prior therapy can have included, but is not restricted to, surgical resection, radiotherapy, and chemotherapy. In some embodiments, these individuals have no clinically measurable tumor. However, they are suspected of being at risk for progression of the disease, either near the original tumor site, or by metastases. This group can be further subdivided into high-risk and low-risk individuals. The subdivision is made on the basis of features observed before or after the initial treatment. These features are known in the clinical arts and are suitably defined for different types of cancers. Features typical of high-risk subgroups are those in which the tumor has invaded neighboring tissues, or who show involvement of lymph nodes.

The subject can have an advanced form of disease, in which case the treatment objective can include mitigation or reversal of disease progression, and/or amelioration of side effects. The subjects can have a history of the condition, for which they have already been treated, in which case the therapeutic objective can be to decrease or delay the risk of recurrence. Additionally, refractory or recurrent malignancies can be treated using the fusion proteins, antibodies or antigen-binding fragments or pharmaceutical compositions disclosed herein.

For treatment, the amount administered is an amount effective for producing the desired effect. An effective amount or therapeutically effective amount is an amount sufficient to provide a beneficial or desired clinical result upon treatment. An effective amount can be provided in a single administration or a series of administrations (one or more doses). An effective amount can be provided in a bolus or by continuous perfusion. In terms of treatment, an effective amount is an amount that is sufficient to palliate, ameliorate, stabilize, reverse or slow the progression of the disease, or otherwise reduce the pathological consequences of the disease. The effective amount can be determined by the physician for a particular subject. Several factors are typically considered when determining an appropriate dosage to achieve an effective amount, including for example, age, sex and weight of the subject, the condition being treated, and the severity of the condition.

A composition described herein can be administered via one or more routes of administration using one or more of a variety of methods known in the art. As will be appreciated by the skilled artisan, the route and/or mode of administration will vary depending upon the desired results. Routes of administration for the fusion proteins, anti-PD-L1 antibodies or antigen-binding fragments, or pharmaceutical compositions described herein can include intravenous, intramuscular, intradermal, intraperitoneal, subcutaneous, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrastemal injection and infusion. Alternatively, the fusion proteins, anti-PD-L1 antibodies or antigen-binding fragments, or pharmaceutical compositions described herein could potentially be administered via a non-parenteral route, such as a topical, epidermal or mucosal route of administration, for example, intranasally, orally, vaginally, rectally, sublingually or topically.

Therapeutic compositions can be administered with medical devices known in the art. For example, in some embodiments, a therapeutic composition described herein can be administered with a needleless hypodermic injection device, such as the devices disclosed in U.S. Pat. Nos. 5,399,163; 5,383,851; 5,312,335; 5,064,413; 4,941,880; 4,790,824; or 4,596,556. Examples of well- known implants and modules for use described herein include: U.S. Pat. No. 4,487,603, which discloses an implantable micro-infusion pump for dispensing medication at a controlled rate; U.S. Pat. No. 4,486,194, which discloses a therapeutic device for administering medicaments through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. These patents are incorporated herein by reference. Many other such implants, delivery systems, and modules are known to those skilled in the art.

Combination therapy using agents with different mechanisms of action can result in additive or synergetic effects. Combination therapy can allow for a lower dose of each agent than is used in monotherapy, thereby reducing toxic side effects and/or increasing the therapeutic index of the agent disclosed herein. Combination therapy can decrease the likelihood that resistant cancer cells will develop. In some embodiments, the additional therapy results in an increase in the therapeutic index of the cells or pharmaceutical compositions described herein. In some embodiments, the additional therapy results in a decrease in the toxicity and/or side effects of cells or pharmaceutical compositions described herein. In some embodiments, the pharmaceutical compositions provided herein can be administered to a subject in need thereof in combination with a second therapy. In some embodiments, the second therapy is a chemotherapy. In some embodiments, the second therapy is a radiation therapy. In some embodiments, the second therapy is an immune therapy. In some embodiments, the second therapy is a cell therapy.

The additional therapy can be administered prior to, concurrently with, or subsequent to administration of the fusion proteins, anti-PD-L1 antibodies or antigen-binding fragments, or pharmaceutical compositions described herein. Combined administration can include coadministration, either in a single pharmaceutical formulation or using separate formulations, or consecutive administration in either order but generally within a time period such that all active agents can exert their biological activities simultaneously. A person skilled in the art can readily determine appropriate regimens for administering a pharmaceutical composition described herein and an additional therapy in combination, including the timing and dosing of an additional agent to be used in a combination therapy, based on the needs of the subject being treated.

It is understood that modifications which do not substantially affect the activity of the various embodiments of this invention are also provided within the definition of the invention provided herein. Accordingly, the following examples are intended to illustrate but not limit the present invention.

### 7. Experimental

The examples provided below are for purposes of illustration only, which are not intended to be limiting unless otherwise specified. Thus, the invention should in no way be construed as being limited to the following examples, but rather, should be construed to encompass any and all variations which become evident as a result of the teaching provided herein.

### Example 1: Preparation of Anti-human PD-L1 Monoclonal Antibody

Human PD-L1 extracellular portion (Phe19-Arg238, SEQ ID NO.1) was used as immunogen, and the anti-human PD-L1 monoclonal antibody was produced by immunizing 7 week-old female BALB/c mice (Changzhou cavens lab animal Co., Ltd). Animals with high antibody titer in serum were selected for cell fusion. Hybridoma cells were obtained by fusing mouse splenocytes with SP2/0 (ATCC, CRL8287TM) through an optimized PEG-mediated fusion procedure. After FACS screening assay, the positive hybridoma cells were selected for breed conservation and a series of subcloning. Anti-PD-L1 antibodies produced by Clones A, B, C, D, F, G, H, J, K, L, N, P and Y were sequenced, and the sequences of respective antibodies are provided in Tables 1 and 2 above.

### Example 2: Humanization of Anti-PD-L1 Antibodies

Selection of the FR regions for humanization of hybridoma clones: The germlines closest to Clones B, C and D were selected from IMGT database to serve as the templates for humanization. Kabat numbering for CDRs were adopted. CDRs from Clones B, C and D were grafted to the corresponding human templates. The sequences of humanized VH and VL regions of Clones B, C and D are provided in Tables 4-6 above.

Cloning and expressing of humanized antibodies: Primers were designed for genetical engineering; VH and VL regions were obtained by PCR and cloned into expression vectors that contained signal peptide and nucleic acid sequences encoding the constant regions (CH1-FC/CL). The heavy chain and light chain of the antibodies were transfected into 293F cells for expression. Affinity was measured using Octet-96. The binding affinities of the humanized antibodies are provided below.

TABLE 8

Binding affinities of humanized antibodies.

| | |
|---|---|
| Clone | KD (M) |
| BhVH2/BhVL1 | 1.13E-09 |
| BhVH3/BhVL2 | 1.32E-09 |
| BhVH3/BhVL3 | 1.29E-09 |
| BhVH5/BhVL6 | 3.22E-09 |
| BhVH2/BhVL7 | 9.42E-10 |
| BhVH2/BhVL8 | 1.18E-09 |
| BhVH5/BhVL7 | 4.00E-10 |
| BhVH5/BhVL8 | 6.19E-10 |
| ChVH1/ChVL1 | 2.55E-10 |
| ChVH2/ChVL2 | 2.01E-10 |
| ChVH3/ChVL1 | 1.60E-10 |
| ChVH5/ChVL1 | 1.17E-09 |
| ChVH2/ChVL3 | 3.29E-10 |
| ChVH3/ChVL3 | 2.42E-10 |
| ChVH4/ChVL3 | 1.77E-10 |
| ChVH5/ChVL3 | 9.01E-10 |
| DhVH-CDR graft/ DhVL-CDR graft | 1.03E-08 |
| DhVH1/DhVL1 | 1.11E-08 |
| DhVH2/DhVL1 | 1.27E-09 |
| DhVH3/DhVL1 | 9.09E-09 |
| DhVH1/DhVL2 | 1.24E-08 |
| DhVH2/DhVL2 | 1.18E-08 |
| DhVH3/DhVL2 | 1.67E-09 |
| Chimeric D | 3.68E-09 |

### Example 3: Binding Kinetics Measurement of PD-L1 Antibody to Human PD-L1 Protein Using Biomolecular Interaction System Gator (Probe Life)

PD-L1 antibodies were coupled to the surface of the HFC sensor (Probe Life), and human PD-L1 recombinant protein as the mobile phase was 2 times gradient diluted. The association and dissociation between PD-L1 protein and the antibodies on the surface of the sensor were measured. Specifically, the HFC sensor was prewetted in SD buffer (PBS containing 0.02% Tween-20 and 0.2% BSA) for 5-10 minutes, and then incubated in SD buffer containing antibodies for 1-3 minutes, allowing antibodies to be coupled to the sensor surface. The antibody-coupled HFC sensor was incubated in the SD buffer solution for 1 minute, and then incubated in the buffer solution containing different concentrations of PD-L1-his for 1-5 minutes to measure the association of PD-L1 protein to captured antibody. Finally, the sensor combining the antigen and antibody was incubated in SD buffer for 2-10 minutes to measure PD-L1 protein’s dissociation from sensor. The measured data were fitted using a 1:1 model, and the association rate (Kon) and the dissociation rate (Koff) were calculated. The equilibrium dissociation constant (kD) was calculated by the ratio Koff/Kon.

TABLE 9

Binding kinetic analysis of anti-PD-L1 antibodies to human PD-L1

| Sample | KD(M) | Kon (1/Ms) | Kdis(1/s) | Full R² |
|---|---|---|---|---|
| B | 5.94E-09 | 1.01E+05 | 6.00E-04 | 0.9961 |
| C | 4.32E-09 | 2.34E+05 | 1.01E-03 | 0.9968 |
| D | 3.68E-09 | 2.25E+05 | 8.26E-04 | 0.9967 |
| N | 3.52E-09 | 1.65E+05 | 5.82E-04 | 0.9963 |

The binding kinetics of humanized anti-PD-L1 antibody to human PD-L1 protein are also measured using the same procedures.

### Example 4: Binding Affinity of PD-L1 Antibody to Tumor Cell Lines of Different Tissue Origin.

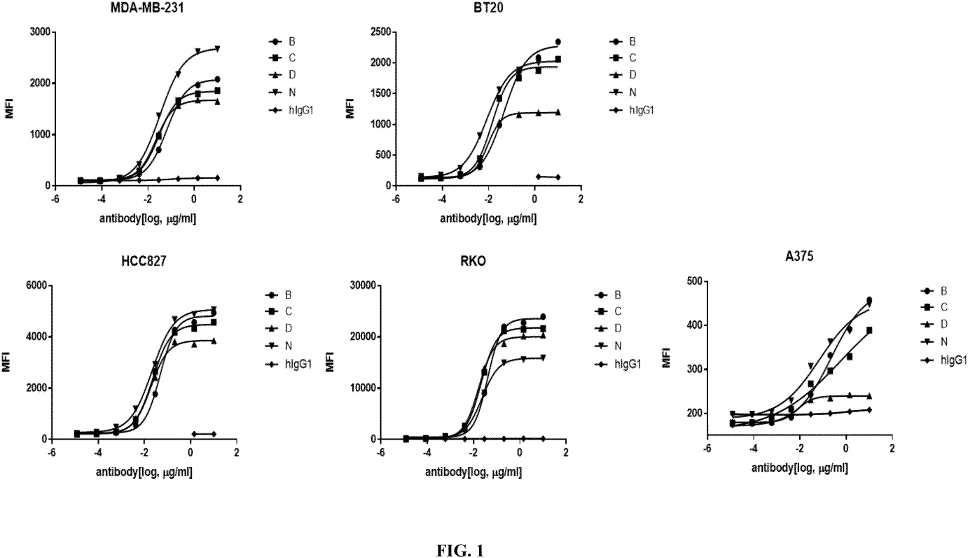

Glycosylation differences in cancer cell lines of different tissue origin may affect the binding affinity between PD-L1 antibody and PD-L1. In this study, five cell lines were selected, namely BT20(Breast), mb-231 (Breast), A375(Melanoma), HCC827(Lung), and RKO(Colon). Cells were re-suspended with FACS buffer (1%BSA in PBS), and 50 µl cell suspension per well was added to the 96-well U-bottom plate (Corning, catalog#3795). The purified antibody to be tested was diluted in gradient, then 50 µl was added to each well and incubated at 4° C. for 1 hour. After washing by FACS buffer for three times, the cells were incubated with PE-labeled fluorescent secondary antibody and incubated at 4° C. for half an hour. Cells were washed three times with FACS buffer and resuspended to 200 uL/ well. The fluorescence signal was detected using Canto II flow cytometry (BD). The binding affinities of clones B, C, D, and N with various cancer cell lines are shown in FIG. 1.

The binding affinities of humanized anti-PD-L1 antibodies to different tumor cell lines are also measured using the same procedures.

### Example 5: Measuring the Blocking Effect of PD-L1 Antibody on the Binding of PD-L1 to its Receptor PD1 or CD80

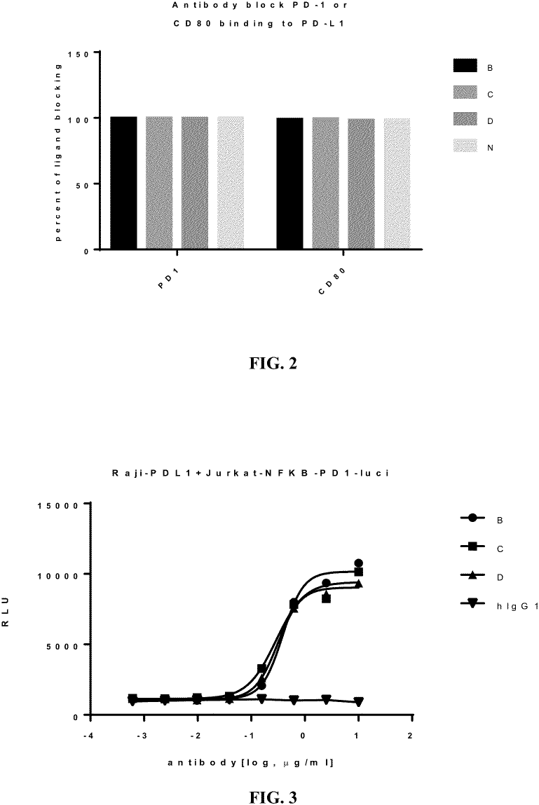

hPD1-Fc or CD80-Fc were diluted with PBS to 2 ug/mL. 50 ul was added to each well of the 96-well plate, which was incubated at 4° C. overnight. The plate was then washed with wash buffer (0.05% Tween in PBS) three times and blocked with 5% skim milk in PBS at 37° C. for two hours. The biotinylated PD-L1 and various antibodies were preincubated at room temperature for half an hour and then added to the plate. The plate was then incubated at room temperature for 1 hour and washed three times with wash buffer. 50 µl streptavidin HRP (1:1000, BD) was then added to each well at room temperature and incubated for about 1 hour. 50ul of TMB substrate was added to each well, and the reaction was then terminated with 50 µl HCL after 1-5 minutes. Absorbance was read at 450 nm and IC50 value was analyzed. As shown in FIG. 2, Clones B, C, D and N all completed blocked the binding of PD-1 or CD80 to PD-L1.

The blocking effect of humanized anti-PD-L1 antibodies on the binding of PD-L1 to its receptor PD1 or CD80 is also measured using the same procedures.

### Example 6: Anti-PD-L1 Antibodies Further Enhance the Jurkat Reporter Cell Activation

The function of various anti-PD-L1 antibodies to block the intercellular PD1/PD-L1 interaction was examined. Exogenous Luciferase reporter gene under the control of a NF-kB promoter with human PD1 were introduced into Jurkat cells. Raji/hPD-L1 cells (antigen presenting cells) and OKT3 (anti-CD3) could co-stimulate the activation of Jurkat/hPD1/NF-KB cells (effector cells), which produced a luciferase signal. The activation was inhibited to some extent by PD-L1 and PD1 interaction, and anti-PD-L1 antibodies that could block the intercellular PD1/PD-L1 interaction would restore the expression of luciferase reporter gene in Jurkat/hPD1/NFKB- Luciferase cells. To measure the activity of the anti-PD-L1 antibodies, Raji/hPD-L1 cells and Jurkat/hPD1/ NFKB-luciferase cells were mixed, OKT3 and gradient-diluted antibody were added to the mixture, and luciferase assay kit (Promega, G7940) was used for detection after the cells were incubated for 4-6 hours. As shown in FIG. 3, addition the anti-PD-L1 antibodies (Clone B, C, or D) enhanced the Jurkat cell activation in a dose-dependent manner, indicating that these antibodies were able to block the intercellular PD1/PD-L1 interaction.

The function of humanized anti-PD-L1 antibodies to block the intercellular PD1/PD-L1 interaction is also examined using the same procedures.

### Example 7: PD-L1 Chimeric Antibody Increased IFN-γ Secretion in Mixed Lymphocyte Reaction (MLR)

The activation of the immune effector cells by the anti-PD-L1 antibodies (Clone B, C, D or N) was also confirmed by cytokine release assays. Specifically, peripheral blood mononuclear cells (PBMC) were isolated from human peripheral blood using density gradient centrifugation (ficoll-paque Premium, GE Healthcare, catalog#17-5442-02). Further, monocytes were isolated from PBMC using the CD14 Cell Isolation Kit (Miltenyi, catalog#130-050-201). The monocytes were induced to dendritic cells by adding GM-CSF (Prospec, catalog# CYt-221) and IL-4 (Prospec, Catalog) to the culture medium, and were supplemented with cytokine every 2-3 days. Cells were harvested 5-6 days later for subsequent experiments. Human CD3+T cells isolated from PBMC by negative selection were mixed with immature dendritic cells, as well as PD-L1 antibodies at different concentrations in 96-well U-bottom plate. After 4-6 days of co-culture, the supernatant was harvested, and the concentration of cytokines was quantitatively detected by ELISA.

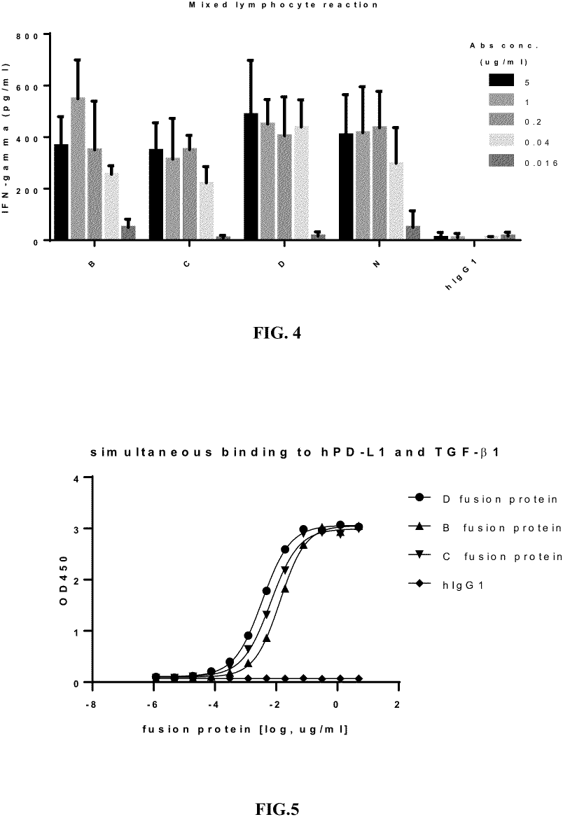

As shown in FIG. 4, Clones B, C, D, and N all induced the cytokine production by CD3+T cells, further confirming their ability to activate immune effector cells.

The activation of the immune effector cells by humanzied anti-PD-L1 antibodies is also measured by cytokine release assays using the same procedures.

### Example 8: Cloning and Expression of anti-PD-L1/TGF-β Trap Fusion Proteins

To produce the fusion proteins, an anti-PD-L1 antibody serving as the targeting domain was linked to the extracellular domain of TGF-β RII (or a truncated or mutated variant thereof), which served as the TGF-β trap. A transferrin linker was used to link the two domains. Sequences of various TGF-β trap domains and transferrin linkers are provided in Table 10 below.

TABLE 10

Sequences of extracellular domain of TGF-β RII and transferrin linkers

| | |
|---|---|
| | Sequences |
| TGF-βRII ECD (1-136) | IPPHVQKSVNNDMIVTDNNGA VKFPQLCKFCDVRFSTCDNQKSCMSN CSITSICEKPQEVCVAVWRKNDENITLETVCHDPKLPYHDFILEDAASP KCIMKEKKKPGETFFMCSCSSDECNDNIIFSEEYNTSNPD (SEQ ID NO:8) |
| TGF-βRII ECD mutant (1-136) | IGGSVQGSVGGSMIVTGGSGAVGFPQLCKFCDVRFSTCDNQKSCMSN CSITSICEKPQEVCVAVWRKNDENITLETVCHDPKLPYHDFILEDAASP KCIMKEKKKPGETFFMCSCSSDECNDNIIFSEEYNTSNPD (SEQ ID NO:9) |
| TGF-βRII ECD mutant N19T (1-136) | IPPHVQKSVNNDMIVTDNTGA VKFPQLCKFCDVRFSTCDNQKSCMSN CSITSICEKPQEVCVAVWRKNDENITLETVCHDPKLPYHDFILEDAASP KCIMKEKKKPGETFFMCSCSSDECNDNIIFSEEYNTSNPD (SEQ ID NO:201) |
| TGF-βRII ECD mutant N19A (1-136) | IPPHVQKSVNNDMIVTDNAGAVKFPQLCKFCDVRFSTCDNQKSCMSN CSITSICEKPQEVCVAVWRKNDENITLETVCHDPKLPYHDFILEDAASP KCIMKEKKKPGETFFMCSCSSDECNDNIIFSEEYNTSNPD (SEQ ID NO:202) |
| TGF-βRII ECD mutant K7G N19A (1-136) | IPPHVQGSVNNDMIVTDNAGAVKFPQLCKFCDVRFSTCDNQKSCMSN CSITSICEKPQEVCVAVWRKNDENITLETVCHDPKLPYHDFILEDAASP KCIMKEKKKPGETFFMCSCSSDECNDNIIFSEEYNTSNPD (SEQ ID NO:203) |
| TGF-βRII ECD N-terminal truncation (20-136) | GAVKFPQLCKFCDVRFSTCDNQKSCMSNCSITSICEKPQEVCVAVWR KNDENITLETVCHDPKLPYHDFILEDAASPKCIMKFKKKPGETFFMCS CSSDECNDNIIFSEEYNTSNPD (SEQ ID NO:10) |
| TGF-βRII ECD C-terminal truncation (1-130) | IPPHVQKSVNNDMIVTDNNGA VKFPQLCKFCDVRFSTCDNQKSCMSN CSITSICEKPQEVCVAVWRKNDENITLETVCHDPKLPYHDFILEDAASP KCIMKEKKKPGETFFMCSCSSDECNDNIIFSEEY (SEQ ID NO:11) |
| TGF-βRII ECD C-terminal truncation (1-127) | IPPHVQKSVNNDMIVTDNNGA VKFPQLCKFCDVRFSTCDNQKSCMSN CSITSICEKPQEVCVAVWRKNDENITLETVCHDPKLPYHDFILEDAASP KCIMKEKKKPGETFFMCSCSSDECNDNIIFS (SEQ ID NO:12) |
| TGF-βRII ECD C-terminal truncation and mutant N19A (1-127) | IPPHVQKSVNNDMIVTDNAGA VKFPQLCKFCDVRFSTCDNQKSCMSN CSITSICEKPQEVCVAVWRKNDENITLETVCHDPKLPYHDFILEDAASP KCIMKEKKKPGETFFMCSCSSDECNDNIIFS (SEQ ID NO:204) |
| TGF-βRII ECD C-terminal truncation and mutant K7G, N19A (1-127) | IPPHVQGSVNNDMIVTDNAGAVKFPQLCKFCDVRFSTCDNQKSCMSN CSITSICEKPQEVCVAVWRKNDENITLETVCHDPKLPYHDFILEDAASP KCIMKEKKKPGETFFMCSCSSDECNDNIIFS (SEQ ID NO:205) |
| TGF-βRII ECD C-terminal truncation and mutant Q6G, K7G, N19A (1-127) | IPPHVGGSVNNDMIVTDNAGAVKFPQLCKFCDVRFSTCDNQKSCMSN CSITSICEKPQEVCVAVWRKNDENITLETVCHDPKLPYHDFILEDAASP KCIMKEKKKPGETFFMCSCSSDECNDNIIFS (SEQ ID NO:232) |
| Transferrin linker | (PEAPTD)m, m=1, 2, 3, 4, or 5 (SEQ ID NO:18) |
| Transferrin linker | (PEAPTDE)n, n=1, 2, 3, 4, or 5 (SEQ ID NO:19) |
| Transferrin linker | (PEAPTD)nP, n=1, 2, 3, 4, or 5 (SEQ ID NO:220) |
| Transferrin linker | (PEAPTD)nPE, n=1, 2, 3, 4, or 5 (SEQ ID NO:221) |
| Transferrin linker | (PEAPTD)nPEA, n=1, 2, 3, 4, or 5 (SEQ ID NO:222) |
| Transferrin linker | (PEAPTD)nPEAP, n=1, 2, 3, 4, or 5 (SEQ ID NO:223) |
| Transferrin linker | (PEAPTD)nPEAPT, n=1, 2, 3, 4, or 5 (SEQ ID NO:224) |
| Transferrin linker | (PEAPTDE)nP, n=1, 2, 3, 4, or 5 (SEQ ID NO:225) |
| Transferrin linker | (PEAPTDE)nPE, n=1, 2, 3, 4, or 5 (SEQ ID NO:226) |
| Transferrin linker | (PEAPTDE)nPEA, n=1, 2, 3, 4, or 5 (SEQ ID NO:227) |
| Transferrin linker | (PEAPTDE)nPEAP, n=1, 2, 3, 4, or 5 (SEQ ID NO:228) |
| Transferrin linker | (PEAPTDE)nPEAPT, n=1, 2, 3, 4, or 5 (SEQ ID NO:229) |
| Transferrin linker | (PEAPTDE)nPEAPTD, n=1, 2, 3, 4, or 5 (SEQ ID NO:230) |

A number of fusion proteins were produced by linking monoclonal anti-PD-L1 antibodies disclosed herein with TGF-β trap domains. Here, the fusion protein included (1) a heavy chain having the heavy chain of an anti-PD-L1 antibody linked with a TGF-β trap domain and (2) a light chain having the light chain of the anti-PD-L1 antibody. Both heavy chain and light chain of the fusion protein were co-transfected into 293 cells to produce the fusion protein provided below.

TABLE 11

Exemplary anti-PD-L1/TGF-β Trap fusion proteins

| Fusion Protein | Structures |
|---|---|
| 1 | Ab-(G4S)4G-ECD(1-136) |
| 2 | Ab-ECD mutant(1-136) |
| 3 | Ab-(PEAPTD)2-ECD(1-136) |
| 4 | Ab-(PEAPTD)3-ECD(1-136) |
| 5 | Ab-(PEAPTD)4-ECD(1-136) |
| 6 | Ab-(PEAPTD)5-ECD(1-136) |
| 7 | Ab-(PEAPTDE)2-ECD(1-136) |
| 8 | Ab-(PEAPTDE)3-ECD(1-136) |
| 9 | Ab-(PEAPTDE)4-ECD(1-136) |
| 10 | Ab-(PEAPTDE)5-ECD(1-136) |
| 11 | Ab-(PEAPTD)2-ECD(20-136) |
| 12 | Ab-(PEAPTD)3-ECD(20-136) |
| 13 | Ab-(PEAPTD)4-ECD(20-136) |
| 14 | Ab-(PEAPTD)5-ECD(20-136) |
| 15 | Ab-(PEAPTDE)2-ECD(20-136) |
| 16 | Ab-(PEAPTDE)3-ECD(20-136) |
| 17 | Ab-(PEAPTDE)4-ECD(20-136) |
| 18 | Ab-(PEAPTDE)5-ECD(20-136) |
| 19 | Ab-(PEAPTD)2-ECD(1-130) |
| 20 | Ab-(PEAPTD)3-ECD(1-130) |
| 21 | Ab-(PEAPTD)4-ECD(1-130) |
| 22 | Ab-(PEAPTD)5-ECD(1-130) |
| 23 | Ab-(PEAPTDE)2-ECD(1-130) |
| 24 | Ab-(PEAPTDE)3-ECD(1-130) |
| 25 | Ab-(PEAPTDE)4-ECD(1-130) |
| 26 | Ab-(PEAPTDE)5-ECD(1-130) |
| 27 | Ab-(PEAPTD)2-ECD(1-127) |
| 28 | Ab-(PEAPTD)3-ECD(1-127) |
| 29 | Ab-(PEAPTD)4-ECD(1-127) |
| 30 | Ab-(PEAPTD)5-ECD(1-127) |
| 31 | Ab-(PEAPTDE)2-ECD(1-127) |
| 32 | Ab-(PEAPTDE)3-ECD(1-127) |
| 33 | Ab-(PEAPTDE)4-ECD(1-127) |
| 34 | Ab-(PEAPTDE)5-ECD(1-127) |

### Example 9: Purification of Anti-PD-L1/TGF-β Trap Fusion Proteins

The culture solution of cells transfected with the fusion protein was centrifuged at high speed and the supernatant was collected. After filtration with a 0.45 µm filter membrane, the first step of purification was carried out by affinity chromatography. Protein A that interacted with Fc or derivative packing (such as GE’s Mabselect) was used as chromatographic medium. 10 times the column volume of equilibration buffer (1xPBS: 5.6 mmol/L Na₂HPO₄, 1.06 mmol/L KH₂PO₄, 154 mmol/L NaCl, pH7.2) was used, and the cell supernatant was then loaded and combined. The flow rate was adjusted such that it took 1 minute for the sample to go through the column. The column was washed with 1xPBS until the UV absorption of A280 dropped to the baseline and eluted with elution buffer (50 mM PBS (50 mmol/L Na₂HPO₄, 1.06 mmol/L KH₂PO₄, 154 mmol/L NaCl, pH 3.0). The sample at the elution peak according to the A280 ultraviolet absorption was collected, neutralized with 1 M NaOH, concentrated by ultrafiltration and filtered with a 0.22 µm membrane. The purity of the fusion protein was determined to be greater than 95% by SEC-HPLC, and the sequence confirmed by LS-MS.

### Example 10: Binding of the Anti-PD-L1/TGF-β Trap Fusion Proteins With PD-L1 and TGF-β1 (ELISA)

The binding between the fusion proteins and PD-L1 or TGF-β1 was measured in vitro by ELISA as follows: a. 96-well plate was coated with 2 µg/ml hPD-L1 (60 µl per well) overnight at 4° C.; b. the plate was washed with 200 µl PBST 3 times, and blocked with 200 µl 1% BSA in PBS at 37° C. for 2 hours; c. the plate was washed 3 times with 200 µl PBST before 0-20000 ng/ml gradient dilution of fusion protein was added, and incubated at 37° C. for 1 h; d. the plate was washed 3 times with 200 µl PBST; 100 ng/ml rhTGF-β was washed and incubate at 37° C. for 1 h; e. the plate was washed 3 times with 200 µl PBST; 200 ng/ml anti-TGF-βbiotinylated antibody was added and incubated at 37° C. for 1 h; f. the plate was washed 3 times with 200 µl PBST; 200 ng/ml anti-TGF-βbiotinylated antibody was added and incubated at 37° C. 1 h; g. the plate was washed with 200 µl PBST 3 times; 1:10000 diluted Streptavidin HRP was added and incubated at room temperature for 15 min; h. the plate was washed with 200 µl PBST 3 times and subject to TMB color development; HCl was used to stop the reaction, and OD450 detected with a microplate reader; Graphpad Prism was used to analyze the binding data. As shown in FIG. 5, the simultaneous binding between fusion proteins and both PD-L1 and TGF-β1 was detected. Fusion proteins used in this study included B fusion (SEQ ID NOs: 155 and 162), C fusion (SEQ ID NOs: 160 and 164), and D fusion (SEQ ID NOs: 159 and 163).

### Example 11: Binding Kinetics of Human PD-L1-His or TGF- 1 to Fusion Protein

Biomolecular interaction system Gator (Probe Life) was used to measure the kinetics between fusion protein and antigens. Fusion protein was coupled to the surface of the HFC sensor (Probe Life), and the PD-L1-his (i.e., PD-L1 with a his-tag) or TGF-1 recombinant protein as the mobile phase was 2 times gradient diluted, the association and dissociation between PD-L1-his or TGF- 1 and fusion protein on the surface of the sensor were measured. Specifically, the HFC sensor was prewetted in SD buffer (PBS containing 0.02%Tween-20 and 0.2%BSA) for 5-10 minutes, and then incubated in a sample buffer containing antibodies for 1-3 minute, allowing fusion protein to be coupled to the sensor surface. The fusion protein-coupled HFC sensor was first incubated in the buffer solution for 1 minute, and then placed in the buffer solution containing different concentrations of PD-L1-his or TGF- 1 and co-incubated for 1-5 minutes to measure the association of the fusion protein to PD-L1 his or TGF- 1. Finally, the sensor combining the antigen and fusion protein was incubated in SD buffer for 2-10 minutes to measure the dissociation of PD-L1-his or TGF- 1 proteins from fusion protein. The data were fitted as a whole using a 1:1 model, and the binding rate (Kon) and the departure rate (Koff) were calculated. The equilibrium dissociation constant (KD) is calculated by the ratio koff/ Kon. Fusion proteins used in this study included B fusion (SEQ ID NOs:155 and 162), C fusion (SEQ ID NOs: 160 and 164), and D fusion (SEQ ID NOs: 159 and 163); B fusion protein-23 (SEQ ID NOs:234 and 162), B fusion protein-24 (SEQ ID NOs:235 and 162), B fusion protein-25 (SEQ ID NOs:236 and 162), and B fusion protein-26 (SEQ ID NOs:237 and 162).

TABLE 12

Binding affinity detection of fusion protein binding to PD-L1 or TGF- Beta 1

| Loading | antigen | KD(M) | kon(1/Ms) | koff(1/s) | FullR2 |
|---|---|---|---|---|---|
| B fusion protein | PD-L1 his | 8.43E-09 | 1.93E+05 | 1.63E-03 | 0.991 |
| | TGF-beta 1 | 6.79E-10 | 2.17E+06 | 1.47E-03 | 0.961 |
| D fusion protein | PD-L1 his | 6.21E-09 | 1.37E+05 | 8.48E-04 | 0.993 |
| | TGF-beta 1 | 7.85E-10 | 2.28E+06 | 1.79E-03 | 0.933 |
| C fusion protein | PD-L1 his | 5.03E-09 | 1.52E+05 | 7.64E-04 | 0.986 |
| | TGF-beta 1 | 7.50E-10 | 2.20E+06 | 1.71E-03 | 0.955 |
| B fusion protein -23 | PD-L1 his | 1.05E-09 | 5.60E+05 | 5.90E-04 | 0.994 |
| | TGF-beta 1 | 3.31E-10 | 3.75E+05 | 1.24E-04 | 0.989 |
| B fusion protein -24 | PD-L1 his | 1.31E-09 | 5.07E+05 | 6.62E-04 | 0.991 |
| | TGF-beta 1 | 7.58E-10 | 3.87E+05 | 2.94E-04 | 0.985 |
| B fusion protein -25 | PD-L1 his | 9.10E-10 | 5.45E+05 | 4.96E-04 | 0.993 |
| | TGF-beta 1 | 4.36E-10 | 5.38E+05 | 2.35E-04 | 0.982 |
| B fusion protein -26 | PD-L1 his | 1.28E-09 | 5.52E+05 | 7.10E-04 | 0.991 |
| | TGF-beta 1 | 7.27E-10 | 4.95E+05 | 3.60E-04 | 0.983 |

Additionally, the binding affinities to FcγRn by the fusion proteins were also measured. The results are shown in the table below.

TABLE 13

Binding affinity detection of fusion protein binding to FcγRn

| Loading | KD(M) | kon(1/Ms) | koff(1/s) | FullR2 |
|---|---|---|---|---|
| B fusion protein - 1 | 1.57E-08 | 7.80E+04 | 1.22E-03 | 0.985 |
| B fusion protein - 24 | 4.43E-09 | 4.08E+05 | 1.81E-03 | 0.983 |
| B fusion protein - 25 | 1.86E-09 | 9.88E+05 | 1.84E-03 | 0.981 |

Fusion proteins used in this study included B fusion protein-24 (SEQ ID NOs:235 and 162), B fusion protein-25 (SEQ ID NOs:236 and 162), and B fusion protein-1 (SEQ ID NOs: 155 and 162).

### Example 12: Inhibition of Smad Signaling Pathway by Fusion Protein as Measured by in Vitro Reporter Gene System

The inhibitory activity on TGFβ signaling pathway by the fusion proteins was evaluated by SMAD luciferase reporter assay. Plasmids containing SMAD binding box were transfected into TGFβ receptor expressing cells, such as tumor cell lines and engineered cell lines (e.g., HEK293T). Transfected cells were incubated with serial diluted fusion proteins followed by TGFβ stimulation to activate SMAD protein and induce the expression of luciferase. The inhibitory effect of fusion proteins on SMAD phosphorylation was shown as reduction of relative light unit (RLU). As shown in FIG. 7, both B fusion protein (SEQ ID NOs:155 and 162) and C fusion protein (SEQ ID NOs: 160 and 164) inhibited SMAD signaling pathway.

### Example 13: Inhibitory Effect of Fusion Proteins on Tumor Growth in Cancer Mouse Models

Tumor cells expressing PD-L1 were inoculated subcutaneously into mice to establish a cancer mouse model. When tumor was around 100 mm³, mice with tumors that were too large or too small were discarded, and the remaining mice were randomized into cohorts and administered with different dosages of fusion proteins. Each cohort contained 6-10 mice. Mice were checked every other day and body weight & tumor volume were recorded. The formula of tumor volume: long diameter × short diameter × short dimeter/2. As shown in FIG. 8, B fusion protein (SEQ ID NOs: 155 and 162) significantly inhibited tumor growth, to a greater extent than Clone B mAb.

### Example 14: Stability of the Fusion Proteins Under Stress

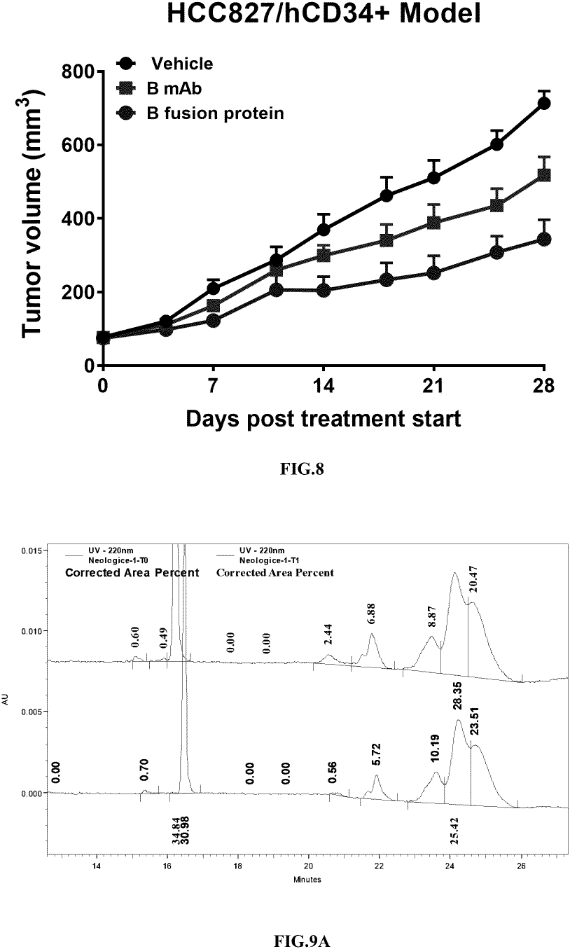

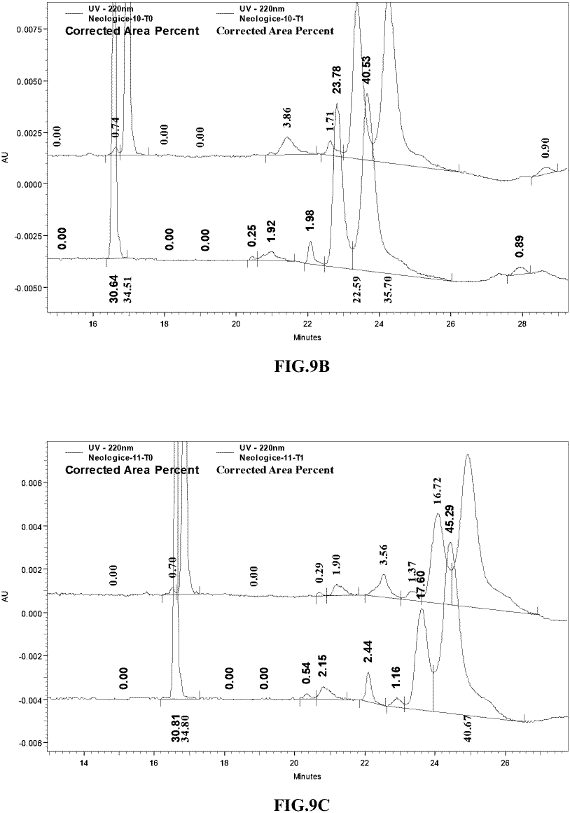

Fusion proteins were diluted to 1 mg/ml, 40° C. for 15 days, detected by capillary electrophoresis. As shown in FIGS. 9A-9C, all three fusion proteins remained stable after 15 days storage under stress. Fusion proteins used in this study included B fusion protein-1 (SEQ ID NOs: 155 and 162; FIG. 9A), B fusion protein-24 (SEQ ID NOs:235 and 162; FIG. 9B), and B fusion protein-25 (SEQ ID NOs:236 and 162; FIG. 9C).

### Example 15: Stability of the Fusion Proteins With Different Linkers

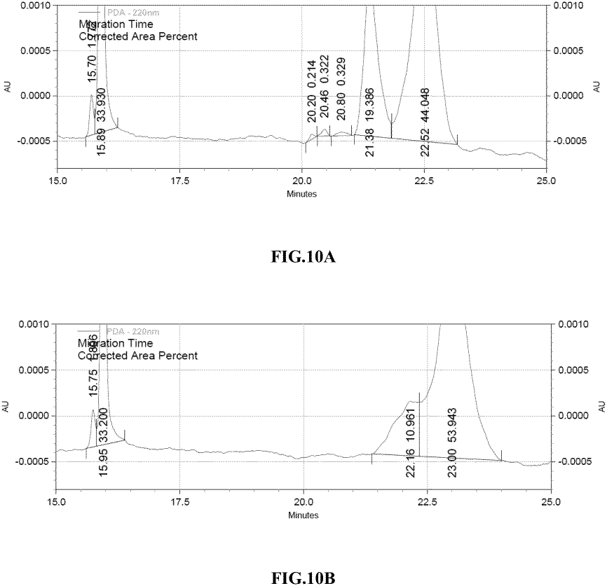

To verify that the rigid linker provided herein could confer stability to fusion proteins, fusion protein M7824 (SEQ ID NOs:241 and 243) was modified to replace its flexible linker (namely, GGGGSGGGGSGGGGSGGGGSG, SEQ ID NO:244) with a rigid linker (PEAPTDPEAPTDPEAPTD, SEQ ID NO:147). Fusion proteins were expressed by CHO cells. After purification, fusion proteins were detected by capillary electrophoresis and Uncle. As shown in FIGS. 10A-10B M7824 with a rigid linker (SEQ ID NOs:242 and 243; FIG. 10B) showed superior stability compared to M7824, which had a flexible linker (SEQ ID NOs:241 and 243; FIG. 10A). Consistently, as shown in Table 14 below, M7824 with rigid linker had higher Tₒₙₛₑₜ and T_{agg} compared to the fusion protein with flexible linker.

TABLE 14

Fusion protein Tₘ, T_{agg}

| Sample | Concentration | Average Tₘ₁ (°C) | Average Tonset (°C) (2%) | Average Tonset (°C) (10%) | Average T_{agg} 266 (°C) |
|---|---|---|---|---|---|
| M7824 | 5 mg/ml | 69.5 | 55.3 | 61.8 | 63.2 |
| M7824 with rigid linker | 5 mg/ml | 69.4 | 58.8 | 61.7 | 64.7 |

What is claimed is:

1-74. (canceled)

75. A fusion protein comprising (1) a first domain comprising an antibody that binds Programmed Death Ligand 1 (PD-L1), or an antigen-binding fragment thereof, (2) a transferrin linker, and (3) a second domain comprising a fragment of transforming growth factor β receptor type 2 (TGFβRII) that binds transforming growth factor β (TGFβ), or a variant thereof.

76. The fusion protein of claim 75, wherein the transferrin linker links the C-terminus of the first domain to the N-terminus of the second domain.

77. The fusion protein of claim 75, wherein the transferrin linker is (PEAPTD)m, m=1, 2, 3, 4, or 5 (SEQ ID NO:18) or (PEAPTDE)n, n=1, 2, 3, 4, or 5 (SEQ ID NO:19), or has an amino acid sequence selected from the group consisting of SEQ ID NOs:220-230; wherein optionally the transferrin linker is (PEAPTD)₃(SEQ ID NO:147).

78. The fusion protein of claim 75, wherein the second domain comprises the ECD of TGFβRII isoform 1 (SEQ ID NO:8) or the ECD of TGFβRII isoform 2 (SEQ ID NO:14), or a variant thereof that has at least 85%, at least 90%, at least 95%, or at least 98% sequence identity to SEQ ID NO:8 or 14; or wherein the second domain has the amino acid sequence of SEQ ID NO:9.

79. The fusion protein of claim 78, wherein the second domain comprises a variant of the ECD of TGFβRII isoform 1 (SEQ ID NO:8), wherein the variant comprises an amino acid mutation at Q6, K7, N19 or G20 of SEQ ID NO:8; and wherein optionally the second domain has an amino acid sequence selected from the group consisting of SEQ ID NOs:201-203.

80. The fusion protein of claim 78, wherein the second domain comprises a truncated form of the ECD of TGFβRII isoform 1 (SEQ ID NO:8), which lacks amino acid residues 1 to n of SEQ ID NO:8, wherein n ranges from 2 to 30, or a variant thereof; and wherein optionally n is 19.

81. The fusion protein of claim 78, wherein the second domain comprises a truncated form of the ECD of TGFβRII isoform 1 (SEQ ID NO:8), which lacks amino acid residues m to 136 of SEQ ID NO:8, wherein m ranges from 80 to 135, or a variant thereof; wherein optionally m is 131; or wherein optionally m is 128, and wherein optionally the second domain has the amino acid sequence selected from the group consisting of SEQ ID NOs:204, 205 and 232.

82. The fusion protein of claim 75, wherein the first domain is a PD-L1 antibody selected from the group consisting of durvalumab, avelumab, atezolizumab, envafolimab, BMS-936559, CK-301, CS-1001, SHR-1316, and BGB-A333.

83. An antibody or antigen-binding fragment thereof that binds PD-L1, comprising: (a) a heavy chain variable region (VH) comprising
(1) a heavy chain CDR1 (VH CDR1) having an amino acid sequence selected from the group consisting of SEQ ID NOs:20, 26, 32, 38, 44, 50, 56, 62, 68, 74, 80, 86, and 92;
(2) a heavy chain CDR2 (VH CDR2) having an amino acid sequence selected from the group consisting of SEQ ID NOs:21, 27, 33, 39, 45, 51, 57, 63, 69, 75, 81, 87 and 93; and
(3) a heavy chain CDR3 (VH CDR3) having an amino acid sequence selected from the group consisting of SEQ ID NOs:22, 28, 34, 40, 46, 52, 58, 64, 70, 76, 82, 88 and 94; or a variant thereof having up to about 5 amino acid substitutions, additions, and/or deletions in the VH CDRs; and/or
(b) a light chain variable region (VL) comprising
(1) a light chain CDR1 (VL CDR1) having an amino acid sequence selected from the group consisting of SEQ ID NOs:23, 29, 35, 41, 47, 53, 59, 65, 71, 77, 83, 89, and 95;
(2) a light chain CDR2 (VL CDR2) having an amino acid sequence selected from the group consisting of SEQ ID NOs:24, 30, 36, 42, 48, 54, 60, 66, 72, 78, 84, 90, and 96; and
(3) a light chain CDR3 (VL CDR3) having an amino acid sequence selected from the group consisting of SEQ ID NOs:25, 31, 37, 43, 49, 55, 61, 67, 73, 79, 85, 91, and 97; or a variant thereof having up to about 5 amino acid substitutions, additions, and/or deletions in the VL CDRs.

84. The antibody or antigen-binding fragment of claim 83, wherein
(1) the VH CDR1, CDR2, and CDR3 have the amino acid sequences of SEQ ID NOs:20, 21, and 22, respectively; and/or the VL CDR1, CDR2, and CDR3 have the amino acid sequences of SEQ ID NOs:23, 24 and 25, respectively;
(2) the VH CDR1, CDR2, and CDR3 have the amino acid sequences of SEQ ID NOs:26, 27, and 28, respectively; and/or the VL CDR1, CDR2, and CDR3 have the amino acid sequences of SEQ ID NOs:29, 30, and 31, respectively;
(3) the VH CDR1, CDR2, and CDR3 have the amino acid sequences of SEQ ID NOs:32, 33, and 34, respectively; and/or the VL CDR1, CDR2, and CDR3 have the amino acid sequences of SEQ ID NOs:35, 36 and 37, respectively;
(4) the VH CDR1, CDR2, and CDR3 have the amino acid sequences of SEQ ID NOs:38, 39, and 40, respectively; and/or the VL CDR1, CDR2, and CDR3 have the amino acid sequences of SEQ ID NOs:41, 42 and 43, respectively;
(5) the VH CDR1, CDR2, and CDR3 have the amino acid sequences of SEQ ID NOs:44, 45 and 46, respectively; and/or the VL CDR1, CDR2, and CDR3 have the amino acid sequences of SEQ ID NOs:47, 48 and 49, respectively;
(6) the VH CDR1, CDR2, and CDR3 have the amino acid sequences of SEQ ID NOs:50, 51, and 52, respectively; and/or the VL CDR1, CDR2, and CDR3 have the amino acid sequences of SEQ ID NOs:53, 54, and 55, respectively;
(7) the VH CDR1, CDR2, and CDR3 have the amino acid sequences of SEQ ID NOs:56, 57, and 58, respectively; and/or the VL CDR1, CDR2, and CDR3 have the amino acid sequences of SEQ ID NOs:59, 60, and 61, respectively;
(8) the VH CDR1, CDR2, and CDR3 have the amino acid sequences of SEQ ID NOs:62, 63, and 64, respectively; and/or the VL CDR1, CDR2, and CDR3 have the amino acid sequences of SEQ ID NOs:65, 66, and 67, respectively;
(9) the VH CDR1, CDR2, and CDR3 have the amino acid sequences of SEQ ID NOs:68, 69, and 70, respectively; and/or the VL CDR1, CDR2, and CDR3 have the amino acid sequences of SEQ ID NOs:71, 72 and 73, respectively;
(10) the VH CDR1, CDR2, and CDR3 have the amino acid sequences of SEQ ID NOs:74, 75, and 76, respectively; and/or the VL CDR1, CDR2, and CDR3 have the amino acid sequences of SEQ ID NOs:77, 78 and 79, respectively;
(11) the VH CDR1, CDR2, and CDR3 have the amino acid sequences of SEQ ID NOs:80, 81, and 82, respectively; and/or the VL CDR1, CDR2, and CDR3 have the amino acid sequences of SEQ ID NOs:83, 84, and 85, respectively;
(12) the VH CDR1, CDR2, and CDR3 have the amino acid sequences of SEQ ID NOs:86, 87, and 88, respectively; and/or the VL CDR1, CDR2, and CDR3 have the amino acid sequences of SEQ ID NOs:89, 90 and 91, respectively; or
(13) the VH CDR1, CDR2, and CDR3 have the amino acid sequences of SEQ ID NOs:92, 93 and 94, respectively; and/or the VL CDR1, CDR2, and CDR3 have the amino acid sequences of SEQ ID NOs:95, 96 and 97, respectively.

85. The antibody or antigen-binding fragment of claim 83, comprising:
(a) a VH having at least 85%, at least 90%, at least 95%, at least 98%, or 100% sequence identity to an amino acid sequence selected from the group consisting of SEQ ID NOs:98-110, 124, 126-128, 131-136, and 174-178; and/or
(b) a VL having at least 85%, at least 90%, at least 95%, at least 98%, or 100% sequence identity to an amino acid sequence selected from the group consisting of SEQ ID NOs:111-123, 125, 129-130, 137-144, and 179-181.

86. The antibody or antigen-binding fragment of claim 83, which binds glycosylated PD-L1; wherein optionally the antibody or antigen-binding fragment is a monoclonal antibody or antigen-binding fragment, or a bispecific or multispecific antibody, or selected from the group consisting of a Fab, a Fab′, a F(ab′)₂, a Fv, a scFv, a (scFv)₂, a single domain antibody (sdAb), and a heavy chain antibody (HCAb), or selected from the group consisting of an IgG1 antibody, an IgG2 antibody, an IgG3 antibody, or an IgG4 antibody; or wherein optionally the antibody or antigen-binding fragment is a chimeric antibody or antigen-binding fragment, a humanized antibody or antigen-binding fragment, or a human antibody or antigen-binding fragment.

87. A fusion protein comprising a first domain comprising the antibody or antigen-binding fragment of claim 83 and a second domain comprising a fragment of TGFβRII that binds TGFβ, or a variant thereof.

88. The fusion protein of claim 87, wherein the first and second domains are linked via a transferrin linker.

89. The fusion protein of claim 88, wherein the transferrin linker links the C-terminus of the first domain to the N-terminus of the second domain.

90. The fusion protein of claim 88, wherein the transferrin linker is (PEAPTD)m, m=1, 2, 3, 4, or 5 (SEQ ID NO:18) or (PEAPTDE)n, n=1, 2, 3, 4, or 5 (SEQ ID NO:19); or has an amino acid sequence selected from the group consisting of SEQ ID NOs:220-230; wherein optionally the transferrin linker is (PEAPTD)₃(SEQ ID NO:147).

91. The fusion protein of claim 87, wherein the second domain comprises the ECD of TGFβRII isoform 1 (SEQ ID NO:8) or the ECD of TGFβRII isoform 2 (SEQ ID NO:14), or a variant thereof that has at least 85%, at least 90%, at least 95%, or at least 98% sequence identity to SEQ ID NO:8 or 14.

92. The fusion protein of claim 91, wherein the second domain comprises (1) a variant of the ECD of TGFβRII isoform 1 (SEQ ID NO:8), wherein the variant comprises an amino acid mutation at Q6, K7, N19 or G20 of SEQ ID NO:8; and wherein optionally the second domain has an amino acid sequence selected from the group consisting of SEQ ID NOs:201-203; (2) a truncated form of the ECD of TGFβRII isoform 1 (SEQ ID NO:8), which lacks amino acid residues 1 to n of SEQ ID NO:8, wherein n ranges from 2 to 30, or a variant thereof; and wherein optionally n is 19; or (3) a truncated form of the ECD of TGFβRII isoform 1 (SEQ ID NO:8), which lacks amino acid residues m to 136 of SEQ ID NO:8, wherein m ranges from 80 to 135, or a variant thereof; wherein optionally m is 131 or 128; and wherein optionally the second domain has the amino acid sequence selected from the group consisting of SEQ ID NOs:204, 205 and 232.

93. The fusion protein of claim 75 comprising a heavy chain that has at least 85%, at least 90%, at least 95%, at least 98% or 100% sequence identity to an amino acid sequence selected from the group consisting of SEQ ID NOs: 155-161, 166-172, 206-212, and 233-240, and/or a light chain that has at least 85%, at least 90%, at least 95%, at least 98%, or 100% sequence identity to an amino acid sequence selected from the group consisting of SEQ ID NOs:162-165.

94. A polynucleotide that encodes the antibody or antigen-binding fragment of claim 83.

95. A vector comprising the polynucleotide of claim 94, wherein optionally the vector is a viral vector.

96. An isolated cell comprising the polynucleotide of claim 94.

97. A pharmaceutical composition comprising a therapeutically effective amount of the antibody or antigen-binding fragment of claim 83 and a pharmaceutically acceptable carrier.

98. A pharmaceutical composition comprising a therapeutically effective amount of the fusion protein of claim 87 and a pharmaceutically acceptable carrier.

99. A method of treating tumor or cancer in a subject in need thereof, comprising administering to the subject a therapeutically effective amount of the pharmaceutical composition of claim 98.

100. The method of claim 99, wherein the tumor or cancer is a PD-L1 expressing tumor or cancer.

101. The method of claim 100, wherein the pharmaceutical composition is administered in combination with a second therapy; wherein optionally the second therapy is a chemotherapy, a radiation therapy, an immune therapy, or a cell therapy.


SEQUENCE LISTING (246 sequences)

SEQ ID NO: 1 - protein, 290 aa, Homo sapiens
  REGION 1..290: note = Human PD-L1 extracellular domain
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 2 - protein, 330 aa, Mus musculus
  REGION 1..330: note = mouse IgG2a
AKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 3 - protein, 330 aa, Homo sapiens [sequence omitted]

SEQ ID NO: 4 - protein, 450 aa, synthetic construct
  REGION 1..450: note = human PD-L1 extracellular portion fused with mouse IgG2a Fc portion
TVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 5 - protein, 450 aa, synthetic construct
  REGION 1..450: note = The human PD-L1 extracellular portion fused with human IgG1 Fc portion
TVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 225 aa, synthetic construct
  REGION 1..225: note = human PD-L1 extracellular portion fused with 6XHIS
TVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERHHHHHH

SEQ ID NO: 7 - protein, 567 aa, Homo sapiens
  REGION 1..567: note = TGF-betaRII isoform 1 full length
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 8 - protein, 136 aa, synthetic construct
  REGION 1..136: note = TGF-betaRII isoform 1 ECD 1-136
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 9 - protein, 136 aa, synthetic construct
  REGION 1..136: note = TGF-betaRII isoform 1ECD mutant 1-136
IGGSVQGSVGGSMIVTGGSGAVGFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 10 - protein, 117 aa, synthetic construct
  REGION 1..117: note = TGF-betaRII isoform 1 ECD N-terminal truncation 20-136
GAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 11 - protein, 130 aa, synthetic construct
  REGION 1..130: note = TGF-betaRII isoform 1: ECD C-terminal truncation 1-130
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEY

SEQ ID NO: 12 - protein, 127 aa, synthetic construct
  REGION 1..127: note = TGF-betaRII isoform 1: ECD C-terminal truncation 1-127
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFS

SEQ ID NO: 13 - protein, 592 aa, Homo sapiens
  REGION 1..592: note = TGF-betaRII isoform 2 full length
MGRGLLRGLWPLHIVLWTRIASTIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 14 - protein, 161 aa, synthetic construct
  REGION 1..161: note = TGF-betaRII isoform 2 ECD
IPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 15 - protein, 117 aa, synthetic construct; identical to SEQ ID NO: 10
  REGION 1..117: note = TGF-betaRII isoform 2 ECD (NT truncation, missing NT 44 aa)

SEQ ID NO: 16 - protein, 127 aa, synthetic construct
  REGION 1..127: note = TGF-betaRII isoform 2 ECD (CT truncation, missing CT 128-161)
IPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKE

SEQ ID NO: 17 - protein, 698 aa, Homo sapiens
  REGION 1..698: note = Transferrin full length
MRLAVGALLVCAVLGLCLAVPDKTVRWCAVSEHEATKCQSFRDHMKSVIPSDGPSVACVKKASYLDCIRAIAANEADAVTLDAGLVYDAYLAPNNLKPVVAEFYGSKEDPQTFYYAVAVVKKDSGFQMNQLRGKKSCHTGLGRSAGWNIPIGLLYCDLPEPRKPLEKAVANFFSGSCAPCADGTDFPQLCQLCPGCGCSTLNQYFGYSGAFKCLKDGAGDVAFVKHSTIFENLANKADRDQYELLCLDNTRKPVDEYKDCHLAQVPSHTVVARSMGGKEDLIWELLNQAQEHFGKDKSKEFQLFSSPHGKDLLFKDSAHGFLKVPPRMDAKMYLGYEYVTAIRNLREGTCPEAPTDECKPVKWCALSHHERLKCDEWSVNSVGKIECVSAETTEDCIAKIMNGEADAMSLDGGFVYIAGKCGLVPVLAENYNKSDNCEDTPEAGYFAIAVVKKSASDLTWDNLKGKKSCHTAVGRTAGWNIPMGLLYNKINHCRFDEFFSEGCAPGSKKDSSLCKLCMGSGLNLCEPNNKEGYYGYTGAFRCLVEKGDVAFVKHQTVPQNTGGKNPDPWAKNLNEKDYELLCLDGTRKPVEEYANCHLARAPNHAVVTRKDKEACVHKILRQQQHLFGSNVTDCSGNFCLFRSETKDLLFRDDTVCLAKLHDRNTYEKYLGEEYVKAVGNLRKCSTSSLLEACTFRRP

SEQ ID NO: 18 - protein, 6 aa, synthetic construct
  REGION 1..6: note = Transferrin linker 1
  REPEAT 1..6: note = (PEAPTD)m, m=1, 2, 3, 4, or 5
PEAPTD

SEQ ID NO: 19 - protein, 7 aa, synthetic construct
  REGION 1..7: note = Transferrin linker 2
  REGION 1..7: note = repeat - (PEAPTDE)n, n=1, 2, 3, 4, or 5
PEAPTDE

SEQ ID NO: 20 - protein, 5 aa, synthetic construct
  REGION 1..5: note = Clone A VH CDR1
DTYMH

SEQ ID NO: 21 - protein, 17 aa, synthetic construct
  REGION 1..17: note = Clone A VH CDR2
RIDPANGDTKYDPKFQG

SEQ ID NO: 22 - protein, 12 aa, synthetic construct
  REGION 1..12: note = Clone A VH CDR3
PRLVRFPYVMDY

SEQ ID NO: 23 - protein, 11 aa, synthetic construct
  REGION 1..11: note = Clone A VL CDR1
RASENVYSYLA

SEQ ID NO: 24 - protein, 7 aa, synthetic construct
  REGION 1..7: note = Clone A VL CDR2
NAKTLAE

SEQ ID NO: 25 - protein, 9 aa, synthetic construct
  REGION 1..9: note = Clone A VL CDR3
QHHYGFPYT

SEQ ID NO: 26 - protein, 5 aa, synthetic construct
  REGION 1..5: note = Clone B VH CDR1
SSYIS

SEQ ID NO: 27 - protein, 17 aa, synthetic construct
  REGION 1..17: note = Clone B VH CDR2
WIYAGTGGTSYNQKFTG

SEQ ID NO: 28 - protein, 10 aa, synthetic construct
  REGION 1..10: note = Clone B VH CDR3
HEGKYWYFDV

SEQ ID NO: 29 - protein, 10 aa, synthetic construct
  REGION 1..10: note = Clone B VL CDR1
SASSSVSYVH

SEQ ID NO: 30 - protein, 7 aa, synthetic construct
  REGION 1..7: note = Clone B VL CDR2
DTSNLAS

SEQ ID NO: 31 - protein, 9 aa, synthetic construct
  REGION 1..9: note = Clone B VL CDR3
HQRSSYPWT

SEQ ID NO: 32 - protein, 5 aa, synthetic construct
  REGION 1..5: note = Clone C VH CDR1
DTYMH

SEQ ID NO: 33 - protein, 17 aa, synthetic construct
  REGION 1..17: note = Clone C VH CDR2
RIDPANGNTKYDPKFQG

SEQ ID NO: 34 - protein, 8 aa, synthetic construct
  REGION 1..8: note = Clone C VH CDR3
GLGRWFAY

SEQ ID NO: 35 - protein, 11 aa, synthetic construct
  REGION 1..11: note = Clone C VL CDR1
KASQDVSNAVA

SEQ ID NO: 36 - protein, 7 aa, synthetic construct
  REGION 1..7: note = Clone C VL CDR2
SASNRYT

SEQ ID NO: 37 - protein, 9 aa, synthetic construct
  REGION 1..9: note = Clone C VL CDR3
QQHDSTPLT

SEQ ID NO: 38 - protein, 5 aa, synthetic construct
  REGION 1..5: note = Clone D VH CDR1
GYTMN

SEQ ID NO: 39 - protein, 17 aa, synthetic construct
  REGION 1..17: note = Clone D VH CDR2
LIIPYNGGISYNQKFKD

SEQ ID NO: 40 - protein, 12 aa, synthetic construct
  REGION 1..12: note = Clone D VH CDR3
LITTAPRDSMDY

SEQ ID NO: 41 - protein, 11 aa, synthetic construct
  REGION 1..11: note = Clone D VL CDR1
RASQDISNYLN

SEQ ID NO: 42 - protein, 7 aa, synthetic construct
  REGION 1..7: note = Clone D VL CDR2
YTSKLHS

SEQ ID NO: 43 - protein, 9 aa, synthetic construct
  REGION 1..9: note = Clone D VL CDR3
QQGDALPWT

SEQ ID NO: 44 - protein, 5 aa, synthetic construct
  REGION 1..5: note = Clone F VH CDR1
SGYWN

SEQ ID NO: 45 - protein, 16 aa, synthetic construct
  REGION 1..16: note = Clone F VH CDR2
YISYSGSTYCIPSLKS

SEQ ID NO: 46 - protein, 11 aa, synthetic construct
  REGION 1..11: note = Clone F VH CDR3
GNWGREAWFAY

SEQ ID NO: 47 - protein, 11 aa, synthetic construct
  REGION 1..11: note = Clone F VL CDR1
RASENIYSYLA

SEQ ID NO: 48 - protein, 7 aa, synthetic construct
  REGION 1..7: note = Clone F VL CDR2
NVKILAE

SEQ ID NO: 49 - protein, 9 aa, synthetic construct
  REGION 1..9: note = Clone F VL CDR3
QHHYGLPYT

SEQ ID NO: 50 - protein, 5 aa, synthetic construct
  REGION 1..5: note = Clone G VH CDR1
STYIS

SEQ ID NO: 51 - protein, 17 aa, synthetic construct
  REGION 1..17: note = Clone G VH CDR2
WIYAGTGGTSYNQKFTG

SEQ ID NO: 52 - protein, 10 aa, synthetic construct
  REGION 1..10: note = Clone G VH CDR3
HYGTYWYFDV

SEQ ID NO: 53 - protein, 11 aa, synthetic construct
  REGION 1..11: note = Clone G VL CDR1
KASQDVSTAVA

SEQ ID NO: 54 - protein, 7 aa, synthetic construct
  REGION 1..7: note = Clone G VL CDR2
SASYRYT

SEQ ID NO: 55 - protein, 9 aa, synthetic construct
  REGION 1..9: note = Clone G VL CDR3
LQHYSTPWT

SEQ ID NO: 56 - protein, 6 aa, synthetic construct
  REGION 1..6: note = Clone H VH CDR1
SSTYIS

SEQ ID NO: 57 - protein, 17 aa, synthetic construct
  REGION 1..17: note = Clone H VH CDR2
WIYAGTGGTSYNQKFTG

SEQ ID NO: 58 - protein, 10 aa, synthetic construct
  REGION 1..10: note = Clone H VH CDR3
HIGNYWYFDI

SEQ ID NO: 59 - protein, 10 aa, synthetic construct
  REGION 1..10: note = Clone H VL CDR1
SASSSVSYIH

SEQ ID NO: 60 - protein, 7 aa, synthetic construct
  REGION 1..7: note = Clone H VL CDR2
DTSNLAS

SEQ ID NO: 61 - protein, 9 aa, synthetic construct
  REGION 1..9: note = Clone H VL CDR3
HQRSSYPWT

SEQ ID NO: 62 - protein, 5 aa, synthetic construct
  REGION 1..5: note = Clone J VH CDR1
DYWMH

SEQ ID NO: 63 - protein, 17 aa, synthetic construct
  REGION 1..17: note = Clone J VH CDR2
AVDTSDRYTTYNQKFRG

SEQ ID NO: 64 - protein, 12 aa, synthetic construct
  REGION 1..12: note = Clone J VH CDR3
GLTGPYYYPMDF

SEQ ID NO: 65 - protein, 11 aa, synthetic construct
  REGION 1..11: note = Clone J VL CDR1
LASQTIGTWLA

SEQ ID NO: 66 - protein, 7 aa, synthetic construct
  REGION 1..7: note = Clone J VL CDR2
AATSLAD

SEQ ID NO: 67 - protein, 9 aa, synthetic construct
  REGION 1..9: note = Clone J VL CDR3
QQVFSSPYT

SEQ ID NO: 68 - protein, 5 aa, synthetic construct
  REGION 1..5: note = Clone K VH CDR1
GYTMN

SEQ ID NO: 69 - protein, 17 aa, synthetic construct
  REGION 1..17: note = Clone K VH CDR2
LIIPHNGGTSYNQKFKD

SEQ ID NO: 70 - protein, 12 aa, synthetic construct
  REGION 1..12: note = Clone K VH CDR3
LMTTAPRDSMDY

SEQ ID NO: 71 - protein, 11 aa, synthetic construct
  REGION 1..11: note = Clone K VL CDR1
LASQTIGTWLA

SEQ ID NO: 72 - protein, 7 aa, synthetic construct
  REGION 1..7: note = Clone K VL CDR2
AATSLAD

SEQ ID NO: 73 - protein, 9 aa, synthetic construct
  REGION 1..9: note = Clone K VL CDR3
QQLFSTPWT

SEQ ID NO: 74 - protein, 5 aa, synthetic construct
  REGION 1..5: note = Clone L VH CDR1
GYTMN

SEQ ID NO: 75 - protein, 17 aa, synthetic construct
  REGION 1..17: note = Clone L VH CDR2
LIIPSNGGTNYNQRFKD

SEQ ID NO: 76 - protein, 12 aa, synthetic construct
  REGION 1..12: note = Clone L VH CDR3
LITTAPRDSMDY

SEQ ID NO: 77 - protein, 11 aa, synthetic construct
  REGION 1..11: note = Clone L VL CDR1
RASQDISNYLN

SEQ ID NO: 78 - protein, 7 aa, synthetic construct
  REGION 1..7: note = Clone L VL CDR2
YTSKLHS

SEQ ID NO: 79 - protein, 9 aa, synthetic construct
  REGION 1..9: note = Clone L VL CDR3
QQGDALPWT

SEQ ID NO: 80 - protein, 5 aa, synthetic construct
  REGION 1..5: note = Clone N VH CDR1
GYTMN

SEQ ID NO: 81 - protein, 17 aa, synthetic construct
  REGION 1..17: note = Clone N VH CDR2
LIIPHNGGTSYNQKFKD

SEQ ID NO: 82 - protein, 12 aa, synthetic construct
  REGION 1..12: note = Clone N VH CDR3
LMTTAPRDSMDY

SEQ ID NO: 83 - protein, 11 aa, synthetic construct
  REGION 1..11: note = Clone N VL CDR1
RASQDISNYLN

SEQ ID NO: 84 - protein, 7 aa, synthetic construct
  REGION 1..7: note = Clone N VL CDR2
YTSRLHS

SEQ ID NO: 85 - protein, 9 aa, synthetic construct
  REGION 1..9: note = Clone N VL CDR3
QQGATLPWT

SEQ ID NO: 86 - protein, 5 aa, synthetic construct
  REGION 1..5: note = Clone P VH CDR1
SGYWN

SEQ ID NO: 87 - protein, 16 aa, synthetic construct
  REGION 1..16: note = Clone P VH CDR2
YISYTGSTYHTPSLKG

SEQ ID NO: 88 - protein, 11 aa, synthetic construct
  REGION 1..11: note = Clone P VH CDR3
GNWGREAWFPY

SEQ ID NO: 89 - protein, 11 aa, synthetic construct
  REGION 1..11: note = Clone P VL CDR1
RASESIYSYLA

SEQ ID NO: 90 - protein, 7 aa, synthetic construct
  REGION 1..7: note = Clone P VL CDR2
NAKTLAE

SEQ ID NO: 91 - protein, 9 aa, synthetic construct
  REGION 1..9: note = Clone P VL CDR3
QHHYGFPYT

SEQ ID NO: 92 - protein, 5 aa, synthetic construct
  REGION 1..5: note = Clone Y VH CDR1
SYWMH

SEQ ID NO: 93 - protein, 17 aa, synthetic construct
  REGION 1..17: note = Clone Y VH CDR2
MIDPSDSETKLNQKFRD

SEQ ID NO: 94 - protein, 11 aa, synthetic construct
  REGION 1..11: note = Clone Y VH CDR3
DYRYDVGAMDY

SEQ ID NO: 95 - protein, 11 aa, synthetic construct
  REGION 1..11: note = Clone Y VL CDR1
RASKSISKFLA

SEQ ID NO: 96 - protein, 7 aa, synthetic construct
  REGION 1..7: note = Clone Y VL CDR2
SGSTLQS

SEQ ID NO: 97 - protein, 9 aa, synthetic construct
  REGION 1..9: note = Clone Y VL CDR3
QQHHEYPWT

SEQ ID NO: 98 - protein, 121 aa, synthetic construct
  REGION 1..121: note = Clone A VH
EVQLQQSGAELVKPGASVKLSCTASGFNIKDTYMHWVKQRPEQGLEWIGRIDPANGDTKYDPKFQGKATITADTSSNTAYLHLSSLTSEDTAVYYCASPRLVRFPYVMDYCGQGTSVTVSS

SEQ ID NO: 99 - protein, 119 aa, synthetic construct
  REGION 1..119: note = Clone B VH
QGQMQQSGAELVKPGASVKLSCKTSGFTFSSSYISWLKQKPGQSLEWIAWIYAGTGGTSYNQKFTGKAQLTVDTPSSTAYMQFSSLTTEDSAIYYCARHEGKYWYFDVWGAGTTVTVSS

SEQ ID NO: 100 - protein, 117 aa, synthetic construct
  REGION 1..117: note = Clone C VH
EVQLQQSGADLVKPGASVKLSCTASGFNIKDTYMHWVKQRPEQGLEWIGRIDPANGNTKYDPKFQGKATITTDTSSNTAYLQLSSLTSEDTAVYYCARGLGRWFAYWGQGTLVTVSA

SEQ ID NO: 101 - protein, 121 aa, synthetic construct
  REGION 1..121: note = Clone D VH
EVQLQQSGPELVKPGASMKISCKASGYSFTGYTMNWVKQSHGKNLEWIGLIIPYNGGISYNQKFKDKATLTVDKSSSTAYMELLSLTSEDSAVYYCASLITTAPRDSMDYWGQGTSVTVSS

SEQ ID NO: 102 - protein, 119 aa, synthetic construct
  REGION 1..119: note = Clone F VH
EVQLQESGPTLVKPSQTLSLTCSVTGDSITSGYWNWIRKFPGNKVEYMGYISYSGSTYCIPSLKSRISITRDTSKNQYYLQLNSVTTADTATYYCARGNWGREAWFAYWGQGTLVTVSA

SEQ ID NO: 103 - protein, 119 aa, synthetic construct
  REGION 1..119: note = Clone G VH
QGQMQQSGAELVKPGASVKLSCKTSGFTFSSTYISWLKQKPGQSLEWIAWIYAGTGGTSYNQKFTGKAQLTVDTSSSTAYMQFSSLTTEDSAIYYCARHYGTYWYFDVWGAGTTVTVSS

SEQ ID NO: 104 - protein, 119 aa, synthetic construct
  REGION 1..119: note = Clone H VH
QGQMQQSGAELVKPGASVKLSCKTSGFTFSSTYISWLKQKPGQSLEWIAWIYAGTGGTSYNQKFTGKAQLTVDTSSSTAYMQFSSLTTEDSAIYYCARHIGNYWYFDIWGAGTTVTVSS

SEQ ID NO: 105 - protein, 121 aa, synthetic construct
  REGION 1..121: note = Clone J VH
QVQLQQPGAELVMPGPSVKMSCKASGYTFTDYWMHWVKQRPGQGLEWIGAVDTSDRYTTYNQKFRGKATMTVDESSSTAYMQLSNLTSEDSAVYYCARGLTGPYYYPMDFWGQGTSVTASS

SEQ ID NO: 106 - protein, 121 aa, synthetic construct
  REGION 1..121: note = Clone K VH
EVQLQQSGPELVKPGASMKISCKASGYSFTGYTMNWVKQSHGKNLEWIGLIIPHNGGTSYNQKFKDKATLTVDKSSRTAYMELLSLTSEDSAVYYCASLMTTAPRDSMDYWGQGTSVTVSS

SEQ ID NO: 107 - protein, 121 aa, synthetic construct
  REGION 1..121: note = Clone L VH
EVHLQQSGPELVKPGASMKISCKASGYSFTGYTMNWVKQSHGKNLEWIGLIIPSNGGTNYNQRFKDKATLSVDKSSSTAYMELLSLTSEDSAVYYCASLITTAPRDSMDYWGQGTSVTVSS

SEQ ID NO: 108 - protein, 121 aa, synthetic construct; identical to SEQ ID NO: 106
  REGION 1..121: note = Clone N VH

SEQ ID NO: 109 - protein, 119 aa, synthetic construct
  REGION 1..119: note = Clone P VH
EVQLQESGPSLVKYSQTLSLTCSVTGDSITSGYWNWIRKFPGNKLEYMGYISYTGSTYHTPSLKGRISITRDTSKNQYYMQLDSVTTEDTATYYCARGNWGREAWFPYWGQGTLVTVSA

SEQ ID NO: 110 - protein, 120 aa, synthetic construct
  REGION 1..120: note = Clone Y VH
QVQLQQSGPQLVRPGASVKISCKASGYSFTSYWMHWVKQRPGHGLEWIGMIDPSDSETKLNQKFRDKATLTVDTSSSTAYMQLSSPTSEDSAVYYCARDYRYDVGAMDYWGQGTSVTVSS

SEQ ID NO: 111 - protein, 107 aa, synthetic construct
  REGION 1..107: note = Clone A VL
DIQMTQSPASLSASVGETVTITCRASENVYSYLAWYQQKQGKSPQLLVYNAKTLAEGVPSRFSGSGSGTQFSLKINSLQPEDFGSYYCQHHYGFPYTFGGGTKLEIK

SEQ ID NO: 112 - protein, 106 aa, synthetic construct
  REGION 1..106: note = Clone B VL
QILLTQSPAIMSASPGEKVTMTCSASSSVSYVHWYQQKPGSSPKPWIYDTSNLASGFPARFSGSGSGTSYSLIISSMEAEDAATYYCHQRSSYPWTFGGGTKLEIK

SEQ ID NO: 113 - protein, 107 aa, synthetic construct
  REGION 1..107: note = Clone C VL
DIVMTQSHKFMSTSVGDRVSITCKASQDVSNAVAWYQQKPGQSPELLIYSASNRYTGVPDRFTGSGSGTDFTFTISSVQAEDLAVYYCQQHDSTPLTFGAGTKLELK

SEQ ID NO: 114 - protein, 107 aa, synthetic construct
  REGION 1..107: note = Clone D VL
DIQMTQTTSSLSASLGDRVTISCRASQDISNYLNWYQQKPDGSVKLLIYYTSKLHSGVPSRFSGSGSGTDYSLTISNLEQEDIATYFCQQGDALPWTFGGGTKLEIK

SEQ ID NO: 115 - protein, 107 aa, synthetic construct
  REGION 1..107: note = Clone F VL
DIQMTQSPASLSASVGETVTITCRASENIYSYLAWYQQKQGKSPQLLVYNVKILAEGVSSRFSGSGSGTQFSLKINSLQPEDFGSYYCQHHYGLPYTFGGGTQLEIK

SEQ ID NO: 116 - protein, 107 aa, synthetic construct
  REGION 1..107: note = Clone G VL
DIVMTQSHKFMSTSVGDRVSITCKASQDVSTAVAWYQQKPGQSPKLLIYSASYRYTGVPDRFTGSGSGTDFTFTISSVQAGDLAVYYCLQHYSTPWTFGGGTKLEIK

SEQ ID NO: 117 - protein, 106 aa, synthetic construct
  REGION 1..106: note = Clone H VL
QILLTQSPAIMSASPGEKVTMTCSASSSVSYIHWYQQKPGSSPKPWIYDTSNLASGFPARFSGSGSGTSYFLIISSMEAEDAATYYCHQRSSYPWTFGGGTKLEIK

SEQ ID NO: 118 - protein, 107 aa, synthetic construct
  REGION 1..107: note = Clone J VL
DIQMTQSPASQSASLGESVTITCLASQTIGTWLAWYQQKPGKSPQLLIYAATSLADGVPSRFSGGGSGTKFSFKISSLQAEDFGSYYCQQVFSSPYTFGGGTKLEIK

SEQ ID NO: 119 - protein, 107 aa, synthetic construct
  REGION 1..107: note = Clone K VL
DIQMTQSPASQSASLGESVTITCLASQTIGTWLAWYQQKPGKSPQLLIYAATSLADGVPSRFSGSGSGTKFSFKISSLQAEDFVSYYCQQLFSTPWTFGGGTKLEIK

SEQ ID NO: 120 - protein, 107 aa, synthetic construct
  REGION 1..107: note = Clone L VL
DIQMTQTPSSLSASLGDRVTISCRASQDISNYLNWYQQKPDGSVKLLIYYTSKLHSGVPSRFSGSGSGTDYSLTINNLDQEDIATYFCQQGDALPWTFGGGTKLEIK

SEQ ID NO: 121 - protein, 107 aa, synthetic construct
  REGION 1..107: note = Clone N VL
DIQMTQTTSSLSASLGDRVTISCRASQDISNYLNWYQQKPDGTVKLLIYYTSRLHSGVPSRFSGSGSGTDYSLTISNLDQEDIATYFCQQGATLPWTFGGGTKLEIK

SEQ ID NO: 122 - protein, 107 aa, synthetic construct
  REGION 1..107: note = Clone P VL
DIQMTQSPASLSASVGETVTITCRASESIYSYLAWYQQKQGKSPQLLVYNAKTLAEGVPSRFSGSGSGTQFSLKINSLQTEDFGTYYCQHHYGFPYTFGGGTKLEIK

SEQ ID NO: 123 - protein, 107 aa, synthetic construct
  REGION 1..107: note = Clone Y VL
DVQITQSPSFLAASPGETITLNCRASKSISKFLAWYQEKPGKTNKVLIYSGSTLQSGIPSRFSGSGSGTDFTLTISSLEPEDFAMYYCQQHHEYPWTFGGGTKLEIK

SEQ ID NO: 124 - protein, 121 aa, synthetic construct
  REGION 1..121: note = Dh VH-CDR graft
QVQLVQSGAEVKKPGASVKVSCKASGYSFTGYTMNWVRQAPGQGLEWMGLIIPYNGGISYNQKFKDRVTMTRDTSISTVYMELSRLRSDDTAVYYCASLITTAPRDSMDYWGQGTLVTVSS

SEQ ID NO: 125 - protein, 107 aa, synthetic construct
  REGION 1..107: note = DhVL-CDR graft
DIQMTQSPSSLSASVGDRVTITCRASQDISNYLNWYQQKPGKAPKLLIYYTSKLHSGVPSRFSGSGSGTDFTFTISSLQPEDIATYYCQQGDALPWTFGGGTKVEIK

SEQ ID NO: 126 - protein, 121 aa, synthetic construct
  REGION 1..121: note = DhVH1
QVQLVQSGAEVKKPGASVKVSCKASGYSFTGYTMNWVKQSHGQGLEWIGLIIPYNGGISYNQKFKDRATLTVDTSSSTAYMELSRLRSDDTAVYYCASLITTAPRDSMDYWGQGTLVTVSS

SEQ ID NO: 127 - protein, 121 aa, synthetic construct
  REGION 1..121: note = DhVH2
QVQLVQSGAEVKKPGASVKVSCKASGYSFTGYTMNWVKQAHGQGLEWMGLIIPYNGGISYNQKFKDRVTMTVDTSISTAYMELSRLRSDDTAVYYCASLITTAPRDSMDYWGQGTLVTVSS

SEQ ID NO: 128 - protein, 121 aa, synthetic construct
  REGION 1..121: note = DhVH3
QVQLVQSGAEVKKPGASVKVSCKASGYSFTGYTMNWVRQAPGQGLEWMGLIIPYNGGISYNQKFKDRVTMTVDTSISTAYMELSRLRSDDTAVYYCASLITTAPRDSMDYWGQGTLVTVSS

SEQ ID NO: 129 - protein, 107 aa, synthetic construct
  REGION 1..107: note = DhVL1
DIQMTQSTSSLSASVGDRVTITCRASQDISNYLNWYQQKPGGAPKLLIYYTSKLHSGVPSRFSGSGSGTDYTLTISSLQQEDIATYYCQQGDALPWTFGGGTKVEIK

SEQ ID NO: 130 - protein, 107 aa, synthetic construct
  REGION 1..107: note = DhVL2
DIQMTQSPSSLSASVGDRVTITCRASQDISNYLNWYQQKPGKAPKLLIYYTSKLHSGVPSRFSGSGSGTDFTLTISSLQPEDIATYYCQQGDALPWTFGGGTKVEIK

SEQ ID NO: 131 - protein, 119 aa, synthetic construct
  REGION 1..119: note = BhVH1
QVQLVQSGAEVKKPGASVKVSCKASGFTFSSSYISWVRQAPGQGLEWMGWIYAGTGGTSYNQKFTGRVTMTTDTSTSTAYMELRSLRSDDTAVYYCARHEGKYWYFDVWGQGTTVTVSS

SEQ ID NO: 132 - protein, 119 aa, synthetic construct
  REGION 1..119: note = BhVH2
QGQLVQSGAEVKKPGASVKVSCKTSGFTFSSSYISWVRQAPGQGLEWMGWIYAGTGGTSYNQKFTGRVTMTVDTSTSTAYMELRSLRSDDTAVYYCARHEGKYWYFDVWGQGTTVTVSS

SEQ ID NO: 133 - protein, 119 aa, synthetic construct
  REGION 1..119: note = BhVH3
QVQLVQSGAEVKKPGASVKVSCKTSGFTFSSSYISWVRQAPGQGLEWMGWIYAGTGGTSYNQKFTGRVTMTVDTSTSTAYMELRSLRSDDTAVYYCARHEGKYWYFDVWGQGTTVTVSS

SEQ ID NO: 134 - protein, 119 aa, synthetic construct
  REGION 1..119: note = BhVH4
QVQLVQSGAEVKKPGSSVKVSCKASGFTFSSSYISWVRQAPGQGLEWMGWIYAGTGGTSYNQKFTGRVTITADKSTSTAYMELSSLRSEDTAVYYCARHEGKYWYFDVWGQGTTVTVSS

SEQ ID NO: 135 - protein, 119 aa, synthetic construct
  REGION 1..119: note = BhVH5
QGQLVQSGAEVKKPGSSVKVSCKTSGFTFSSSYISWVRQAPGQGLEWMGWIYAGTGGTSYNQKFTGRVTITVDTSTSTAYMELSSLRSEDTAVYYCARHEGKYWYFDVWGQGTTVTVSS

SEQ ID NO: 136 - protein, 119 aa, synthetic construct
  REGION 1..119: note = BhVH6
QVQLVQSGAEVKKPGSSVKVSCKTSGFTFSSSYISWVRQAPGQGLEWMGWIYAGTGGTSYNQKFTGRVTITVDTSTSTAYMELSSLRSEDTAVYYCARHEGKYWYFDVWGQGTTVTVSS

SEQ ID NO: 137 - protein, 106 aa, synthetic construct
  REGION 1..106: note = BhVL1
DIQMTQSPSSLSASVGDRVTITCSASSSVSYVHWYQQKPGKAPKPWIYDTSNLASGFPSRFSGSGSGTDYTLTISSLQPEDFATYYCHQRSSYPWTFGGGTKVEIK

SEQ ID NO: 138 - protein, 106 aa, synthetic construct
  REGION 1..106: note = BhVL2
EIVLTQSPATLSLSPGERATLSCSASSSVSYVHWYQQKPGQAPRPWIYDTSNLASGFPARFSGSGSGTDYTLTISSLEPEDAAVYYCHQRSSYPWTFGGGTKVEIK

SEQ ID NO: 139 - protein, 106 aa, synthetic construct
  REGION 1..106: note = BhVL3
DIQMTQSPSTLSASVGDRVTITCSASSSVSYVHWYQQKPGKAPKLLIYDTSNLASGVPSRFSGSGSGTEFTLTISSLQPDDFATYYCHQRSSYPWTFGGGTKVEIK

SEQ ID NO: 140 - protein, 106 aa, synthetic construct
  REGION 1..106: note = BhVL4
DIQMTQSPSTLSASVGDRVTITCSASSSVSYVHWYQQKPGKAPKPWIYDTSNLASGFPSRFSGSGSGTEYTLTISSLQPDDFATYYCHQRSSYPWTFGGGTKVEIK

SEQ ID NO: 141 - protein, 106 aa, synthetic construct
  REGION 1..106: note = BhVL5
DIVMTQSPLSLPVTPGEPASISCSASSSVSYVHWYLQKPGQSPQLLIYDTSNLASGVPDRFSGSGSGTDFTLKISRVEAEDVGVYYCHQRSSYPWTFGGGTKVEIK

SEQ ID NO: 142 - protein, 106 aa, synthetic construct
  REGION 1..106: note = BhVL6
DIVMTQSPLSLPVTPGEPASISCSASSSVSYVHWYLQKPGQSPQPWIYDTSNLASGFPDRFSGSGSGTDYTLKISRVEAEDVGVYYCHQRSSYPWTFGGGTKVEIK

SEQ ID NO: 143 - protein, 106 aa, synthetic construct
  REGION 1..106: note = BhVL7
EIVMTQSPATLSVSPGERATLSCSASSSVSYVHWYQQKPGQAPRLLIYDTSNLASGIPARFSGSGSGTEFTLTISSLQSEDFAVYYCHQRSSYPWTFGGGTKVEIK

SEQ ID NO: 144 - protein, 106 aa, synthetic construct
  REGION 1..106: note = BhVL8
EIVMTQSPATLSVSPGERATLSCSASSSVSYVHWYQQKPGQAPRPWIYDTSNLASGFPARFSGSGSGTEYTLTISSLQSEDAAVYYCHQRSSYPWTFGGGTKVEIK

SEQ ID NO: 145 - protein, 6 aa, synthetic construct
  REGION 1..6: note = Transferrin linker
PEAPTD

SEQ ID NO: 146 - protein, 12 aa, synthetic construct
  REGION 1..12: note = Transferrin linker
PEAPTDPEAPTD

SEQ ID NO: 147 - protein, 18 aa, synthetic construct
  REGION 1..18: note = Transferrin linker
PEAPTDPEAPTDPEAPTD

SEQ ID NO: 148 - protein, 24 aa, synthetic construct
  REGION 1..24: note = Transferrin linker
PEAPTDPEAPTDPEAPTDPEAPTD

SEQ ID NO: 149 - protein, 30 aa, synthetic construct
  REGION 1..30: note = Transferrin linker
PEAPTDPEAPTDPEAPTDPEAPTDPEAPTD

SEQ ID NO: 150 - protein, 7 aa, synthetic construct
  REGION 1..7: note = Transferrin linker
PEAPTDE

SEQ ID NO: 151 - protein, 14 aa, synthetic construct
  REGION 1..14: note = Transferrin linker
PEAPTDEPEAPTDE

SEQ ID NO: 152 - protein, 21 aa, synthetic construct
  REGION 1..21: note = Transferrin linker
PEAPTDEPEAPTDEPEAPTDE

SEQ ID NO: 153 - protein, 28 aa, synthetic construct
  REGION 1..28: note = Transferrin linker
PEAPTDEPEAPTDEPEAPTDEPEAPTDE

SEQ ID NO: 154 - protein, 35 aa, synthetic construct
  REGION 1..35: note = Transferrin linker
PEAPTDEPEAPTDEPEAPTDEPEAPTDEPEAPTDE

SEQ ID NO: 155 - protein, 594 aa, synthetic construct
  REGION 1..594: note = Fusion protein - heavy chain 1
QGQLVQSGAEVKKPGSSVKVSCKTSGFTFSSSYISWVRQAPGQGLEWMGWIYAGTGGTSYNQKFTGRVTITVDTSTSTAYMELSSLRSEDTAVYYCARHEGKYWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAPEAPTDPEAPTDPEAPTDIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFS

SEQ ID NO: 156 - protein, 603 aa, synthetic construct
  REGION 1..603: note = Fusion protein - heavy chain 2
QGQLVQSGAEVKKPGSSVKVSCKTSGFTFSSSYISWVRQAPGQGLEWMGWIYAGTGGTSYNQKFTGRVTITVDTSTSTAYMELSSLRSEDTAVYYCARHEGKYWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAPEAPTDPEAPTDPEAPTDIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 157 - protein, 588 aa, synthetic construct
  REGION 1..588: note = Fusion protein - heavy chain 3
QGQLVQSGAEVKKPGSSVKVSCKTSGFTFSSSYISWVRQAPGQGLEWMGWIYAGTGGTSYNQKFTGRVTITVDTSTSTAYMELSSLRSEDTAVYYCARHEGKYWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAPEAPTDPEAPTDIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFS

SEQ ID NO: 158 - protein, 600 aa, synthetic construct
  REGION 1..600: note = Fusion protein - heavy chain 4
QGQLVQSGAEVKKPGSSVKVSCKTSGFTFSSSYISWVRQAPGQGLEWMGWIYAGTGGTSYNQKFTGRVTITVDTSTSTAYMELSSLRSEDTAVYYCARHEGKYWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAPEAPTDPEAPTDPEAPTDPEAPTDIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFS

SEQ ID NO: 159 - protein, 596 aa, synthetic construct
  REGION 1..596: note = Fusion protein - heavy chain 5
QVQLVQSGAEVKKPGASVKVSCKASGYSFTGYTMNWVKQAHGQGLEWMGLIIPYNGGISYNQKFKDRVTMTVDTSISTAYMELSRLRSDDTAVYYCASLITTAPRDSMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAPEAPTDPEAPTDPEAPTDIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFS

SEQ ID NO: 160 - protein, 592 aa, synthetic construct
  REGION 1..592: note = Fusion protein - heavy chain 6
QVQLVQSGAEVKKPGASVKVSCKASGFNIKDTYMHWVRQAPGQGLEWIGRIDPANGNTKYDPKFQGRVTITTDTSASTAYLELSSLRSEDTAVYYCARGLGRWFAYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAPEAPTDPEAPTDPEAPTDIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFS

SEQ ID NO: 161 - protein, 596 aa, synthetic construct
  REGION 1..596: note = Fusion protein - heavy chain 7
EVQLQQSGPELVKPGASMKISCKASGYSFTGYTMNWVKQSHGKNLEWIGLIIPHNGGTSYNQKFKDKATLTVDKSSRTAYMELLSLTSEDSAVYYCASLMTTAPRDSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAPEAPTDPEAPTDPEAPTDIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFS

SEQ ID NO: 162 - protein, 213 aa, synthetic construct
  REGION 1..213: note = Fusion protein - light chain 1
EIVMTQSPATLSVSPGERATLSCSASSSVSYVHWYQQKPGQAPRPWIYDTSNLASGFPARFSGSGSGTEYTLTISSLQSEDAAVYYCHQRSSYPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 163 - protein, 214 aa, synthetic construct
  REGION 1..214: note = Fusion protein - light chain 2
DIQMTQSPSSLSASVGDRVTITCRASQDISNYLNWYQQKPGKAPKLLIYYTSKLHSGVPSRFSGSGSGTDFTLTISSLQPEDIATYYCQQGDALPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 164 - protein, 214 aa, synthetic construct
  REGION 1..214: note = Fusion protein - light chain: 3
DIQMTQSPSSLSASVGDRVTITCQASQDVSNAVAWYQQKPGKAPKLLIYSASNLYTGVPSRFSGSGSGTDFTFTISSLQPEDIATYYCQQHDSTPLTFGQGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 165 - protein, 214 aa, synthetic construct
  REGION 1..214: note = Fusion protein - light chain: 4
DIQMTQTTSSLSASLGDRVTISCRASQDISNYLNWYQQKPDGTVKLLIYYTSRLHSGVPSRFSGSGSGTDYSLTISNLDQEDIATYFCQQGATLPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 166 - protein, 591 aa, synthetic construct
  REGION 1..591: note = Fusion protein - heavy chain 8
QGQLVQSGAEVKKPGSSVKVSCKTSGFTFSSSYISWVRQAPGQGLEWMGWIYAGTGGTSYNQKFTGRVTITVDTSTSTAYMELSSLRSEDTAVYYCARHEGKYWYFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAPEAPTDPEAPTDPEAPTDIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFS

SEQ ID NO: 167 - protein, 600 aa, synthetic construct
  REGION 1..600: note = Fusion protein - heavy chain 9
QGQLVQSGAEVKKPGSSVKVSCKTSGFTFSSSYISWVRQAPGQGLEWMGWIYAGTGGTSYNQKFTGRVTITVDTSTSTAYMELSSLRSEDTAVYYCARHEGKYWYFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAPEAPTDPEAPTDPEAPTDIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 168 - protein, 585 aa, synthetic construct
  REGION 1..585: note = Fusion protein - heavy chain 10
QGQLVQSGAEVKKPGSSVKVSCKTSGFTFSSSYISWVRQAPGQGLEWMGWIYAGTGGTSYNQKFTGRVTITVDTSTSTAYMELSSLRSEDTAVYYCARHEGKYWYFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAPEAPTDPEAPTDIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFS

SEQ ID NO: 169 - protein, 597 aa, synthetic construct
  REGION 1..597: note = Fusion protein - heavy chain 11
QGQLVQSGAEVKKPGSSVKVSCKTSGFTFSSSYISWVRQAPGQGLEWMGWIYAGTGGTSYNQKFTGRVTITVDTSTSTAYMELSSLRSEDTAVYYCARHEGKYWYFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAPEAPTDPEAPTDPEAPTDPEAPTDIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFS

SEQ ID NO: 170 - protein, 593 aa, synthetic construct
  REGION 1..593: note = Fusion protein - heavy chain 12
QVQLVQSGAEVKKPGASVKVSCKASGYSFTGYTMNWVKQAHGQGLEWMGLIIPYNGGISYNQKFKDRVTMTVDTSISTAYMELSRLRSDDTAVYYCASLITTAPRDSMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAPEAPTDPEAPTDPEAPTDIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFS

SEQ ID NO: 171 - protein, 589 aa, synthetic construct
  REGION 1..589: note = Fusion protein - heavy chain 13
QVQLVQSGAEVKKPGASVKVSCKASGFNIKDTYMHWVRQAPGQGLEWIGRIDPANGNTKYDPKFQGRVTITTDTSASTAYLELSSLRSEDTAVYYCARGLGRWFAYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAPEAPTDPEAPTDPEAPTDIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFS

SEQ ID NO: 172 - protein, 593 aa, synthetic construct
  REGION 1..593: note = Fusion protein - heavy chain 14
EVQLQQSGPELVKPGASMKISCKASGYSFTGYTMNWVKQSHGKNLEWIGLIIPHNGGTSYNQKFKDKATLTVDKSSRTAYMELLSLTSEDSAVYYCASLMTTAPRDSMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAPEAPTDPEAPTDPEAPTDIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFS

SEQ ID NO: 173 - protein, 327 aa, Homo sapiens
  REGION 1..327: note = IgG4 heavy chain
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGA

SEQ ID NO: 174 - protein, 117 aa, synthetic construct
  REGION 1..117: note = Clone C- ChVH1
QVQLVQSGAEVKKPGASVKLSCTASGFNIKDTYMHWVKQRPGQGLEWIGRIDPANGNTKYDPKFQGKATITTDTSTNTAYLELSSLRSEDTAVYYCARGLGRWFAYWGQGTTVTVSS

SEQ ID NO: 175 - protein, 117 aa, synthetic construct
  REGION 1..117: note = Clone C- ChVH2
QVQLVQSGAEVKKPGASVKVSCKASGFNIKDTYMHWVRQAPGQGLEWIGRIDPANGNTKYDPKFQGRATITTDTSTNTAYLELSSLRSEDTAVYYCARGLGRWFAYWGQGTTVTVSS

SEQ ID NO: 176 - protein, 117 aa, synthetic construct
  REGION 1..117: note = Clone C- ChVH3
QVQLVQSGAEVKKPGASVKVSCKASGFNIKDTYMHWVRQAPGQGLEWIGRIDPANGNTKYDPKFQGRVTITTDTSANTAYMELSSLRSEDTAVYYCARGLGRWFAYWGQGTTVTVSS

SEQ ID NO: 177 - protein, 117 aa, synthetic construct
  REGION 1..117: note = Clone C- ChVH4
QVQLVQSGAEVKKPGASVKVSCKASGFNIKDTYMHWVRQAPGQGLEWIGRIDPANGNTKYDPKFQGRVTITTDTSASTAYLELSSLRSEDTAVYYCARGLGRWFAYWGQGTTVTVSS

SEQ ID NO: 178 - protein, 117 aa, synthetic construct
  REGION 1..117: note = Clone C- ChVH5
QVQLVQSGAEVKKPGASVKVSCKASGFNIKDTYMHWVRQAPGQRLEWIGRIDPANGNTKYSQKFQGRVTITRDTSASTAYMELSSLRSEDTAVYYCARGLGRWFAYWGQGTTVTVSS

SEQ ID NO: 179 - protein, 107 aa, synthetic construct
  REGION 1..107: note = Clone C- ChVL1
DIQMTQSPSSLSASVGDRVTITCKASQDVSNAVAWYQQKPGKAPKLLIYSASNRYTGVPDRFSGSGSGTDFTFTISSLQPEDIATYYCQQHDSTPLTFGQGTKLELK

SEQ ID NO: 180 - protein, 107 aa, synthetic construct
  REGION 1..107: note = Clone C- ChVL2
DIQMTQSPSSLSASVGDRVTITCKASQDVSNAVAWYQQKPGKAPKLLIYSASNRYTGVPSRFSGSGSGTDFTFTISSLQPEDIATYYCQQHDSTPLTFGQGTKLELK

SEQ ID NO: 181 - protein, 107 aa, synthetic construct
  REGION 1..107: note = Clone C- ChVL3
DIQMTQSPSSLSASVGDRVTITCQASQDVSNAVAWYQQKPGKAPKLLIYSASNLYTGVPSRFSGSGSGTDFTFTISSLQPEDIATYYCQQHDSTPLTFGQGTKLELK

SEQ ID NO: 182 - protein, 5 aa, synthetic construct
  REGION 1..5: note = linker
GGGGS

SEQ ID NO: 183 - DNA, 1782 bp, synthetic construct
  misc_feature 1..1782: note = Fusion protein - heavy chain 1
  source 1..1782: mol_type = other DNA
caaggtcaactagtgcaaagtggtgcagaagtgaagaagcctggaagctccgtgaaggtgtcctgtaagacatccggattcaccttctccagttcctatatctcctgggtgcggcaggcccctggccagggactggagtggatgggctggatctacgccggcacaggcggcacctcctacaaccagaaattcaccggcagggtaaccattacagtggacacatctacctccaccgcctacatggaactgagctctctgagatccgaggataccgctgtgtactactgcgccagacacgagggcaagtactggtatttcgacgtgtggggccagggcaccaccgtgaccgtgtcttctgcctcgaccaagggcccttctgtgttccctctggccccctcctccaagagcacatctggcggaaccgctgctctgggatgtctggtgaaggactactttcctgagcctgtgactgtttcttggaacagcggcgccctgacctccggagtgcataccttccctgccgtgctgcagtcttccggtctgtatagcctgtccagcgtggtgaccgtgcctagcagctctctgggcacacagacatatatttgtaacgtgaatcacaagccttctaacaccaaggtggacaagcgggttgagcctaagtcctgcgacaagacccacacgtgtcctccttgccctgctcctgagctgctcggaggcccttctgtgtttctgttcccaccaaaacccaaggacaccctgatgatctcccggacccctgaagtgacctgcgtggtggtggacgtgtctcacgaggaccccgaagtcaagttcaactggtacgtggacggcgtggaagtgcacaacgccaagaccaagcccagagaagagcagtacaattctacctacagagtggtctccgtgctgaccgtgctgcaccaggactggctgaacggcaaagagtacaagtgcaaggtctccaataaggccctgcctgctcctatcgagaagaccatctctaaggccaaaggccagcccagggaacctcaagtgtacacactgcccccaagccgcgaggaaatgaccaagaaccaggtgtccctgacatgtctcgtgaagggcttctaccctagcgacatcgccgtggagtgggagtccaatggccagcccgagaacaactacaagaccacaccacctgtgctggactccgatggctccttcttcctgtactccaagctgaccgtggataagtctagatggcagcagggcaacgtgttcagctgttctgtgatgcacgaggctctgcacaaccactacacccagaaatctctgtccctgtctcctggagctcctgaggctccaaccgaccctgaagcccctaccgacccagaggcccccaccgatatccctcctcacgtgcagaagtccgtgaacaacgacatgatcgtgaccgacaacaacggcgccgttaagtttccccagctgtgcaagttctgcgatgtgcggttctctacctgtgataatcagaagtcctgcatgtccaactgctctatcacctccatctgcgagaagcctcaagaggtgtgtgtggctgtctggcggaagaacgatgagaacatcaccctggaaacagtgtgccacgatcccaagctgccttaccacgacttcatcctggaagatgctgcctctcctaagtgcatcatgaaagaaaagaaaaaacccggcgagacattcttcatgtgctcctgctcctctgacgagtgcaacgacaacatcatcttctct

SEQ ID NO: 184 - DNA, 1809 bp, synthetic construct
  misc_feature 1..1809: note = Fusion protein - heavy chain 2
  source 1..1809: mol_type = other DNA
cagggacagctggtgcagagcggcgccgaggtgaagaagcctggctccagcgtgaaggtgtcctgcaagaccagcggctttaccttttcctcctcctatatctcctgggtgcggcaggcccccggccagggactggagtggatgggatggatctacgccggcaccggcggcaccagctacaaccagaagtttaccggcagggtgaccatcaccgtggataccagcacctccaccgcctatatggagctgtcctccctgcggtccgaggataccgctgtgtactactgtgctcggcacgagggcaagtactggtattttgacgtgtggggccagggcaccaccgtgaccgtgagcagcgcttccaccaagggccctagcgtgtttcctctggccccttccagcaagtccaccagcggcggcaccgccgctctgggatgtctggtgaaggattatttccccgagcccgtgaccgtgtcctggaacagcggcgctctgaccagcggcgtgcacacctttcctgctgtgctgcagagcagcggcctgtatagcctgtccagcgtggtgaccgtgcccagcagctccctgggcacccagacctatatctgtaacgtgaatcacaagccctccaataccaaggtggacaagagggtggagcctaagagctgcgacaagacccacacctgtcccccttgccctgctcctgagctgctgggcggcccttccgtgttcctgtttccccctaagcccaaggacaccctgatgatctcccggacccccgaggtgacctgcgtggtggtggacgtgtcccacgaggaccccgaggtgaagtttaactggtacgtggacggcgtggaggtgcacaacgccaagaccaagcctagggaggagcagtacaacagcacctacagggtggtgtccgtgctgaccgtgctgcaccaggactggctgaatggcaaggagtacaagtgtaaggtgagcaacaaggccctgcctgctcctatcgagaagaccatcagcaaggctaagggccagcctagggagccccaggtgtataccctgcctcccagcagggaggagatgaccaagaatcaggtgtccctgacctgcctggtgaagggcttctaccccagcgacatcgctgtggagtgggagagcaatggccagcccgagaacaattacaagaccaccccccccgtgctggattccgacggctccttcttcctgtattccaagctgaccgtggataagagcaggtggcagcagggcaatgtgttctcctgctccgtgatgcacgaggctctgcacaatcactatacccagaagagcctgagcctgtcccctggcgcccccgaagctcctaccgatcctgaggctcccaccgaccctgaggccccaaccgacatccctccccacgtgcagaagtccgtgaacaacgacatgatcgtgaccgataataatggcgccgtgaagtttcctcagctgtgcaagttttgcgacgtgcggttttccacctgtgacaaccagaagtcctgtatgagcaactgtagcatcacctccatctgtgagaagccccaggaggtgtgcgtggccgtgtggaggaagaacgacgagaatatcaccctggagaccgtgtgtcacgaccctaagctgccttatcacgattttatcctggaggatgctgccagccctaagtgtatcatgaaggagaagaagaagcctggagagaccttctttatgtgtagctgtagcagcgatgagtgcaatgacaacatcatcttttccgaggagtacaacacctccaaccctgac

SEQ ID NO: 185 - DNA, 1764 bp, synthetic construct
  misc_feature 1..1764: note = Fusion protein - heavy chain 3
  source 1..1764: mol_type = other DNA
cagggacagctggtgcagagcggcgctgaggtgaagaagcctggcagctccgtgaaggtgtcctgcaagaccagcggcttcacctttagcagcagctatatctcctgggtgcggcaggctcccggccagggattggagtggatgggctggatctacgctggcaccggcggcaccagctataaccagaagttcaccggccgggtgaccatcaccgtggatacctccacctccaccgcttatatggagctgtccagcctgcggagcgaggacaccgccgtgtactattgcgcccggcacgagggcaagtactggtactttgacgtgtggggccagggcaccaccgtgaccgtgagcagcgcctccaccaagggccccagcgtgttccctctggctccctcctccaagtccaccagcggcggcaccgccgctctgggatgtctggtgaaggactatttccccgagcccgtgaccgtgtcctggaactccggcgccctgaccagcggcgtgcacacattccctgctgtgctgcagagcagcggcctgtattccctgtccagcgtggtgaccgtgcctagctcctccctgggcacccagacctatatctgcaacgtgaatcacaagccttccaataccaaggtggataagcgggtggagcctaagagctgtgataagacccacacctgccctccttgccccgccccagagctgctgggaggacctagcgtgtttctgtttccccctaagcctaaggacaccctgatgatcagcaggacccccgaggtgacctgtgtggtggtggacgtgtcccacgaggaccctgaggtgaagtttaactggtatgtggacggcgtggaggtgcacaatgccaagaccaagccccgggaggagcagtataattccacctatagggtggtgagcgtgctgaccgtgctgcaccaggattggctgaacggcaaggagtacaagtgtaaggtgtccaacaaggctctgcccgcccccatcgagaagaccatctccaaggctaagggccagcccagggagccccaggtgtataccctgcctccctccagggaggagatgaccaagaaccaggtgtccctgacctgcctggtgaagggcttttatccttccgacatcgccgtggagtgggagtccaacggccagcccgagaataactataagaccacccctcccgtgctggacagcgatggcagcttctttctgtacagcaagctgaccgtggacaagagccggtggcagcagggcaatgtgttctcctgcagcgtgatgcacgaggctctgcacaaccactatacccagaagtccctgtccctgagccccggcgcccctgaagctccaaccgatcccgaggcccccaccgatatccctcctcacgtgcagaagagcgtgaacaacgatatgatcgtgaccgataataatggcgccgtgaagttccctcagctgtgtaagttttgtgacgtgaggttcagcacctgcgataaccagaagagctgtatgtccaattgcagcatcaccagcatctgcgagaagccccaggaggtgtgcgtggccgtgtggaggaagaacgatgagaacatcaccctggagaccgtgtgtcacgaccccaagctgccttaccacgattttatcctggaggatgctgcttcccccaagtgcatcatgaaggagaagaagaagcccggcgagaccttcttcatgtgcagctgcagcagcgacgagtgcaatgataatatcatcttttcc

SEQ ID NO: 186 - DNA, 1800 bp, synthetic construct
  misc_feature 1..1800: note = Fusion protein - heavy chain 4
  source 1..1800: mol_type = other DNA
cagggacagctggtgcagagcggcgctgaggtgaagaagcccggctccagcgtgaaggtgtcctgcaagacctccggcttcaccttctcctccagctatatctcctgggtgcggcaggcccccggccagggactggagtggatgggatggatctacgctggcaccggcggcacctcctacaaccagaagttcaccggcagggtgaccatcaccgtggatacctccaccagcaccgcttatatggagctgtcctccctgcggtccgaggataccgccgtgtactactgcgcccggcacgagggcaagtattggtactttgacgtgtggggccagggcaccaccgtgaccgtgagcagcgctagcaccaagggcccttccgtgttccccctggctccctccagcaagagcaccagcggcggcaccgctgctctgggatgcctggtgaaggactatttccctgagcctgtgaccgtgtcctggaacagcggcgctctgaccagcggcgtgcacaccttccctgccgtgctgcagtccagcggcctgtattccctgagcagcgtggtgaccgtgccttccagctccctgggcacccagacctacatctgtaacgtgaaccacaagcctagcaacaccaaggtggacaagcgggtggagcccaagtcctgtgataagacccacacctgccctccctgtcctgctcctgagctgctgggcggcccctctgtgtttctgtttcctcccaagcctaaggataccctgatgatctccaggacccccgaggtgacctgcgtggtggtggacgtgtcccacgaggaccctgaggtgaagtttaattggtatgtggacggcgtggaggtgcacaacgctaagaccaagcccagggaggagcagtacaactccacctatagggtggtgtccgtgctgaccgtgctgcaccaggattggctgaatggcaaggagtacaagtgcaaggtgagcaacaaggccctgcccgctcctatcgagaagaccatctccaaggctaagggccagccccgggagcctcaggtgtataccctgccccctagccgggaggagatgaccaagaatcaggtgtccctgacctgtctggtgaagggcttctatccctccgatatcgctgtggagtgggagagcaacggccagcccgagaataactataagaccacccctcccgtgctggatagcgacggctcctttttcctgtatagcaagctgaccgtggataagagcaggtggcagcagggcaacgtgtttagctgctccgtgatgcacgaggctctgcacaaccactacacccagaagagcctgtccctgtcccccggcgctcctgaggctcctaccgaccctgaggcccctaccgatcctgaggctcccaccgatcccgaggcccctacagacatccctccccacgtgcagaagtccgtgaataacgatatgatcgtgaccgacaacaacggcgctgtgaagtttccccagctgtgcaagttttgcgacgtgcggttcagcacctgcgataaccagaagagctgtatgagcaattgctccatcacctccatctgcgagaagcctcaggaggtgtgtgtggccgtgtggcggaagaacgacgagaacatcaccctggagaccgtgtgccacgaccccaagctgccttatcacgatttcatcctggaggacgccgccagccccaagtgtatcatgaaggagaagaagaagcctggcgagacctttttcatgtgcagctgctcctccgatgagtgcaatgataacatcatcttctcc

SEQ ID NO: 187 - DNA, 1788 bp, synthetic construct
  misc_feature 1..1788: note = Fusion protein - heavy chain 5
  source 1..1788: mol_type = other DNA
caggtgcagctggtgcagagcggcgctgaggtgaagaagcccggcgcttccgtgaaggtgtcctgtaaggccagcggctacagcttcaccggctacaccatgaactgggtgaagcaggctcacggccagggcctggagtggatgggcctgatcatcccttacaacggcggcatcagctataatcagaagttcaaggaccgggtgaccatgaccgtggataccagcatcagcaccgcctacatggagctgtccaggctgcggagcgatgataccgctgtgtattactgcgccagcctgatcaccaccgctcctagggatagcatggactattggggccagggcaccctggtgaccgtgtccagcgcttccaccaagggcccttccgtgttccctctggctccttccagcaagagcacctccggcggcaccgccgctctgggatgtctggtgaaggattatttccccgagcccgtgaccgtgagctggaacagcggcgccctgacctccggcgtgcacacattccctgccgtgctgcagagcagcggcctgtacagcctgtccagcgtggtgaccgtgccctccagcagcctgggcacccagacctatatctgcaatgtgaatcacaagcctagcaacaccaaggtggacaagagggtggagcctaagtcctgcgataagacccacacctgtcctccttgccccgcccctgagctgctgggaggaccttccgtgtttctgttccctcccaagcctaaggataccctgatgatcagccggacccctgaggtgacctgcgtggtggtggatgtgagccacgaggaccctgaggtgaagttcaactggtacgtggatggcgtggaggtgcacaatgccaagaccaagcccagggaggagcagtacaacagcacctatagggtggtgagcgtgctgaccgtgctgcaccaggattggctgaacggcaaggagtataagtgcaaggtgtccaacaaggctctgcccgctcctatcgagaagaccatctccaaggctaagggccagcctcgggagcctcaggtgtataccctgcctcctagccgggaggagatgaccaagaaccaggtgagcctgacctgcctggtgaagggcttctatcctagcgacatcgctgtggagtgggagtccaacggccagcccgagaacaactacaagaccaccccccccgtgctggatagcgatggctccttcttcctgtatagcaagctgaccgtggacaagagccggtggcagcagggcaacgtgttctcctgtagcgtgatgcacgaggccctgcacaaccactatacccagaagtccctgtccctgtcccctggcgcccctgaggctccaaccgatcctgaggcccccaccgaccctgaggccccaacagacatcccccctcacgtgcagaagtccgtgaacaacgacatgatcgtgaccgataacaacggcgctgtgaagttccctcagctgtgcaagttttgcgatgtgaggttcagcacctgcgacaatcagaagagctgcatgagcaactgcagcatcacctccatctgtgagaagccccaggaggtgtgcgtggctgtgtggcggaagaacgatgagaacatcaccctggagaccgtgtgtcacgaccctaagctgccttatcacgattttatcctggaggacgccgcctcccccaagtgtatcatgaaggagaagaagaagcctggcgagaccttcttcatgtgttcctgcagcagcgatgagtgcaatgataatatcatcttcagc

SEQ ID NO: 188 - DNA, 1776 bp, synthetic construct
  misc_feature 1..1776: note = Fusion protein - heavy chain 6
  source 1..1776: mol_type = other DNA
caggtgcagctggtgcagtccggcgctgaggtgaagaagcctggcgccagcgtgaaggtgtcctgtaaggcttccggcttcaacatcaaggacacctatatgcactgggtgcggcaggctcccggccagggattggagtggatcggccggatcgatcctgccaacggcaacaccaagtatgatcccaagttccagggccgggtgaccatcaccaccgacaccagcgccagcaccgcttacctggagctgagcagcctgcggtccgaggataccgccgtgtattactgtgccaggggcctgggcaggtggtttgcctattggggccagggcaccaccgtgaccgtgtccagcgctagcaccaagggcccctccgtgttccctctggctccttccagcaagtccaccagcggcggcaccgccgctctgggatgtctggtgaaggactacttccccgagcccgtgaccgtgagctggaactccggcgccctgacctccggcgtgcacacatttcctgccgtgctgcagagcagcggcctgtattccctgagcagcgtggtgaccgtgcctagctccagcctgggcacccagacctacatctgcaacgtgaatcacaagccctccaacaccaaggtggataagcgggtggagcccaagtcctgtgacaagacccacacctgccccccttgtcccgctcctgagctgctgggcggcccttctgtgtttctgttcccccctaagcctaaggacaccctgatgatctcccggacccctgaggtgacctgcgtggtggtggacgtgtcccacgaggaccctgaggtgaagtttaattggtacgtggatggcgtggaggtgcacaacgctaagaccaagcctagggaggagcagtataatagcacctatcgggtggtgagcgtgctgaccgtgctgcaccaggactggctgaacggcaaggagtacaagtgcaaggtgtccaataaggccctgcctgctcctatcgagaagaccatcagcaaggctaagggccagcccagggagccccaggtgtataccctgcccccttcccgggaggagatgaccaagaaccaggtgagcctgacctgtctggtgaaaggcttttaccccagcgatatcgccgtggagtgggagagcaatggccagcctgagaacaattataagaccaccccccctgtgctggacagcgatggctcctttttcctgtacagcaagctgaccgtggataagagcaggtggcagcagggcaatgtgttctcctgttccgtgatgcacgaggccctgcacaatcactacacccagaagtccctgtccctgagccccggcgccccagaagctcctaccgacccagaggcccctaccgaccccgaggctccaaccgacatccctcctcacgtgcagaagtccgtgaacaacgacatgatcgtgaccgataataatggcgccgtgaagtttcctcagctgtgcaagttctgtgatgtgcggtttagcacctgcgataaccagaagtcctgtatgtccaattgctccatcaccagcatctgcgagaagccccaggaggtgtgcgtggctgtgtggcggaagaatgatgagaatatcaccctggagaccgtgtgccacgaccccaagctgccctatcacgacttcatcctggaggatgctgccagccccaagtgcatcatgaaggagaagaagaagcctggagagaccttcttcatgtgtagctgcagcagcgatgagtgcaatgataatatcatcttcagc

SEQ ID NO: 189 - DNA, 1788 bp, synthetic construct
  misc_feature 1..1788: note = Fusion protein - heavy chain 7
  source 1..1788: mol_type = other DNA
gaggtgcagctgcagcagtccggccccgagctggtgaagcccggagctagcatgaagatcagctgcaaggcctccggctacagctttaccggctacaccatgaactgggtgaagcagagccacggcaagaacctggagtggatcggcctgatcatccctcacaatggcggcaccagctacaatcagaagttcaaggataaggccaccctgaccgtggacaagtccagcaggaccgcctacatggagctgctgtccctgaccagcgaggacagcgccgtgtactactgcgccagcctgatgaccaccgcccccagggacagcatggattattggggccagggcaccagcgtgaccgtgtccagcgctagcaccaagggcccttccgtgttccctctggccccttccagcaagtccaccagcggcggcaccgctgctctgggatgcctggtgaaggattacttccccgagcccgtgaccgtgtcttggaatagcggcgctctgacctccggcgtgcacacctttcctgccgtgctgcagtcctccggcctgtattccctgagcagcgtggtgaccgtgcccagcagctccctgggcacccagacctatatctgcaatgtgaaccacaagcccagcaataccaaggtggacaagcgggtggagcccaagtcctgcgacaagacccacacctgccccccctgtcctgctcccgagctgctgggaggccctagcgtgttcctgttcccccccaagcccaaggacaccctgatgatcagccggacccctgaggtgacctgtgtggtggtggacgtgagccacgaggaccctgaggtgaagtttaattggtacgtggacggcgtggaggtgcacaatgctaagaccaagcccagggaggagcagtacaacagcacctatagggtggtgagcgtgctgaccgtgctgcaccaggactggctgaacggcaaggagtataagtgtaaggtgtccaacaaggccctgcccgcccctatcgagaagaccatcagcaaggctaagggccagcctcgggagccccaggtgtataccctgcctccctcccgggaggagatgaccaagaaccaggtgagcctgacctgcctggtgaaaggcttctatccttccgacatcgccgtggagtgggagagcaacggccagcccgagaacaactacaagaccacccctcccgtgctggatagcgacggctccttctttctgtatagcaagctgaccgtggataagtccaggtggcagcagggcaatgtgtttagctgctccgtgatgcacgaggccctgcacaatcactacacccagaagtccctgagcctgtcccctggcgcccccgaagctccaaccgacccagaggcccctaccgatcctgaggcccccaccgatatcccccctcacgtgcagaagagcgtgaacaatgacatgatcgtgaccgacaacaacggcgccgtgaagttccctcagctgtgcaagttttgcgatgtgcggttctccacctgcgacaaccagaagagctgcatgtccaactgctccatcacctccatctgtgagaagcctcaggaggtgtgtgtggctgtgtggaggaagaacgacgagaacatcaccctggagaccgtgtgtcacgatcctaagctgccttaccacgactttatcctggaggacgccgctagccccaagtgcatcatgaaggagaagaagaagcctggcgagaccttctttatgtgctcctgcagcagcgacgagtgcaatgacaatatcatcttcagc

SEQ ID NO: 190 - DNA, 639 bp, synthetic construct
  misc_feature 1..639: note = Fusion protein - light chain 1
  source 1..639: mol_type = other DNA
gagatcgtgatgacccagagccccgccaccctgtccgtgtccccaggagagcgggccaccctgtcttgtagcgcttctagctccgtgagctatgtgcactggtatcaacagaagcccggccaggctcccaggccctggatctatgataccagcaatctggcctccggcttccccgctaggttctccggctccggcagcggaaccgagtataccctgaccatctcctccctgcagtccgaggacgccgctgtgtattattgccaccagaggagctcctatccctggaccttcggcggcggcaccaaggtggagatcaagaggaccgtggccgctccctccgtgttcatcttccccccctccgatgagcagctgaagagcggcaccgccagcgtggtgtgcctgctgaacaacttctaccctagggaggctaaggtgcagtggaaggtggacaacgctctgcagtccggcaatagccaggagagcgtgaccgagcaggatagcaaggacagcacctattccctgtcctccaccctgaccctgtccaaggctgactacgagaagcacaaggtgtacgcttgtgaggtgacccaccagggcctgtccagccccgtgaccaagtcctttaatcggggcgagtgt

SEQ ID NO: 191 - DNA, 642 bp, synthetic construct
  misc_feature 1..642: note = Fusion protein - light chain 2
  source 1..642: mol_type = other DNA
gacatccagatgacccagagcccttccagcctgagcgccagcgtgggcgacagggtgaccatcacctgcagggctagccaggatatcagcaattacctgaactggtatcaacagaagcccggcaaggctcctaagctgctgatctactacaccagcaagctgcactccggcgtgcctagcaggtttagcggctccggctccggcaccgattttaccctgaccatcagcagcctgcagcctgaggatatcgctacctattactgccagcagggcgatgccctgccttggacctttggcggcggcaccaaggtggagatcaagaggaccgtggccgctccttccgtgtttatcttcccccctagcgacgagcagctgaagagcggcaccgcttccgtggtgtgtctgctgaataacttttaccctcgggaggctaaggtgcagtggaaggtggacaacgctctgcagagcggcaactcccaggagagcgtgaccgagcaggactccaaggacagcacctactccctgtccagcaccctgaccctgagcaaggccgattacgagaagcacaaggtgtacgcctgtgaggtgacccaccagggcctgtcctcccccgtgaccaagtcctttaataggggcgagtgc

SEQ ID NO: 192 - DNA, 642 bp, synthetic construct
  misc_feature 1..642: note = Fusion protein - light chain 3
  source 1..642: mol_type = other DNA
gacatccagatgacccagtcccccagcagcctgagcgcttccgtgggcgaccgggtgaccatcacctgccaggcctcccaggatgtgagcaatgctgtggcctggtatcaacagaagcccggcaaggcccctaagctgctgatctatagcgctagcaacctgtacaccggcgtgcccagcaggttcagcggcagcggatctggcaccgattttaccttcaccatcagcagcctgcagcccgaggacatcgctacctactactgccagcagcacgacagcacccccctgaccttcggccagggcaccaagctggagctgaagaggaccgtggccgcccccagcgtgttcatcttccccccttccgacgagcagctgaagagcggcaccgctagcgtggtgtgcctgctgaataatttttaccctagggaggctaaggtgcagtggaaggtggacaacgctctgcagagcggcaatagccaggagtccgtgaccgagcaggacagcaaggacagcacctatagcctgagcagcaccctgaccctgtccaaggccgactatgagaagcacaaggtgtacgcctgtgaggtgacccaccagggcctgtccagccctgtgaccaagtccttcaatcggggcgagtgc

SEQ ID NO: 193 - DNA, 642 bp, synthetic construct
  misc_feature 1..642: note = Fusion protein - light chain 4
  source 1..642: mol_type = other DNA
gatatccagatgacccagaccacctcctccctgtccgccagcctgggcgatagggtgaccatctcctgcagggccagccaggatatctccaactatctgaactggtatcaacagaagcctgatggcaccgtgaagctgctgatctactacaccagccggctgcactccggcgtgccctctaggttcagcggcagcggcagcggaaccgactatagcctgaccatcagcaacctggatcaggaggacatcgctacctacttctgtcagcagggcgccaccctgccctggacctttggcggaggcaccaagctggagatcaagcggaccgtggctgctcccagcgtgttcatcttccctccttccgatgagcagctgaagtccggcaccgcctccgtggtgtgtctgctgaacaacttctatcctagggaggccaaggtgcagtggaaggtggacaacgccctgcagagcggcaactcccaggagagcgtgaccgagcaggatagcaaggatagcacctattccctgtccagcaccctgaccctgtccaaggctgactacgagaagcacaaggtgtacgcctgtgaggtgacccaccagggcctgtccagccctgtgaccaagagcttcaaccggggcgagtgt

SEQ ID NO: 194 - DNA, 1773 bp, synthetic construct
  misc_feature 1..1773: note = Fusion protein - heavy chain 8
  source 1..1773: mol_type = other DNA
cagggacagctggtgcagagcggcgccgaggtgaagaagcctggcagcagcgtgaaggtgtcctgcaagacctccggctttacctttagctcctcctatatctcctgggtgcggcaggctcccggccagggattggagtggatgggctggatctacgccggcaccggcggcacatcctacaaccagaagtttaccggccgggtgaccatcaccgtggacaccagcacctccaccgcttatatggagctgtccagcctgcggtccgaggataccgctgtgtactactgcgcccggcacgagggcaagtactggtactttgatgtgtggggccagggcaccaccgtgaccgtgtcctccgcctccaccaagggccctagcgtgtttcctctggctccctgtagcaggagcacctccgagagcaccgccgccctgggatgtctggtgaaggattattttcccgagcctgtgaccgtgagctggaatagcggcgctctgaccagcggcgtgcacacctttcctgctgtgctgcagagcagcggcctgtattccctgtcctccgtggtgaccgtgcctagctcctccctgggcaccaagacctacacctgcaacgtggatcacaagccttccaataccaaggtggacaagcgggtggagagcaagtacggccccccctgtcccccttgtcccgctcctgagtttctgggcggcccttccgtgttcctgtttccccccaagcctaaggacaccctgatgatctcccggacccctgaggtgacctgcgtggtggtggatgtgtcccaggaggaccctgaggtgcagttcaactggtatgtggacggcgtggaggtgcacaacgctaagaccaagcctcgggaggagcagttcaactccacctacagggtggtgtccgtgctgaccgtgctgcaccaggactggctgaatggcaaggagtataagtgtaaggtgtccaataagggcctgccttcctccatcgagaagaccatcagcaaggctaagggccagcccagggagccccaggtgtataccctgcctcccagccaggaggagatgaccaagaaccaggtgagcctgacctgcctggtgaagggcttctacccctccgatatcgccgtggagtgggagtccaacggccagcctgagaacaactacaagaccacccctcctgtgctggactccgacggctcctttttcctgtacagccggctgaccgtggataagagcaggtggcaggagggcaatgtgttcagctgttccgtgatgcacgaggctctgcacaaccactacacccagaagagcctgagcctgtccctgggcgcccctgaggctcctaccgatcccgaggctcccaccgatcccgaagctcccaccgacatcccccctcacgtgcagaagtccgtgaacaacgatatgatcgtgaccgataacaatggcgccgtgaagtttccccagctgtgtaagttctgcgatgtgaggtttagcacctgtgacaatcagaagagctgcatgagcaactgctccatcaccagcatctgtgagaagccccaggaggtgtgtgtggctgtgtggaggaagaacgatgagaacatcaccctggagaccgtgtgccacgatcccaagctgccctaccacgatttcatcctggaggatgctgccagccctaagtgtatcatgaaggagaagaagaagcctggagagaccttcttcatgtgcagctgctccagcgacgagtgcaacgataacatcatcttctcc

SEQ ID NO: 195 - DNA, 1800 bp, synthetic construct
  misc_feature 1..1800: note = Fusion protein - heavy chain 9
  source 1..1800: mol_type = other DNA
cagggacagctggtgcagagcggcgctgaggtgaagaagcctggcagcagcgtgaaggtgtcctgtaagacctccggcttcacctttagctccagctacatcagctgggtgcggcaggcccctggccagggattggagtggatgggctggatctacgctggcaccggcggcacctcctacaaccagaagttcaccggcagggtgaccatcaccgtggatacctccacctccaccgcttacatggagctgagcagcctgcggtccgaggacaccgccgtgtattactgtgctcggcacgagggcaagtactggtatttcgatgtgtggggccagggcaccaccgtgaccgtgtcctccgcttccaccaagggcccctccgtgtttcctctggccccttgctcccggtccaccagcgagtccaccgccgctctgggctgtctggtgaaggattattttcctgagcctgtgaccgtgtcttggaacagcggcgctctgacctccggcgtgcacaccttccctgccgtgctgcagagcagcggcctgtacagcctgtccagcgtggtgaccgtgcctagctcctccctgggcaccaagacctatacctgcaacgtggatcacaagccttccaacaccaaggtggataagagggtggagagcaagtacggccccccttgtcccccttgtccagctcctgagttcctgggcggcccctccgtctttctgttccctcccaagcctaaggataccctgatgatctccaggacccctgaggtgacctgtgtggtggtggatgtgagccaggaggaccctgaggtgcagttcaactggtacgtggacggcgtggaggtgcacaatgccaagaccaagcctagggaggagcagtttaatagcacctacagggtggtgagcgtgctgaccgtgctgcaccaggattggctgaacggcaaggagtataagtgtaaggtgagcaataagggcctgccctccagcatcgagaagaccatctccaaggctaagggccagccccgggagccccaggtttataccctgccccccagccaggaggagatgaccaagaatcaggtgagcctgacctgcctggtgaagggcttttatccctccgacatcgctgtggagtgggagagcaacggccagcctgagaacaactacaagaccacccctcctgtgctggacagcgacggcagctttttcctgtacagccggctgaccgtggacaagtcccggtggcaggagggcaatgtgtttagctgctccgtgatgcacgaggccctgcacaatcactatacccagaagtccctgagcctgtccctgggcgctcctgaggccccaaccgaccctgaggctcccaccgatcccgaggcccctaccgacatcccccctcacgtgcagaagtccgtgaacaatgacatgatcgtgaccgacaacaatggcgctgtgaagtttccccagctgtgcaagttctgcgacgtgaggttttccacctgtgacaatcagaagtcctgtatgtccaattgcagcatcacctccatctgcgagaagccccaggaggtgtgcgtggctgtgtggaggaagaatgatgagaatatcaccctggagaccgtgtgtcacgatcctaagctgccttatcacgattttatcctggaggatgccgccagccccaagtgcatcatgaaggagaagaagaagcccggcgagacctttttcatgtgcagctgttccagcgacgagtgcaatgataacatcatcttcagcgaggagtataacacctccaaccctgac

SEQ ID NO: 196 - DNA, 1755 bp, synthetic construct
  misc_feature 1..1755: note = Fusion protein - heavy chain 10
  source 1..1755: mol_type = other DNA
cagggacagctggtgcagtccggcgccgaggtgaagaagcccggcagcagcgtgaaggtgtcctgcaagaccagcggctttacctttagctccagctacatcagctgggtgaggcaggctcccggccagggactggagtggatgggctggatctatgctggcaccggcggcacctcctacaatcagaagtttaccggccgggtgaccatcaccgtggacaccagcacctccaccgcttacatggagctgtcctccctgcggtccgaggataccgccgtgtactactgtgcccggcacgagggcaagtactggtactttgacgtgtggggccagggcaccaccgtgaccgtgtcctccgccagcaccaagggcccttccgtgtttcccctggctccttgcagccggtccacctccgagagcaccgccgctctgggctgtctggtgaaggactacttccctgagcccgtgaccgtgagctggaatagcggcgccctgacctccggcgtgcacacatttcccgctgtgctgcagagcagcggcctgtattccctgtccagcgtggtgaccgtgccctcctccagcctgggcaccaagacctatacctgcaacgtggaccacaagccctccaataccaaggtggataagcgggtggagtccaagtatggccccccttgccccccctgtcctgcccctgaatttctgggcggccccagcgtgttcctgttccccccaaagcccaaggacaccctgatgatctccaggacccctgaggtgacctgcgtggtggtggatgtgagccaggaggaccctgaggtgcagttcaattggtatgtggacggcgtggaggtgcacaatgccaagaccaagcctcgggaggagcagttcaactccacctacagggtggtgagcgtgctgaccgtgctgcaccaggattggctgaatggcaaggagtataagtgcaaggtgtccaacaagggcctgccttcctccatcgagaagaccatctccaaggccaagggccagccccgggagcctcaggtttacaccctgccccctagccaggaggagatgaccaagaaccaggtgagcctgacctgcctggtgaagggcttttaccccagcgatatcgccgtggagtgggagtccaacggccagcccgagaacaattacaagaccacccctcccgtgctggactccgatggcagctttttcctgtacagccggctgaccgtggacaagtcccggtggcaggagggcaacgtgttttcctgctccgtgatgcacgaggccctgcacaaccactatacccagaagagcctgtccctgtccctgggcgctcccgaggctccaaccgacccagaggcccctaccgatatccctcctcacgtgcagaagagcgtgaacaacgatatgatcgtgaccgataataatggcgccgtgaagttccctcagctgtgtaagttttgcgacgtgaggttcagcacctgcgacaatcagaagagctgtatgagcaattgtagcatcacctccatctgcgagaagccccaggaggtgtgcgtggctgtgtggcggaagaatgatgagaacatcaccctggagaccgtgtgccacgatcccaagctgccctaccacgattttatcctggaggatgctgcctcccccaagtgcatcatgaaggagaagaagaagcccggagagaccttcttcatgtgcagctgtagctccgatgagtgtaatgacaatatcatcttttcc

SEQ ID NO: 197 - DNA, 1791 bp, synthetic construct
  misc_feature 1..1791: note = Fusion protein - heavy chain 11
  source 1..1791: mol_type = other DNA
cagggacagctggtgcagagcggcgccgaggtgaagaagcctggctccagcgtgaaggtgtcctgcaagaccagcggcttcaccttttccagctcctacatcagctgggtgcggcaggctcccggccagggattggagtggatgggctggatctatgccggcaccggcggcaccagctacaatcagaagttcaccggccgggtgaccatcaccgtggacacctccacctccaccgcctacatggagctgtccagcctgaggagcgaggataccgctgtgtactactgcgccaggcacgagggcaagtattggtatttcgacgtgtggggccagggcaccaccgtgaccgtgtcctccgcctccaccaagggcccctccgtgtttcccctggccccctgtagcaggtccaccagcgagagcaccgccgctctgggctgcctggtgaaggactacttccctgagcctgtgaccgtgagctggaactccggcgccctgaccagcggcgtgcacacattccctgccgtgctgcagagcagcggcctgtactccctgagcagcgtggtgaccgtgcctagcagctccctgggcaccaagacctatacctgtaatgtggatcacaagcctagcaataccaaggtggacaagcgggtggagtccaagtacggccccccttgccccccctgcccagctcctgaatttctgggcggcccttccgtgttcctgtttcctcccaagcccaaggacaccctgatgatcagccggacccctgaggtgacctgtgtggtggtggatgtgagccaggaggaccctgaggtgcagttcaattggtacgtggacggcgtggaggtgcacaacgccaagaccaagcccagggaggagcagttcaacagcacctacagggtggtgtccgtgctgaccgtgctgcaccaggattggctgaatggcaaggagtacaagtgtaaggtgagcaataagggcctgccttccagcatcgagaagaccatcagcaaggccaagggccagcctagggagccccaggtgtacaccctgccccccagccaggaggagatgaccaagaatcaggtgagcctgacctgtctggtgaagggcttctatcccagcgatatcgctgtggagtgggagagcaacggccagcctgagaacaattacaagaccacccctcctgtgctggattccgatggctcctttttcctgtactcccggctgaccgtggataagagccggtggcaggagggcaacgtgtttagctgtagcgtgatgcacgaggctctgcacaaccactacacccagaagagcctgagcctgagcctcggcgcccctgaggctccaaccgacccagaggcccctaccgatcccgaggcccctacagatcctgaggcccccaccgatatccccccccacgtgcagaagtccgtgaataacgacatgatcgtgaccgataacaacggcgctgtgaagttccctcagctgtgcaagttctgtgatgtgaggtttagcacctgtgacaaccagaagagctgcatgagcaactgctccatcacctccatctgcgagaagcctcaggaggtgtgtgtggccgtgtggcggaagaatgatgagaatatcaccctggagaccgtgtgccacgaccccaagctgccctaccacgacttcatcctggaggatgctgctagccccaagtgtatcatgaaggagaagaagaagcctggagagaccttcttcatgtgtagctgcagctccgatgagtgtaacgataatatcatcttctcc

SEQ ID NO: 198 - DNA, 1779 bp, synthetic construct
  misc_feature 1..1779: note = Fusion protein - heavy chain 12
  source 1..1779: mol_type = other DNA
caggtgcagctggtgcagagcggcgctgaggtgaagaagcccggcgcttccgtgaaggtgtcctgcaaggcttccggctactcctttaccggctacaccatgaactgggtgaagcaggcccacggccagggcctggagtggatgggactgatcatcccctacaacggcggcatctcctacaaccagaagttcaaggatcgggtgaccatgaccgtggacaccagcatcagcaccgcttatatggagctgagcaggctgcggtccgacgacaccgccgtgtactactgcgcctccctgatcaccaccgcccctcgggacagcatggattactggggccagggcaccctggtgaccgtgagcagcgccagcaccaagggcccctccgtgttccctctggccccctgtagcaggtccaccagcgagtccaccgccgctctgggctgcctggtgaaggactactttcccgagcccgtgaccgtgtcttggaactccggcgctctgacctccggcgtgcacacctttcccgctgtgctgcagtccagcggcctgtacagcctgagcagcgtggtgaccgtgcctagctccagcctgggcaccaagacctatacctgtaatgtggaccacaagcccagcaacaccaaggtggacaagcgggtggagagcaagtacggccccccttgccctccttgtcctgccccagagtttctgggcggccccagcgtgtttctgtttccccccaagcctaaggacaccctgatgatctcccggacccctgaggtgacctgcgtggtggtggatgtgagccaggaggaccctgaggtgcagttcaattggtacgtggacggcgtggaggtgcacaatgctaagaccaagcctagggaggagcagtttaactccacctatagggtggtgagcgtgctgaccgtgctgcaccaggattggctgaatggcaaggagtacaagtgcaaggtgagcaacaagggcctgccctccagcatcgagaagaccatctccaaggctaagggccagcccagggagccccaggtgtacaccctgccccccagccaggaggagatgaccaagaaccaggtgagcctgacctgtctggtgaagggcttttatcctagcgatatcgctgtggagtgggagtccaacggccagcccgagaacaactacaagaccacccctcctgtgctggatagcgatggcagcttcttcctgtacagccggctgaccgtggataagagcaggtggcaggagggcaacgtgttcagctgttccgtgatgcacgaggctctgcacaaccactacacccagaagtccctgtccctgagcctgggcgcccctgaggctccaaccgatcctgaggctcctaccgaccccgaggctcctacagacatccctcctcacgtgcagaagtccgtgaataacgacatgatcgtgaccgataacaacggcgctgtgaagttcccccagctgtgtaagttctgcgatgtgcggttcagcacctgtgacaatcagaagtcctgtatgtccaactgtagcatcaccagcatctgtgagaagccccaggaggtgtgcgtggctgtgtggcggaagaatgacgagaatatcaccctggagaccgtgtgtcacgaccctaagctgccctatcacgattttatcctggaggacgccgcctcccccaagtgcatcatgaaggagaagaagaagcctggcgagaccttcttcatgtgtagctgctcctccgatgagtgcaatgacaacatcatctttagc

SEQ ID NO: 199 - DNA, 1767 bp, synthetic construct
  misc_feature 1..1767: note = Fusion protein - heavy chain 13
  source 1..1767: mol_type = other DNA
caggtgcagctggtgcagagcggcgccgaggtgaagaagcctggcgcttccgtgaaggtgagctgtaaggctagcggcttcaatatcaaggacacctatatgcactgggtgcggcaggcccctggccagggattggagtggatcggccggatcgatcctgctaatggcaacaccaagtatgatcccaagttccagggcagggtgaccatcaccaccgacaccagcgcctccaccgcctatctggagctgagcagcctgcggtccgaggacaccgccgtgtattattgtgctcggggcctgggcaggtggttcgcctactggggccagggcaccaccgtgaccgtgagcagcgcttccaccaagggcccctccgtgttccctctggctccctgcagcaggtccaccagcgagtccaccgccgccctgggatgtctggtgaaggactactttcccgagcccgtgaccgtgtcttggaatagcggcgctctgaccagcggcgtgcacacctttcccgccgtgctgcagagcagcggcctgtactccctgtccagcgtggtgaccgtgccctcctccagcctgggcaccaagacctacacctgcaacgtggaccacaagcctagcaacaccaaggtggataagcgggtggagagcaagtacggccctccttgtcccccctgtcccgctccagagttcctgggcggccctagcgtgttcctgttcccccccaagcccaaggataccctgatgatcagccggacccctgaggtgacctgcgtggtggtggatgtgagccaggaggaccccgaggtgcagtttaattggtacgtggatggcgtggaggtgcacaatgctaagaccaagcctagggaggagcagttcaatagcacctacagggtggtgagcgtgctgaccgtgctgcaccaggattggctgaacggcaaggagtataagtgcaaggtgagcaacaagggcctgcctagctccatcgagaagaccatcagcaaggctaagggccagcctcgggagccccaggtgtacaccctgcctcctagccaggaggagatgaccaagaatcaggtgtccctgacctgtctggtgaaaggcttctatccttccgatatcgctgtggagtgggagagcaacggccagcctgagaataattataagaccacccctcctgtgctggatagcgacggcagctttttcctgtacagccggctgaccgtggacaagtcccggtggcaggagggcaacgtgttttcctgttccgtgatgcacgaggccctgcacaaccactacacccagaagagcctgtccctgagcctgggcgctcccgaggctcctaccgatcctgaggcccctaccgatcccgaggctccaaccgacatccccccccacgtgcagaagagcgtgaataatgacatgatcgtgaccgacaataacggcgccgtgaagttccctcagctgtgcaagttctgcgacgtgcggtttagcacctgcgacaaccagaagagctgtatgtccaactgctccatcacctccatctgtgagaagccccaggaggtgtgcgtggccgtgtggaggaagaatgatgagaatatcaccctggagaccgtgtgtcacgaccccaagctgccttaccacgatttcatcctggaggacgctgctagccctaagtgtatcatgaaggagaagaagaagcctggagagaccttctttatgtgtagctgcagcagcgacgagtgcaatgacaacatcatcttctcc

SEQ ID NO: 200 - DNA, 1779 bp, synthetic construct
  misc_feature 1..1779: note = Fusion protein - heavy chain 14
  source 1..1779: mol_type = other DNA
gaggtgcagctgcagcagtccggccccgagctggtgaagcccggagctagcatgaagatctcctgcaaggcctccggctactcctttaccggctacaccatgaattgggtgaagcagagccacggcaagaatctggagtggatcggcctgatcatccctcacaatggcggcacctcctacaaccagaagttcaaggacaaggctaccctgaccgtggacaagtcctccaggaccgcttatatggagctgctgtccctgaccagcgaggatagcgccgtgtactattgcgcctccctgatgaccaccgcccctagggacagcatggactactggggccagggcacctccgtgaccgtgagcagcgcctccaccaagggccccagcgtgttccccctggctccctgtagcaggtccacctccgagagcaccgctgccctgggctgtctggtgaaggactactttcccgagcccgtgaccgtgtcctggaatagcggcgctctgacctccggcgtgcacaccttccccgccgtgctgcagtccagcggcctgtacagcctgtccagcgtggtgaccgtgccttcctccagcctgggcaccaagacctacacctgtaacgtggaccacaagcccagcaataccaaggtggacaagagggtggagtccaagtatggccccccctgtcctccctgccccgctcctgaatttctgggcggcccctccgtgttcctgtttccccccaagcccaaggacaccctgatgatcagcaggacccccgaggtgacctgtgtggtggtggacgtgtcccaggaggaccccgaggtgcagttcaactggtacgtggacggcgtggaggtgcacaatgccaagaccaagcctcgggaggagcagttcaatagcacctatcgggtggtgagcgtgctgaccgtgctgcaccaggattggctgaacggcaaggagtacaagtgcaaggtgtccaacaagggcctgcccagctccatcgagaagaccatctccaaggctaagggccagccccgggagccccaggtttacaccctgcctcctagccaggaggagatgaccaagaaccaggtgtccctgacctgtctggtgaaaggcttttatcctagcgatatcgccgtggagtgggagagcaatggccagcccgagaacaattacaagaccacccctcctgtgctggacagcgatggcagcttctttctgtacagccggctgaccgtggataagagcaggtggcaggagggcaacgtgttctcctgtagcgtgatgcacgaggctctgcacaatcactacacccagaagtccctgagcctgtccctgggcgcccccgaagctcctaccgatcccgaggctcccaccgaccccgaggctcctaccgacatcccccctcacgtgcagaagtccgtgaacaacgatatgatcgtgaccgataacaacggcgctgtgaagttcccccagctgtgtaagttctgtgacgtgaggttttccacctgtgataaccagaagagctgtatgtccaattgcagcatcaccagcatctgtgagaagcctcaggaggtgtgcgtggccgtgtggcggaagaacgatgagaatatcaccctggagaccgtgtgccacgaccctaagctgccctaccacgatttcatcctggaggatgctgccagccctaagtgcatcatgaaggagaagaagaagcctggcgagacctttttcatgtgcagctgtagcagcgacgagtgtaacgacaatatcatcttcagc

SEQ ID NO: 201 - protein, 136 aa, synthetic construct
  REGION 1..136: note = TGF-betaRII isoform 1ECD 1-136 N19T mutant
IPPHVQKSVNNDMIVTDNTGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 202 - protein, 136 aa, synthetic construct
  REGION 1..136: note = TGF-betaRII ECD mutant N19A (1-136)
IPPHVQKSVNNDMIVTDNAGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 203 - protein, 136 aa, synthetic construct
  REGION 1..136: note = TGF-betaRII ECD mutant K7G N19A (1-136)
IPPHVQGSVNNDMIVTDNAGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 204 - protein, 127 aa, synthetic construct
  REGION 1..127: note = TGF-betaRII ECD C-terminal truncation and mutant N19A (1-127)
IPPHVQKSVNNDMIVTDNAGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFS

SEQ ID NO: 205 - protein, 127 aa, synthetic construct
  REGION 1..127: note = TGF-betaRII ECD C-terminal truncation and mutant K7G N19A (1-127)
IPPHVQGSVNNDMIVTDNAGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFS

SEQ ID NO: 206 - protein, 590 aa, synthetic construct
  REGION 1..590: note = Fusion protein - heavy chain 15
QGQLVQSGAEVKKPGSSVKVSCKTSGFTFSSSYISWVRQAPGQGLEWMGWIYAGTGGTSYNQKFTGRVTITVDTSTSTAYMELSSLRSEDTAVYYCARHEGKYWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAPEAPTDEPEAPTDEIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFS

SEQ ID NO: 207 - protein, 597 aa, synthetic construct
  REGION 1..597: note = Fusion protein - heavy chain 16
QGQLVQSGAEVKKPGSSVKVSCKTSGFTFSSSYISWVRQAPGQGLEWMGWIYAGTGGTSYNQKFTGRVTITVDTSTSTAYMELSSLRSEDTAVYYCARHEGKYWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAPEAPTDEPEAPTDEPEAPTDEIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFS

SEQ ID NO: 208 - protein, 604 aa, synthetic construct
  REGION 1..604: note = Fusion protein - heavy chain 17
QGQLVQSGAEVKKPGSSVKVSCKTSGFTFSSSYISWVRQAPGQGLEWMGWIYAGTGGTSYNQKFTGRVTITVDTSTSTAYMELSSLRSEDTAVYYCARHEGKYWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAPEAPTDEPEAPTDEPEAPTDEPEAPTDEIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFS

SEQ ID NO: 209 - protein, 597 aa, synthetic construct
  REGION 1..597: note = Fusion protein - heavy chain 18
QGQLVQSGAEVKKPGSSVKVSCKTSGFTFSSSYISWVRQAPGQGLEWMGWIYAGTGGTSYNQKFTGRVTITVDTSTSTAYMELSSLRSEDTAVYYCARHEGKYWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAPEAPTDEPEAPTDEPEAPTDEIPPHVQGSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFS

SEQ ID NO: 210 - protein, 597 aa, synthetic construct
  REGION 1..597: note = Fusion protein - heavy chain 19
QGQLVQSGAEVKKPGSSVKVSCKTSGFTFSSSYISWVRQAPGQGLEWMGWIYAGTGGTSYNQKFTGRVTITVDTSTSTAYMELSSLRSEDTAVYYCARHEGKYWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAPEAPTDEPEAPTDEPEAPTDEIPPHVQGSVNNDMIVTDNAGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFS

SEQ ID NO: 211 - protein, 590 aa, synthetic construct
  REGION 1..590: note = Fusion protein - heavy chain 20
QGQLVQSGAEVKKPGSSVKVSCKTSGFTFSSSYISWVRQAPGQGLEWMGWIYAGTGGTSYNQKFTGRVTITVDTSTSTAYMELSSLRSEDTAVYYCARHEGKYWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAPEAPTDEPEAPTDEIPPHVQGSVNNDMIVTDNAGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFS

SEQ ID NO: 212 - protein, 593 aa, synthetic construct
  REGION 1..593: note = Fusion protein - heavy chain 21
QGQLVQSGAEVKKPGSSVKVSCKTSGFTFSSSYISWVRQAPGQGLEWMGWIYAGTGGTSYNQKFTGRVTITVDTSTSTAYMELSSLRSEDTAVYYCARHEGKYWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAPEAPTDEPEAPTDEPEAIPPHVQGSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFS

SEQ ID NO: 213 - DNA, 1770 bp, synthetic construct
  misc_feature 1..1770: note = Fusion protein - heavy chain 15
  source 1..1770: mol_type = other DNA
caaggtcaactagtgcaaagtggtgcagaagtgaagaagcctggaagctccgtgaaggtgtcctgtaagacatccggattcaccttctccagttcctatatctcctgggtgcggcaggcccctggccagggactggagtggatgggctggatctacgccggcacaggcggcacctcctacaaccagaaattcaccggcagggtaaccattacagtggacacatctacctccaccgcctacatggaactgagctctctgagatccgaggataccgctgtgtactactgcgccagacacgagggcaagtactggtatttcgacgtgtggggccagggcaccaccgtgaccgtgtcttctgcctcgaccaagggcccttctgtgttccctctggccccctcctccaagagcacatctggcggaaccgctgctctgggatgtctggtgaaggactactttcctgagcctgtgactgtttcttggaacagcggcgccctgacctccggagtgcataccttccctgccgtgctgcagtcttccggtctgtatagcctgtccagcgtggtgaccgtgcctagcagctctctgggcacacagacatatatttgtaacgtgaatcacaagccttctaacaccaaggtggacaagcgggttgagcctaagtcctgcgacaagacccacacgtgtcctccttgccctgctcctgagctgctcggaggcccttctgtgtttctgttcccaccaaaacccaaggacaccctgatgatctcccggacccctgaagtgacctgcgtggtggtggacgtgtctcacgaggaccccgaagtcaagttcaactggtacgtggacggcgtggaagtgcacaacgccaagaccaagcccagagaagagcagtacaattctacctacagagtggtctccgtgctgaccgtgctgcaccaggactggctgaacggcaaagagtacaagtgcaaggtctccaataaggccctgcctgctcctatcgagaagaccatctctaaggccaaaggccagcccagggaacctcaagtgtacacactgcccccaagccgcgaggaaatgaccaagaaccaggtgtccctgacatgtctcgtgaagggcttctaccctagcgacatcgccgtggagtgggagtccaatggccagcccgagaacaactacaagaccacaccacctgtgctggactccgatggctccttcttcctgtactccaagctgaccgtggataagtctagatggcagcagggcaacgtgttcagctgttctgtgatgcacgaggctctgcacaaccactacacccagaaatctctgtccctgtctcctggagctcctgaggctccaaccgacgagcctgaagcccctaccgacgagatccctcctcacgtgcagaagtccgtgaacaacgacatgatcgtgaccgacaacaacggcgccgttaagtttccccagctgtgcaagttctgcgatgtgcggttctctacctgtgataatcagaagtcctgcatgtccaactgctctatcacctccatctgcgagaagcctcaagaggtgtgtgtggctgtctggcggaagaacgatgagaacatcaccctggaaacagtgtgccacgatcccaagctgccttaccacgacttcatcctggaagatgctgcctctcctaagtgcatcatgaaagaaaagaaaaaacccggcgagacattcttcatgtgctcctgctcctctgacgagtgcaacgacaacatcatcttctct

SEQ ID NO: 214 - DNA, 1791 bp, synthetic construct
  misc_feature 1..1791: note = Fusion protein - heavy chain 16
  source 1..1791: mol_type = other DNA
caaggtcaactagtgcaaagtggtgcagaagtgaagaagcctggaagctccgtgaaggtgtcctgtaagacatccggattcaccttctccagttcctatatctcctgggtgcggcaggcccctggccagggactggagtggatgggctggatctacgccggcacaggcggcacctcctacaaccagaaattcaccggcagggtaaccattacagtggacacatctacctccaccgcctacatggaactgagctctctgagatccgaggataccgctgtgtactactgcgccagacacgagggcaagtactggtatttcgacgtgtggggccagggcaccaccgtgaccgtgtcttctgcctcgaccaagggcccttctgtgttccctctggccccctcctccaagagcacatctggcggaaccgctgctctgggatgtctggtgaaggactactttcctgagcctgtgactgtttcttggaacagcggcgccctgacctccggagtgcataccttccctgccgtgctgcagtcttccggtctgtatagcctgtccagcgtggtgaccgtgcctagcagctctctgggcacacagacatatatttgtaacgtgaatcacaagccttctaacaccaaggtggacaagcgggttgagcctaagtcctgcgacaagacccacacgtgtcctccttgccctgctcctgagctgctcggaggcccttctgtgtttctgttcccaccaaaacccaaggacaccctgatgatctcccggacccctgaagtgacctgcgtggtggtggacgtgtctcacgaggaccccgaagtcaagttcaactggtacgtggacggcgtggaagtgcacaacgccaagaccaagcccagagaagagcagtacaattctacctacagagtggtctccgtgctgaccgtgctgcaccaggactggctgaacggcaaagagtacaagtgcaaggtctccaataaggccctgcctgctcctatcgagaagaccatctctaaggccaaaggccagcccagggaacctcaagtgtacacactgcccccaagccgcgaggaaatgaccaagaaccaggtgtccctgacatgtctcgtgaagggcttctaccctagcgacatcgccgtggagtgggagtccaatggccagcccgagaacaactacaagaccacaccacctgtgctggactccgatggctccttcttcctgtactccaagctgaccgtggataagtctagatggcagcagggcaacgtgttcagctgttctgtgatgcacgaggctctgcacaaccactacacccagaaatctctgtccctgtctcctggagctcctgaggctccaaccgacgagcctgaagcccctaccgacgagccagaggcccccaccgatgagatccctcctcacgtgcagaagtccgtgaacaacgacatgatcgtgaccgacaacaacggcgccgttaagtttccccagctgtgcaagttctgcgatgtgcggttctctacctgtgataatcagaagtcctgcatgtccaactgctctatcacctccatctgcgagaagcctcaagaggtgtgtgtggctgtctggcggaagaacgatgagaacatcaccctggaaacagtgtgccacgatcccaagctgccttaccacgacttcatcctggaagatgctgcctctcctaagtgcatcatgaaagaaaagaaaaaacccggcgagacattcttcatgtgctcctgctcctctgacgagtgcaacgacaacatcatcttctct

SEQ ID NO: 215 - DNA, 1812 bp, synthetic construct
  misc_feature 1..1812: note = Fusion protein - heavy chain 17
  source 1..1812: mol_type = other DNA
caaggtcaactagtgcaaagtggtgcagaagtgaagaagcctggaagctccgtgaaggtgtcctgtaagacatccggattcaccttctccagttcctatatctcctgggtgcggcaggcccctggccagggactggagtggatgggctggatctacgccggcacaggcggcacctcctacaaccagaaattcaccggcagggtaaccattacagtggacacatctacctccaccgcctacatggaactgagctctctgagatccgaggataccgctgtgtactactgcgccagacacgagggcaagtactggtatttcgacgtgtggggccagggcaccaccgtgaccgtgtcttctgcctcgaccaagggcccttctgtgttccctctggccccctcctccaagagcacatctggcggaaccgctgctctgggatgtctggtgaaggactactttcctgagcctgtgactgtttcttggaacagcggcgccctgacctccggagtgcataccttccctgccgtgctgcagtcttccggtctgtatagcctgtccagcgtggtgaccgtgcctagcagctctctgggcacacagacatatatttgtaacgtgaatcacaagccttctaacaccaaggtggacaagcgggttgagcctaagtcctgcgacaagacccacacgtgtcctccttgccctgctcctgagctgctcggaggcccttctgtgtttctgttcccaccaaaacccaaggacaccctgatgatctcccggacccctgaagtgacctgcgtggtggtggacgtgtctcacgaggaccccgaagtcaagttcaactggtacgtggacggcgtggaagtgcacaacgccaagaccaagcccagagaagagcagtacaattctacctacagagtggtctccgtgctgaccgtgctgcaccaggactggctgaacggcaaagagtacaagtgcaaggtctccaataaggccctgcctgctcctatcgagaagaccatctctaaggccaaaggccagcccagggaacctcaagtgtacacactgcccccaagccgcgaggaaatgaccaagaaccaggtgtccctgacatgtctcgtgaagggcttctaccctagcgacatcgccgtggagtgggagtccaatggccagcccgagaacaactacaagaccacaccacctgtgctggactccgatggctccttcttcctgtactccaagctgaccgtggataagtctagatggcagcagggcaacgtgttcagctgttctgtgatgcacgaggctctgcacaaccactacacccagaaatctctgtccctgtctcctggagctcctgaggctccaaccgacgagcctgaagcccctaccgacgagccagaggcccccaccgatgagccagaggcccccaccgatgagatccctcctcacgtgcagaagtccgtgaacaacgacatgatcgtgaccgacaacaacggcgccgttaagtttccccagctgtgcaagttctgcgatgtgcggttctctacctgtgataatcagaagtcctgcatgtccaactgctctatcacctccatctgcgagaagcctcaagaggtgtgtgtggctgtctggcggaagaacgatgagaacatcaccctggaaacagtgtgccacgatcccaagctgccttaccacgacttcatcctggaagatgctgcctctcctaagtgcatcatgaaagaaaagaaaaaacccggcgagacattcttcatgtgctcctgctcctctgacgagtgcaacgacaacatcatcttctct

SEQ ID NO: 216 - DNA, 1791 bp, synthetic construct
  misc_feature 1..1791: note = Fusion protein - heavy chain 18
  source 1..1791: mol_type = other DNA
caaggtcaactagtgcaaagtggtgcagaagtgaagaagcctggaagctccgtgaaggtgtcctgtaagacatccggattcaccttctccagttcctatatctcctgggtgcggcaggcccctggccagggactggagtggatgggctggatctacgccggcacaggcggcacctcctacaaccagaaattcaccggcagggtaaccattacagtggacacatctacctccaccgcctacatggaactgagctctctgagatccgaggataccgctgtgtactactgcgccagacacgagggcaagtactggtatttcgacgtgtggggccagggcaccaccgtgaccgtgtcttctgcctcgaccaagggcccttctgtgttccctctggccccctcctccaagagcacatctggcggaaccgctgctctgggatgtctggtgaaggactactttcctgagcctgtgactgtttcttggaacagcggcgccctgacctccggagtgcataccttccctgccgtgctgcagtcttccggtctgtatagcctgtccagcgtggtgaccgtgcctagcagctctctgggcacacagacatatatttgtaacgtgaatcacaagccttctaacaccaaggtggacaagcgggttgagcctaagtcctgcgacaagacccacacgtgtcctccttgccctgctcctgagctgctcggaggcccttctgtgtttctgttcccaccaaaacccaaggacaccctgatgatctcccggacccctgaagtgacctgcgtggtggtggacgtgtctcacgaggaccccgaagtcaagttcaactggtacgtggacggcgtggaagtgcacaacgccaagaccaagcccagagaagagcagtacaattctacctacagagtggtctccgtgctgaccgtgctgcaccaggactggctgaacggcaaagagtacaagtgcaaggtctccaataaggccctgcctgctcctatcgagaagaccatctctaaggccaaaggccagcccagggaacctcaagtgtacacactgcccccaagccgcgaggaaatgaccaagaaccaggtgtccctgacatgtctcgtgaagggcttctaccctagcgacatcgccgtggagtgggagtccaatggccagcccgagaacaactacaagaccacaccacctgtgctggactccgatggctccttcttcctgtactccaagctgaccgtggataagtctagatggcagcagggcaacgtgttcagctgttctgtgatgcacgaggctctgcacaaccactacacccagaaatctctgtccctgtctcctggagctcctgaggctccaaccgacgagcctgaagcccctaccgacgagccagaggcccccaccgatgagatccctcctcacgtgcagggctccgtgaacaacgacatgatcgtgaccgacaacaacggcgccgttaagtttccccagctgtgcaagttctgcgatgtgcggttctctacctgtgataatcagaagtcctgcatgtccaactgctctatcacctccatctgcgagaagcctcaagaggtgtgtgtggctgtctggcggaagaacgatgagaacatcaccctggaaacagtgtgccacgatcccaagctgccttaccacgacttcatcctggaagatgctgcctctcctaagtgcatcatgaaagaaaagaaaaaacccggcgagacattcttcatgtgctcctgctcctctgacgagtgcaacgacaacatcatcttctct

SEQ ID NO: 217 - DNA, 1791 bp, synthetic construct
  misc_feature 1..1791: note = Fusion protein - heavy chain 19
  source 1..1791: mol_type = other DNA
caaggtcaactagtgcaaagtggtgcagaagtgaagaagcctggaagctccgtgaaggtgtcctgtaagacatccggattcaccttctccagttcctatatctcctgggtgcggcaggcccctggccagggactggagtggatgggctggatctacgccggcacaggcggcacctcctacaaccagaaattcaccggcagggtaaccattacagtggacacatctacctccaccgcctacatggaactgagctctctgagatccgaggataccgctgtgtactactgcgccagacacgagggcaagtactggtatttcgacgtgtggggccagggcaccaccgtgaccgtgtcttctgcctcgaccaagggcccttctgtgttccctctggccccctcctccaagagcacatctggcggaaccgctgctctgggatgtctggtgaaggactactttcctgagcctgtgactgtttcttggaacagcggcgccctgacctccggagtgcataccttccctgccgtgctgcagtcttccggtctgtatagcctgtccagcgtggtgaccgtgcctagcagctctctgggcacacagacatatatttgtaacgtgaatcacaagccttctaacaccaaggtggacaagcgggttgagcctaagtcctgcgacaagacccacacgtgtcctccttgccctgctcctgagctgctcggaggcccttctgtgtttctgttcccaccaaaacccaaggacaccctgatgatctcccggacccctgaagtgacctgcgtggtggtggacgtgtctcacgaggaccccgaagtcaagttcaactggtacgtggacggcgtggaagtgcacaacgccaagaccaagcccagagaagagcagtacaattctacctacagagtggtctccgtgctgaccgtgctgcaccaggactggctgaacggcaaagagtacaagtgcaaggtctccaataaggccctgcctgctcctatcgagaagaccatctctaaggccaaaggccagcccagggaacctcaagtgtacacactgcccccaagccgcgaggaaatgaccaagaaccaggtgtccctgacatgtctcgtgaagggcttctaccctagcgacatcgccgtggagtgggagtccaatggccagcccgagaacaactacaagaccacaccacctgtgctggactccgatggctccttcttcctgtactccaagctgaccgtggataagtctagatggcagcagggcaacgtgttcagctgttctgtgatgcacgaggctctgcacaaccactacacccagaaatctctgtccctgtctcctggagctcctgaggctccaaccgacgagcctgaagcccctaccgacgagccagaggcccccaccgatgagatccctcctcacgtgcagggctccgtgaacaacgacatgatcgtgaccgacaacgctggcgccgttaagtttccccagctgtgcaagttctgcgatgtgcggttctctacctgtgataatcagaagtcctgcatgtccaactgctctatcacctccatctgcgagaagcctcaagaggtgtgtgtggctgtctggcggaagaacgatgagaacatcaccctggaaacagtgtgccacgatcccaagctgccttaccacgacttcatcctggaagatgctgcctctcctaagtgcatcatgaaagaaaagaaaaaacccggcgagacattcttcatgtgctcctgctcctctgacgagtgcaacgacaacatcatcttctct

SEQ ID NO: 218 - DNA, 1770 bp, synthetic construct
  misc_feature 1..1770: note = Fusion protein - heavy chain 20
  source 1..1770: mol_type = other DNA
caaggtcaactagtgcaaagtggtgcagaagtgaagaagcctggaagctccgtgaaggtgtcctgtaagacatccggattcaccttctccagttcctatatctcctgggtgcggcaggcccctggccagggactggagtggatgggctggatctacgccggcacaggcggcacctcctacaaccagaaattcaccggcagggtaaccattacagtggacacatctacctccaccgcctacatggaactgagctctctgagatccgaggataccgctgtgtactactgcgccagacacgagggcaagtactggtatttcgacgtgtggggccagggcaccaccgtgaccgtgtcttctgcctcgaccaagggcccttctgtgttccctctggccccctcctccaagagcacatctggcggaaccgctgctctgggatgtctggtgaaggactactttcctgagcctgtgactgtttcttggaacagcggcgccctgacctccggagtgcataccttccctgccgtgctgcagtcttccggtctgtatagcctgtccagcgtggtgaccgtgcctagcagctctctgggcacacagacatatatttgtaacgtgaatcacaagccttctaacaccaaggtggacaagcgggttgagcctaagtcctgcgacaagacccacacgtgtcctccttgccctgctcctgagctgctcggaggcccttctgtgtttctgttcccaccaaaacccaaggacaccctgatgatctcccggacccctgaagtgacctgcgtggtggtggacgtgtctcacgaggaccccgaagtcaagttcaactggtacgtggacggcgtggaagtgcacaacgccaagaccaagcccagagaagagcagtacaattctacctacagagtggtctccgtgctgaccgtgctgcaccaggactggctgaacggcaaagagtacaagtgcaaggtctccaataaggccctgcctgctcctatcgagaagaccatctctaaggccaaaggccagcccagggaacctcaagtgtacacactgcccccaagccgcgaggaaatgaccaagaaccaggtgtccctgacatgtctcgtgaagggcttctaccctagcgacatcgccgtggagtgggagtccaatggccagcccgagaacaactacaagaccacaccacctgtgctggactccgatggctccttcttcctgtactccaagctgaccgtggataagtctagatggcagcagggcaacgtgttcagctgttctgtgatgcacgaggctctgcacaaccactacacccagaaatctctgtccctgtctcctggagctcctgaggctccaaccgacgagcctgaagcccctaccgacgagatccctcctcacgtgcagggctccgtgaacaacgacatgatcgtgaccgacaacgctggcgccgttaagtttccccagctgtgcaagttctgcgatgtgcggttctctacctgtgataatcagaagtcctgcatgtccaactgctctatcacctccatctgcgagaagcctcaagaggtgtgtgtggctgtctggcggaagaacgatgagaacatcaccctggaaacagtgtgccacgatcccaagctgccttaccacgacttcatcctggaagatgctgcctctcctaagtgcatcatgaaagaaaagaaaaaacccggcgagacattcttcatgtgctcctgctcctctgacgagtgcaacgacaacatcatcttctct

SEQ ID NO: 219 - DNA, 1779 bp, synthetic construct
  misc_feature 1..1779: note = Fusion protein - heavy chain 21
  source 1..1779: mol_type = other DNA
caaggtcaactagtgcaaagtggtgcagaagtgaagaagcctggaagctccgtgaaggtgtcctgtaagacatccggattcaccttctccagttcctatatctcctgggtgcggcaggcccctggccagggactggagtggatgggctggatctacgccggcacaggcggcacctcctacaaccagaaattcaccggcagggtaaccattacagtggacacatctacctccaccgcctacatggaactgagctctctgagatccgaggataccgctgtgtactactgcgccagacacgagggcaagtactggtatttcgacgtgtggggccagggcaccaccgtgaccgtgtcttctgcctcgaccaagggcccttctgtgttccctctggccccctcctccaagagcacatctggcggaaccgctgctctgggatgtctggtgaaggactactttcctgagcctgtgactgtttcttggaacagcggcgccctgacctccggagtgcataccttccctgccgtgctgcagtcttccggtctgtatagcctgtccagcgtggtgaccgtgcctagcagctctctgggcacacagacatatatttgtaacgtgaatcacaagccttctaacaccaaggtggacaagcgggttgagcctaagtcctgcgacaagacccacacgtgtcctccttgccctgctcctgagctgctcggaggcccttctgtgtttctgttcccaccaaaacccaaggacaccctgatgatctcccggacccctgaagtgacctgcgtggtggtggacgtgtctcacgaggaccccgaagtcaagttcaactggtacgtggacggcgtggaagtgcacaacgccaagaccaagcccagagaagagcagtacaattctacctacagagtggtctccgtgctgaccgtgctgcaccaggactggctgaacggcaaagagtacaagtgcaaggtctccaataaggccctgcctgctcctatcgagaagaccatctctaaggccaaaggccagcccagggaacctcaagtgtacacactgcccccaagccgcgaggaaatgaccaagaaccaggtgtccctgacatgtctcgtgaagggcttctaccctagcgacatcgccgtggagtgggagtccaatggccagcccgagaacaactacaagaccacaccacctgtgctggactccgatggctccttcttcctgtactccaagctgaccgtggataagtctagatggcagcagggcaacgtgttcagctgttctgtgatgcacgaggctctgcacaaccactacacccagaaatctctgtccctgtctcctggagctcctgaggctccaaccgacgagcctgaagcccctaccgacgagccagaggccatccctcctcacgtgcagggctccgtgaacaacgacatgatcgtgaccgacaacgctggcgccgttaagtttccccagctgtgcaagttctgcgatgtgcggttctctacctgtgataatcagaagtcctgcatgtccaactgctctatcacctccatctgcgagaagcctcaagaggtgtgtgtggctgtctggcggaagaacgatgagaacatcaccctggaaacagtgtgccacgatcccaagctgccttaccacgacttcatcctggaagatgctgcctctcctaagtgcatcatgaaagaaaagaaaaaacccggcgagacattcttcatgtgctcctgctcctctgacgagtgcaacgacaacatcatcttctct

SEQ ID NO: 220 - protein, 7 aa, synthetic construct
  REGION 1..7: note = Transferrin linker
  REPEAT 1..6: note = (PEAPTD)nP, n=1, 2, 3, 4, or 5; (PEAPTD) can be repeated 1, 2, 3, 4, or 5 times
PEAPTDP

SEQ ID NO: 221 - protein, 8 aa, synthetic construct
  REGION 1..8: note = Transferrin linker
  REPEAT 1..6: note = (PEAPTD)nPE, n=1, 2, 3, 4, or 5; (PEAPTD) can be repeated 1, 2, 3, 4, or 5 times
PEAPTDPE

SEQ ID NO: 222 - protein, 9 aa, synthetic construct
  REGION 1..9: note = Transferrin linker
  REPEAT 1..6: note = (PEAPTD)nPEA, n=1, 2, 3, 4, or 5; (PEAPTD) can be repeated 1, 2, 3, 4, or 5 times
PEAPTDPEA

SEQ ID NO: 223 - protein, 10 aa, synthetic construct
  REGION 1..10: note = Transferrin linker
  REGION 1..6: note = repeat - (PEAPTD)nPEAP, n=1, 2, 3, 4, or 5; (PEAPTD) can be repeated 1, 2, 3, 4, or 5 times
PEAPTDPEAP

SEQ ID NO: 224 - protein, 11 aa, synthetic construct
  REGION 1..11: note = Transferrin linker
  REGION 1..6: note = repeat - (PEAPTD)nPEAPT, n=1, 2, 3, 4, or 5; (PEAPTD) can be repeated 1, 2, 3, 4, or 5 times
PEAPTDPEAPT

SEQ ID NO: 225 - protein, 8 aa, synthetic construct
  REGION 1..8: note = Transferrin linker
  REGION 1..7: note = repeat - (PEAPTDE)nP, n=1, 2, 3, 4, or 5; (PEAPTDE) can be repeated 1, 2, 3, 4, or 5 times
PEAPTDEP

SEQ ID NO: 226 - protein, 9 aa, synthetic construct
  REGION 1..9: note = Transferrin linker
  REGION 1..7: note = repeat - (PEAPTDE)nPE, n=1, 2, 3, 4, or 5; (PEAPTDE) can be repeated 1, 2, 3, 4, or 5 times
PEAPTDEPE

SEQ ID NO: 227 - protein, 10 aa, synthetic construct
  REGION 1..10: note = Transferrin linker
  REGION 1..7: note = repeat - (PEAPTDE)nPEA, n=1, 2, 3, 4, or 5; (PEAPTDE) can be repeated 1, 2, 3, 4, or 5 times
PEAPTDEPEA

SEQ ID NO: 228 - protein, 11 aa, synthetic construct
  REGION 1..11: note = Transferrin linker
  REGION 1..7: note = repeat - (PEAPTDE)nPEAP, n=1, 2, 3, 4, or 5; (PEAPTDE) can be repeated 1, 2, 3, 4, or 5 times
PEAPTDEPEAP

SEQ ID NO: 229 - protein, 12 aa, synthetic construct
  REGION 1..12: note = Transferrin linker
  REGION 1..7: note = repeat - (PEAPTDE)nPEAPT, n=1, 2, 3, 4, or 5; (PEAPTDE) can be repeated 1, 2, 3, 4, or 5 times
PEAPTDEPEAPT

SEQ ID NO: 230 - protein, 13 aa, synthetic construct
  REGION 1..13: note = Transferrin linker
  REGION 1..7: note = repeat - (PEAPTDE)nPEAPTD, n=1, 2, 3, 4, or 5; (PEAPTDE) can be repeated 1, 2, 3, 4, or 5 times
PEAPTDEPEAPTD

SEQ ID NO: 231 - protein, 15 aa, synthetic construct
  REGION 1..15: note = Transferrin linker
PEAPTDPEAPTDPEA

SEQ ID NO: 232 - protein, 127 aa, synthetic construct
  REGION 1..127: note = TGF- RII ECD C-terminal truncation and mutant Q6G, K7G N19A (1-127)
IPPHVGGSVNNDMIVTDNAGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFS

SEQ ID NO: 233 - protein, 594 aa, synthetic construct
  REGION 1..594: note = Fusion protein 22 - heavy chain
QGQLVQSGAEVKKPGSSVKVSCKTSGFTFSSSYISWVRQAPGQGLEWMGWIYAGTGGTSYNQKFTGRVTITVDTSTSTAYMELSSLRSEDTAVYYCARHEGKYWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAPEAPTDPEAPTDPEAPTDIPPHVQGSVNNDMIVTDNAGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFS

SEQ ID NO: 234 - protein, 597 aa, synthetic construct
  REGION 1..597: note = Fusion protein 23 - heavy chain
QGQLVQSGAEVKKPGSSVKVSCKTSGFTFSSSYISWVRQAPGQGLEWMGWIYAGTGGTSYNQKFTGRVTITVDTSTSTAYMELSSLRSEDTAVYYCARHEGKYWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAPEAPTDPEAPTDPEAPTDPEAIPPHVQGSVNNDMIVTDNAGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFS

SEQ ID NO: 235 - protein, 576 aa, synthetic construct
  REGION 1..576: note = Fusion protein 24-heavy chain
QGQLVQSGAEVKKPGSSVKVSCKTSGFTFSSSYISWVRQAPGQGLEWMGWIYAGTGGTSYNQKFTGRVTITVDTSTSTAYMELSSLRSEDTAVYYCARHEGKYWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAIPPHVGGSVNNDMIVTDNAGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFS

SEQ ID NO: 236 - protein, 593 aa, synthetic construct
  REGION 1..593: note = Fusion protein 25-heavy chain
QGQLVQSGAEVKKPGSSVKVSCKTSGFTFSSSYISWVRQAPGQGLEWMGWIYAGTGGTSYNQKFTGRVTITVDTSTSTAYMELSSLRSEDTAVYYCARHEGKYWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAPEAPTDEPEAPTDEPEAIPPHVGGSVNNDMIVTDNAGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFS

SEQ ID NO: 237 - protein, 597 aa, synthetic construct; identical to SEQ ID NO: 207
  REGION 1..597: note = Fusion protein 26-heavy chain

SEQ ID NO: 238 - protein, 590 aa, synthetic construct; identical to SEQ ID NO: 211
  REGION 1..590: note = Fusion protein 27-heavy chain

SEQ ID NO: 239 - protein, 590 aa, synthetic construct
  REGION 1..590: note = Fusion protein 28 - heavy chain
QGQLVQSGAEVKKPGSSVKVSCKTSGFTFSSSYISWVRQAPGQGLEWMGWIYAGTGGTSYNQKFTGRVTITVDTSTSTAYMELSSLRSEDTAVYYCARHEGKYWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAPEAPTDEPEAPTDEIPPHVGGSVNNDMIVTDNAGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFS

SEQ ID NO: 240 - protein, 597 aa, synthetic construct
  REGION 1..597: note = Fusion protein 29 - heavy chain
QGQLVQSGAEVKKPGSSVKVSCKTSGFTFSSSYISWVRQAPGQGLEWMGWIYAGTGGTSYNQKFTGRVTITVDTSTSTAYMELSSLRSEDTAVYYCARHEGKYWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAPEAPTDEPEAPTDEPEAPTDEIPPHVGGSVNNDMIVTDNAGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFS

SEQ ID NO: 241 - protein, 607 aa, synthetic construct
  REGION 1..607: note = M7824 heavy chain
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSGGGGSGIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 242 - protein, 604 aa, synthetic construct
  REGION 1..604: note = M7824 with rigid linker - heavy chain
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAPEAPTDPEAPTDPEAPTDIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 243 - protein, 216 aa, synthetic construct
  REGION 1..216: note = M7824 light chain
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 244 - protein, 21 aa, synthetic construct
  REGION 1..21: note = linker
GGGGSGGGGSGGGGSGGGGSG

SEQ ID NO: 245 - protein, 5 aa, synthetic construct
  REGION 1..5: note = linker
  REPEAT 1..5: note = (GGGGS)n, n=1, 2, 3, 4, or 5
GGGGS

SEQ ID NO: 246 - protein, 5 aa, synthetic construct
  REGION 1..5: note = linker
  REPEAT 1..5: note = (EAAAK)n, n=1,2, 3, 4, or 5
EAAAK